(19) United States
(12) Patent Application Publication
(10) Pub. No.: US 2013/0116277 A1
(43) Pub. Date: May 9, 2013

(54) ALDO-KETO REDUCTASE SUBFAMILY 1C3 (AKR1C3) INHIBITORS

(71) Applicants: James T. DALTON, Lakeland, TN (US); Duane D. Miller, Germantown, TN (US); Ramesh Narayanan, Cordova, TN (US); Muralimohan Yepuru, Bartlett, TN (US); Christopher C. Coss, Bartlett, TN (US); Michael L. Mohler, Memphis, TN (US); Zhongzhi Wu, Collierville, TN (US)

(72) Inventors: James T. DALTON, Lakeland, TN (US); Duane D. Miller, Germantown, TN (US); Ramesh Narayanan, Cordova, TN (US); Muralimohan Yepuru, Bartlett, TN (US); Christopher C. Coss, Bartlett, TN (US); Michael L. Mohler, Memphis, TN (US); Zhongzhi Wu, Collierville, TN (US)

(21) Appl. No.: 13/607,633

(22) Filed: Sep. 7, 2012

Related U.S. Application Data
(63) Continuation-in-part of application No. 12/773,515, filed on May 4, 2010.
Application No. 12/773,515 is a continuation-in-part of application No. 12/010,225, filed on Jan. 22, 2008.
(60) Provisional application No. 60/881,476, filed on Jan. 22, 2007.
(60) Provisional application No. 60/907,754, filed on Apr. 16, 2007.
(60) Provisional application No. 61/177,214, filed on May 11, 2009.
(60) Provisional application No. 61/613,771, filed on Mar. 21, 2012.

(51) Int. Cl. C07D 217/24

(52) U.S. Cl. CPC C07D 217/24
USPC 514/309; 546/141

(57) ABSTRACT

The present invention relates to a novel class of AKR1C3 inhibitors, to compositions containing them, to methods for their preparation, and to methods of use thereof. The AKR1C3 inhibitors may be useful in the treatment of, for example, prostate cancer, benign prostate hyperplasia (BPH), lung cancer, acne, seborrhea, hirsuitism, baldness, alopecia, precocious puberty, adrenal hypertrophy, polycystic ovary syndrome, breast cancer, uterine cancer, uterine fibroids, endometriosis, myeloma and leiomyoma.

### CROSS REFERENCE TO RELATED APPLICATIONS

This application is Continuation-In-Part application of U.S. patent application Ser. No. 12/773,515, filed May 4, 2010, which is a Continuation-in-Part application of U.S. patent application Ser. No. 12/010,225, filed Jan. 22, 2008 which claims the benefit of United-States Provisional Patent Application Ser. No. 60/881,476, filed Jan. 22, 2007 and U.S. Provisional Patent Application Ser. No. 60/907,754, filed Apr. 16, 2007; and this application claims the benefit of U.S. Provisional Patent Application Ser. No. 61/177,214, filed May 11, 2009; and this application claims the benefit of U.S. Provisional Patent Application Ser. No. 61/613,771, filed Mar. 21, 2012, all of which are hereby incorporated by reference in their entirety.

### FIELD OF THE INVENTION

The present invention relates to a novel class of aldo-keto reductase inhibitors, aldo-keto reductase subfamily 1C3 (AKR1C3) inhibitors and to compositions containing AKR1C3 inhibitors, to methods for preparation of AKR1C3 inhibitors, and to methods of use thereof. The AKR1C3 inhibitors are useful in the treatment of, for example, prostate cancer, benign prostate hyperplasia (BPH), lung cancer, non-small cell lung cancer (NSCLC), acne, seborrhea, hirsuitism, baldness, alopecia, precocious puberty, adrenal hypertrophy, polycystic ovary syndrome, breast cancer, endometriosis, myeloma and leiomyoma. AKR1C3 inhibitors may also provide a function as inhibitors of AKR1C3-mediated androgen-dependent androgen receptor-transactivation, i.e., inhibitors of an AKR1C3 co-activation function, as described herein.

### BACKGROUND OF THE INVENTION

Androgens and estrogens, through their intracellular receptors, promote physiological and pathological developments. Both these classes of steroid hormones are critical for the growth of secondary sexual characteristics, bone, muscle and for the function of neurological and other tissues. These hormones, their receptors and associated proteins have also been implicated in the transformation of normal reproductive tissues into cancerous tissues. A large proportion of androgens in men (40%), and the majority of estrogens in women (75% before menopause and close to 100% after menopause), are synthesized in peripheral target tissues from precursor steroids of adrenal origin. The genes encoding the enzymes responsible for the formation and metabolism of androgens and estrogens are expressed in a large series of peripheral tissues, thus providing the basis for a promising new area in hormone action, namely intracrinology. These steroidogenic and steroid metabolizing enzymes should become a major target of novel therapies for steroid-sensitive diseases, particularly breast and prostate cancer.

Testosterone and dihydrotestosterone (DHT), the active circulating androgens, and estradiol, the potent circulating estrogen, are synthesized from the weak adrenally synthesized precursors androstenedione and estrone, respectively, by steroidogenic enzymes belonging to the class of hydroxysteroid dehydrogenase (HSDs). Enzymes belonging to the class of 17β-hydroxysteroid dehydrogenases (17β-HSDs) and aldo-keto reductases (AKR) play important roles in the synthesis of potent androgens and estrogens. Potent endogenous androgens (testosterone and DHT) are synthesized by type 3 (testicular) and type 5 (peripheral tissues such as adrenal, prostate, etc,) 17β-HSDs.

The human AKR1C isozymes are hydroxysteroid dehydrogenases, and are involved in the pre-receptor regulation of steroid hormone action. AKR1C isozymes regulate the concentration of active and inactive androgens, estrogens and progestins in target tissues by catalyzing the reduction of ketosteroids at positions C3, C17 or C20. AKR1C isozymes regulate the ligand occupancy and transactivation of steroid hormone receptors such as the androgen receptor, estrogen receptor, and progesterone receptor by altering the intracellular steroid hormone formation rates and thus the steroid hormone concentration in target tissues.

Hormone dependent cancers, such as breast and prostate cancer, are dependent on the local milieu of estrogens or androgens, respectively. The AKR1C enzymes support the synthesis of these hormones locally. Microarray analysis of prostate cancer tissues has shown that the AKR1C3 isoform is over-expressed in advanced or metastatic prostate cancers compared to organ-confined prostate cancer. In addition, it has also been shown that androgen biosynthesis continues in peripheral tissues (i.e., not in testes) and supports the post-castration proliferation of prostate cancer (e.g. castration resistant prostate cancer (CRPC)) and that the AKR1C3 is the key enzyme that mediates this peripheral androgen synthesis.

There are four isozymes in the aldo-keto reductase family 1 member C (AKR1C) family, which share more than 85% amino acid sequence identity: AKR1C1 (20α-HSD), AKR1C2 (type 3 3α-HSD), AKR1C3 (type 5 17β-HSD), and AKR1C4 (type 1 3α-HSD). Despite their high sequence identity, the four isozymes display different substrate preferences, inhibition profiles and tissue specific expression patterns. In addition to prostate cancer, alterations in the expression and function of AKR1C isoforms have been reported in other cancers, such as breast cancer, small cell lung cancer and myeloma, suggesting a role for these isozymes in the development of other cancers. Drugs that increase the function of AKR1C1 and AKR1C2 or drugs that decrease the function of AKR1C3 may be useful in treating various malignancies related to steroid hormones.

AKR1C3 is a 37 kDa cytosolic enzyme of the NAD dependent aldo-keto reductase family. AKR1C3 is also known as 17β-hydroxysteroid dehydrogenase type 5 (17βHSD5), 3αHSD2, and prostaglandin F synthase. Isoforms AKR1C1-4 have overlapping function depending on substrate concentration and tissue distribution. AKR1C enzymes catalyze multiple enzymatic reactions to include: conversion of the potent progestin progesterone to the weak progestin 20α-hydroxyprogesterone (AKR1C1, AKR1C3); conversion of the most potent endogenous androgen 5α-dihydrotestosterone (DHT) to the weak androgen 5α-androstane-3α,17β-diol (3α-diol) (AKR1C2); conversion of the weak adrenal androgen androstene-3,17-dione (also known as androstenedione, A'dione, or 4′ dione) to the potent androgen testosterone (AKR1C3; also done by 17βHSD3 in Leydig cells); conversion of the weak androgen 5α-androstanedione (5α-dione) to the most active androgen DHT (AKR1C3) (FIG. 1); conversion of the weak estrogen estrone to 17β-estradiol (AKR1C3); and conversion of differentiative prostaglandin PGD2 to the proliferative prostaglandin PGF2α (AKR1C3). Therefore, inhibition of AKR1C3 activity may reduce the level of end products as described above. For example, inhibition of AKR1C3 activity may reduce the level of the potent androgens testosterone and 5α-dihydrotestosterone (DHT), and/or the potent estrogen 17β-estradiol and/or the proliferative prostaglandin PGF2α. Additionally, inhibition of AKR1C3 activity may increase the level of starting product as described above. For example, inhibition of AKR1C3 activity may increase the level of the potent progestin progesterone.

AKR1C3 is expressed in numerous tissues including liver, prostate, testes, adrenals, uterus, breast, lung, kidney, bladder, ovary, adipose, and brain. AKR1C3 is considered an activating enzyme for the androgen receptor (AR) through biosynthesis of testosterone, the estrogen receptor (ER) through biosynthesis of 17β-estradiol, and a deactivating enzyme of the peroxiosome proliferator-activated receptor gamma (PPARγ) through the synthesis of PGF2α.

AKR1C3 inhibitors may be advantageous in the treatment of prostate cancer. Numerous studies suggest that treatment of prostate cancer patients with gonadotrophin releasing hormone agonist (e.g., leuprolide acetate)-antiandrogen combinations often fails due to increased intratumoral androgen biosynthesis. CYP17A1 (17,20-lyase; 17α-hydroxylase) inhibitors such as ketoconazole and abiraterone inhibit conversion of pregnenolone and progesterone to DHEA and androstenedione, respectively, and exhibit moderate efficacy in CRPC. Unlike CYP17A1, AKR1C3 is upregulated in CRPC and represents a more specific target for preventing localized androgen biosynthesis (i.e. lyase inhibitors also prevent cortisol synthesis leading to mineralocorticoid excess, especially abiraterone). In addition, other enzymatic roles of AKR1C3 promote prostate tumor growth. These include the to effects on prostaglandin metabolism which increase prostate growth and tumor vascularity by decreasing PGJ2 resulting in reduction in PPARγ activity and up-regulation of COX-2. Conversion of DHT to 3α-diol is thought to increase prostate cell proliferation through an AR-independent pathway involving epidermal growth factor-like pathway. Hence, inhibition of AKR1C3 may have direct anti-proliferative effects via decreases in estrogen and androgen synthesis intratumorally (as well as in adjacent tissues), which would limit the occupation of AR and ER. Further, indirect anti-proliferative effects of AKR1C3 inhibition may arise from increased levels of pro-differentiative ligands, e.g., PPARγ ligands and decreased levels of 3α-diol (EGF-like pathway).

AKR1C3 inhibitors may be advantageous for the treatment of breast cancer. Selective estrogen receptor modulators (SERMs) and aromatase inhibitors are widely used in treatment of ER positive breast cancer, which includes about 75% of the cases. Studies have shown that AKR1C3 is consistently over expressed in breast cancer ductal carcinoma in situ and invasive breast cancer ductal carcinoma, as well as being an indicator of poor prognosis for breast cancer. Multiple mechanisms of AKR1C3 promotion of breast tumor growth exist such as: a) conversion of androstenedione to testosterone provides a substrate for CYP19 aromatase to create 17β-estradiol; b) conversion of estrone (weak estrogen) to 17β-estradiol; c) reduction of the anti-proliferative effect of PGD2; and d) decrease in progesterone by inactivation to 20α-progesterone which further increases ER:PR ratio. Thus inhibition of AKR1C3 should be therapeutic in breast cancer as inhibition of mechanisms a) and b) above should decrease intratumoral ER occupancy, and inhibition of mechanism c) should increase intratumoral occupancy of PPARγ. Further support for use of AKR1C3 inhibitors comes from a report of anti-proliferative effects in stably transfected MCF7 cells (Chemico-Biological Interactions 178 (2009), 221-227). AKR1C3 inhibitors may be advantageous for the treatment of AR-positive and ER-positive breast cancers. CRPC and refractory breast cancer are common cancers with low survival rates (less than 50% at 5 yrs). Currently there are no AKR1C3 inhibitors approved or in clinical trials.

AKR1C3 inhibitors may be useful for androgen-dependent conditions. Non limiting examples of such uses include: (a) treatment of adrenal adenomas, carcinoma, or hyperplasia; (b) treatment of Leydig cell tumors in men; (c) treatment of arrhenoblastomas in women; (d) treatment of polycystic ovarian syndrome (PCOS) in women; e) treatment, prevention, decreasing the incidence of, halting and/or causing a regression of prostate cancer; f) other clinical, therapeutic, and/or diagnostic areas; or g) treatment and/or prevention of acne, seborrhea, hirsuitism, baldness and alopecia.

AKR1C3 inhibitors may be useful for estrogen-dependent conditions. Non limiting examples of such uses include (a) treating, suppressing, inhibiting or reducing the amount of precancerous precursors of prostate adenocarcinoma, for example, those having benign prostatic hyperplasia, prostatic intraepithelial neoplasia (PIN) or an abnormally high level of circulating prostate specific antibody (PSA), or who have a family history of prostate cancer; (b) treating, preventing, suppressing, inhibiting, or reducing the incidence of osteoporosis, hot flashes, gynecomastia, and/or hair loss in male human subjects having prostate cancer; (c) treating, suppressing, inhibiting or reducing the risk of developing prostate cancer; or (d) treating, suppressing, inhibiting or reducing the risk of developing breast cancer in a subject.

AKR1C3 inhibitors may be useful for prostaglandin-dependent diseases. AKR1C3 is the only known PGF2 synthase in humans. Diseases related to increased PGF2 levels (or increased PGF2 synthase (AKR1C3) activity) include: endometriosis, inflammatory tachycardia, lung cancer, asthma and airway inflammation, type 2 diabetes, obesity, multiple inflammatory diseases, diseases related to oxidative stress, dysmenorrhea, and renal cell carcinoma. These diseases could result from direct increases in PGF2 levels or from decreases in PPAR-γ activity (as AKR1C3 sequesters the precursor of endogenous PPAR-γ ligand, which is PGJ2, to form PGF2). AKR1C3 also catalyzes the reduction of prostaglandin (PG) H(2) to PGF(2α) and PGD(2) to 9α,11β-PGF(2), which will limit the formation of anti-proliferative prostaglandins, including 15-deoxy-Δ(12,14)-PGJ(2), and contribute to proliferative signaling. AKR1C3 is overexpressed in a wide variety of cancers, including breast and prostate cancer. There is considerable interest in the development of an inhibitor of aldo-keto reductase (AKR) 1C3 (type 5 17β-hydroxysteroid dehydrogenase and prostaglandin F synthase) as a potential therapeutic for both hormone-dependent and hormone-independent cancers.

Consistent with the potential role for AKR1C3 in the initiation of parturition, indomethacin, which is a potent and isoform selective inhibitor of AKR1C3, has long been used for tocolysis (i.e., also called anti-contraction medications or labour repressants) or medications used to suppress premature labor (from the Greek tokos, childbirth, and lytic, capable of dissolving).

Non-steroidal anti-inflammatory drugs (NSAIDs) such as indomethacin and flufenamic acid are known commercial inhibitors of AKR1C3. However, NSAIDs have significant cross-reactivity with cyclooxygenase enzymes (COX-1 and COX-2), which leads to side effects such as gastric irritation, ulcers, cardiovascular problems and others. Hence, there is a need in the art to develop new HSD inhibitors, for example, HSD inhibitors that are specific to AKR1C3 and that lack cross-reactivity with, for example, other AKR1C isoforms and HSD isoforms, and with other enzymes, such as COX enzymes. The AKR1C3 inhibitors of this invention do not cross-react or have significantly reduced cross-reactivity with respect to CYP17A1, COX-1, COX-2, other AKR1C enzymes, 17βHSD3, and are not agonist or antagonists for steroid hormone receptors such as AR, ER, and PR. Yet, AKR1C3 inhibitors may inhibit the down-stream activities of steroid receptors, as AKR1C3 inhibitors may regulate the formation of ligands for these receptors. For example, an AKR1C3 inhibitor may decrease or inhibit the activity of the AR receptor by decreasing the amount of available testosterone and DHT. Additionally, an AKR1C3 inhibitor may decrease or inhibit the activity of an ER receptor (ER-alpha or ER-beta) by decreasing the amount of available estradiol. Further, an AKR1C3 inhibitor may increase or augment the activity of the PR receptor by decreasing the amount of available progesterone.

### SUMMARY OF THE PRESENT INVENTION

In one aspect, this invention provides an AKR1C3 inhibitor compound of Formula XIII:

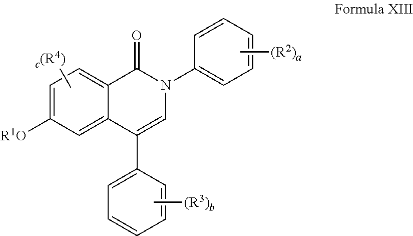

wherein

R¹ is H, alkyl or -alkylene-CO₂Rx, in which Rx is H or alkyl;

R² is H, substituted or unsubstituted alkyl, substituted or unsubstituted alkenyl, substituted or unsubstituted alkyl, substituted or unsubstituted cycloalkyl, alkoxy, haloalkyl, hydroxyl, hydroxymethyl, CONH₂, CONHR^{y}, substituted or unsubstituted alkylene-CO₂R^{y}, in which R^{y} is H or alkyl;

R³ is, in each case, independently selected from hydrogen, alkoxy, COOH, hydroxyl, halogen, haloalkyl, C(O)NH₂, CF₂OMe, CN, carboxyl, SO₂R^{Z} or SO₂NHR^{Z} in which R^{Z} is, in each case, independently, H or alkyl;

R⁴ is, in each case, independently selected from hydrogen, alkyl, hydroxyl, halogen, haloalkyl, CN, carboxyl, CONH₂, CONHR^{Z}, SO₂R^{Z} or SO₂NHR^{Z} in which R^{Z} is, in each case, independently, H or alkyl;

a=1, 2, 3, 4 or 5;

b=1, 2, 3, 4 or 5; and

c=1, 2 or 3;

or a prodrug, isomer, tautomer, metabolite, pharmaceutically acceptable salt, polymorph, crystal, N-oxide, hydrate, or any combination thereof.

In another aspect, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a compound represented by the structure of formula I, or its isomer, tautomer, pharmaceutically acceptable salt, polymorph, crystal, N-oxide, hydrate or any combination thereof:

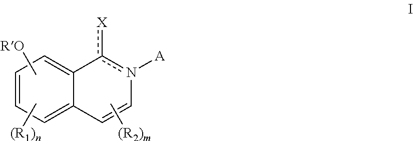

wherein
A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic rings are optionally substituted by 1 to 5 substituents independently selected from R₃ or OR″; and X is O or S; or
A is nothing, N forms a double bond with the cyclic carbon and X is OH or OCH₂CH₂-heterocycle in which the heterocycle is a 3-7 membered saturated or unsaturated substituted or unsubstituted heterocyclic ring;
R₁, R₂ and R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OHSH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring;
   R is alkyl, hydrogen, haloalkyl, dihaloalkyl, trihaloalkyl, CH₂F, CHF₂, CF₃, CF₂CF₃, aryl, heteroaryl, phenyl, benzyl, -Ph-CF₃, -Ph-CH₂F, -Ph-CHF₂, -Ph-CF₂CF₃, halogen, alkenyl, CN, NO₂, or OH;
R′ is hydrogen, Alk, or COR;
R″ is hydrogen, Alk, or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or

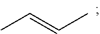

Q is SO₃H, CO₂H, CO₂R, NO₂, tetrazole, SO₂NH₂ or SO₂NHR;
n is an integer of between 1-3;
m is an integer between 1-2; and
Alk is a linear alkyl of 1-7 carbons, branched alkyl of 1-7 carbons, or cyclic alkyl of 3-8 carbons.

In another embodiment, the methods of this invention include administering a compound of formula XI, or its isomer, pharmaceutically acceptable salt, pharmaceutical product, polymorph, crystal, N-oxide, ester, hydrate or any combination thereof:

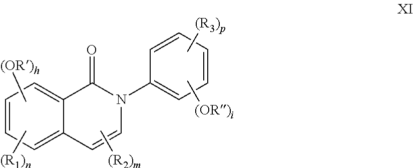

wherein:
R₁, R₂, R₃ are each, independently, hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, cycloalkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;
R is alkyl, cycloalkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
R′ is hydrogen, Alk or COR;
R″ is hydrogen, Alk or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;

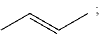

Z is O, NH, CH₂ or
Q is SO₃H, CO₂H, CO₂R, NO₂, tetrazole, SO₂NH₂ or SO₂NHR;
h is 0, 1, 2 or 3;
i is 0, 1, 2, 3 or 4;
n is 1, 2, 3 or 4;
m is 1 or 2;
p is 0, 1, 2, 3, 4 or 5; and
Alk is a linear alkyl of 1-7 carbons, branched alkyl of 1-7 carbons, or cycloalkyl of 3-8 carbons.

In another embodiment, the methods of this invention include administering a compound of formula XIII, or its isomer, pharmaceutically acceptable salt, pharmaceutical product, polymorph, crystal, N-oxide, ester, hydrate or any combination thereof:

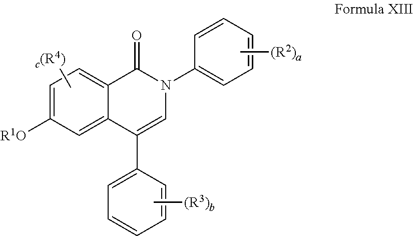

wherein

R¹ is H, alkyl or -alkylene-CO₂R^{x}, in which R^{x} is H or alkyl;

R² is H, substituted or unsubstituted alkyl, substituted or unsubstituted alkenyl, substituted or unsubstituted alkyl, substituted or unsubstituted cycloalkyl, alkoxy, haloalkyl, hydroxyl, hydroxymethyl, CONH₂, CONHR^{y}, substituted or unsubstituted alkylene-CO₂R^{y}, in which R^{y} is H or alkyl;

R³ is, in each case, independently selected from hydrogen, alkoxy, COOH, hydroxyl, halogen, haloalkyl, CF₂OMe, C(O)NH₂, CN, carboxyl, SO₂R^{Z} or SO₂NHR^{Z} in which R^{Z} is, in each case, independently, H or alkyl;

R⁴ is, in each case, independently selected from hydrogen, alkyl, hydroxyl, halogen, haloalkyl, CN, carboxyl, CONH₂, CONHR^{Z}, SO₂R^{Z} or SO₂NHR^{Z} in which R^{Z} is, in each case, independently, H or alkyl;

a=1, 2, 3, 4 or 5;

b=1, 2, 3, 4 or 5; and
c=1, 2 or 3.

In another embodiment, the methods of this invention include administering a compound selected from:
6-hydroxy-2-(4-hydroxyphenyl)-4-phenylisoquinolin-1(2H)-one (15a),
6,8-dihydroxy-2-(4-hydroxyphenyl)-4-(4-methoxyphenyl)isoquinolin-1(2H)-one (15g),
6,8-dihydroxy-2-(4-hydroxyphenyl)-4-phenylisoquinolin-1(2H)-one (15h),
(E)-3-(6,8-dihydroxy-2-(4-hydroxyphenyl)-1-oxo-1,2-dihydroisoquinolin-4-yl)acrylic acid (151),
2-(4-bromomethyl)phenyl-6-hydroxy-4-(4-hydroxyphenyl)isoquinolin-1(2H)-one (11),
6-hydroxy-2-(4-hydroxyphenyl)-4-(4-(trifluoromethyl)phenylisoquinolin-1(2H)-one (13),
6-hydroxy-2-(4-(hydroxymethyl)phenyl)-4-(4-hydroxyphenyl)isoquinolin-1(2H)-one (14),
2-(4-(bromomethyl)-3-hydroxyphenyl)-6-hydroxy-4-(4-(trifluoromethyl)phenyl)isoquinolin-1(2H)-one (26),
6-hydroxy-2-(4-hydroxyphenyl)-1-oxo-4-(3,4,5-trifluorophenyl)-1,2-dihydroisoquinoline-8-carbonitrile (85),
3-(4-(3-fluoro-4-(trifluoromethyl)phenyl)-6-hydroxy-1-oxoisoquinolin-2(1H)-yl)benzamide (214) and
4-(4-(3-fluoro-4-(trifluoromethyl)phenyl)-6-hydroxy-1-oxoisoquinolin-2(1H)-yl)benzamide (215).

In one embodiment, this invention is directed to a compound 3-(4-(3-fluoro-4-(trifluoromethyl)phenyl)-6-hydroxy-1-oxoisoquinolin-2(1H)-yl)benzamide (214) or its isomer, pharmaceutically acceptable salt, pharmaceutical product, polymorph, crystal, N-oxide, ester, hydrate or any combination thereof.

In one embodiment, this invention is directed to a compound 4-(4-(3-fluoro-4-(trifluoromethyl)phenyl)-6-hydroxy-1-oxoisoquinolin-2(1H)-yl)benzamide (215) or its isomer, pharmaceutically acceptable salt, pharmaceutical product, polymorph, crystal, N-oxide, ester, hydrate or any combination thereof.

In one embodiment, the compound of this invention is an AKR1C3 inhibitor. In one embodiment.

In a further aspect, the present invention relates to a method of treating, suppressing, to inhibiting or reducing the incidence of, or delaying progression of a disorder or condition that responds to AKR1C3 inhibition comprising administering to a patient in need thereof a therapeutically effective amount of a compound of this invention or its isomer, pharmaceutically acceptable salt, polymorph, metabolite, prodrug, crystal, N-oxide, hydrate or any combination thereof. In one embodiment, such disorders and conditions include, but are not limited to prostate cancer, advanced prostate cancer, precancerous precursors of prostate adenocarcinoma, prostate intraepithelial neoplasia (PIN), castration resistant prostate cancer (CRPC), benign prostate hyperplasia (BPH), lung cancer, non-small cell lung cancer (NSCLC), acne, seborrhea, hirsuitism, baldness, alopecia, precocious puberty, adrenal hypertrophy, polycystic ovary syndrome, breast cancer, metastatic breast cancer, refractory breast cancer, AR-positive breast cancer, ER-alpha positive breast cancer, ER-beta positive breast cancer, uterine cancer including endometrial and cervical cancers, uterine fibroids including myomas, endometriosis, myeloma and leiomyoma.

In one embodiment, this invention provides a method of lowering total serum testosterone levels in a male subject comprising administering a therapeutically effective amount of a compound of this invention, wherein the lowering of total serum testosterone is independent of a reduction of serum luteinizing hormone levels. In another embodiment, this invention provides a method of lowering serum testosterone levels in a male subject comprising administering a therapeutically effective amount of a compound of this invention, wherein the lowering of serum free testosterone is independent of a reduction of serum luteinizing hormone levels. In yet another embodiment, this invention provides a method of lowering serum free testosterone levels in a male subject comprising administering a therapeutically effective amount of a compound of this invention, wherein the lowering of serum free testosterone is independent of a reduction of serum luteinizing hormone levels. In still another embodiment, this invention provides a method of lowering serum PSA levels in a male subject comprising administering a therapeutically effective amount of a compound of this invention, wherein the lowering of serum PSA is independent of a reduction of serum luteinizing hormone levels.

In one embodiment, this invention provides a method of increasing survival of a subject with advanced prostate cancer comprising administering a therapeutically effective amount of a compound of this invention. In another embodiment, this invention provides a method of increasing survival of a subject with castration-resistant prostate cancer (CRPC) comprising administering a therapeutically effective amount of a compound of this invention.

In one embodiment, this invention provides a method of prolonging progression-free to survival of a subject with advanced prostate cancer comprising administering a therapeutically effective amount of a compound of this invention. In another embodiment, this invention provides a method of prolonging progression-free survival of a subject with castration-resistant prostate cancer (CRPC) comprising administering a therapeutically effective amount of a compound of this invention.

In one embodiment, this invention provides a method of lowering total serum estradiol levels in a subject comprising administering a therapeutically effective amount of a compound of this invention. In another embodiment, this invention provides a method of lowering serum free estradiol in a subject comprising administering a therapeutically effective amount of a compound of this invention. In one embodiment, a subject is a male subject. In another embodiment, a subject is a female subject.

In one embodiment, this invention provides a method of increasing survival of a subject with advanced breast cancer comprising administering a therapeutically effective amount of a compound of this invention. In another embodiment, this invention provides a method of increasing survival of a subject with refractory breast cancer comprising administering a therapeutically effective amount of a compound of this invention. In another embodiment, this invention provides a method of increasing survival of a subject with AR-positive or ER-positive breast cancer comprising administering a therapeutically effective amount of a compound of this invention.

In one embodiment, this invention provides a method of prolonging progression-free survival of a subject with advanced breast cancer comprising administering a therapeutically effective amount of a compound of this invention. In another embodiment, this invention provides a method of prolonging progression-free survival of a subject with refractory breast cancer comprising administering a therapeutically effective amount of a compound of this invention. In another embodiment, this invention provides a method of prolonging progression-free survival of a subject with AR-positive or ER-positive breast cancer comprising administering a therapeutically effective amount of a compound of this invention.

In one embodiment, this invention provides a method of treating, increasing survival and/or prolonging progression-free survival of a subject with uterine cancer comprising administering a therapeutically effective amount of a compound of this invention.

In one embodiment, this invention provides a method of treating a subject with uterine fibroids comprising administering a therapeutically effective amount of a compound of this invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1: depicts steroidogenic pathways involved in androgen synthesis. Grey colored arrows indicate back door steroidogenic pathway.

FIG. 2: depicts the effect of AKR1C3 inhibitors on Testosterone Synthesis, including activity of compound 215 compared to REF 1 and REF2

FIG. 3: depicts AKR1C3-dependent AR transactivation in cells transfected with AKR1C3.

FIG. 4 depicts that AKR1C3 augmented A'dione-dependent AR trans activation. ∘ AKR1C3; •Vector.

FIG. 5 shows AKR1C3-dependent androgen-induced AR transactivation is not cell type dependent. FIGS. 5A-E depict AKR1C3 increased active androgen-induced AR transactivation in COS-1 cells with a titration of the androgens: A'dione (5A); testosterone (5B), DHT (5C), R1881 (5D), and a SARM (5E), compared with vector; ∘ AKR1C3; •Vector (pCR3.1).

FIG. 6 shows AKR1C3-dependent increase in transactivation is specific to AR. FIG. 6 illustrate specificity of AKR1C3-dependent transactivation with a titration of steroid receptors: A'dione-AR (6A), R1881-AR (6B), Dex-GR (6C), Prog-PR (6D), Estrogen-ERα (6E), Aldosterone-MR (6F), and Rosi-PPARγ (6G), compared with vector; ∘ AKR1C3; • Vector (pCR3.1). A'dione-androstenedione; Dex-dexamethasone; Prog-progesterone; estrogen-17β-estradiol; Rosi-rosiglitazone.

FIG. 7: shows AKR1C3 physically interacts with AR, depicting immunoprecipitation of AR and AKR1C3. Analysis of the immunoprecipitant produced by immunoprecipitation (IP) of AKR1C3 demonstrated immunoreactivity with AR on immunoblot (IB).

FIG. 8: shows that AKR1C3 is recruited to PSA enhancer.

FIG. 9A: depicts crystals of purified AKR1C3.

FIG. 9B: depicts crystal structure of AKR1C3 in complex with compounds 45 and 26 in FIGS. 9B-1 and 9B-2, respectively. The AKR1C3 crystal structures with 45 and 26 are superimposed in FIG. 9B-3.

FIG. 10: depict concentration dependent increase of AR activation, individually (FIG. 10A, SRC-2; FIG. 10B, AKR1C3) and together (FIG. 10C, SRC-2 and AKR1C3), demonstrating cotransfection synergistically increased AR transactivation.

FIG. 11: depicts AKR1C3 (FIG. 11A) and cyclophilin (FIG. 11B) expression after siRNA transfection.

FIG. 12: depicts AKR1C3 translocation to nucleus requires AR. FIG. 12A shows NIH3T3 cells stably transfected with AKR1C3, were infected with adenovirus LacZ, treated with 10 nM R1881, and the expression of AKR1C3 was detected by immunofluorescence using laser confocal microscopy. FIG. 12B shows NIH3T3 cells stably transfected with AKR1C3 were infected with adenovirus AR and were treated with 10 nM R1881. Cells were fixed and AR (green) and AKR1C3 (red) were detected by immunofluorescence using laser confocal microscopy.

FIG. 13: depicts AKR1C3 migrates with AR. LNCaP-AKR1C3 cells were treated with 0.1 nM R1881 (top panels) or 0.1 nM R1881 and 10 μM SNARE-1. Cells were fixed and AR (green) and AKR1C3 (red) were detected by immunofluorescence using a laser confocal microscopy. (Key: SNARE-1-Selective Nuclear Androgen Receptor Exporter-1.)

FIG. 14: shows AKR1C3 enhanced androgen signaling and prostate cancer xenograft growth. FIGS. 14A-14C show AKR1C3 siRNA inhibited AR function in LNCaP cells. Cyclophilin (Cyclo.); no siRNA control (−) FIGS. 14D, 14E, FIG. 14F and FIG. 14G show AKR1C3 transfection increased androgen induced PSA gene expression. FIG. 14D is the PSA gene expression with a titration of 4′dione and FIG. 14E is with a titration of R1881. In both the figures, solid lines are vector pCR3.1 transfected and broken lines are AKR1C3 transfected. FIG. 14F shows AKR1C3 increased DHT-induced LNCaP tumor xenograft growth. Numbers within brackets indicate the number of animals with tumor uptake. FIG. 14G shows AKR1C3 expression in cells tranfected with AKR1C3 (closed bars) or vector (open bars; not visible in the figure) transfected cells. FIG. 14H shows AKR1C3 enhanced androgen signaling in tumor xenografts. Open bars are LNCaP-Vector tumors (n=3) and filled bars are LNCaP-AKR1C3 tumors (n=6). FIG. 14I shows FKBP51 protein, derived from an AR dependent gene, is increased in LNCaP-AKR1C3 xenografts. FIG. 14J depicts over-expression of AKR1C3 increased LNCaP xenograft growth. FIG. 14K depicts AR target FKBP51 protein expression (right panel) is increased in LNCaP-AKR1C3 xenograft tumors, despite little to no change in AR levels

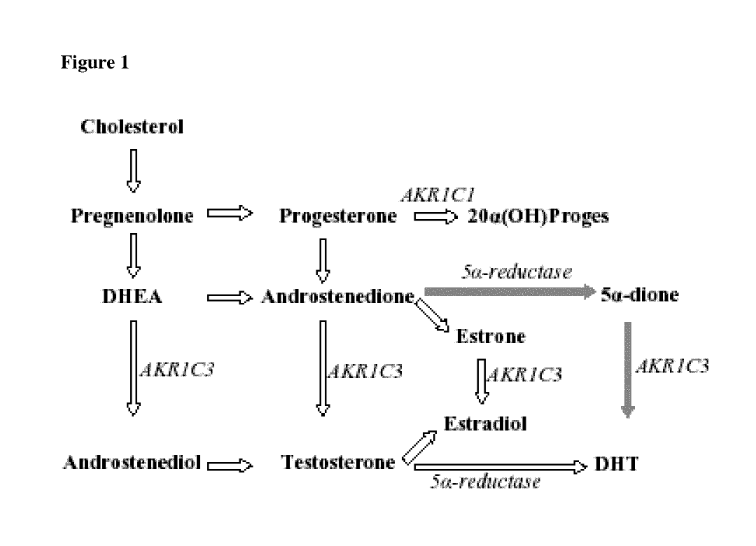

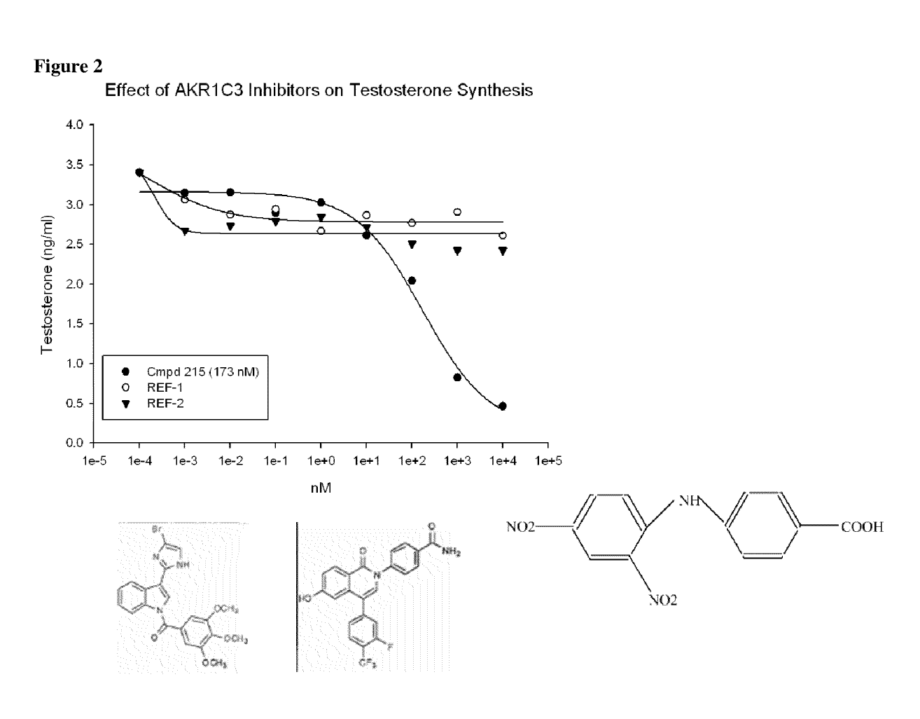

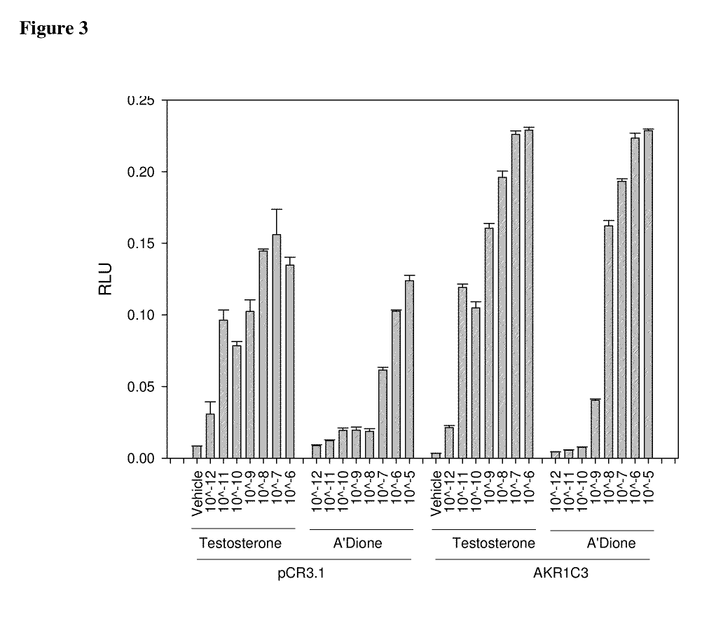

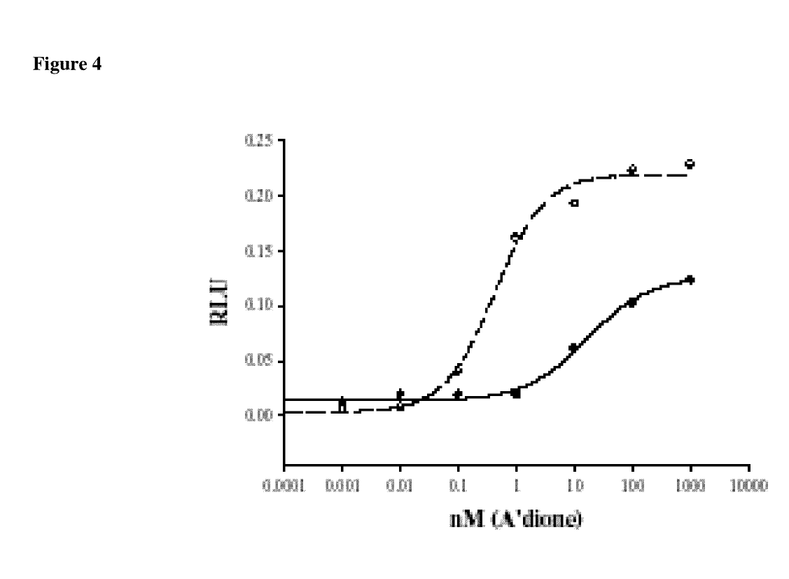

### DETAILED DESCRIPTION OF THE PRESENT INVENTION

In one aspect, this invention provides an AKR1C3 inhibitor of Formula XIII:

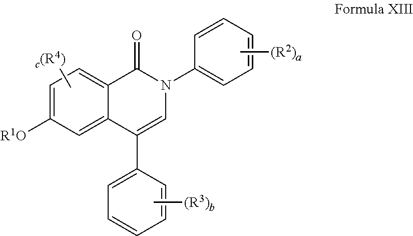

wherein

R¹ is H, alkyl or -alkylene-CO₂R^{x}, in which R^{x} is H or alkyl;

R² is H, substituted or unsubstituted alkyl, substituted or unsubstituted alkenyl, substituted or unsubstituted alkyl, substituted or unsubstituted cycloalkyl, alkoxy, haloalkyl, hydroxyl, hydroxymethyl, CONH₂, CONHR^{y}, substituted or unsubstituted alkylene-CO₂R^{y}, in which R^{y} is H or alkyl;

R³ is, in each case, independently selected from hydrogen, alkoxy, COOH, hydroxyl, halogen, haloalkyl, CF₂OMe, C(O)NH₂, CN, carboxyl, SO₂R^{Z} or SO₂NHR^{Z} in which R^{Z} is, in each case, independently, H or alkyl;

R⁴ is, in each case, independently selected from hydrogen, alkyl, hydroxyl, halogen, haloalkyl, CN, carboxyl, CONH₂, CONHR^{Z}, SO₂R^{Z} or SO₂NHR^{Z} in which R^{Z} is, in each case, to independently, H or alkyl;

a=1, 2, 3, 4 or 5;

b=1, 2, 3, 4 or 5; and

c=1, 2 or 3;

or a prodrug, isomer, tautomer, metabolite, pharmaceutically acceptable salt, polymorph, crystal, N-oxide, hydrate, or any combination thereof.

In one embodiment, R¹ of formula XIII is hydrogen. In one embodiment, R¹ of formula

XIII is alkyl.

In one embodiment, R² of formula XIII is, in each case, independently hydroxyl. In one embodiment, R² of formula XIII is, in each case, independently hydrogen. In another embodiment, R² of formula XIII is, in each case, independently CH₂-halogen. In another embodiment, R² of formula XIII is, in each case, independently CH₂—Br. In another embodiment, R² of formula XIII is, in each case, independently CH₂OH. In another embodiment, R² of formula XIII is, in each case, independently C(O)NH₂. In another embodiment, R² of formula XIII is, in each case, independently CF₃. In another embodiment, R² of formula XIII is, in each case, independently halogen. In another embodiment, R² of formula XIII is, in each case, independently F. In another embodiment, R² of formula XIII is, in each case, independently Cl. In another embodiment, R² of formula XIII is, in each case, independently Br. In another embodiment, R² of formula XIII is, in each case, independently I.

In one embodiment, R³ of formula XIII is, in each case, independently hydrogen. In another embodiment, R³ of formula XIII is, in each case, independently alkoxy. In another embodiment, R³ of formula XIII is, in each case, independently OMe. In another embodiment, R³ of formula XIII is, in each case, independently CF₃. In another embodiment, R³ of formula XIII is, in each case, independently CONH₂H. In another embodiment, R³ of formula XIII is, in each case, independently C(O)OH. In another embodiment, R³ of formula XIII is, in each case, independently CF₃. In another embodiment, R³ of formula XIII is, in each case, independently halogen. In another embodiment, R³ of formula XIII is, in each case, independently F. In another embodiment, R³ of formula XIII is, in each case, independently Cl. In another embodiment, R³ of formula XIII is, in each case, independently Br. In another embodiment, R³ of formula XIII is, in each case, independently I. In another embodiment, R³ of formula XIII is, in each case, independently SO₂CH₃.

In one embodiment, R⁴ of formula XIII is, in each case, independently hydrogen. In another embodiment, R⁴ of formula XIII is, in each case, independently hydroxyl. In another embodiment, R⁴ of formula XIII is, in each case, independently CN. In another embodiment, R⁴ of formula XIII is, in each case, independently halogen. In another embodiment, R⁴ of formula XIII is, in each case, independently F. In another embodiment, R⁴ of formula XIII is, in each case, independently Cl. In another embodiment, R⁴ of formula XIII is, in each case, independently Br. In another embodiment, R⁴ of formula XIII is, in each case, independently I. In another embodiment, R⁴ of formula XIII is, in each case, independently SO₂NHCH₃.

In another embodiment an AKR1C3 inhibitor of Formula XIII of this invention is 6-hydroxy-2,4-bis(4-hydroxyphenyl)isoquinolin-1(2H)-one (6); In another embodiment an AKR1C3 inhibitor of Formula XIII of this invention is 2-(4-(hydroxymethyl)phenyl)-6-methoxy-4-(4-methoxyphenyl)isoquinolin-1(2H)-one (10); In another embodiment an AKR1C3 inhibitor of Formula XIII of this invention is 2-(4-bromomethyl)phenyl-6-hydroxy-4-(4-hydroxyphenyl)isoquinolin-1(2H)-one (11); In another embodiment an AKR1C3 inhibitor of Formula XIII of this invention is 6-hydroxy-2-(4-hydroxyphenyl)-4-(4-(trifluoromethyl)phenyl)isoquinolin-1(2H)-one (13); In another embodiment an AKR1C3 inhibitor of Formula XIII of this invention is 6-hydroxy-2-(4-(hydroxymethyl)phenyl)-4-(4-hydroxyphenyl)isoquinolin-1(2H)-one (14); In another embodiment an AKR1C3 inhibitor of Formula XIII of this invention is 2-(4-(hydroxymethyl)-3-methoxyphenyl)-6-methoxy-4-(4-(trifluoromethyl)phenyl)isoquinolin-1(2H)-one (25); In another embodiment an AKR1C3 inhibitor of Formula XIII of this invention is 2-(4-(bromomethyl)-3-hydroxyphenyl)-6-hydroxy-4-(4-(trifluoromethyl)phenyl)isoquinolin-1(2H)-one (26); In another embodiment an AKR1C3 inhibitor of Formula XIII of this invention is 6-hydroxy-2,4-bis(4-(trifluoromethyl)phenyl)isoquinolin-1(2H)-one (30); In another embodiment an AKR1C3 inhibitor of Formula XIII of this invention is 2-(4-fluorophenyl)-6-hydroxy-4-(4-(trifluoromethyl)phenyl)isoquinolin-1(2H)-one (34); In another embodiment an AKR1C3 inhibitor of Formula XIII of this invention is 6-methoxy-2-(4-methoxyphenyl)-4-(4-(trifluoromethyl)phenyl)isoquinolin-1(2H)-one (35); In another embodiment an AKR1C3 inhibitor of Formula XIII of this invention is 4-(6-hydroxy-2-(4-hydroxyphenyl)-1-oxo-1,2-dihydroisoquinolin-4-yl)-N-methyl benzenesulfonamide (36); In another embodiment an AKR1C3 inhibitor of Formula XIII of this invention is 6-hydroxy-2-(4-hydroxyphenyl)-4-(4-(methylsulfonyl)phenyl)isoquinolin-1(2H)-one (37); In another embodiment an AKR1C3 inhibitor of Formula XIII of this invention is 4-(6-hydroxy-2-(4-hydroxyphenyl)-1-oxo-1,2-dihydroisoquinolin-4-yl)benzoic acid (43). In another embodiment an AKR1C3 inhibitor of Formula XIII of this invention is 2-benzyl-6-hydroxy-4-(3,4,5-trifluorophenyl)isoquinolin-1(2H)-one (75). In another embodiment an AKR1C3 inhibitor of Formula XIII of this invention is 6-hydroxy-2-(4-hydroxyphenyl)-4-(3,4,5-trifluorophenyl)isoquinolin-1(2H)-one (79); In another embodiment an AKR1C3 inhibitor of Formula XIII of this invention is 4-(3,4,5-trifluorophenyl)isoquinolin-1(2H)-one (90); In another embodiment an AKR1C3 inhibitor of Formula XIII of this invention is methyl 6-hydroxy-2-(4-hydroxyphenyl)-1-oxo-4-(3,4,5-trifluorophenyl)-1,2-dihydroisoquinoline-8-carbimidate (100); In another embodiment an AKR1C3 inhibitor of Formula XIII of this invention is 6-hydroxy-2-(4-hydroxyphenyl)-1-oxo-4-(3,4,5-trifluorophenyl)-1,2-dihydroisoquinoline-8-carboxamide (100A); In another embodiment an AKR1C3 inhibitor of Formula XIII of this invention is 4-(3-fluoro-4-(trifluoromethyl)phenyl)-6-hydroxy-2-(4-hydroxyphenyl)-1-oxo-1,2-dihydroisoquinoline-8-carbonitrile (102); In another embodiment an AKR1C3 inhibitor of Formula XIII of this invention is methyl-4-(3-fluoro-4-(trifluoromethyl)phenyl)-6-hydroxy-2-(4-hydroxyphenyl)-1-oxo-1,2-dihydroisoquinoline-8-carbimidate (102A); In another embodiment an AKR1C3 inhibitor of Formula XIII of this invention is 4-(3-fluoro-4-(trifluoromethyl)phenyl)-6-hydroxy-2-(4-hydroxyphenyl)-1-oxo-1,2-dihydroisoquinoline-8-carboxamide (102B); 6-hydroxy-2-(1-oxo-1,3-dihydroisobenzofuran-5-yl)isoquinolin-1(2H)-one (104); In another embodiment an AKR1C3 inhibitor of Formula XIII of this invention is methyl 2-(bromomethyl)-4-(6-hydroxy-1-oxoisoquinolin-2(1H)-yl)benzoate (104A); In another embodiment an AKR1C3 inhibitor of Formula XIII of this invention is 4-bromo-6,8-dihydroxy-2-(3-hydroxyphenyl)isoquinolin-1(2H)-one (213); or their prodrug, isomer, metabolite, pharmaceutically acceptable salt, polymorph, crystal, N-oxide, hydrate or any combination thereof.

In another aspect, the present invention relates to compounds that act as hydroxysteroid dehydrogenase inhibitors (HDIs), such as compounds that act as inhibitors of 17β-hydroxysteroid hydrogenases (17β-HSDs), for example, and/or compounds that act as selective inhibitors of AKR1C3 (type 5 17β-HSD or 17β-HSD5). In another aspect, the present invention relates to compounds that act as inhibitors of AKR1C3 co-activation function, for example, inhibitors of AKR1C3 co-activation of androgen-dependent Androgen Receptor (AR)-transactivation.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula I, or its isomer, tautomer, pharmaceutically acceptable salt, polymorph, crystal, N-oxide, hydrate or any combination thereof:

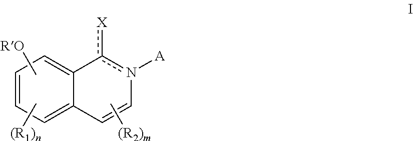

wherein

A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic rings are optionally substituted by 1 to 5 substituents independently selected from R₃ or OR″; and X is O or S; or

A is nothing, N forms a double bond with the cyclic carbon and X is OH or OCH₂CH₂-heterocycle in which the heterocycle is a 3-7 membered saturated or unsaturated substituted or unsubstituted heterocyclic ring;

R₁, R₂ and R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OHSH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring;

R is alkyl, hydrogen, haloalkyl, dihaloalkyl, trihaloalkyl, CH₂F, CHF₂, CF₃, CF₂CF₃, aryl, heteroaryl, phenyl, benzyl, -Ph-CF₃, -Ph-CH₂F, -Ph-CHF₂, -Ph-CF₂CF₃, halogen, alkenyl, CN, to NO₂, or OH;
R′ is hydrogen, Alk, or COR;
R″ is hydrogen, Alk, or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or

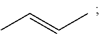

Q is SO₃H, CO₂H, CO₂R, NO₂, tetrazole, SO₂NH₂ or SO₂NHR;
n is an integer of between 1-3;
m is an integer between 1-2; and
Alk is a linear alkyl of 1-7 carbons, branched alkyl of 1-7 carbons, or cyclic alkyl of 3-8 carbons.

In one embodiment, A is

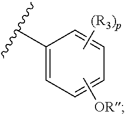

p is an integer between 1-4; R″ is hydrogen, Alk, or COR; R₃ is hydrogen, aldehyde, COOH, C(═N)—OH, CHNOH, CH═CHCO₂H, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring.

In one embodiment of the compound of Formula I, A is nothing, N forms a double bond with the cyclic carbon and X is OCH₂CH₂-heterocycle in which the heterocycle is a 3-7 membered heterocycloalkyl. In one embodiment, when X is OCH₂CH₂-heterocycle, the heterocycle is substituted or unsubstituted piperidine, pyrrolidine, morpholine or piperazine. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, wherein R is as defined for Formula I.

In another embodiment of the compound of Formula I, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH. In another embodiment, R₂ is —CH═CH—CH₃. In another embodiment, R₂ is —CH═CH—COOEt. In another embodiment R₁ is a hydroxyl group. In another embodiment R₁ is O—(CO)-Ph-CF₃. In another embodiment R₁ is COOH. In another embodiment R₁ is COOMe. In another embodiment R₁ is hydrogen. In another embodiment R₁ is a hydroxyl group and n is 1. In another embodiment R₁ is in position 8 of the isoquinolinone group. In another embodiment R₃ is halogen. In another embodiment R₃ is fluoride. In another embodiment R₃ is chloride. In another embodiment R₃ is bromide. In another embodiment R₃ is iodide. In another embodiment R₃ is hydrogen. In another embodiment R′ is H. In another embodiment R′ is a methyl group. In another embodiment R′ is a COMe group. In another embodiment R″ is H. In another embodiment R″ is a methyl group. In another embodiment R″ is a COMe group.

In another embodiment, this invention provides (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound or its prodrug, analog, isomer, metabolite, derivative, pharmaceutically acceptable salt, pharmaceutical product, polymorph, crystal, impurity, N-oxide, ester, hydrate or any combination thereof, represented by the structure of Formula II:

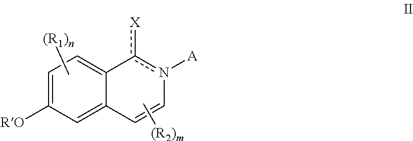

wherein

A is a 5-14 membered saturated or unsaturated, substituted or unsubstituted carbocyclic or to heterocyclic ring which is optionally a fused ring system, or a combination thereof; wherein the saturated or unsaturated carbocyclic or heterocyclic ring are optionally substituted by 1 to 5 substituents independently selected from R₃ or OR″; and X is O or S; or

A is nothing, N forms a double bond with the cyclic carbon and X is OH or OCH₂CH₂-heterocycle in which the heterocycle is a 3-7 membered saturated or unsaturated, substituted or unsubstituted heterocyclic ring;

R₁, R₂, R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

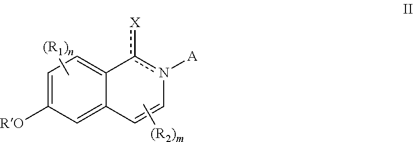

Q is SO₃H, CO₂H, CO₂R, NO₂, tetrazole, SO₂NH₂ or SO₂NHR;
n is an integer between 1-3;
m is an integer between 1-2;
p is an integer between 1-4; and
Alk is a linear alkyl of 1-7 carbons, branched alkyl of 1-7 carbons, or cyclic alkyl of 3-8 carbons.

In one embodiment, A is

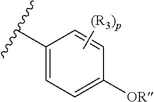

wherein p is an integer between 1-4; R″ is hydrogen, Alk, or COR; R₃ is hydrogen, aldehyde, COOH, C(═N)—OH, CHNOH, CH═CHCO₂H, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring;

In one embodiment of the compound of Formula II, A is nothing, N forms a double bond with the cyclic carbon and X is OCH₂CH₂-heterocycle, in which the heterocycle is a 3-7 membered heterocycloalkyl. In one embodiment, when X is OCH₂CH₂-heterocycle, the heterocycle is substituted or unsubstituted piperidine, pyrrolidine, morpholine or piperazine. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, wherein R is as defined for Formula II.

In another embodiment of the compound of Formula II, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In another embodiment R₁ is O—(CO)-Ph-CF₃. In another embodiment R₁ is COOH. In another embodiment R₁ is COOMe. In another embodiment R₁ is a hydroxyl group. In another embodiment R₁ is hydrogen. In another embodiment R₁ is a hydroxyl group and n is 1. In another embodiment R₁ is in position 8 of the isoquinolinone group. In another embodiment R₃ is halogen. In another embodiment R₃ is fluoride. In another embodiment R₃ is chloride. In another embodiment R₃ is bromide. In another embodiment R₃ is iodide. In another embodiment R₃ is hydrogen. In another embodiment R′ is H. In another embodiment R′ is a methyl group. In another embodiment R′ is a COMe group. In another embodiment R″ is H. In another embodiment R″ is a methyl group. In another embodiment R″ is a COMe group.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula III:

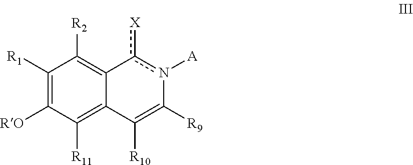

wherein

A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic ring are optionally substituted by 1 to 5 substituents independently selected from R₃ or OR″; and X is O or S; or

A is nothing and N forms a double bond with the cyclic carbon and X is OH or OCH₂CH₂-heterocycle in which the heterocycle is a 3-7 membered saturated or unsaturated, substituted or unsubstituted heterocyclic ring;

R₁, R₂, R₃, R₉, R₁₀, R₁₁ are independently selected from hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂, or OH;

R′ is hydrogen, Alk, or COR;

R″ is hydrogen, Alk, or COR

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂, or

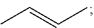

Q is SO₃H, CO₂H, CO₂R, NO₂, tetrazole, SO₂NH₂, or SO₂NHR; and
Alk is a linear alkyl of 1-7 carbons, branched alkyl of 1-7 carbons, or cyclic alkyl of 3-8 carbons.

In one embodiment, A is

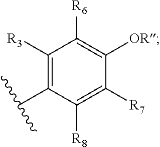

R₃, R₆, R₇, R₈, are independently selected from hydrogen, aldehyde, COOH, —C(═NH)—OH CHNOH, CH═CHCO₂H, CH═CHCO₂R—CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring; R″ is hydrogen, Alk, or COR;

In another embodiment, if A is

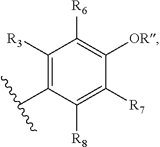

X is an oxo group and R₁₀ is a benzene ring, then R₉ is not COOR, if R is an ester residue or CONR₄R₅. In one embodiment of the compound of Formula III, A is nothing, N forms a double bond with the cyclic carbon and X is OCH₂CH₂-heterocycle in which the heterocycle is a 3-7 membered heterocycloalkyl. In one embodiment, when X is OCH₂CH₂-heterocycle, the heterocycle is substituted or unsubstituted piperidine, pyrrolidine, morpholine or piperazine. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, wherein R is as defined for Formula III.

In another embodiment of the compound of Formula III, R₁₀ is a halogen. In another embodiment R₁₀ is a bromide. In another embodiment R₁₀ is a chloride. In another embodiment R₁₀ is a fluoride. In another embodiment R₁₀ is an iodide. In another embodiment R₁₀ is hydrogen. In another embodiment R₁₀ is a cyano. In another embodiment, R₁₀ is a phenyl. In another embodiment, R₁₀ is —CH═CH—CH₃. In another embodiment, R₁₀ is —CH═CH₂. In another embodiment, R₁₀ is —CH═CH—COOEt. In another embodiment R₂ is a hydroxyl group. In another embodiment R₂ is hydrogen. In another embodiment R₂ is O—(CO)-Ph-CF₃. In another embodiment R₂ is COOH. In another embodiment R₂ is COOMe. In another embodiment R₇ is a halogen. In another embodiment R₇ is fluoride. In another embodiment R₇ is chloride. In another embodiment R₇ is bromide. In another embodiment R₇ is iodide. In another embodiment R₃, R₆, R₇ and R₈ are hydrogens. In another embodiment R′ is H. In another embodiment R′ is a methyl group. In another embodiment R′ is a COMe. In another embodiment R″ is H. In another embodiment R″ is a methyl group. In another embodiment R″ is COMe. In another embodiment R₁, R₃, R₆, R₇, R₈, R₉ and R₁₁ are hydrogens.

In one embodiment, this invention provides: ((1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula IV:

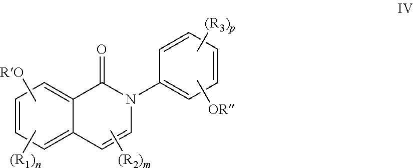

wherein

R₁, R₂, R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

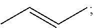

Q is SO₃H, CO₂H, CO₂R, NO₂, tetrazole, SO₂NH₂ or SO₂NHR;
n is an integer between 1-3;
m is an integer between 1-2;
p is an integer between 1-4; and
Alk is a linear alkyl of 1-7 carbons, branched alkyl of 1-7 carbons, or cyclic alkyl of 3-8 carbons.

In another embodiment of the compound of Formula IV, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In another embodiment R₁ is O—(CO)-Ph-CF₃. In another embodiment R₁ is COOH. In another embodiment R₁ is COOMe. In another embodiment R₁ is a hydroxyl group. In another embodiment R₁ is hydrogen. In another embodiment R₃ is halogen. In another embodiment R₃ is fluoride. In another embodiment R₃ is chloride. In another embodiment R₃ is bromide. In another embodiment R₃ is iodide. In another embodiment R₃ is hydrogen. In another embodiment R′ is H. In another embodiment R′ is a methyl group. In another embodiment R′ is COMe. In another embodiment R″ is H. In another embodiment R″ is a methyl group. In another embodiment R″ is COMe. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, the heterocycles are optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, wherein R is as defined for Formula IV.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula V:

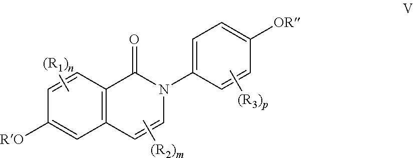

wherein

R₁, R₂, R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

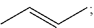

Q is SO₃H, CO₂H, CO₂R, NO₂, tetrazole, SO₂NH₂ or SO₂NHR;
n is an integer between 1-3;
m is an integer between 1-2;
p is an integer between 1-4; and
Alk is a linear alkyl of 1-7 carbons, branched alkyl of 1-7 carbons or cyclic alkyl of 3-8 carbons.

In another embodiment of the compound of Formula V, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In another embodiment R₁ is O—(CO)-Ph-CF₃. In another embodiment R₁ is COOH. In another embodiment R₁ is COOMe. In another embodiment R₁ is a hydroxyl group. In another embodiment R₁ is hydrogen. In another embodiment R₃ is halogen. In another embodiment R₃ is fluoride. In another embodiment R₃ is chloride. In another embodiment R₃ is bromide. In another embodiment R₃ is iodide. In another embodiment R₃ is hydrogen. In another embodiment R′ is H. In another embodiment R′ is a methyl group. In another embodiment R′ is a COMe group In another embodiment R″ is H. In another embodiment R″ is a methyl group. In another embodiment R″ is a COMe. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy; and R is as defined for Formula V.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula VI:

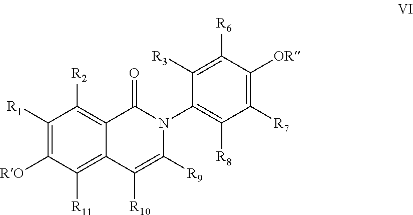

wherein

R₁, R₂, R₃, R₆, R₇, R₈, R₉, R₁₀, R₁₁ are independently selected from hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

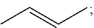

Q is SO₃H, CO₂H, CO₂R, NO₂, tetrazole, SO₂NH₂ or SO₂NHR;
R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH and; and Alk is a linear alkyl of 1-7 carbons, branched alkyl of 1-7 carbons or cyclic alkyl of 3-8 carbons.

In another embodiment of the compound of Formula VI, R₁₀ is a halogen. In another embodiment R₁₀ is a bromide. In another embodiment R₁₀ is a chloride. In another embodiment R₂ is a fluoride. In another embodiment R₁₀ is an iodide. In another embodiment R₁₀ is hydrogen. In another embodiment R₁₀ is a cyano. In another embodiment, R₁₀ is a phenyl. In another embodiment, R₁₀ is —CH═CH—CH₃. In another embodiment, R₁₀ is —CH═CH₂. In another embodiment, R₁₀ is —CH═CH—COOEt. In another embodiment R₂ is a hydroxyl group. In another embodiment R₂ is hydrogen. In another embodiment R₂ is O—(CO)-Ph-CF₃. In another embodiment R₂ is COOH. In another embodiment R₂ is COOMe. In another embodiment R₇ is a halogen. In another embodiment R₇ is fluoride. In another embodiment R₇ is chloride. In another embodiment R₇ is bromide. In another embodiment R₇ is iodide. In another embodiment R₃, R₆, R₇ and R₈ are hydrogens. In another embodiment R′ is H. In another embodiment R′ is a methyl group. In another embodiment R′ is a COMe. In another embodiment R″ is H. In another embodiment R″ is a methyl group. In another embodiment R″ is COMe. In another embodiment R₁, R₃, R₆, R₇, R₈, R₉ and R₁₁ are hydrogens. In another embodiment, when R₁, R₂, R₃, R₆, R₇, R₈, R₉, R₁₀, R₁₁ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, wherein R is as defined for Formula VI.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula VII:

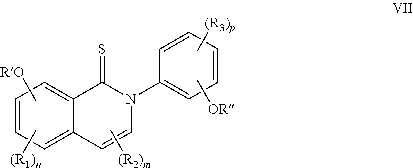

wherein

R₁, R₂, R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

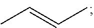

Q is SO₃H, CO₂H, CO₂R, NO₂, tetrazole, SO₂NH₂ or SO₂NHR;
R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
n is an integer between 1-3;
m is an integer between 1-2;
p is an integer between 1-4; and
Alk is a linear alkyl of 1-7 carbons, branched alkyl of 1-7 carbons or cyclic alkyl of 3-8 carbons.

In another embodiment of the compound of Formula VII, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In another embodiment R₁ is O—(CO)-Ph-CF₃. In another embodiment R₁ is COOH. In another embodiment R₁ is COOMe. In another embodiment R₁ is a hydroxyl group. In another embodiment R₁ is hydrogen. In another embodiment R₃ is halogen. In another embodiment R₃ is fluoride. In another embodiment R₃ is chloride. In another embodiment R₃ is bromide. In another embodiment R₃ is iodide. In another embodiment R₃ is hydrogen. In another embodiment R′ is H. In another embodiment R′ is a methyl group. In another embodiment R′ is COMe. In another embodiment R″ is H. In another embodiment R″ is a methyl group. In another embodiment R″ is a COMe. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, and R is as defined for Formula VII.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula VIII:

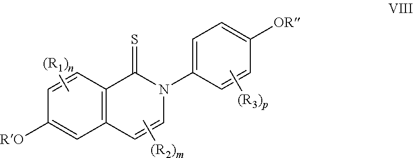

wherein

R₁, R₂, R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro,

CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

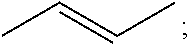

Q is SO₃H, CO₂H, CO₂R, NO₂, tetrazole, SO₂NH₂ or SO₂NHR;
R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
n is an integer between 1-3;
m is an integer between 1-2;
p is an integer between 1-4; and
Alk is a linear alkyl of 1-7 carbons, branched alkyl of 1-7 carbons or cyclic alkyl of 3-8 carbons.

In another embodiment of the compound of Formula VIII, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In another embodiment R₁ is O—(CO)-Ph-CF₃. In another embodiment R₁ is COOH. In another embodiment R₁ is COOMe. In another embodiment R₁ is a hydroxyl group. In another embodiment R₁ is hydrogen. In another embodiment R₃ is hydrogen. In another embodiment R₃ is halogen. In another embodiment R₃ is fluoride. In another embodiment R₃ is chloride. In another embodiment R₃ is bromide. In another embodiment R₃ is iodide. In another embodiment R′ is H. In another embodiment R′ is a methyl group. In another embodiment R′ is COMe. In another embodiment R″ is H. In another embodiment R″ is a methyl group. In another embodiment R″ is COMe. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, and R is as defined for Formula VIII.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula IX:

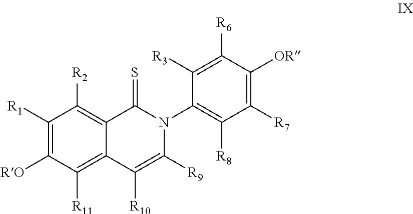

wherein

R₁, R₂, R₃, R₆, R₇, R₈, R₉, R₁₀, R₁₁ are independently selected from hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

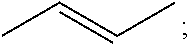

Q is SO₃H, CO₂H, CO₂R, NO₂, tetrazole, SO₂NH₂ or SO₂NHR;
R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH; and
Alk is a linear alkyl of 1-7 carbons, branched alkyl of 1-7 carbons or cyclic alkyl of 3-8 carbons.

In another embodiment of the compound of Formula IX, R₁₀ is a halogen. In another embodiment R₁₀ is a bromide. In another embodiment R₁₀ is a chloride. In another embodiment R₂ is a fluoride. In another embodiment R₁₀ is an iodide. In another embodiment R₁₀ is hydrogen. In another embodiment R₁₀ is a cyano. In another embodiment, R₁₀ is a phenyl. In another embodiment, R₁₀ is —CH═CH—CH₃. In another embodiment, R₁₀ is —CH═CH₂. In another embodiment, R₁₀ is —CH═CH—COOEt. In another embodiment R₂ is a hydroxyl group. In another embodiment R₂ is hydrogen. In another embodiment R₂ is O—(CO)-Ph-CF₃. In another embodiment R₂ is COOH. In another embodiment R₂ is COOMe. In another embodiment R₇ is a halogen. In another embodiment R₇ is fluoride. In another embodiment R₇ is chloride. In another embodiment R₇ is bromide. In another embodiment R₇ is iodide. In another embodiment R₃, R₆, R₇ and R₈ are hydrogens. In another embodiment R′ is H. In another embodiment R′ is a methyl group. In another embodiment R′ is a COMe. In another embodiment R″ is H. In another embodiment R″ is a methyl group. In another embodiment R″ is COMe. In another embodiment R₁, R₃, R₆, R₇, R₈, R₉ and R₁₁ are hydrogens. In another embodiment, when R₁, R₂, R₃, R₆, R₇, R₈, R₉, R₁₀, R₁₁ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, and R is as defined for Formula IX.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula X:

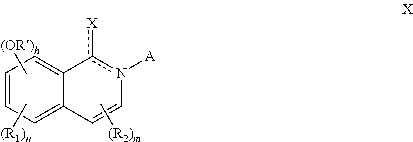

wherein

A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic ring are optionally substituted by 1 to 5 substituents independently selected from R₃ or OR″; and X is O or S; or

A is nothing, N forms a double bond with the cyclic carbon and X is OH or OCH₂CH₂-heterocycle in which the heterocycle is a 3-7 membered saturated or unsaturated, substituted or unsubstituted heterocyclic ring;

R₁, R₂, R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

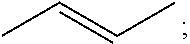

Q is SO₃H, CO₂H, CO₂R, NO₂, tetrazole, SO₂NH₂ or SO₂NHR;
R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
h is an integer between 0-3;
n is an integer between 1-4;
m is an integer between 1-2; and
Alk is a linear alkyl of 1-7 carbons, branched alkyl of 1-7 carbons or cyclic alkyl of 3-8 carbons.

In one embodiment, A is

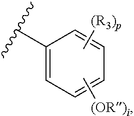

p is an integer between 1-5; i is an integer between 0-4; R″ is hydrogen, Alk or COR; and R₃ is hydrogen, aldehyde, COOH, C(═NH)—OH, CHNOH, CH═CHCO₂H, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring.

In one embodiment of the compound of Formula X, A is nothing, N forms a double bond with the cyclic carbon and X is OCH₂CH₂-heterocycle in which the heterocycle is a 3-7 membered heterocycloalkyl. In one embodiment, when X is OCH₂CH₂-heterocycle, the heterocycle is substituted or unsubstituted piperidine, pyrrolidine, morpholine or piperazine. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, haloalkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, and R is as defined for Formula X.

In another embodiment of the compound of Formula X, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In another embodiment R₁ is O—(CO)-Ph-CF₃. In another embodiment R₁ is COOH. In another embodiment R₁ is COOMe. In another embodiment R₁ is a hydroxyl group. In another embodiment R₁ is hydrogen. In another embodiment R₃ is halogen. In another embodiment R₃ is fluoride. In another embodiment R₃ is chloride. In another embodiment R₃ is bromide. In another embodiment R₃ is iodide. In another embodiment R₃ is hydrogen. In another embodiment R′ is H. In another embodiment R′ is a methyl group. In another embodiment R′ is COMe. In another embodiment R″ is H. In another embodiment R″ is a methyl group. In another embodiment R″ is COMe.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula XI:

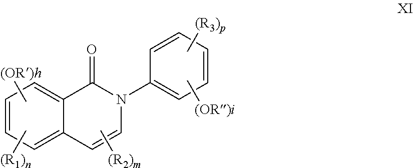

wherein
R₁, R₂, R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;
R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;

R′ is hydrogen, Alk or COR;

R″ is hydrogen, Alk or COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

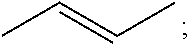

Q is SO₃H, CO₂H, CO₂R, NO₂, tetrazole, SO₂NH₂ or SO₂NHR;
h is an integer between 0-3;
i is an integer between 0-4;
n is an integer between 1-4;
m is an integer between 1-2;
p is an integer between 0-5; and
Alk is a linear alkyl of 1-7 carbons, branched alkyl of 1-7 carbons, or cyclic alkyl of 3-8 carbons.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula XIa:

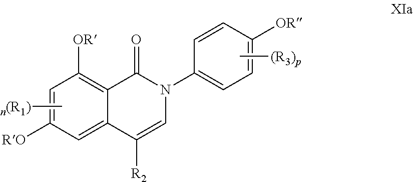

wherein
n is 1 or 2;
p is 0, 1, 2, 3 or 4; and
R₁, R₂, R₃, R′ and R″ are as described above for Formula I,

or its prodrug, analog, isomer, metabolite, derivative, pharmaceutically acceptable salt, pharmaceutical product, polymorph, crystal, impurity, N-oxide, ester, hydrate or any combination thereof.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula XIb:

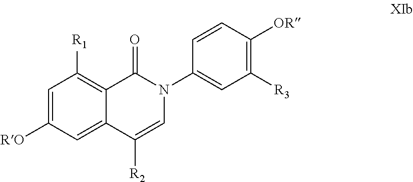

wherein R₁, R₂, R₃, R′ and R″ are as described above for Formula XI;

or its prodrug, analog, isomer, metabolite, derivative, pharmaceutically acceptable salt, pharmaceutical product, polymorph, crystal, impurity, N-oxide, ester, hydrate or any combination thereof.

In one embodiment, R₂ of formula XI, XIa and XIb is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In one embodiment R₁ of formula XI, XIa and XIb is O—(CO)-Ph-CF₃. In another embodiment R₁ is COOH. In another embodiment R₁ is COOMe. In another embodiment R₁ is a hydroxyl group. In another embodiment R₁ is a hydrogen. In one embodiment R₃ of formula XI, XIa and XIb is a hydrogen. In another embodiment R₃ is a halogen. In another embodiment R₃ is fluoride. In another embodiment R₃ is chloride. In another embodiment R₃ is bromide. In another embodiment R₃ is iodide. In one embodiment R′ of formula XI, XIa and XIb is H. In another embodiment R′ is a methyl group. In another embodiment R′ is a COMe. In one embodiment R″ of formula XI, XIa and XIb is H. In another embodiment R″ is a methyl group. In another embodiment R″ is a COMe. In one embodiment h of formula XI, XIa and XIb is 1. In another embodiment h is 2. In one embodiment, when R₁, R₂, R₃ of formula XI, XIa and XIb are independently Z-Alk-heterocycle or, in another embodiment, OCH₂CH₂-heterocycle, either heterocycle may be substituted or unsubstituted piperidine, pyrrolidine, morpholine or piperazine. In one embodiment, when R₄ and R₅ of formula XI, XIa and XIb are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, and R of formula XI, XIa and XIb is as defined for Formula XI.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula XII:

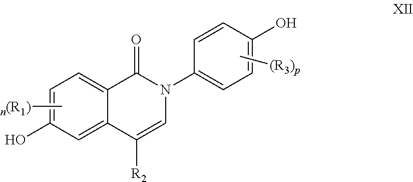

wherein
R₁, R₂ and R₃ are independently hydrogen, aldehyde, COOH, C(═NH)—OH, CHNOH, CH═CHCO₂H, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;

### Z is O, NH, CH₂ or

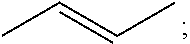

Q is SO₃H, CO₂H, CO₂R, NO₂, tetrazole, SO₂NH₂ or SO₂NHR;
R is alkyl, hydrogen, haloalkyl, dihaloalkyl, trihaloalkyl, CH₂F, CHF₂, CF₃, CF₂CF₃, aryl, phenyl, -Ph-CF₃, -Ph-CH₂F, -Ph-CHF₂, -Ph-CF₂CF₃, halogen, alkenyl, CN, NO₂ or OH;
n is an integer between 1-3;
p is an integer between 1-4; and
Alk is a linear alkyl of 1-7 carbons, branched alkyl of 1-7 carbons, or cyclic alkyl of 3-8 carbons.

In another embodiment of the compound of Formula XII, R₂ is a halogen. In another embodiment R₂ is a bromide. In another embodiment R₂ is a chloride. In another embodiment R₂ is a fluoride. In another embodiment R₂ is an iodide. In another embodiment R₂ is hydrogen. In another embodiment R₂ is a cyano. In another embodiment, R₂ is a phenyl. In another embodiment, R₂ is —CH═CH—CH₃. In another embodiment, R₂ is —CH═CH₂. In another embodiment, R₂ is —CH═CH—COOEt. In another embodiment R₁ is O—(CO)-Ph-CF₃. In another embodiment R₁ is COOH. In another embodiment R₁ is COOMe. In another embodiment R₁ is an hydroxyl group. In another embodiment R₁ is hydrogen. In another embodiment R₃ is halogen. In another embodiment R₃ is fluoride. In another embodiment R₃ is chloride. In another embodiment R₃ is bromide. In to another embodiment R₃ is iodide. In another embodiment R₃ is hydrogen. In another embodiment p is 1. In another embodiment, when R₁, R₂, R₃ are independently Z-Alk-heterocycle or, in another embodiment, OCH₂CH₂-heterocycle, either heterocycle may be substituted or unsubstituted piperidine, pyrrolidine, morpholine or piperazine. In another embodiment, when R₄ and R₅ are independently a 3 to 7 membered heterocycloalkyl, either heterocycle may be substituted or unsubstituted piperidine, pyrrolidine, morpholine or piperazine. In another embodiment, any heterocycle is optionally substituted by one or more substituents comprising halogen, cyano, nitro, COOH, COOR, NHCOR, hydroxyl, amine, alkyl, cycloalkyl, heterocycloalkyl, alkenyl, alkynyl, alkanoyl, alkylthio, alkylamino, N,N dialkylamino, aminoalkyl, haloalkyl, aryl, heteroaryl, alkoxy or haloalkoxy, and R is as defined for Formula XII.

In one embodiment, this invention provides: (1) a method of inhibiting a hydroxysteroid dehydrogenase in a patient in need thereof; (2) a method of treating a disorder that responds to a hydroxysteroid dehydrogenase inhibitor; (3) a method of lowering serum testosterone levels in a male subject; (4) a method of lowering serum estradiol levels in a subject; (5) a method of suppressing androgen-dependent androgen receptor activation using a hydroxysteroid dehydrogenase inhibitor; comprising administering to the patient a hydroxysteroid dehydrogenase inhibitor (HDI) compound represented by the structure of formula XIII:

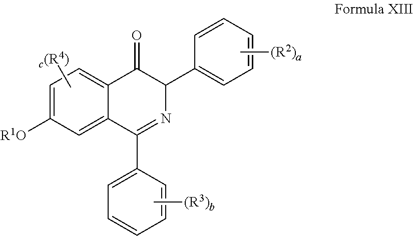

wherein

R¹ is H, alkyl or -alkylene-CO₂Rx, in which Rx is H or alkyl;

R² is H, substituted or unsubstituted alkyl, substituted or unsubstituted alkenyl, substituted or unsubstituted alkyl, substituted or unsubstituted cycloalkyl, alkoxy, haloalkyl, hydroxyl, hydroxymethyl, CONH₂, CONHR^{y}, substituted or unsubstituted alkylene-CO₂R^{y}, in which R^{y} is H or alkyl;

R³ is, in each case, independently selected from hydrogen, alkoxy, COOH, hydroxyl, halogen, haloalkyl, CF₂OMe, CONH₂, CN, carboxyl, SO₂R^{Z} or SO₂NHR^{Z} in which R^{Z} is, in each case, independently, H or alkyl;

R⁴ is, in each case, independently selected from hydrogen, alkyl, hydroxyl, halogen, haloalkyl, CN, carboxyl, CONH₂, CONHR^{Z}, SO₂R^{Z} or SO₂NHR^{Z} in which R^{Z} is, in each case, independently, H or alkyl;

a=1, 2, 3, 4 or 5;

b=1, 2, 3, 4 or 5; and

c=1, 2 or 3;

or a prodrug, isomer, tautomer, metabolite, pharmaceutically acceptable salt, polymorph, crystal, N-oxide, hydrate, or any combination thereof.

In one embodiment, R¹ of formula XIII is hydrogen. In one embodiment, R¹ of formula XIII is alkyl.

In one embodiment, R² of formula XIII is, in each case, independently hydroxyl. In one embodiment, R² of formula XIII is, in each case, independently hydrogen. In another embodiment, R² of formula XIII is, in each case, independently CH₂-halogen. In another embodiment, R² of formula XIII is, in each case, independently CH₂—Br. In another embodiment, R² of formula XIII is, in each case, independently CH₂OH. In another embodiment, R² of formula XIII is, in each case, independently C(O)NH₂. In another embodiment, R² of formula XIII is, in each case, independently CF₃. In another embodiment, R² of formula XIII is, in each case, independently halogen. In another embodiment, R² of formula XIII is, in each case, independently F. In another embodiment, R² of formula XIII is, in each case, independently Cl. In another embodiment, R² of formula XIII is, in each case, independently Br. In another embodiment, R² of formula XIII is, in each case, independently I.

In one embodiment, R³ of formula XIII is, in each case, independently hydrogen. In another embodiment, R³ of formula XIII is, in each case, independently alkoxy. In another embodiment, R³ of formula XIII is, in each case, independently OMe. In another embodiment, R³ of formula XIII is, in each case, independently CONH₂. In another embodiment, R³ of formula XIII is, in each case, independently CF₃. In another embodiment, R³ of formula XIII is, in each case, independently C(O)OH. In another embodiment, R³ of formula XIII is, in each case, independently CF₃. In another embodiment, R³ of formula XIII is, in each case, independently halogen. In another embodiment, R³ of formula XIII is, in each case, independently F. In another embodiment, R³ of formula XIII is, in each case, independently Cl. In another embodiment, R³ of formula XIII is, in each case, independently Br. In another embodiment, R³ of formula XIII is, in each case, independently I. In another embodiment, R³ of formula XIII is, in each case, independently SO₂CH₃.

In one embodiment, R⁴ of formula XIII is, in each case, independently hydrogen. In another embodiment, R⁴ of formula XIII is, in each case, independently hydroxyl. In another embodiment, R⁴ of formula XIII is, in each case, independently CN. In another embodiment, R⁴ of formula XIII is, in each case, independently halogen. In another embodiment, R⁴ of formula XIII is, in each case, independently F. In another embodiment, R⁴ of formula XIII is, in each case, independently Cl. In another embodiment, R⁴ of formula XIII is, in each case, independently Br. In another embodiment, R⁴ of formula XIII is, in each case, independently I. In another embodiment, R⁴ of formula XIII is, in each case, independently SO₂NHCH₃.

In one embodiment an AKR1C3 inhibitor of Formula XIII of this invention is 6-hydroxy-2,4-bis(4-hydroxyphenyl)isoquinolin-1(2H)-one (6); In another embodiment an AKR1C3 inhibitor of Formula XIII of this invention is 2-(4-(hydroxymethyl)phenyl)-6-methoxy-4-(4-methoxyphenyl)isoquinolin-1(2H)-one (10); In another embodiment an AKR1C3 inhibitor of Formula XIII of this invention is 2-(4-bromomethyl)phenyl-6-hydroxy-4-(4-hydroxyphenyl)isoquinolin-1(2H)-one (11); In another embodiment an AKR1C3 inhibitor of Formula XIII of this invention is 6-hydroxy-2-(4-hydroxyphenyl)-4-(4-(trifluoromethyl)phenyl)isoquinolin-1(2H)-one (13); In another embodiment an AKR1C3 inhibitor of Formula XIII of this invention is 6-hydroxy-2-(4-(hydroxymethyl)phenyl)-4-(4-hydroxyphenyl)isoquinolin-1(2H)-one (14); In another embodiment an AKR1C3 inhibitor of Formula XIII of this invention is 2-(4-(hydroxymethyl)-3-methoxyphenyl)-6-methoxy-4-(4-(trifluoromethyl)phenyl)isoquinolin-1(2H)-one (25); In another embodiment an AKR1C3 inhibitor of Formula XIII of this invention is 2-(4-(bromomethyl)-3-hydroxyphenyl)-6-hydroxy-4-(4-(trifluoromethyl)phenyl)isoquinolin-1(2H)-one (26); In another embodiment an AKR1C3 inhibitor of Formula XIII of this invention is 6-hydroxy-2,4-bis(4-(trifluoromethyl)phenyl)isoquinolin-1(2H)-one (30); In another embodiment an AKR1C3 inhibitor of Formula XIII of this invention is 2-(4-fluorophenyl)-6-hydroxy-4-(4-(trifluoromethyl)phenyl)isoquinolin-1(2H)-one (34); In another embodiment an AKR1C3 inhibitor of Formula XIII of this invention is 6-methoxy-2-(4-methoxyphenyl)-4-(4-(trifluoromethyl)phenyl)isoquinolin-1(2H)-one (35); In another embodiment an AKR1C3 inhibitor of Formula XIII of this invention is 4-(6-hydroxy-2-(4-hydroxyphenyl)-1-oxo-1,2-dihydroisoquinolin-4-yl)-N-methyl benzenesulfonamide (36); In another embodiment an AKR1C3 inhibitor of Formula XIII of this invention is 6-hydroxy-2-(4-hydroxyphenyl)-4-(4-(methylsulfonyl)phenyl)isoquinolin-1(2H)-one (37); In another embodiment an AKR1C3 inhibitor of Formula XIII of this invention is 4-(6-hydroxy-2-(4-hydroxyphenyl)-1-oxo-1,2-dihydroisoquinolin-4-yl)benzoic acid (43). In another embodiment an AKR1C3 inhibitor of Formula XIII of this invention is 2-benzyl-6-hydroxy-4-(3,4,5-trifluorophenyl)isoquinolin-1(2H)-one (75); In another embodiment an AKR1C3 inhibitor of Formula XIII of this invention is 6-hydroxy-2-(4-hydroxyphenyl)-4-(3,4,5-trifluorophenyl)isoquinolin-1(2H)-one (79); In another embodiment an AKR1C3 inhibitor of Formula XIII of this invention is 4-(3,4,5-trifluorophenyl)isoquinolin-1(2H)-one (90); In another embodiment an AKR1C3 inhibitor of Formula XIII of this invention is methyl 6-hydroxy-2-(4-hydroxyphenyl)-1-oxo-4-(3,4,5-trifluorophenyl)-1,2-dihydroisoquinoline-8-carbimidate (100); In another embodiment an AKR1C3 inhibitor of Formula XIII of this invention is 6-hydroxy-2-(4-hydroxyphenyl)-1-oxo-4-(3,4,5-trifluorophenyl)-1,2-dihydroisoquinoline-8-carboxamide (100A); In another embodiment an AKR1C3 inhibitor of Formula XIII of this invention is 4-(3-fluoro-4-(trifluoromethyl)phenyl)-6-hydroxy-2-(4-hydroxyphenyl)-1-oxo-1,2-dihydroisoquinoline-8-carbonitrile (102); In another embodiment an AKR1C3 inhibitor of Formula XIII of this invention is methyl-4-(3-fluoro-4-(trifluoromethyl)phenyl)-6-hydroxy-2-(4-hydroxyphenyl)-1-oxo-1,2-dihydroisoquinoline-8-carbimidate (102A); In another embodiment an AKR1C3 inhibitor of Formula XIII of this invention is 4-(3-fluoro-4-(trifluoromethyl)phenyl)-6-hydroxy-2-(4-hydroxyphenyl)-1-oxo-1,2-dihydroisoquinoline-8-carboxamide (102B); 6-hydroxy-2-(1-oxo-1,3-dihydroisobenzofuran-5-yl)isoquinolin-1(2H)-one (104); In another embodiment an AKR1C3 inhibitor of Formula XIII of this invention is methyl 2-(bromomethyl)-4-(6-hydroxy-1-oxoisoquinolin-2(1H)-yl)-benzoate (104A); In to another embodiment an AKR1C3 inhibitor of Formula XIII of this invention is 4-bromo-6,8-dihydroxy-2-(3-hydroxyphenyl)isoquinolin-1(2H)-one (213) or their prodrug, isomer, metabolite, pharmaceutically acceptable salt, polymorph, crystal, N-oxide, hydrate or any combination thereof.

In one embodiment, an HDI compound of this invention is 4-cyano-6,8-dihydroxy-2-(4-hydroxyphenyl)isoquinolin-1(2H)-one. In another embodiment an HDI compound of this invention is 4-bromo-6,8-dihydroxy-2-(4-hydroxyphenyl)isoquinolin-1(2H)-one. In another embodiment an HDI compound of this invention is 1-(2-(piperidin-1-yl)ethoxy)isoquinolin-6-ol. In another embodiment an HDI compound of this invention is 6-hydroxy-2-(4-hydroxyphenyl)isoquinolin-1(2H)-one. In another embodiment an HDI compound of this invention is 4-bromo-6-hydroxy-2-(4-hydroxyphenyl)isoquinolin-1(2H)-one. In another embodiment an HDI compound of this invention is 4-bromo-2-(4-hydroxyphenyl)-6-methoxyisoquinolin-1(2H)-one. In another embodiment an HDI compound of this invention is 4-bromo-2-(3-fluoro-4-hydroxyphenyl)-6-hydroxyisoquinolin-1(2H)-one. In another embodiment an HDI compound of this invention is 4-bromo-2-(4-fluorophenyl)-6-hydroxyisoquinolin-1(2H)-one. In another embodiment an HDI compound of this invention is 4-chloro-6-hydroxy-2-(4-hydroxyphenyl)isoquinolin-1(2H)-one. In another embodiment an HDI compound of this invention is 4-chloro-2-(3-fluoro-4-hydroxyphenyl)-6-hydroxyisoquinolin-1(2H)-one. In another embodiment an HDI compound of this invention is 6-hydroxy-2-(4-hydroxyphenyl)-4-iodoisoquinolin-1(2H)-one. In another embodiment an HDI compound of this invention is 4-bromo-6-hydroxy-2-(3-hydroxyphenyl)isoquinolin-1(2H)-one. In another embodiment an HDI compound of this invention is 8-hydroxy-2-(4-hydroxyphenyl)-6-methoxy-isoquinolin-1(2H)-one. In another embodiment an HDI compound of this invention is 5-bromo-8-hydroxy-2-(4-hydroxyphenyl)-6-methoxy-isoquinolin-1(2H)-one. In another embodiment an HDI compound of this invention is 6,8-dihydroxy-2-(4-hydroxyphenyl)-isoquinolin-1(2H)-one. In another embodiment an HDI compound of this invention is 5-bromo-6,8-dihydroxy-2-(4-hydroxyphenyl)isoquinolin-1(2H)-one. In another embodiment an HDI compound of this invention is 2-(3-fluoro-4-hydroxyphenyl)-6-hydroxy-4-iodoisoquinolin-1(2H)-one. In another embodiment an HDI compound of this invention is 4-bromo-6-hydroxy-2-(4-hydroxy-3-methylphenyl)isoquinolin-1(2H)-one. In another embodiment an HDI compound of this invention is 2-(4-hydroxyphenyl)-6,8-dihydroxy-isoquinoline-1(2H)-thione. In another embodiment an HDI compound of this invention is 8-hydroxy-2-(4-hydroxyphenyl)-6-methoxy-1-oxo-1,2-dihydroisoquinoline-5-carbonitrile. In another embodiment an HDI compound of this invention is 4-bromo-6-hydroxy-2-(4-hydroxyphenyl)isoquinoline-1(2H)-thione. In another embodiment an HDI compound of this invention is 2-(3-fluoro-4-hydroxyphenyl)-6,8-dihydroxyisoquinolin-1(2H)-one. In another embodiment an HDI compound of this invention is 2-(3-fluoro-4-hydroxyphenyl)-8-hydroxy-6-methoxyisoquinolin-1(2H)-one. In another embodiment an HDI compound of this invention is 4-bromo-8-hydroxy-2-(4-hydroxyphenyl)-6-methoxyisoquinolin-1(2H)-one. In another embodiment an HDI compound of this invention is 4-chloro-6,8-dihydroxy-2-(4-hydroxyphenyl)isoquinolin-1(2H)-one. In another embodiment an HDI compound of this invention is 4-bromo-6,8-dihydroxy-2-(3-fluoro-4-hydroxyphenyl)isoquinolin-1(2H)-one. In another embodiment an HDI compound of this invention is 4,5-dibromo-2-(3,5-dibromo-4-hydroxyphenyl)-6-hydroxyisoquinolin-1(2H)-one. In another embodiment an HDI compound of this invention is 6,8-dihydroxy-2-(4-hydroxyphenyl)-5-(trifluoromethylsulfonyl)isoquinolin-1(2H)-one. In another embodiment an HDI compound of this invention is 4-(1,2-dibromoethyl)-6-hydroxy-2-(4-hydroxyphenyl)isoquinolin-1(2H)-one. In another embodiment an HDI compound of this invention is 6-methoxy-2-(4-methoxyphenyl)-1-oxo-1,2-dihydroisoquinolin-8-yltrifluoromethanesulfonate. In another embodiment an HDI compound of this invention is 4,5-dibromo-6,8-dihydroxy-2-(4-hydroxyphenyl)isoquinolin-1(2H)-one. In another embodiment an HDI compound of this invention is 6-hydroxy-2-(4-hydroxyphenyl)-4-vinylisoquinolin-1(2H)-one. In another embodiment an HDI compound of this invention is 6-methoxy-2-(4-methoxyphenyl)-1-oxo-1,2-dihydroisoquinoline-4-carbonitrile. In another embodiment an HDI compound of this invention is 6-hydroxy-2-(4-hydroxyphenyl)-1-oxo-1,2-dihydroisoquinoline-4-carbonitrile. In another embodiment an HDI compound of this invention is 6-methoxy-2-(4-methoxyphenyl)-1-oxo-1,2-dihydroisoquinoline-8-carbonitrile. In another embodiment an HDI compound of this invention is 4-bromo-6-methoxy-2-(4-methoxyphenyl)-1-oxo-1,2-dihydroisoquinoline-8-carbonitrile. In another embodiment an HDI compound of this invention is 4-bromo-6-hydroxy-2-(4-hydroxyphenyl)-1-oxo-1,2-dihydroisoquinoline-8-carbonitrile. In another embodiment an HDI compound of this invention is 6,8-dihydroxy-2-(4-hydroxyphenyl)-4-vinylisoquinolin-1(2H)-one. In another embodiment an HDI compound of this invention is 6,8-dihydroxy-2-(4-hydroxyphenyl)-1-oxo-1,2-dihydroisoquinoline-4-carbonitrile or 4-cyano-6,8-dihydroxy-2-(4-hydroxyphenyl)isoquinolin-1(2H)-one. In another embodiment an HDI compound of this invention is 6-hydroxy-2-(4-hydroxyphenyl)-1-oxo-1,2-dihydroisoquinoline-8-carbonitrile. In another embodiment an HDI compound of this invention is 6-hydroxy-2-(4-hydroxyphenyl)-1-oxo-4-vinyl-1,2-dihydroisoquinoline-8-carbonitrile. In another embodiment an HDI compound of this invention is 4-chloro-6-hydroxy-2-(4-hydroxyphenyl)-1-oxo-1,2-dihydroisoquinoline-8-carbonitrile. In another embodiment an HDI compound of this invention is 4-bromo-6-methoxy-2-(4-methoxyphenyl)isoquinolin-1(2H)-one. In another embodiment an HDI compound of this invention is 8-hydroxy-6-methoxy-2-(4-methoxyphenyl)isoquinolin-1(2H)-one. In another embodiment an HDI compound of this invention is 4-chloro-6-methoxy-2-(4-methoxyphenyl)-1-oxo-1,2-dihydroisoquinolin-8-yl trifluoromethanesulfonate. In another embodiment an HDI compound of this invention is 4-chloro-6-methoxy-2-(4-methoxyphenyl)-1-oxo-1,2-dihydroisoquinoline-8-carbonitrile. In another embodiment an HDI compound of this invention is isoquinoline-1,6-diol. In another embodiment an HDI compound of this invention is 4-bromo-6-hydroxy-2-(4-methoxyphenyl)isoquinolin-1(2H)-one. In another embodiment an HDI compound of this invention is 4-(6-acetoxy-4-bromo-1-oxoisoquinolin-2(1H)-yl)phenyl acetate. In another embodiment an HDI compound of this invention is 4-(4-bromo-6-methoxy-1-oxoisoquinolin-2(1H)-yl)phenyl acetate. In another embodiment an HDI compound of this invention is 4-bromo-6-hydroxy-2-(4-hydroxyphenyl)-1-oxo-1,2-dihydroisoquinoline-8-carbimidic acid. In another embodiment an HDI compound of this invention is methyl 4-bromo-6-hydroxy-2-(4-hydroxyphenyl)-1-oxo-1,2-dihydroisoquinoline-8-carboxylate. In another embodiment an HDI compound of this invention is 4-bromo-6-hydroxy-2-(4-hydroxyphenyl)-1-oxo-1,2-dihydroisoquinoline-8-carboxylic acid. In another embodiment an HDI compound of this invention is 6-hydroxy-2-(4-hydroxyphenyl)-4-phenylisoquinolin-1(2H)-one. In another embodiment an HDI compound of this invention is 6-hydroxy-2-(4-hydroxyphenyl)-4-(4-methoxyphenyl)isoquinolin-1(2H)-one. In another embodiment an HDI compound of this invention is 2-(3-fluoro-4-hydroxyphenyl)-6,8-dihydroxy-4-vinylisoquinolin-1(2H)-one. In another embodiment an HDI compound of this invention is 2-(3-fluoro-4-hydroxyphenyl)-6,8-dihydroxy-1-oxo-1,2-dihydroisoquinoline-4-carbonitrile. In another embodiment an HDI compound of this invention is 6-hydroxy-2-(4-hydroxyphenyl)-8-vinylisoquinolin-1(2H)-one. In another embodiment an HDI compound of this invention is 4-bromo-6-hydroxy-2-(4-hydroxyphenyl)-8-vinylisoquinolin-1(2H)-one. In another embodiment an HDI compound of this invention is 6,8-dihydroxy-2-(4-hydroxyphenyl)-4-(4-methoxyphenyl)isoquinolin-1(2H)-one. In another embodiment an HDI compound of this invention is 6,8-dihydroxy-2-(4-hydroxyphenyl)-4-phenylisoquinolin-1(2H)-one. In another embodiment an HDI compound of this invention is (E)-6,8-dihydroxy-2-(4-hydroxyphenyl)-4-(prop-1-enyl)isoquinolin-1(2H)-one. In another embodiment an HDI compound of this invention is (E)-ethyl 3-(8-hydroxy-6-methoxy-2-(4-methoxyphenyl)-1-oxo-1,2-dihydroisoquinolin-4-yl)acrylate. In another embodiment an HDI compound of this invention is (E)-3-(6-hydroxy-2-(4-hydroxy phenyl)-1-oxo-1,2-dihydroisoquinolin-4-yl)acrylic acid. In another embodiment an HDI compound of this invention is (E)-3-(6,8-dihydroxy-2-(4-hydroxyphenyl)-1-oxo-1,2-dihydroisoquinolin-4-yl)acrylic acid. In another embodiment, an HDI compound of this inventions is 2-(4-bromomethyl)phenyl-6-hydroxy-4-(4-hydroxyphenyl)isoquinolin-1(2H)-one. In another embodiment and HDI compound of this invention is 6-hydroxy-2-(4-hydroxyphenyl)-4-(4-(trifluoromethyl)phenylisoquinolin-1(2H)-one. In another embodiment an HDI compound of this invention is 6-hydroxy-2-(4-(hydroxymethyl)phenyl)-4-(4-hydroxyphenyl)isoquinolin-1(2H)-one. In another embodiment an HDI compound of this invention is 2-(4-(bromomethyl)-3-hydroxyphenyl)-6-hydroxy-4-(4-(trifluoromethyl)phenyl)isoquinolin-1(2H)-one. In another embodiment an HDI compound of this invention is 4-chloro-6-methoxy-2-(4-methoxyphenyl)-1-oxo-1,2-dihydroisoquinolin-8-yl 4-(trifluoromethyl)benzoate. In another embodiment an HDI compound of this invention is 6-hydroxy-2,4-bis(4-hydroxyphenyl)isoquinolin-1(2H)-one (6); In another embodiment an HDI compound of this invention is 2-(4-(hydroxymethyl)phenyl)-6-methoxy-4-(4-methoxyphenyl)isoquinolin-1(2H)-one (10); In another embodiment an HDI compound of this invention is 2-(4-bromomethyl)phenyl-6-hydroxy-4-(4-hydroxyphenyl)isoquinolin-1(2H)-one (11); In another embodiment an HDI compound of this invention is 6-hydroxy-2-(4-hydroxyphenyl)-4-(4-(trifluoromethyl)phenyl)isoquinolin-1(2H)-one (13); In another embodiment an HDI compound of this invention is 6-hydroxy-2-(4-(hydroxymethyl)phenyl)-4-(4-hydroxyphenyl)isoquinolin-1(2H)-one (14); In another embodiment an HDI compound of this invention is 2-(4-(hydroxymethyl)-3-methoxyphenyl)-6-methoxy-4-(4-(trifluoromethyl)phenyl)isoquinolin-1(2H)-one (25); In another embodiment an HDI compound of this invention is 2-(4-(bromomethyl)-3-hydroxyphenyl)-6-hydroxy-4-(4-(trifluoromethyl)phenyl)isoquinolin-1(2H)-one (26); In another embodiment an HDI compound of this invention is 6-hydroxy-2,4-bis(4-(trifluoromethyl)phenyl)isoquinolin-1(2H)-one (30); In another embodiment an HDI compound of this invention is 2-(4-fluorophenyl)-6-hydroxy-4-(4-(trifluoromethyl)phenyl)isoquinolin-1(2H)-one (34); In another embodiment an HDI compound of this invention is 6-methoxy-2-(4-methoxyphenyl)-4-(4-(trifluoromethyl)phenyl)isoquinolin-1(2H)-one (35); In another embodiment an HDI compound of this invention is 4-(6-hydroxy-2-(4-hydroxyphenyl)-1-oxo-1,2-dihydroisoquinolin-4-yl)-N-methyl benzenesulfonamide (36); In another embodiment an HDI compound of this invention is 6-hydroxy-2-(4-hydroxyphenyl)-4-(4-(methylsulfonyl)phenyl)isoquinolin-1(2H)-one (37); In another embodiment an HDI compound of this invention is 4-(6-hydroxy-2-(4-hydroxyphenyl)-1-oxo-1,2-dihydroisoquinolin-4-yl)benzoic acid (43). In another embodiment an HDI compound of this invention is 2-benzyl-6-hydroxy-4-(3,4,5-trifluorophenyl)isoquinolin-1(2H)-one (75); In another embodiment an HDI compound of this invention is 6-hydroxy-2-(4-hydroxyphenyl)-4-(3,4,5-trifluorophenyl)isoquinolin-1(2H)-one (79); In another embodiment an HDI compound of this invention is 4-(3,4,5-trifluorophenyl)isoquinolin-1(2H)-one (90); In another embodiment an HDI compound of this invention is 2-benzyl-6-hydroxy-4-(3,4,5-trifluorophenyl)isoquinolin-1(2H)-one (75); In another embodiment an HDI compound of this invention is methyl 6-hydroxy-2-(4-hydroxyphenyl)-1-oxo-4-(3,4,5-trifluorophenyl)-1,2-dihydroisoquinoline-8-carbimidate (100); In another embodiment an HDI compound of this invention is 6-hydroxy-2-(4-hydroxyphenyl)-1-oxo-4-(3,4,5-trifluorophenyl)-1,2-dihydroisoquinoline-8-carboxamide (100A); In another embodiment an HDI compound of this invention is 4-(3-fluoro-4-(trifluoromethyl)phenyl)-6-hydroxy-2-(4-hydroxyphenyl)-1-oxo-1,2-dihydroisoquinoline-8-carbonitrile (102); In another embodiment an HDI compound of this invention is methyl-4-(3-fluoro-4-(trifluoromethyl)phenyl)-6-hydroxy-2-(4-hydroxyphenyl)-1-oxo-1,2-dihydroisoquinoline-8-carbimidate (102A); In another embodiment an HDI compound of this invention is 4-(3-fluoro-4-(trifluoromethyl)phenyl)-6-hydroxy-2-(4-hydroxyphenyl)-1-oxo-1,2-dihydroisoquinoline-8-c arboxamide (102B); 6-hydroxy-2-(1-oxo-1,3-dihydroisobenzofuran-5-yl)isoquinolin-1(2H)-one (104); In another embodiment an HDI compound of this invention is methyl 2-(bromomethyl)-4-(6-hydroxy-1-oxoisoquinolin-2(1H)-yl)-benzoate (104A); In another embodiment an HDI compound of this invention is 4-bromo-6,8-dihydroxy-2-(3-hydroxyphenyl)isoquinolin-1(2H)-one (213);

or their prodrug, isomer, metabolite, pharmaceutically acceptable salt, polymorph, crystal, N-oxide, hydrate or any combination thereof.

In one embodiment, an HDI compound of this invention is 4-(3-fluoro-4-(trifluoromethyl)phenyl)-6-((2-(trimethylsilyl)ethoxy)methoxy)isoquinolin-1(2H)-one (214), or its prodrug, analog, isomer, metabolite, dermivative, pharmaceutically acceptable salt, pharmaceutical product, polymorph, crystal, impurity, N-oxide, ester, hydrate, or any combination thereof.

In one embodiment, an HDI compound of this invention is 4-(4-(3-fluoro-4-(trifluoromethyl)phenyl)-6-hydroxy-1-oxoisoquinolin-2(1H)-yl)benzamide (215), or its prodrug, analog, isomer, metabolite, dermivative, pharmaceutically acceptable salt, pharmaceutical product, polymorph, crystal, impurity, N-oxide, ester, hydrate, or any combination thereof.

In one embodiment, the HDI compound is 6-hydroxy-2-(4-hydroxyphenyl)-1-oxo-4-(3,4,5-trifluorophenyl)-1,2-dihydroisoquinoline-8-carbonitrile (85), or its prodrug, analog, isomer, metabolite, derivative, pharmaceutically acceptable salt, pharmaceutical product, polymorph, crystal, impurity, N-oxide, ester, hydrate, or any combination thereof.

In one embodiment, the HDI compound is 6-hydroxy-2-(4-hydroxyphenyl)-4-phenylisoquinolin-1(2H)-one, (15a) or its prodrug, analog, isomer, metabolite, derivative, pharmaceutically acceptable salt, pharmaceutical product, polymorph, crystal, impurity, N-oxide, ester, hydrate, or any combination thereof.

In one embodiment, the HDI compound is 6,8-dihydroxy-2-(4-hydroxyphenyl)-4-(4-methoxyphenyl)isoquinolin-1(2H)-one, (15g) or its prodrug, analog, isomer, metabolite, derivative, pharmaceutically acceptable salt, pharmaceutical product, polymorph, crystal, impurity, N-oxide, ester, hydrate, or any combination thereof.

In one embodiment, the HDI compound is 6,8-dihydroxy-2-(4-hydroxyphenyl)-4-phenylisoquinolin-1(2H)-one, (15h) or its prodrug, analog, isomer, metabolite, derivative, pharmaceutically acceptable salt, pharmaceutical product, polymorph, crystal, impurity, N-oxide, ester, hydrate, or any combination thereof.

In one embodiment, the HDI compound is (E)-3-(6,8-dihydroxy-2-(4-hydroxyphenyl)-1-oxo-1,2-dihydroisoquinolin-4-yl)acrylic acid, (151) or its prodrug, analog, isomer, metabolite, derivative, pharmaceutically acceptable salt, pharmaceutical product, polymorph, crystal, impurity, N-oxide, ester, hydrate, or any combination thereof.

In one embodiment, the HDI compound is 2-(4-bromomethyl)phenyl-6-hydroxy-4-(4-hydroxyphenyl)isoquinolin-1(2H)-one, (11) or its prodrug, analog, isomer, metabolite, derivative, pharmaceutically acceptable salt, pharmaceutical product, polymorph, crystal, impurity, N-oxide, ester, hydrate, or any combination thereof.

In one embodiment, the HDI compound is 6-hydroxy-2-(4-hydroxyphenyl)-4-(4-(trifluoromethyl)phenylisoquinolin-1(2H)-one, (13) or its prodrug, analog, isomer, metabolite, derivative, pharmaceutically acceptable salt, pharmaceutical product, polymorph, crystal, impurity, N-oxide, ester, hydrate, or any combination thereof.

In one embodiment, the HDI compound is 6-hydroxy-2-(4-(hydroxymethyl)phenyl)-4-(4-hydroxyphenyl)isoquinolin-1(2H)-one, (14) or its prodrug, analog, isomer, metabolite, derivative, pharmaceutically acceptable salt, pharmaceutical product, polymorph, crystal, impurity, N-oxide, to ester, hydrate, or any combination thereof.

In one embodiment, the HDI compound is 2-(4-(bromomethyl)-3-hydroxyphenyl)-6-hydroxy-4-(4-(trifluoromethyl)phenyl)isoquinolin-1(2H)-one, (26) or its prodrug, analog, isomer, metabolite, derivative, pharmaceutically acceptable salt, pharmaceutical product, polymorph, crystal, impurity, N-oxide, ester, hydrate, or any combination thereof.

In some embodiments, an HDI compound of this invention, compositions of this invention or uses thereof may comprise any combinations of such HDI compound as described herein.

One of ordinary skill in the art will recognize that some of the compounds of this invention may exist in different tautomeric forms.

In certain embodiments, the compound of Formula I is selected from:

6-hydroxy-2-(4-hydroxyphenyl)-4-phenylisoquinolin-1(2H)-one (15a); 6,8-dihydroxy-2-(4-hydroxyphenyl)-4-(4-methoxyphenyl)isoquinolin-1(2H)-one (15g); 6,8-dihydroxy-2-(4-hydroxyphenyl)-4-phenylisoquinolin-1(2H)-one (15h); (E)-3-(6,8-dihydroxy-2-(4-hydroxyphenyl)-1-oxo-1,2-dihydroisoquinolin-4-yl)acrylic acid (15l); 2-(4-bromomethyl)phenyl-6-hydroxy-4-(4-hydroxyphenyl)isoquinolin-1(2H)-one (11); 6-hydroxy-2-(4-hydroxyphenyl)-4-(4-(trifluoromethyl)phenylisoquinolin-1(2H)-one (13); 6-hydroxy-2-(4-(hydroxymethyl)phenyl)-4-(4-hydroxyphenyl)isoquinolin-1(2H)-one (14); 2-(4-(bromomethyl)-3-hydroxyphenyl)-6-hydroxy-4-(4-(trifluoromethyl)phenyl)isoquinolin-1(2H)-one (26); 6-hydroxy-2-(4-hydroxyphenyl)-1-oxo-4-(3,4,5-trifluorophenyl)-1,2-dihydroisoquinoline-8-carbonitrile (85); 3-(4-(3-fluoro-4-(trifluoromethyl)phenyl)-6-hydroxy-1-oxoisoquinolin-2(1H)-yl)benzamide (214); and 4-(4-(3-fluoro-4-(trifluoromethyl)phenyl)-6-hydroxy-1-oxoisoquinolin-2(1H)-yl)benzamide (215).

In one embodiment, this invention provides a compound of Formula I-XIII, or a prodrug, isomer, tautomer, metabolite, pharmaceutically acceptable salt, polymorph, crystal, N-oxide, hydrate or any combination thereof. In one embodiment, this invention provides a compound of Formula I-XII, or an isomer, tautomer, metabolite, pharmaceutically acceptable salt, polymorph, N-oxide, hydrate or any combination thereof. In one embodiment, this invention provides a compound of Formula I-XIII, or an isomer, tautomer, pharmaceutically acceptable salt, polymorph, N-oxide, hydrate or any combination thereof. In one embodiment, this invention provides a compound of Formula I-XIII, or an isomer, tautomer, pharmaceutically acceptable salt, N-oxide, hydrate or any combination thereof. In one embodiment, this invention provides a compound of Formula I-XIII, or an isomer, pharmaceutically acceptable salt, or any combination thereof.

In another embodiment, this invention provides an isomer of a compound of Formula I-XIII In another embodiment, this invention provides a metabolite of a compound of Formula I-XIII In another embodiment, this invention provides a pharmaceutically acceptable salt of a compound of Formula I-XIII In another embodiment, this invention provides a hydrate of a compound of Formula I-XIII In another embodiment, this invention provides a tautomer of a compound of Formula I-XIII In another embodiment, this invention provides an N-oxide of a compound of Formula I-XIII In another embodiment, this invention provides a prodrug of a compound of Formula I-XIII In another embodiment, this invention provides a polymorph of a compound of Formula I-XIII In another embodiment, this invention provides a crystal of a compound of Formula I-XIII

In one embodiment, a compound of this invention is a hydroxysteroid dehydrogenase inhibitor (HSDi). In one embodiment, an HSDi is an aldo-keto reductase inhibitor. In one embodiment, an HSDi, is a AKR1C3 inhibitor.

In certain embodiments, the compounds of the present invention are active as selective hydroxysteroid dehydrogenase inhibitors (HSDi). In one embodiment, the compounds of the present invention are active as selective inhibitors of AKR1C. In another embodiment, the compounds of the present invention are active as selective inhibitors of AKR1C3.

In one embodiment, a selective inhibitor of AKR1C3 is 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof.

In another embodiment, a selective inhibitor of AKR1C3 is 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof.

In yet another embodiment, a selective inhibitor of AKR1C3 is 6,8-dihydroxy-2-(4-hydroxyphenyl)-4-phenylisoquinolin-1(2H)-one e (15h), or its prodrug, isomer, tautomer, metabolite, pharmaceutically acceptable salt, polymorph, crystal, N-oxide, hydrate or any combination thereof.

In still another embodiment, a selective inhibitor of AKR1C3 is (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof.

In a further embodiment, a selective inhibitor of AKR1C3 is 2-(4-bromomethyl)phenyl-6-hydroxy-4-(4-hydroxyphenyl) isoquinolin-1(2H)-one (11), or its prodrug, isomer, tautomer, metabolite, pharmaceutically acceptable salt, polymorph, crystal, N-oxide, hydrate or any combination thereof.

In one embodiment, a selective inhibitor of AKR1C3 is 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof.

In one embodiment, a selective inhibitor of AKR1C3 is 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof.

In one embodiment, a selective inhibitor of AKR1C3 is 6-hydroxy-2-(4-(hydroxymethyl)phenyl)-4-(4-hydroxyphenyl) isoquinolin-1(2H)-one (14), or its prodrug, isomer, tautomer, metabolite, pharmaceutically acceptable salt, polymorph, crystal, N-oxide, hydrate or any combination thereof.

In one embodiment, a selective inhibitor of AKR1C3 is 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof.

In one embodiment, a selective inhibitor of AKR1C3 is 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof.

In one embodiment, a selective inhibitor of AKR1C3 is 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or to any combination thereof.

The term “alkyl” refers, in one embodiment, to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. In another embodiment, the cyclic alkyl group has 3-8 carbons. In another embodiment, the cyclic alkyl group has 3-12 carbons. In another embodiment, the branched alkyl is an alkyl substituted by alkyl side chains of 1 to 5 carbons. In another embodiment, the branched alkyl is an alkyl substituted by haloalkyl side chains of 1 to 5 carbons. The alkyl group may be unsubstituted or substituted by a halogen, haloalkyl, hydroxyl, cyano, alkoxy carbonyl, amido, alkylamido, dialkylamido, nitro, amino, alkylamino, dialkylamino, carboxyl, thio and/or thioalkyl.

An “alkenyl” group refers, in another embodiment, to an unsaturated hydrocarbon, including straight chain, branched chain and cyclic groups having one or more double bonds. The alkenyl group may have one double bond, two double bonds, three double bonds, etc. In another embodiment, the alkenyl group has 2-12 carbons. In another embodiment, the alkenyl group has 2-6 carbons. In another embodiment, the alkenyl group has 2-4 carbons. In another embodiment the alkenyl group is vinyl (—CH═CH₂). Examples of alkenyl groups are vinyl, propenyl, butenyl, cyclohexenyl, etc. The alkenyl group may be unsubstituted or substituted by a halogen, hydroxy, cyano, alkoxy carbonyl, amido, alkylamido, dialkylamido, nitro, amino, alkylamino, dialkylamino, carboxyl, thio and/or thioalkyl.

The term “cycloalkyl” refers to a monocyclic, bicyclic or tricyclic nonaromatic saturated hydrocarbon radical having 3 to 10 carbon atoms, such as 3 to 8 carbon atoms, for example, 3 to 6 carbon atoms. Non limiting examples of cycloalkyl groups include cyclopropyl, cyclobutyl, cyclopentyl, cyclohexyl, cycloheptyl, cyclooctyl, norbornyl, 1-decalin, adamant-1-yl, and adamant-2-yl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl.

A “haloalkyl” group refers, in another embodiment, to an alkyl group as defined above, which is substituted by one or more halogen atoms, e.g. by F, Cl, Br or I.

An “aryl” group refers, in another embodiment, to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers, in another embodiment, to an OH group. In some embodiments, when R₁, R₂ or R₃ of the compounds of the present invention is OR, then R is not OH.

In one embodiment, the term “halo” refers to a halogen, such as F, Cl, Br or I.

In another embodiment, the phrase “phenol” refers to an alcohol (OH) derivative of benzene.

A “heterocycle” group refers, in one embodiment, to a ring structure comprising in addition to carbon atoms, sulfur, oxygen, nitrogen or any combination thereof, as part of the ring. In another embodiment the heterocycle is a 3-12 membered ring. In another embodiment the heterocycle is a 6 membered ring. In another embodiment the heterocycle is a 5-7 membered ring. In another embodiment the heterocycle is a 4-8 membered ring. In another embodiment, the heterocycle group may be unsubstituted or substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the heterocycle ring may be fused to another saturated or unsaturated cycloalkyl or heterocyclic 3-8 membered ring. In another embodiment, the heterocyclic ring is a saturated ring. In another embodiment, the heterocyclic ring is an unsaturated ring. Examples of a heterocycle group comprise pyridine, piperidine, morpholine, piperazine, thiophene, pyrrole or indole.

In one embodiment the 5-14 member saturated or unsaturated, substituted or unsubstituted carbocyclic or heterocyclic ring comprises a phenyl, naphthalene, anthracene, pyridine, piperidine, thiophene, morpholine, piperazine, pyrimidine, cyclohexyl, cycloheptyl, pyrrole, pyrazole, furan, oxazole, quinoline, pyrazine or indole groups.

In one embodiment unsaturated cycloalkyl or heterocycloalkyl groups refer to cycloalkyl or heterocycloalkyl comprising at list one double bond. In another embodiment unsaturated cycloalkyl or heterocycloalkyl refer to an aryl or heteroaryl group.

In some embodiments, protected hydroxyl includes the incorporation of a substituent bonded to an oxygen atom bound to a benzene ring, wherein the substituent may be readily removed. In some embodiments, phenolic protecting groups may comprise a: methyl ether, methoxymethyl (MOM) ether, benzoyloxymethyl (BOM) ether, methoxyethoxymethyl (MEM) to ether, 2-(trimethylsilyl)ethoxymethyl(SEM) ether, methylthiomethyl (MTM) ether, phenylthiomethyl (PTM) ether, azidomethyl ether, cyanomethyl ether, 2,2-dichloro-1,1-difluoroethyl ether, 2-chloroethyl ether, 2-bromoethyl ether, tetrahydropyranyl (THP) ether, 1-ethoxyethyl (EE) ether, phenacyl ether, 4-bromophenacyl ether, cyclopropylmethyl ether, allyl ether, propargyl ether, isopropyl ether, cyclohexyl ether, t-butyl ether, benzyl ether, 2,6-dimethylbenzyl ether, 4-methoxybenzyl ether, o-nitrobenzyl ether, 2,6-dichlorobenzyl ether, 3,4-dichlorobenzyl ether, 4-(dimethylamino)carbonylbenzyl ether, 4-methylsulfinylbenzyl ether, 4-anthrylmethyl ether, 4-picolyl ether, heptafluoro-p-tolyl, tetrafluoro-4-pyridyl ether, trimethylsilyl (TMS) ether, t-butyldimethylsilyl (TBDMS) ether, t-butyldiphenylsilyl (TBDPS) ether, triisopropylsilyl (TIPS) ether, aryl formate, arylacetate, aryl levulinate, arylpivaloate, aryl benzoate, aryl 9-fluorencarboxylate, aryl methyl carbonate, 1-adamantyl carbonate, t-butyl carbonate, 4-methylsulfinylbenzyl carbonate, 2,4-dimethylpent-3-yl carbonate, aryl-2,2,2-trichloroethyl carbonate, aryl benzyl carbonate, aryl carbamate, dimethylphosphinyl ester (Dmp-OAr), dimethylphosphinothionyl ester (Mpt-OAr), diphenylphosphinothionyl ester (Dpt-OAr), aryl methanesulfonate, aryl toluenesulfonate or aryl 2-formylbenzenesulfonate.

One of ordinary skill in the art will recognize that some compounds of Formula I-XIII can exist in different tautomeric and/or conformational and/or geometrical isomeric and/or optical isomeric forms. All of these forms, including cis isomers, trans isomers, diastereomic mixtures, racemates, nonracemic mixtures of enantiomers, substantially pure, and pure enantiomers, are within the scope of the present invention. Substantially pure enantiomers contain no more than 5% w/w of the corresponding opposite enantiomer, e.g., no more than 2%, such as no more than 1%.

In one embodiment, the term “isomer” is meant to encompass optical isomers of the compound. In one embodiment, the term “isomer” is meant to encompass stereoisomers of the compound. In one embodiment, the term “isomer” is meant to encompass conformational isomers. one embodiment, the term “isomer” is meant to encompass tautomers. It is to be understood that the present invention encompasses any optically-active, or stereroisomeric form, or mixtures thereof, and use of these for any application is to be considered within the scope of this invention.

In one embodiment, the compounds of Formula I-XIII are substantially pure (E)-isomers. In another embodiment, the compounds of Formula I-XIII are substantially pure (Z)-isomers. In another embodiment, the compounds of Formula I-XIII are a mixture of (E) and the (Z) isomers. In one embodiment, the compounds of Formula I-XIII are pure (E)-isomers. In another embodiment, the compounds of Formula I-XIII are pure (Z)-isomers. In one embodiment, the compounds of Formula I-XIII are substantially pure (R)-isomers. In another embodiment, the compounds of Formula I-XIII are substantially pure (S)-isomers. In another embodiment, the compounds of Formula I-XIII are a mixture of (R) and the (S) isomers. In one embodiment, the compounds of Formula I-XIII are pure (R)-isomers. In another embodiment, the compounds of Formula I-XIII are pure (S)-isomers.

As is known to an ordinary skilled artisan, optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of Formula I-XIII can likewise be obtained by utilizing optically active starting materials in chiral synthesis processes under reaction conditions which do not cause racemization.

In addition, one of ordinary skill in the art will recognize that the compounds can be used in different enriched isotopic forms, e.g., enriched in the content of ²H, ³H, ¹¹C¹³C and/or ¹⁴C. In one particular embodiment, the compounds are deuterated. Such deuterated forms can be made via the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in, for example, Dean, Dennis C.; Editor. Recent Advances in the Synthesis and applications of Radio labeled Compounds for Drug Discovery and Development. [In: Curr. Pharm. Des., 2000; 6(10)] (2000), 110 pp. CAN 133:68895 AN 2000:473538 CAPLUS; Kabalka, George W.; Varma, Rajender S. The synthesis of radio labeled compounds via organometallic intermediates. Tetrahedron (1989), 45(21), 6601-21, CODEN: TETRAB ISSN: 0040-4020. CAN 112:20527 AN 1990:20527 CAPLUS; and Evans, E. Anthony. Synthesis of radio labeled compounds, J. Radioanal. Chem. (1981), 64(1-2), 9-32. CODEN: JRACBN ISSN: 0022-4081, CAN 95:76229 AN 1981:476229 CAPLUS.

Where applicable, the present invention also relates to useful forms of the compounds as disclosed herein, such as base free forms, and pharmaceutically acceptable salts or prodrugs of all the compounds of the present invention for which salts or prodrugs can be prepared. Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, a hydrobromide, a hydroformate, a maleate or a sodium salt.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formula I-XIII can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in crystal structure as crystalline structures of two different chemical compounds.

In one embodiment, the compounds of Formula I-XIII can exist in different solvate forms. Solvates of the compounds of the invention may form when solvent molecules are incorporated into the crystalline lattice structure of the compound molecule during the crystallization process. For example, a compound of Formula I-XIII may exist in the form of a hydrate, such as, for example, a monohydrate, hemihydrate, sesquihydrate, dihydrate, trihydrate, or any combination thereof.

The term “prodrug” means a compound that is a drug precursor which upon administration to a subject undergoes chemical conversion by metabolic or chemical processes to yield a compound of the present invention. Since prodrugs are known to enhance numerous desirable qualities of pharmaceuticals (e.g., solubility, bioavailability, manufacturing, etc.) the compounds of the present invention may be delivered in prodrug form. Thus, the present invention includes prodrugs of the disclosed compounds and methods of delivering the same. Prodrugs of a compound of the present invention may be prepared by modifying functional groups present in the compound in such a way that the modifications are cleaved, either in routine manipulation or in vivo, to the parent compound. Accordingly, prodrugs include, for example, compounds of the present invention wherein a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, isopropyl, butyl, isobutyl, sec-butyl, tert-butyl, cyclopropyl, phenyl, benzyl, and phenethyl esters, and the like. Such prodrugs are considered to be within the scope of this invention.

This invention provides, in other embodiments, metabolites of a compound of Formula I-XIII In one embodiment, the term “metabolite” refers to any substance produced from another substance by metabolism or a metabolic process.

In another aspect, the present invention relates to methods for preparing the compounds of Formula I-XIII The compounds of the present invention may be prepared by conventional methods, known to one or ordinary skill in the art. For example, some of the processes that can be used are given in the general reaction schemes outlined below. Modifications to these exemplary reaction schemes will be readily apparent to those skilled in the art upon reading the present disclosure and examples which follow. All starting materials are commercially available or can be conventionally prepared from known starting materials, unless otherwise indicated.

### General Scheme for Synthesis of HSDi Compounds.

As used herein, the term “HSDi” may be used interchangeably with “HDI”.

In one embodiment, this invention provides general and specific synthetic routes for embodiments of isoquinolinones and isoquinolin-6-ols.

Some embodiments of a synthetic procedure for some of the HSDIs are provided below:

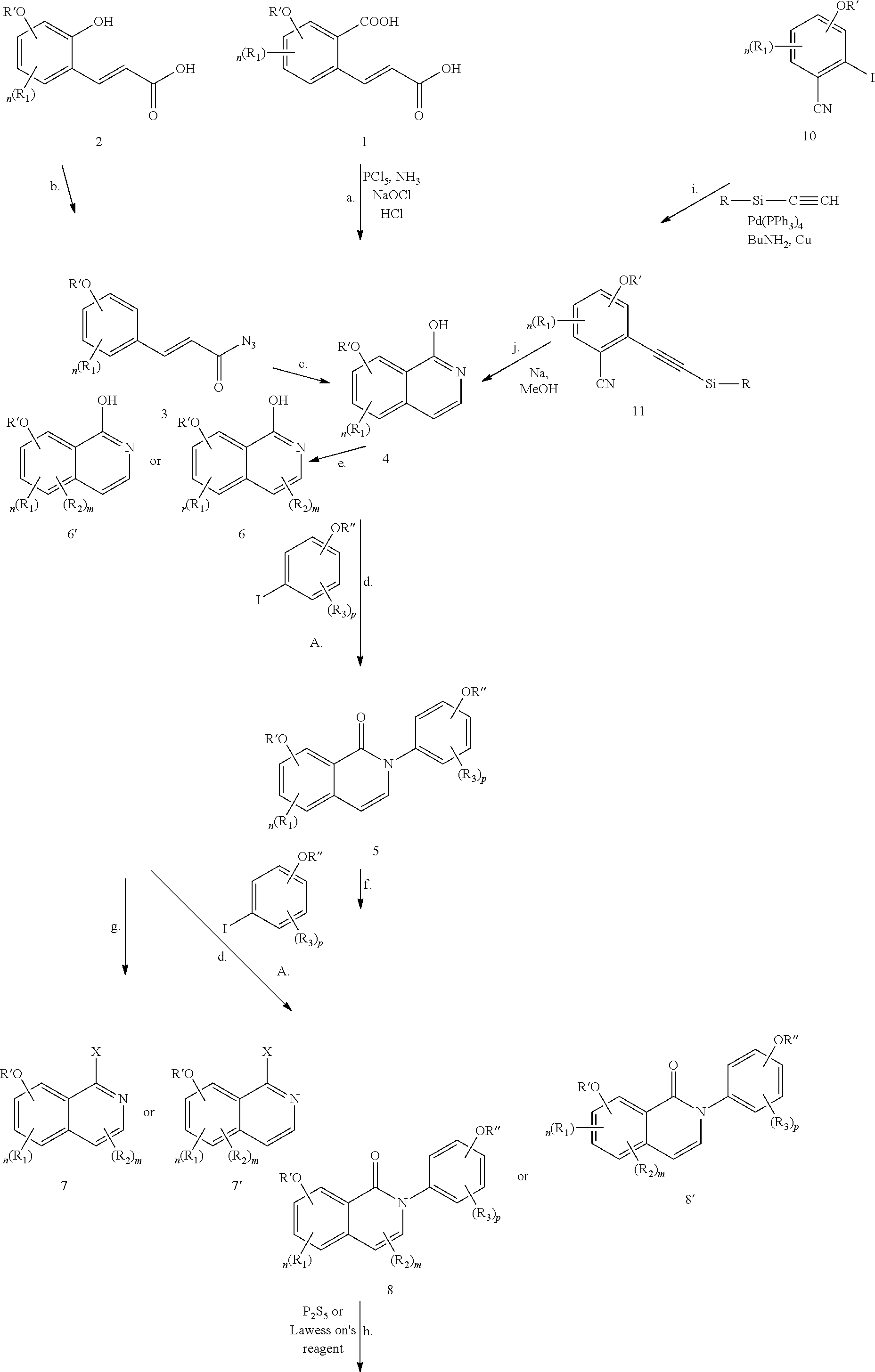

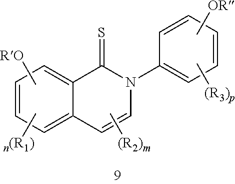

Intermediate compound 4 can be prepared by three different paths starting from 2-(2-carboxy-vinyl)benzoic acid (compound 1) via step a; or starting with 3-phenyl-acrylic acid, (compound 2) together with sodium azide (step b) to obtain an acyl derivative of compound 3, followed by Curtius rearrangement and a cyclization step (step c) in the presence of diphenyl ether and tributylamine at 230° C. to obtain compound 4; or starting with 2-iodo benzonitrile (compound 10) via the Sonogashira reaction (step i) followed by methanolysis (step j) to obtain compound 4.

Compound 4 is further coupled with an iodo substituted formula A (step d), yielding compound 5, which may be further brominated, chlorinated, or iodinated (using NBS, NCS, or NIS, respectively) followed by further substitutions to obtain the desired R₂ group (step f) compound 8 or compound 8′, or obtain the sulfone compound 9 using P₂S₅ reagent (step h). Compounds 8 or 9 can be optionally demethylated with BBr₃ to yield the phenolic products, however if step h is executed, then the phenol must be protected.

Alternatively, compound 4 may be brominated, chlorinated, or iodinated (using NBS, NCS, or NIS, respectively) and further substituted (step e) to obtain the desired R₂ of compound 6 or 6′. Compound 6 or 6′ may be coupled together with an iodo substituted formula A (step d), yielding compound 8 or 8′, or the OH group of compound 6 or 6′ is further substituted (step g) to obtain the desired X group of compound 7 or compound 7′.

In some embodiments this invention provides synthetic route for embodiments of 4-halogenated isoquinolinones. For example, one embodiment of a synthetic procedure for a compound of this invention, 4-bromo-6-hydroxy-2-(4-hydroxyphenyl)isoquinolin-1(2H)-one, is as follows:

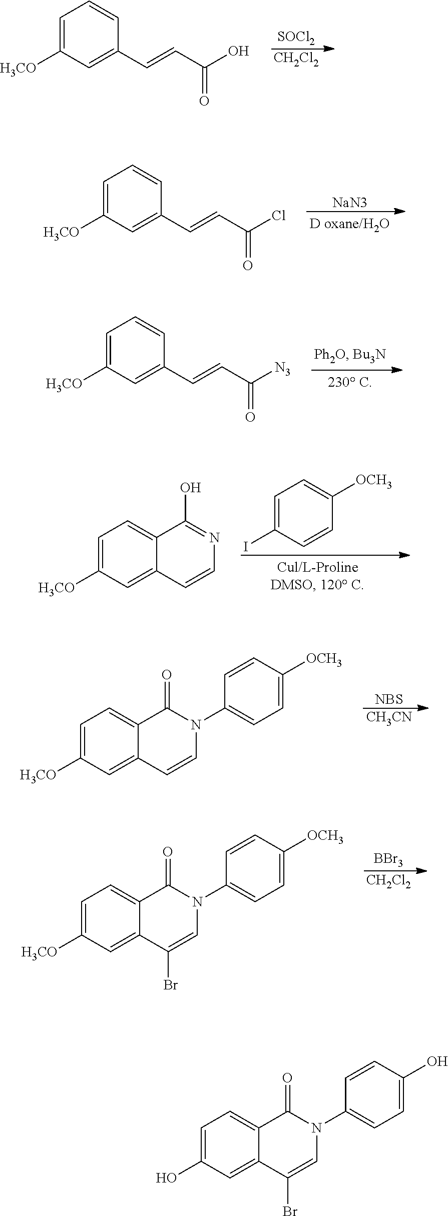

In some embodiments this invention provides synthetic route for embodiments of 6,8-dihydroxy-isoquinolinones. An example of these embodiments of this invention provides a synthetic route for 4-bromo-6,8-dihydroxy-2-(4-hydroxyphenyl)isoquinolin-1(2H)-one (12u).

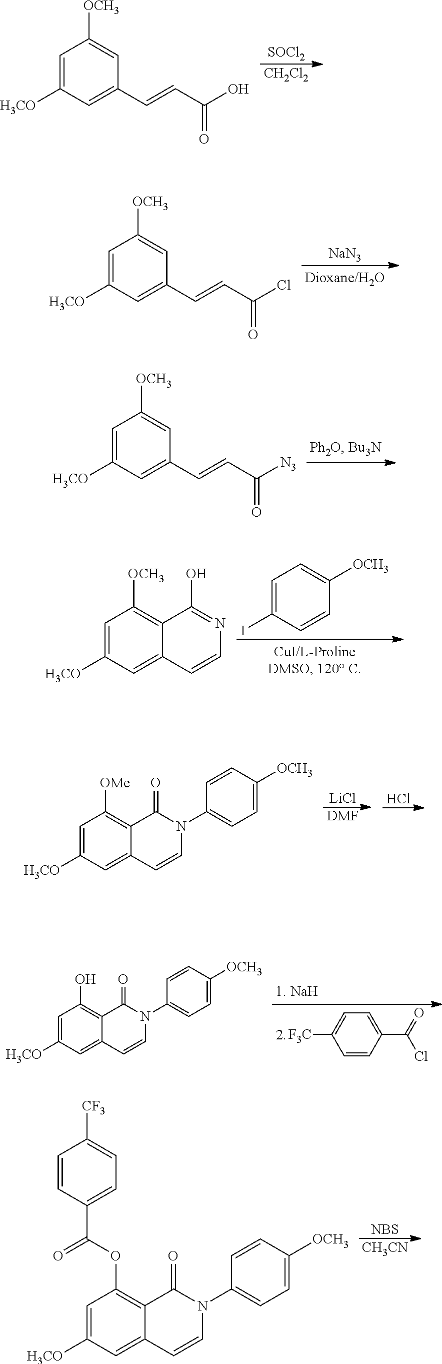

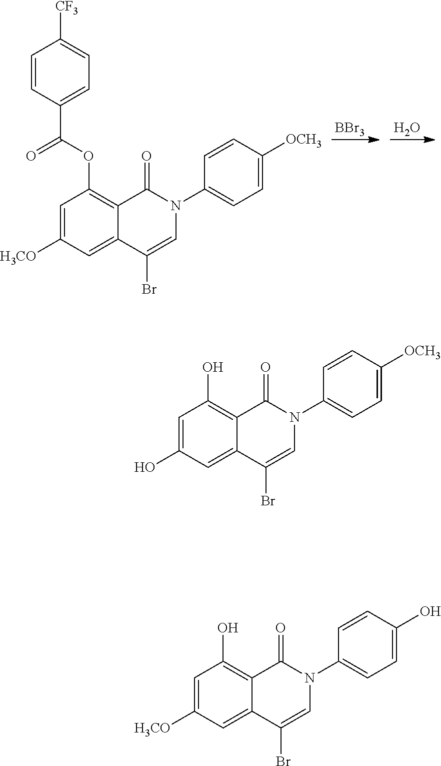

In some embodiments this invention provides synthetic route for embodiments of 4-alkenyl isoquinolinones. An example of these embodiments of this invention provides a synthetic route for 6-hydroxy-2-(4-hydroxyphenyl)-4-vinylisoquinolin-1(2H)-one (14f) compound.

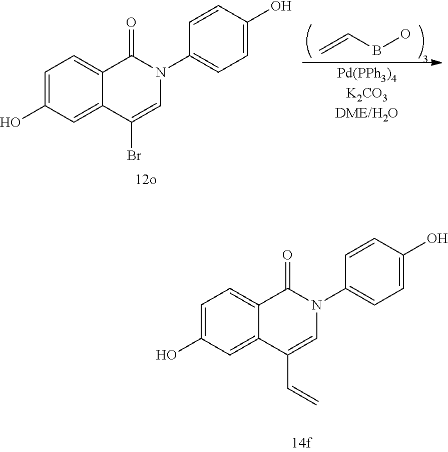

In some embodiments this invention provides synthetic route for embodiments of 4-carbonitrile derivatives of 1-oxo-1,2-dihydroisoquinolines. For example, this invention provides synthetic routes for 6-hydroxy-2-(4-hydroxyphenyl)-1-oxo-1,2-dihydroisoquinoline-4-carbonitrile (14h).

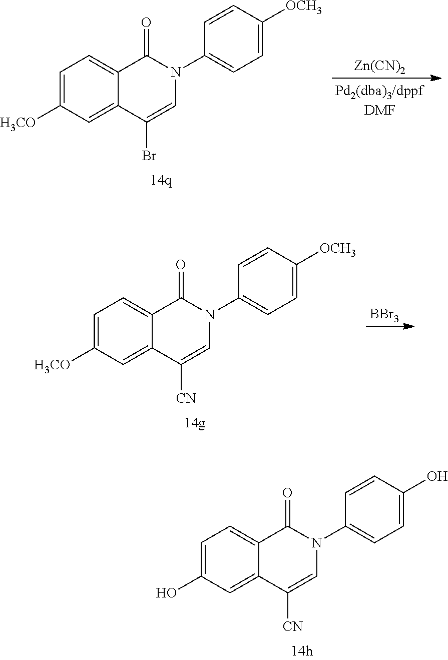

In some embodiments this invention provides synthetic route for embodiments of 8-carbonitrile derivatives of 1-oxo-1,2-dihydroisoquinolines. For example, this invention provides synthetic routes for 4-bromo-6-hydroxy-2-(4-hydroxyphenyl)-1-oxo-1,2-dihydroisoquinoline-8-carbonitrile (14k):

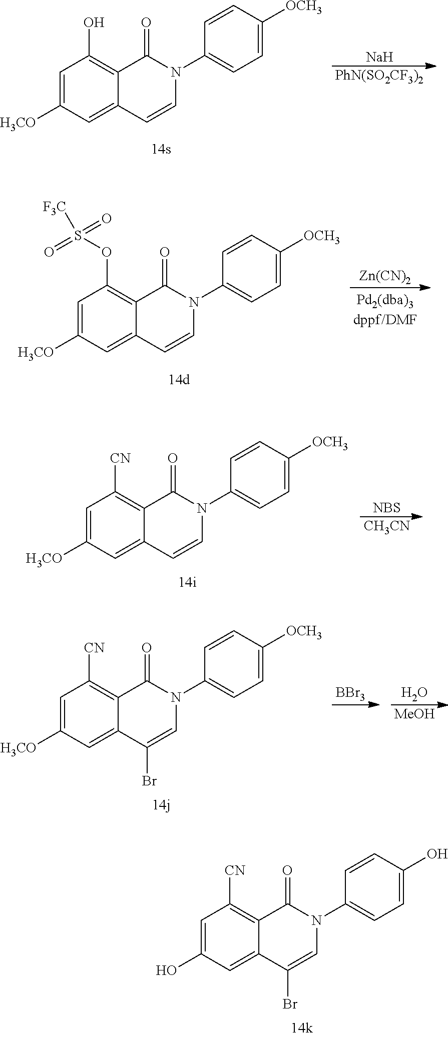

In some embodiments this invention provides synthetic route for 14o compound

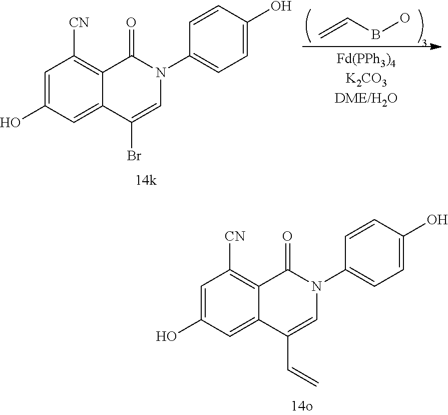

In some embodiments this invention provides synthetic route for 14p compound

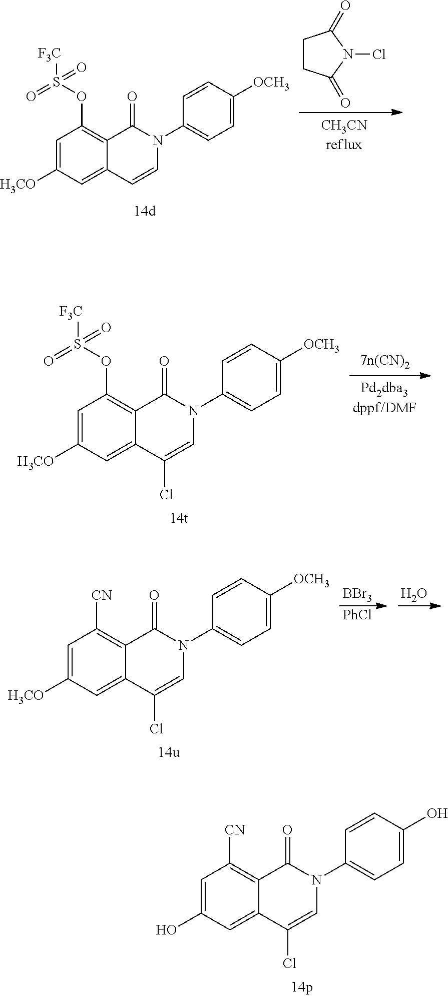

In some embodiments this invention provides synthetic routes for 14xME, 14xME_AC and 14xAC compounds.

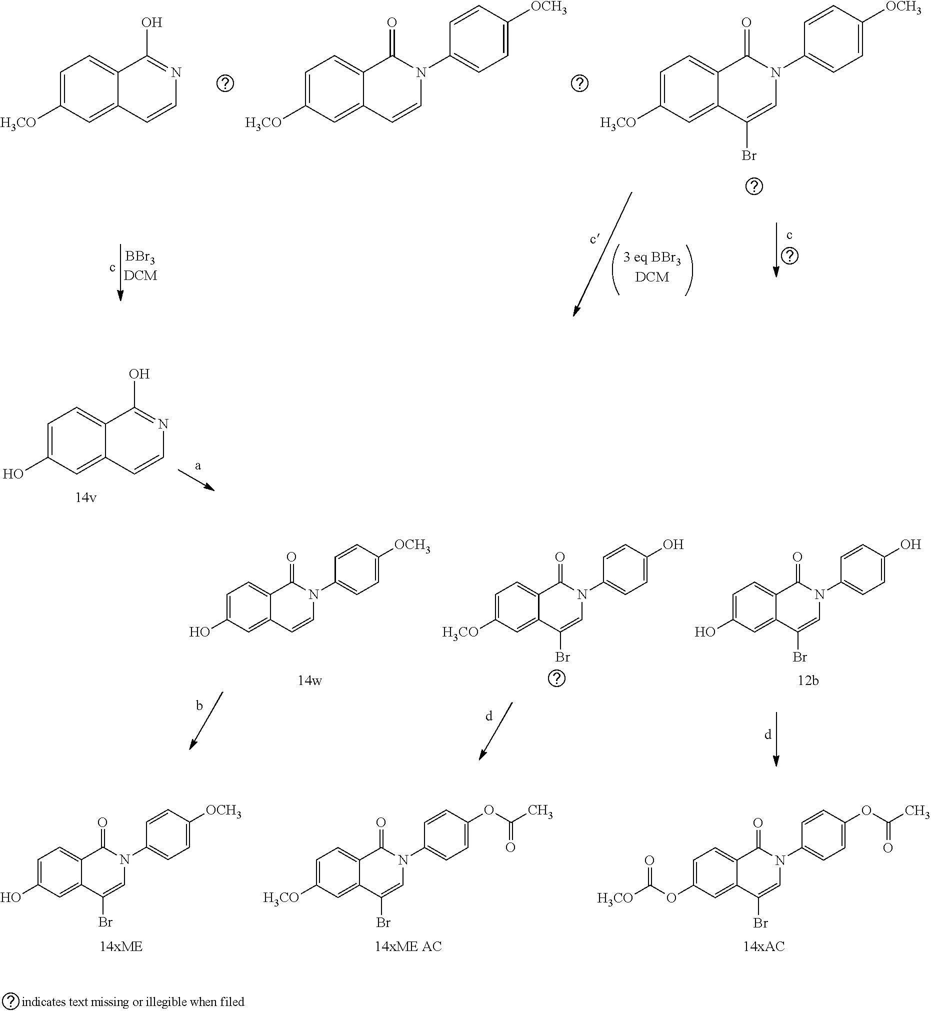

In some embodiments this invention provides synthetic routes for 4-bromo-6-hydroxy-2-(4-hydroxyphenyl)-1-oxo-1,2-dihydroisoquinoline-8-carbimidic acid (14yAM), methyl 4-bromo-6-hydroxy-2-(4-hydroxyphenyl)-1-oxo-1,2-dihydroisoquinoline-8-carboxylate (14yME), and 4-bromo-6-hydroxy-2-(4-hydroxyphenyl)-1-oxo-1,2-dihydroisoquinoline-8-carboxylic acid (14z) compounds.

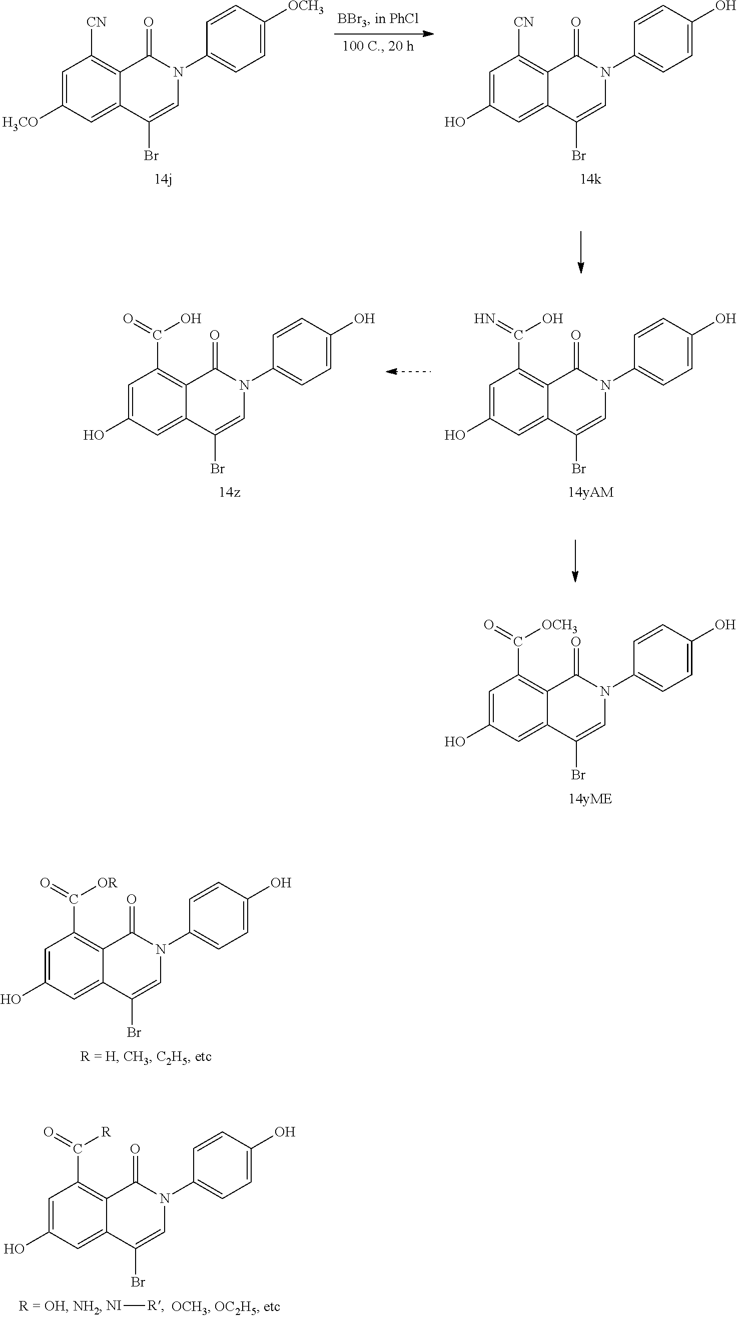

In some embodiments this invention provides synthetic routes for 6-hydroxy-2-(4-hydroxyphenyl)-4-phenylisoquinolin-1(2H)-one (15a).

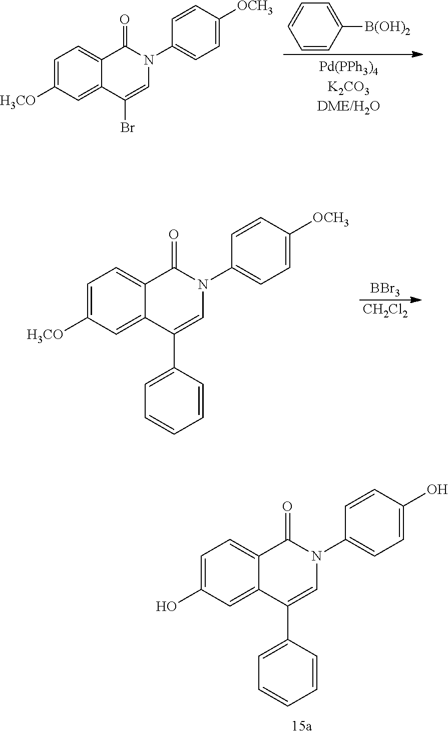 [SMILES: COc1ccc(-n2cc(Br)c3cc(OC)ccc3c2=O)cc1 ; OB(O)c1ccccc1 ; COc1ccc(-n2cc(-c3ccccc3)c3cc(OC)ccc3c2=O)cc1 ; "CH2Cl2" ; "BBr3" ; O=c1c2ccc(O)cc2c(-c2ccccc2)cn1-c1ccc(O)cc1]

In some embodiments the following compounds are synthesized via Suzuki coupling reactions as described for compound 15a.

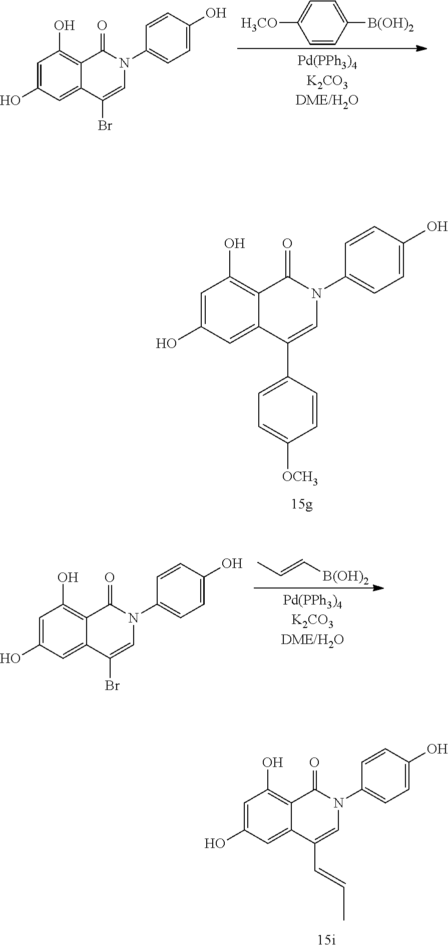

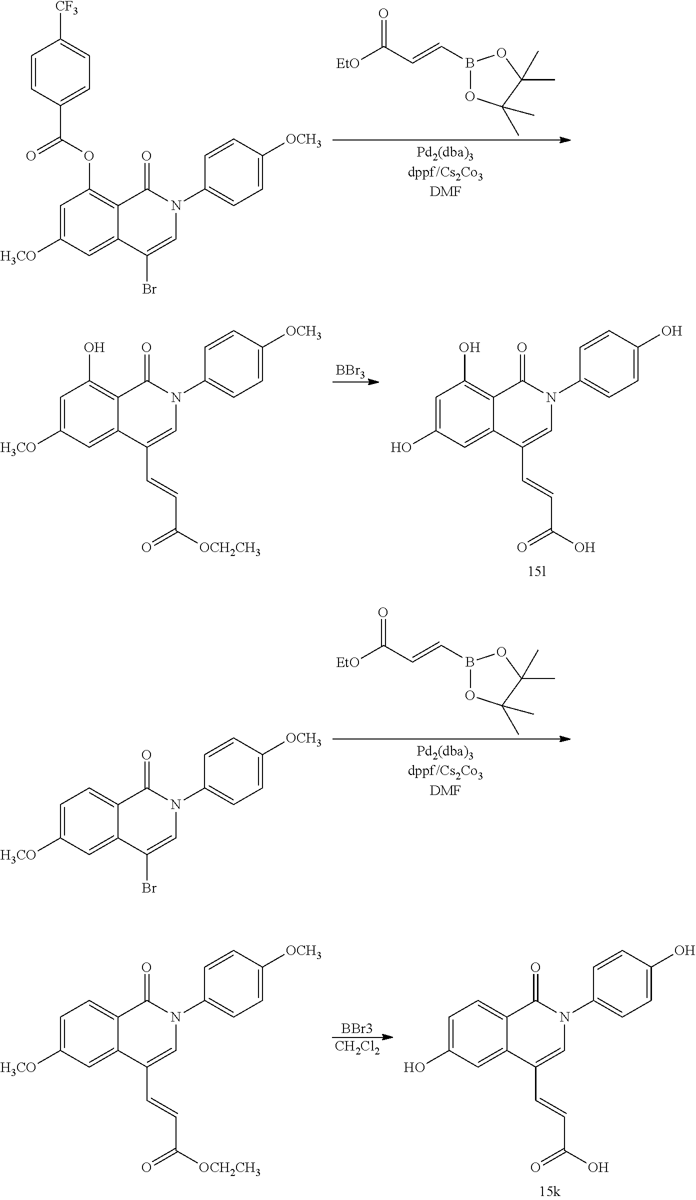

### Pharmaceutical Compositions

The compounds of the invention can be administered alone or as an active ingredient of a formulation. Thus, the present invention also includes pharmaceutical compositions of compounds of formula I-XIII, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage forms is closely related to the therapeutic amounts of the compounds or compositions which are desirable and efficacious for the given treatment application.

Suitable dosage forms include but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. Formulations suitable for oral administration are preferred.

To prepare such pharmaceutical dosage forms, the active ingredient may be mixed with a pharmaceutical carrier according to conventional pharmaceutical compounding techniques. The carrier may take a wide variety of forms depending on the form of preparation desired for administration.

In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients, for example, ingredients that aid solubility or for preservation, may be included. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Treatment methods of the present invention using formulations suitable for oral administration may be presented as discrete units such as capsules, cachets, tablets, or lozenges, each containing a predetermined amount of the active ingredient as, for example, a powder or granules. Optionally, a suspension in an aqueous liquor or a non-aqueous liquid may be employed, to such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration may comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more accessory ingredient(s) selected from, for example, diluents, buffers, flavoring agents, binders, disintegrants, surface active agents, thickeners, lubricants, preservatives (including antioxidants), and the like.

The formulations of the present invention can have immediate release, sustained release, delayed-onset release or any other release profile known to one skilled in the art.

### Methods of Treatment

According to other aspects of the present invention, methods for treating a condition that responds to a hydroxysteroid dehydrogenase inhibitor are provided. In certain embodiments, the compounds of the present invention may be useful as hydroxysteroid dehydrogenase inhibitors. For example, the compounds of the present invention may be useful as AKR1C inhibitors, for example AKR1C3 inhibitors. In one embodiment, the compounds of this invention may be useful as selective inhibitors of AKR1C3.

For example, some embodiments provide methods of treating a condition that responds to a hydroxysteroid dehydrogenase inhibitor comprising administering to a patient in need thereof an effective amount of a compound of the present invention.

In certain embodiments, the present invention provides methods of treatment of conditions related to cancer. For example, the present invention, in certain embodiments, provides methods of treatment of hormone dependent or hormone independent cancers. Non-limiting examples of hormone dependent cancers include prostate cancer, breast cancer uterine fibroids including myomas and uterine cancer including cervical and endometrial cancers. Non-limiting examples of hormone independent cancers include lung cancer including non-small cell lung cancer, bladder cancer, colon cancer, leukemias including acute myelogenous leukemia, and/or lymphoma.

In one embodiment of this invention, a method of treating a condition is the method of treating prostate cancer. As used herein, the term “prostate cancer” refers to prostate cancer, primary prostate cancer, advanced prostate cancer, metastatic prostate cancer, hormone naïve prostate cancer, refractory prostate cancer and/or castration resistant prostate cancer (CRPC) or any combination thereof.

In another embodiment of this invention, a method of treating a condition is the to method of treating breast cancer. As used herein, the term “breast cancer” refers to breast cancer; metastatic breast cancer; advanced breast cancer; refractory breast cancer; AR-positive breast cancer; ER-positive breast cancer, wherein ER-positive breast cancer may refer to ER-alpha-positive breast cancer and/or ER-beta-positive breast cancer; AR-positive refractory breast cancer; ER-positive refractory breast cancer; AR-positive metastatic breast cancer; ER-positive metastatic breast cancer; triple negative breast cancer; and/or breast cancer that has failed SERM (tamoxifen, toremifene), aromatase inhibitor, trastuzumab (Herceptin®), exemestane (Aromasin®), bevacizumab (Avastin®), and/or fulvestrant treatment, or any combination thereof.

As used herein, the term “metastasis” refers to the transfer of a disease from one organ or part thereof to another not directly connected with it. Metastasis can occur for example as a result of transfer of malignant cells from one organ (for example breast) to other organs.

In one embodiment, this invention provides a method of treating a subject suffering from prostate cancer, comprising the step of administering to said subject a compound of this invention, or its isomer, pharmaceutically acceptable salt, pharmaceutical product, crystal, hydrate, N-oxide, prodrug, metabolite or any combination thereof, or a composition comprising the same in an amount effective to treat prostate cancer in the subject.

As used herein, the term “treating” refers to treating, preventing, suppressing, inhibiting or delaying the progression of.

In one embodiment, the methods of this invention are directed to treating prostate cancer. In one embodiment, the methods of this invention are directed to suppressing, reducing the incidence, reducing the severity, or inhibiting prostate cancer. In one embodiment, the methods of this invention are directed to palliative treatment of prostate cancer. In another embodiment, this invention is directed to suppressing prostate cancer. In another embodiment, this invention is directed to reducing the incidence of prostate cancer. In another embodiment, this invention is directed to reducing the severity of prostate cancer. In another embodiment, this invention is directed to inhibiting prostate cancer. In another embodiment, this invention is directed to increasing the survival of a subject with prostate cancer.

In one embodiment, the methods of this invention are directed to treating primary prostate cancer. In one embodiment, the methods of this invention are directed to suppressing, reducing the incidence, reducing the severity, or inhibiting primary prostate cancer. In one embodiment, the methods of this invention are directed to palliative treatment of primary prostate cancer. In another embodiment, this invention is directed to suppressing primary prostate cancer. In another embodiment, this invention is directed to reducing the incidence of primary prostate cancer.

In another embodiment, this invention is directed to reducing the severity of primary prostate cancer. In another embodiment, this invention is directed to inhibiting primary prostate cancer. In another embodiment, this invention is directed to increasing the survival of a subject with primary prostate cancer.

In one embodiment, the methods of this invention are directed to treating naïve hormone prostate cancer. In one embodiment, the methods of this invention are directed to suppressing, reducing the incidence, reducing the severity, or inhibiting hormone naïve prostate cancer. In one embodiment, the methods of this invention are directed to palliative treatment of hormone naïve prostate cancer. In another embodiment, this invention is directed to suppressing hormone naïve prostate cancer. In another embodiment, this invention is directed to reducing the incidence of hormone naïve prostate cancer. In another embodiment, this invention is directed to reducing the severity of hormone naïve prostate cancer. In another embodiment, this invention is directed to inhibiting hormone naïve prostate cancer. In another embodiment, this invention is directed to increasing the survival of a subject with hormone naïve prostate cancer.

In one embodiment, the methods of this invention are directed to treating advanced prostate cancer. In one embodiment, the methods of this invention are directed to suppressing, reducing the incidence, reducing the severity, or inhibiting advanced prostate cancer. In one embodiment, the methods of this invention are directed to palliative treatment of advanced prostate cancer. In another embodiment, this invention is directed to suppressing advanced prostate cancer. In another embodiment, this invention is directed to reducing the incidence of advanced prostate cancer. In another embodiment, this invention is directed to reducing the severity of advanced prostate cancer. In another embodiment, this invention is directed to inhibiting advanced prostate cancer. In another embodiment, this invention is directed to increasing the survival of a subject with advanced prostate cancer.

In one embodiment, the methods of this invention are directed to treating refractory prostate cancer. In one embodiment, the methods of this invention are directed to suppressing, reducing the incidence, reducing the severity, or inhibiting refractory prostate cancer. In one embodiment, the methods of this invention are directed to palliative treatment of refractory prostate cancer. In another embodiment, this invention is directed to suppressing refractory prostate cancer. In another embodiment, this invention is directed to reducing the incidence of refractory prostate cancer. In another embodiment, this invention is directed to reducing the severity of refractory prostate cancer. In another embodiment, this invention is directed to inhibiting refractory prostate cancer. In another embodiment, this invention is directed to increasing the survival of a subject with refractory prostate cancer.

In one embodiment, the methods of this invention are directed to treating metastatic prostate cancer. In one embodiment, the methods of this invention are directed to suppressing, reducing the incidence, reducing the severity, or inhibiting metastatic prostate cancer. In one embodiment, the methods of this invention are directed to palliative treatment of metastatic prostate cancer. In another embodiment, this invention is directed to suppressing metastatic prostate cancer. In another embodiment, this invention is directed to reducing the incidence of metastatic prostate cancer. In another embodiment, this invention is directed to reducing the severity of metastatic prostate cancer. In another embodiment, this invention is directed to inhibiting metastatic prostate cancer. In another embodiment, this invention is directed to increasing the survival of a subject with metastatic prostate cancer.

In one embodiment, the methods of this invention are directed to treating castration resistant prostate cancer. In one embodiment, the methods of this invention are directed to suppressing, reducing the incidence, reducing the severity, or inhibiting castration resistant prostate cancer. In one embodiment, the methods of this invention are directed to palliative treatment of castration resistant prostate cancer. In another embodiment, this invention is directed to suppressing castration resistant prostate cancer. In another embodiment, this invention is directed to reducing the incidence of castration resistant prostate cancer. In another embodiment, this invention is directed to reducing the severity of castration resistant prostate cancer. In another embodiment, this invention is directed to inhibiting castration resistant prostate cancer. In another embodiment, this invention is directed to increasing the survival of a subject with castration resistant prostate cancer.

In one embodiment, this invention provides a method of delaying the progression of prostate cancer in a subject suffering from prostate cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of prostate cancer in the subject. In another embodiment, this invention provides a method of delaying the progression of primary prostate cancer in a subject suffering from primary prostate cancer, comprising the step of administering to said subject a compound of this invention or its isomer, tautomer, pharmaceutically acceptable salt, pharmaceutical product, crystal, N-oxide, hydrate, to prodrug or metabolite or any combination thereof, or a composition comprising the same in an amount effective to delay the progression of primary prostate cancer in the subject. In still another embodiment, this invention provides a method of delaying the progression of hormone naïve prostate cancer in a subject suffering from prostate cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of prostate cancer in the subject. In another embodiment, this invention provides a method of delaying the progression of advanced prostate cancer in a subject suffering from advanced prostate cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of advanced prostate cancer in the subject. In one embodiment, this invention provides a method of delaying the progression of metastatic prostate cancer in a subject suffering from metastatic prostate cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of metastatic prostate cancer in the subject. In another embodiment, this invention provides a method of delaying the progression of refractory prostate cancer in a subject suffering from refractory prostate cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of refractory prostate cancer in the subject. In one embodiment, this invention provides a method of delaying the progression of castration resistant prostate cancer in a subject suffering from castration resistant prostate cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of castration resistant prostate cancer in the subject.

In one embodiment, the methods of this invention make use of compounds of Formula I. In another embodiment, the methods of this invention make use of compounds of Formula II. In yet another embodiment, the methods of this invention make use of compounds of Formula III. In still another embodiment, the methods of this invention make use of compounds of Formula IV. In a further embodiment, the methods of this invention make use of compounds of Formula V. In another embodiment, the methods of this invention make use of compounds of Formula VI. In yet another another embodiment, the methods of this invention make use of compounds of Formula VII. In still another embodiment, the methods of this invention make use of compounds of Formula VIII. In a further embodiment, the methods of this invention make use of compounds of Formula IX. In another embodiment, the methods of this invention make use of compounds of Formula X. In yet another embodiment, the methods of this invention make use of compounds of Formula XI. In still another embodiment, the methods of this invention make use of compounds of Formula XII. In still another embodiment, the methods of this invention make use of compounds of Formula XIII In a further embodiment, the methods of this invention make use of compounds of Formula I-XIII in combination with LH-RH agonist. In still another embodiment, the methods of this invention make use of compounds of Formula I-XIII in combination with an anti-androgen. In a further embodiment, the methods of this invention make use of compounds of Formula I-XIII in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of compounds of Formula I-XI, and/or XII in combination with leuprolide acetate)(Lupron®).

In one embodiment, the methods of treating prostate cancer make use of a compound of Formula I. In another embodiment, the methods of treating prostate cancer make use of a compound of Formula II. In another embodiment, the methods of treating prostate cancer make use of a compound of Formula III. In another embodiment, the methods of treating prostate cancer make use of a compound of Formula IV. In another embodiment, the methods of treating prostate cancer make use of a compound of Formula V. In another embodiment, the methods of treating prostate cancer make use of a compound of Formula VI. In another embodiment, the methods of treating prostate cancer make use of a compound of Formula VII. In another embodiment, the methods of treating prostate cancer make use of a compound of Formula VIII. In another embodiment, the methods of treating prostate to cancer make use of a compound of Formula IX. In another embodiment, the methods of treating prostate cancer make use of a compound of Formula X. In another embodiment, the methods of treating prostate cancer make use of a compound of Formula XI. In another embodiment, the methods of treating prostate cancer make use of a compound of Formula XII. In another embodiment, the methods of treating prostate cancer make use of a compound of Formula XIII

In one embodiment, the methods of treating prostate cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating prostate cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating prostate cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating prostate cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating prostate cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating prostate cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating prostate cancer make use of 6-hydroxy-2-(4-(hydroxymethyl)-phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating prostate cancer make use of 2-(4-(bromomethyl)-3-hydroxyphenyl)-6-hydroxy-4-(4-(trifluoromethyl)-phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating prostate cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating prostate cancer make use of 3-(4-(3-fluoro-4-(trifluoromethyl)-phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof. In yet another embodiment, the methods of treating prostate cancer make use of 4-(4-(3-fluoro-4-(trifluoromethyl)-phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating primary prostate cancer make use of a compound of Formula I. In another embodiment, the methods of treating primary prostate cancer make use of a compound of Formula II. In another embodiment, the methods of treating primary prostate cancer make use of a compound of Formula III. In another embodiment, the methods of treating primary prostate cancer make use of a compound of Formula IV. In another embodiment, the methods of treating primary prostate cancer make use of a compound of Formula V. In another embodiment the methods of treating primary prostate cancer make use of a compound of Formula VI. In another embodiment, the methods of treating primary prostate cancer make use of a compound of Formula VII.

In another embodiment, the methods of treating primary prostate cancer make use of a compound of Formula VIII. In another embodiment, the methods of treating primary prostate cancer make use of a compound of Formula IX. In another embodiment, the methods of treating primary prostate cancer make use of a compound of Formula X. In another embodiment, the methods of treating primary prostate cancer make use of a compound of Formula XI. In another embodiment, the methods of treating primary prostate cancer make use of a compound of Formula XII. In another embodiment, the methods of treating primary prostate cancer make use of a compound of Formula XIII

In one embodiment, the methods of treating primary prostate cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating primary prostate cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating primary prostate cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating primary prostate cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating primary prostate cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating primary prostate cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating primary prostate cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating primary prostate cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating primary prostate cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating primary prostate cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating primary prostate cancer make use of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof.

In one embodiment, the methods of treating hormone naïve prostate cancer make use of a compound of Formula I. In another embodiment, the methods of treating hormone naïve prostate cancer make use of a compound of Formula II. In another embodiment, the methods of treating to hormone naïve prostate cancer make use of a compound of Formula III. In another embodiment, the methods of treating hormone naïve prostate cancer make use of a compound of Formula IV. In another embodiment, the methods of treating hormone naïve prostate cancer make use of a compound of Formula V. In another embodiment the methods of treating hormone naïve prostate cancer make use of a compound of Formula VI. In another embodiment, the methods of treating hormone naïve prostate cancer make use of a compound of Formula VII. In another embodiment, the methods of treating hormone naïve prostate cancer make use of a compound of Formula VIII. In another embodiment, the methods of treating hormone naïve prostate cancer make use of a compound of Formula IX. In another embodiment, the methods of treating hormone naïve prostate cancer make use of a compound of Formula X. In another embodiment, the methods of treating hormone naïve prostate cancer make use of a compound of Formula XI. In another embodiment, the methods of treating hormone naïve prostate cancer make use of a compound of Formula XII. In another embodiment, the methods of treating hormone naïve prostate cancer make use of a compound of Formula XIII

In one embodiment, the methods of treating hormone naïve prostate cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating hormone naïve prostate cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating hormone naïve prostate cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating hormone naïve prostate cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating hormone naïve prostate cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating hormone naïve prostate cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, to crystal, N-oxide, hydrate or any combination thereof. In yet another embodiment, the methods of treating hormone naïve prostate cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating hormone naïve prostate cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating hormone naïve prostate cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating hormone naïve prostate cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating hormone naïve prostate cancer make use of 4-(4-(3-fluoro-4-(trifluoromethyl)phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating advanced prostate cancer make use of a compound of Formula I. In another embodiment, the methods of treating advanced prostate cancer make use of a compound of Formula II. In another embodiment, the methods of treating advanced prostate cancer make use of a compound of Formula III. In another embodiment, the methods of treating advanced prostate cancer make use of a compound of Formula IV. In another embodiment, the methods of treating advanced prostate cancer make use of a compound of Formula V. In another embodiment the methods of treating advanced prostate cancer make use of a compound of Formula VI. In another embodiment, the methods of treating advanced prostate cancer make use of a compound of Formula VII. In another embodiment, the methods of treating advanced prostate cancer make use of a compound of Formula VIII. In another embodiment, the methods of treating advanced prostate cancer make use of a compound of Formula IX. In another embodiment, the methods of treating advanced prostate cancer make use of a compound of Formula X. In another embodiment, the methods of treating advanced prostate cancer make use of a compound of Formula XI. In another embodiment, the methods of treating advanced prostate cancer make use of a compound of Formula XII. In another to embodiment, the methods of treating advanced prostate cancer make use of a compound of Formula XIII

In one embodiment, the methods of treating advanced prostate cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating advanced prostate cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating advanced prostate cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating advanced prostate cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating advanced prostate cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating advanced prostate cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating advanced prostate cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating advanced prostate cancer make use of 2-(4-(bromomethyl)-3-hydroxyphenyl)-6-hydroxy-4-(4-(trifluoromethyl)-phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating advanced prostate cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating advanced prostate to cancer make use of 3-(4-(3-fluoro-4-(trifluoromethyl)-phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof. In yet another embodiment, the methods of treating advanced prostate cancer make use of 4-(4-(3-fluoro-4-(trifluoromethyl)-phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating metastatic prostate cancer make use of a compound of Formula I. In another embodiment, the methods of treating metastatic prostate cancer make use of a compound of Formula II. In another embodiment, the methods of treating metastatic prostate cancer make use of a compound of Formula III. In another embodiment, the methods of treating metastatic prostate cancer make use of a compound of Formula IV. In another embodiment, the methods of treating metastatic prostate cancer make use of a compound of Formula V. In another embodiment the methods of treating metastatic prostate cancer make use of a compound of Formula VI. In another embodiment, the methods of treating metastatic prostate cancer make use of a compound of Formula VII. In another embodiment, the methods of treating metastatic prostate cancer make use of a compound of Formula VIII. In another embodiment, the methods of treating metastatic prostate cancer make use of a compound of Formula IX. In another embodiment, the methods of treating metastatic prostate cancer make use of a compound of Formula X. In another embodiment, the methods of treating metastatic prostate cancer make use of a compound of Formula XI. In another embodiment, the methods of treating metastatic prostate cancer make use of a compound of Formula XII. In another embodiment, the methods of treating metastatic prostate cancer make use of a compound of Formula XIII

In one embodiment, the methods of treating metastatic prostate cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating metastatic prostate cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating metastatic prostate cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating metastatic prostate cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating metastatic prostate cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating metastatic prostate cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating metastatic prostate cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating metastatic prostate cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating metastatic prostate cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating metastatic prostate cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating metastatic prostate cancer make use of 4-(4-(3-fluoro-4-(trifluoromethyl)phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating refractory prostate cancer make use of a compound of Formula I. In another embodiment, the methods of treating refractory prostate cancer make use of a compound of Formula II. In another embodiment, the methods of treating refractory prostate cancer make use of a compound of Formula III. In another embodiment, the methods of treating refractory prostate cancer make use of a compound of Formula IV. In another embodiment, the to methods of treating refractory prostate cancer make use of a compound of Formula V. In another embodiment the methods of treating refractory prostate cancer make use of a compound of Formula VI. In another embodiment, the methods of treating refractory prostate cancer make use of a compound of Formula VII. In another embodiment, the methods of treating refractory prostate cancer make use of a compound of Formula VIII. In another embodiment, the methods of treating refractory prostate cancer make use of a compound of Formula IX. In another embodiment, the methods of treating refractory prostate cancer make use of a compound of Formula X. In another embodiment, the methods of treating refractory prostate cancer make use of a compound of Formula XI. In another embodiment, the methods of treating refractory prostate cancer make use of a compound of Formula XII. In another embodiment, the methods of treating refractory prostate cancer make use of a compound of Formula XIII.

In one embodiment, the methods of treating refractory prostate cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating refractory prostate cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating refractory prostate cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating refractory prostate cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating refractory prostate cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating refractory prostate cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating refractory prostate cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating refractory prostate cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating refractory prostate cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating refractory prostate cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating refractory prostate cancer make use of 4-(4-(3-fluoro-4-(trifluoromethyl)phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating castration resistant prostate cancer make use of a compound of Formula I. In another embodiment, the methods of treating castration resistant prostate cancer make use of a compound of Formula II. In another embodiment, the methods of treating castration resistant prostate cancer make use of a compound of Formula III. In another embodiment, the methods of treating castration resistant prostate cancer make use of a compound of Formula IV. In another embodiment, the methods of treating castration resistant prostate cancer make use of a compound of Formula V. In another embodiment the methods of treating castration resistant prostate cancer make use of a compound of Formula VI. In another embodiment, the methods of treating castration resistant prostate cancer make use of a compound of Formula VII. In another embodiment, the methods of treating castration resistant prostate cancer make use of a compound of Formula VIII. In another embodiment, the methods of treating castration resistant prostate cancer make use of a compound of Formula IX. In another embodiment, the methods of treating castration resistant prostate cancer make use of a compound of Formula X. In another embodiment, the methods of treating castration resistant prostate cancer make use of a compound of Formula XI. In another embodiment, the methods of treating castration resistant prostate cancer make use of a compound of Formula XII. In another embodiment, the methods of treating castration resistant prostate cancer make use of a compound of Formula XIII

In one embodiment, the methods of treating castration resistant prostate cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating castration resistant prostate cancer make use of 6,8-dihydroxy-2-(4-hydroxyphenyl)-4-(4-methoxyphenyl)isoquinolin-1(2H)-one (15 g), or its prodrug, isomer, tautomer, metabolite, pharmaceutically acceptable salt, polymorph, crystal, N-oxide, hydrate or any combination thereof. In yet another embodiment, the methods of treating castration resistant prostate cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating castration resistant prostate cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating castration resistant prostate cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating castration resistant prostate cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating castration resistant prostate cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating castration resistant prostate cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating castration resistant prostate cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating castration resistant prostate cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating castration resistant prostate cancer make use of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof.

In one embodiment, the methods of this invention make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use of 6-hydroxy-2-(4-hydroxyphenyl)-4-phenylisoquinolin-1(2H)-one (15a), or a prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof, in combination with an LH-RH agonist. In another embodiment, the methods of this invention make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof, in combination with leuprolide to acetate (Lupron®). In still another embodiment, the methods of this invention make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone). In still another embodiment, the methods of this invention make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof, in combination with leuprolide acetate) (Lupron®. In still another embodiment, the methods of this invention make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 6-hydroxy-2-(4-hydroxyphenyl)-4-(4-(trifluoromethyl)phenylisoquinolin-1(2H)-one (13), or its to prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof, in combination with leuprolide acetate) (Lupron®. In still another embodiment, the methods of this invention make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use 2-(4-(bromomethyl)-3-hydroxyphenyl)-6-hydroxy-4-(4-(trifluoromethyl)phenyl) isoquinolin-1(2H)-one (26), or its prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

In one embodiment, the methods of this invention make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone). In one embodiment, the methods of this invention make use of 3-(4-(3-fluoro-4-(trifluoromethyl)phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of 3-(4-(3-fluoro-4-(trifluoromethyl)phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 3-(4-(3-fluoro-4-(trifluoromethyl)phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone). In one embodiment, the methods of this invention make use of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof, in combination with LH-RH agonist. In another embodiment, the methods of this invention make use 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof, in combination with leuprolide acetate (Lupron®). In still another embodiment, the methods of this invention make use of 4-(4-(3-fluoro-4-(trifluoromethyl)phenyl)-6-hydroxy-1-oxoisoquinolin-2(1H)-yl)benzamide (215), or its to prodrug, isomer, tautomer, metabolite, pharmaceutically acceptable salt, polymorph, crystal, N-oxide, hydrate or any combination thereof, in combination with an anti-androgen. In a further embodiment, the methods of this invention make use of 4-(4-(3-fluoro-4-(trifluoromethyl)phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof, in combination with gonadotropin releasing hormone agonists (e.g., leuprolide) or antagonists (degarelix), anti-androgens (e.g., bicalutamide, nilutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) or a lyase inhibitor (abiraterone).

As used herein, the term “primary prostate cancer” refers to prostate cancer that develops in the prostate.

As used herein, the term “hormone naïve prostate cancer” refers to prostate cancer that has not been treated with hormones such as estrogens or androgens, agents that block hormones such antiandrogens and antiestrogens, or agents that manipulate hormone levels such as LH-RH agonists or antagonists like leuprolide acetate (Lupron®) and degarelix, respectively.

As used herein, the term “refractory prostate cancer” refers to a prostate cancer that has not responded to treatment. In another embodiment, a “refractory prostate cancer” is a prostate cancer resistant to treatment. In one embodiment, refractory prostate cancer is refractory metastatic prostate cancer. In one embodiment, refractory prostate cancer has not responded to chemical/surgical castration, i.e., the reduction of available androgen/testosterone/DHT by chemical or surgical means. As used herein, “refractory prostate cancer”, may in some embodiments be referred to as “androgen-independent prostate cancer”. As used herein, “refractory prostate cancer”, may in some embodiment be referred to as castration resistant prostate cancer”.

As used herein, the term “castration resistant prostate cancer” (CRPC) refers to advanced prostate cancer which was developed despite ongoing androgen deprivation therapy (ADT) and/or surgical castration. In another embodiment, ADT refers to treatment consisting leuprolide acetate (Lupron®).

As used herein, the term “advanced prostate cancer” refers to metastatic cancer having originated in the prostate, and having widely metastasized to beyond the prostate such as the surrounding tissues to include the seminal vesicles the pelvic lymph nodes or bone, or to other parts of the body. Prostate cancer pathologies are graded with a Gleason grading from 1 to 5 in order of increasing malignancy. In another embodiment, patients with significant risk of progressive disease and/or death from prostate cancer should be included in the definition and that any patient with cancer outside the prostate capsule with disease stages as low as IIB clearly has “advanced” disease.

As used herein, the term “metastatic prostate cancer” refers to prostate cancer that has spread from the place where it first started to another place in the body. Other places in the body include but are not limited to, the lungs, liver and bone.

In further embodiments, the present invention provides methods of treatment of conditions related to the prostate. For example, the present invention provides methods of treatment of treatment of prostatic dysplasia, prostatic hyperplasia, benign prostate hyperplasia (BPH), and prostatitis. The present invention also provides methods of treatment of precancerous precursors of prostate adenocarcinoma. In one embodiment, the precancerous precursor of prostate adenocarcinoma is prostate intraepithelial neoplasia (PIN). In one embodiment, the PIN is high-grade PIN (HGPIN).

In some embodiments, the present invention provides methods of treatment conditions such as, but not limited to, prostate cancer, benign prostate hyperplasia (BPH), lung cancer, acne, seborrhea, hirsuitism, baldness, alopecia, precocious puberty, adrenal hypertrophy, polycystic ovary syndrome, breast cancer, endometriosis, myeloma and leiomyoma.

In one embodiment, this invention provides a method of treating a subject suffering from breast cancer, comprising the step of administering to said subject a compound of this invention, or its isomer, tautomer, pharmaceutically acceptable salt, pharmaceutical product, crystal, hydrate, N-oxide, prodrug, metabolite or any combination thereof, or a composition comprising the same in an amount effective to treat breast cancer in the subject.

In one embodiment, the compounds of this invention are useful for a) treating a subject suffering from breast cancer; b) treating a subject suffering from metastatic breast cancer; c) treating a subject suffering from refractory breast cancer; d) treating a subject suffering from AR-positive breast cancer; e) treating a subject suffering from AR-positive refractory breast cancer; f) treating a subject suffering from AR-positive metastatic breast cancer; g) treating a subject suffering from triple negative breast cancer; h) treating a subject suffering from advanced breast cancer; i) treating a subject suffering breast cancer that have failed SERM (tamoxifen, toremifene), aromatase to inhibitor, trastuzumab (Herceptin®), exemestane (Aromasin®), bevacizumab (Avastin®), and/or fulvestrant treatment; j) treating, preventing, suppressing or inhibiting metastasis in a subject suffering from breast cancer; k) prolonging survival of a subject with breast cancer, and/or 1) prolonging the progression-free survival of a subject with breast cancer.

In one embodiment, a “refractory breast cancer” is a breast cancer that has not responded to treatment. In another embodiment, a “refractory breast cancer” is a breast cancer resistant to treatment. In one embodiment, refractory breast cancer is refractory metastatic breast cancer. In one embodiment, refractory breast cancer has not responded to treatment with anthracyclines, taxanes, capecitabine, ixabepilone, SERM (tamoxifen, toremifene), aromatase inhibitor, trastuzumab (Herceptin®), exemestane (Aromasin®), bevacizumab (Avastin®), fulvestrant or a combination thereof.

In one embodiment, a “triple negative breast cancer” is defined by lack of expression of estrogen, progesterone, and ErbB2 (also known as human epidermal growth factor receptor 2 (HER2)) receptors. This subgroup accounts for 15% of all types of breast cancer. This subtype of breast cancer is clinically characterized as more aggressive and less responsive to standard treatment and associated with poorer overall patient prognosis.

In one embodiment, the methods of this invention are directed to treating a subject suffering from AR-positive breast cancer, regardless of grade, stage or prior treatments.

In one embodiment, the methods of this invention are directed to treating a subject suffering from ER-positive breast cancer, regardless of grade, stage or prior treatments.

In one embodiment, the methods of this invention are first, second, third, or fourth line therapies for breast cancer. A first line therapy refers to a medical therapy recommended for the initial treatment of a disease, sign or symptom. A second line therapy is given when initial treatment (first-line therapy) does not work, or stops working. Third line therapy is given when both initial treatment (first-line therapy) and subsequent treatment (second-line therapy) does not work, or stop working, etc.

In one embodiment, this invention provides a method of delaying the progression of breast cancer in a subject suffering from breast cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of to breast cancer in the subject. In another embodiment, this invention provides a method of delaying the progression of advanced breast cancer in a subject suffering from advanced breast cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of advanced breast cancer in the subject. In one embodiment, this invention provides a method of delaying the progression of metastatic breast cancer in a subject suffering from metastatic breast cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of metastatic breast cancer in the subject. In one embodiment, this invention provides a method of delaying the progression of hormone-resistant breast cancer in a subject suffering from hormone-resistant breast cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of hormone-resistant breast cancer in the subject.

In one embodiment, the methods of treating breast cancer make use of a compound of Formula I. In another embodiment, the methods of treating breast cancer make use of a compound of Formula II. In another embodiment, the methods of treating breast cancer make use of a compound of Formula III. In another embodiment, the methods of treating breast cancer make use of a compound of Formula IV. In another embodiment, the methods of treating breast cancer make use of a compound of Formula V. In another embodiment the methods of treating breast cancer make use of a compound of Formula VI. In another embodiment, the methods of treating breast cancer make use of a compound of Formula VII. In another embodiment, the methods of treating breast cancer make use of a compound of Formula VIII. In another embodiment, the methods of treating breast cancer make use of a compound of Formula IX. In another embodiment, the methods of treating breast cancer make use of a compound of Formula X. In another embodiment, the methods of treating breast cancer make use of a compound of Formula XI. In another embodiment, the methods of treating breast cancer make use of a compound of Formula XII. In another embodiment, the methods of treating breast cancer make use of a compound of Formula XIII

In one embodiment, the methods of treating breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating breast cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating breast cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating breast cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating breast cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating breast cancer make use of 3-(4-(3-fluoro-4-(trifluoromethyl)-phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating breast cancer make use of 4-(4-(3-fluoro-4-(trifluoromethyl)-phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating advanced breast cancer make use of a compound of Formula I. In another embodiment, the methods of treating advanced breast cancer make use of a compound of Formula II. In another embodiment, the methods of treating advanced breast cancer make use of a compound of Formula III. In another embodiment, the methods of treating advanced breast cancer make use of a compound of Formula IV. In another embodiment, the methods of treating advanced breast cancer make use of a compound of Formula V. In another embodiment the methods of treating advanced breast cancer make use of a compound of Formula VI. In another embodiment, the methods of treating advanced breast cancer make use of a compound of Formula VII.

In another embodiment, the methods of treating advanced breast cancer make use of a compound of Formula VIII. In another embodiment, the methods of treating advanced breast cancer make use of a compound of Formula IX. In another embodiment, the methods of treating advanced breast cancer make use of a compound of Formula X. In another embodiment, the methods of treating advanced breast cancer make use of a compound of Formula XI. In another embodiment, the methods of treating advanced breast cancer make use of a compound of Formula XII. In another embodiment, the methods of treating advanced breast cancer make use of a compound of Formula XIII In one embodiment, the methods of treating advanced breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating advanced breast cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating advanced breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating advanced breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating advanced breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating advanced breast cancer make use of 6-hydroxy-2-(4-hydroxyphenyl)-4-(4-(trifluoromethyl)phenylisoquinolin-[(211]-one (13), or its prodrug, isomer, tautomer, metabolite, pharmaceutically acceptable salt, polymorph, crystal, N-oxide, hydrate or any combination thereof. In yet another embodiment, the methods of treating advanced breast cancer make use of 6-hydroxy-2-(4-(hydroxymethyl)phenyl)-4-(4-hydroxyphenyl)isoquinolin-[(211]-one (14), or its prodrug, isomer, tautomer, metabolite, pharmaceutically acceptable salt, polymorph, crystal, N-oxide, hydrate or any combination thereof. In still another embodiment, the methods of treating advanced breast cancer make use of 2-(4-(bromomethyl)-3-hydroxyphenyl)-6-hydroxy-4-(4-(trifluoromethyl)phenyl)isoquinolin-[(211]-one (26), or its prodrug, isomer, tautomer, metabolite, pharmaceutically acceptable salt, polymorph, crystal, N-oxide, hydrate or any combination thereof. In a further embodiment, the methods of treating advanced breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating advanced breast cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating advanced breast cancer make use of 4-(4-(3-fluoro-4-(trifluoromethyl)phenyl)-6-hydroxy-1-oxoisoquinolin-2(1H-yl)benzamide (215), or its prodrug, isomer, tautomer, metabolite, pharmaceutically acceptable salt, polymorph, crystal, N-oxide, hydrate or any combination thereof.

In one embodiment, the methods of treating metastatic breast cancer make use of a compound of Formula I. In another embodiment, the methods of treating metastatic breast cancer make use of a compound of Formula II. In another embodiment, the methods of treating metastatic breast cancer make use of a compound of Formula III. In another embodiment, the methods of treating metastatic breast cancer make use of a compound of Formula IV. In another embodiment, the methods of treating metastatic breast cancer make use of a compound of Formula V. In another embodiment the methods of treating metastatic breast cancer make use of a compound of Formula VI. In another embodiment, the methods of treating metastatic breast cancer make use of a compound of Formula VII. In another embodiment, the methods of treating metastatic breast cancer make use of a compound of Formula VIII. In another embodiment, the methods of treating metastatic breast cancer make use of a compound of Formula IX. In another embodiment, the methods of treating metastatic breast cancer make use of a compound of Formula X. In another embodiment, the methods of treating metastatic breast cancer make use of a compound of Formula XI. In another embodiment, the methods of treating metastatic breast cancer make use of a compound of Formula XII. In another embodiment, the methods of treating metastatic breast cancer make use of a compound of Formula XIII

In one embodiment, the methods of treating metastatic breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating metastatic breast cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating metastatic breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating metastatic breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating metastatic breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating metastatic breast cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating metastatic breast cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating metastatic breast cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating metastatic breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating metastatic breast cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating metastatic breast cancer make use of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof.

In one embodiment, the methods of treating refractory breast cancer make use of a compound of Formula I. In another embodiment, the methods of treating refractory breast cancer make use of a compound of Formula II. In another embodiment, the methods of treating refractory breast cancer make use of a compound of Formula III. In another embodiment, the methods of treating refractory breast cancer make use of a compound of Formula IV. In another embodiment, the methods of treating refractory breast cancer make use of a compound of Formula V. In another embodiment the methods of treating refractory breast cancer make use of a compound of Formula VI. In another embodiment, the methods of treating refractory breast cancer make use of a compound of Formula VII. In another embodiment, the methods of treating refractory breast cancer make use of a compound of Formula VIII. In another embodiment, the methods of treating refractory breast cancer make use of a compound of Formula IX. In another embodiment, the methods of treating refractory breast cancer make use of a compound of Formula X. In another embodiment, the methods of treating refractory breast cancer make use of a compound of Formula XI. In another embodiment, the methods of treating refractory breast cancer make use of a compound of Formula XII. In another embodiment, the methods of treating refractory breast cancer make use of a compound of Formula XIII

In one embodiment, the methods of treating refractory breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating refractory breast cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating refractory breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating refractory breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating refractory breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating refractory breast cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating refractory breast cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating refractory breast cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating refractory breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating refractory breast cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating refractory breast cancer make use of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof.

In one embodiment, the methods of treating AR-positive breast cancer make use of a compound of Formula I. In another embodiment, the methods of treating AR-positive breast cancer make use of a compound of Formula II. In another embodiment, the methods of treating AR-positive to breast cancer make use of a compound of Formula III. In another embodiment, the methods of treating AR-positive breast cancer make use of a compound of Formula IV. In another embodiment, the methods of treating AR-positive breast cancer make use of a compound of Formula V. In another embodiment the methods of treating AR-positive breast cancer make use of a compound of Formula VI. In another embodiment, the methods of treating AR-positive breast cancer make use of a compound of Formula VII. In another embodiment, the methods of treating AR-positive breast cancer make use of a compound of Formula VIII. In another embodiment, the methods of treating AR-positive breast cancer make use of a compound of Formula IX. In another embodiment, the methods of treating AR-positive breast cancer make use of a compound of Formula X. In another embodiment, the methods of treating AR-positive breast cancer make use of a compound of Formula XI. In another embodiment, the methods of treating AR-positive breast cancer make use of a compound of Formula XII. In another embodiment, the methods of treating AR-positive breast cancer make use of a compound of Formula XIII

In one embodiment, the methods of treating AR-positive breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating AR-positive breast cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating AR-positive breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating AR-positive breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating AR-positive breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating AR-positive breast cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating AR-positive breast cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating AR-positive breast cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating AR-positive breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating AR-positive breast cancer make use of 3-(4-(3-fluoro-4-(trifluoromethyl)phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof. In yet another embodiment, the methods of treating AR-positive breast cancer make use of 4-(4-(3-fluoro-4-(trifluoromethyl)phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating AR-positive refractory breast cancer make use of a compound of Formula I. In another embodiment, the methods of treating AR-positive refractory breast cancer make use of a compound of Formula II. In another embodiment, the methods of treating AR-positive refractory breast cancer make use of a compound of Formula III. In another embodiment, the methods of treating AR-positive refractory breast cancer make use of a compound of Formula IV. In another embodiment, the methods of treating AR-positive refractory breast cancer make use of a compound of Formula V. In another embodiment the methods of treating AR-positive refractory breast cancer make use of a compound of Formula VI. In another embodiment, the methods of treating AR-positive refractory breast cancer make use of a compound of Formula VII. In another embodiment, the methods of treating AR-positive refractory breast cancer make use of a compound of Formula VIII. In another embodiment, the methods of treating AR-positive refractory breast cancer make use of a compound of Formula IX. In another embodiment, the methods of treating AR-positive refractory breast cancer make use of a compound of Formula X. In another embodiment, the methods of treating AR-positive refractory breast cancer make use of a compound of Formula XI. In another embodiment, the methods of treating AR-positive refractory breast cancer make use of a compound of Formula XII. In another embodiment, the methods of treating AR-positive refractory breast cancer make use of a compound of Formula XIII

In one embodiment, the methods of treating AR-positive refractory breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating AR-positive refractory breast cancer make use of 6,8-dihydroxy-2-(4-hydroxyphenyl)-4-(4-methoxyphenyl)isoquinolin-1(2H)-one (15 g), or its prodrug, isomer, tautomer, metabolite, pharmaceutically acceptable salt, polymorph, crystal, N-oxide, hydrate or any combination thereof. In yet another embodiment, the methods of treating AR-positive refractory breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating AR-positive refractory breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating AR-positive refractory breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating AR-positive refractory breast cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating AR-positive refractory breast cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating AR-positive refractory breast cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating AR-positive refractory breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating AR-positive refractory breast cancer make use of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof. In yet another embodiment, the methods of treating AR-positive refractory breast cancer make use of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof.

In one embodiment, the methods of treating AR-positive metastatic breast cancer make use of a compound of Formula I. In another embodiment, the methods of treating AR-positive metastatic breast cancer make use of a compound of Formula II. In another embodiment, the methods of treating AR-positive metastatic breast cancer make use of a compound of Formula III. In another embodiment, the methods of treating AR-positive metastatic breast cancer make use of a compound of Formula IV. In another embodiment, the methods of treating AR-positive metastatic breast cancer make use of a compound of Formula V. In another embodiment the methods of treating AR-positive metastatic breast cancer make use of a compound of Formula VI. In another embodiment, the methods of treating AR-positive metastatic breast cancer make use of a compound of Formula VII. In another embodiment, the methods of treating AR-positive metastatic breast cancer make use of a compound of Formula VIII. In another embodiment, the methods of treating AR-positive metastatic breast cancer make use of a compound of Formula IX. In another embodiment, the methods of treating AR-positive metastatic breast cancer make use of a compound of Formula X. In another embodiment, the methods of treating AR-positive metastatic breast cancer make use of a compound of Formula XI. In another embodiment, the methods of treating AR-positive metastatic breast cancer make use of a compound of Formula XII. In another embodiment, the methods of treating AR-positive metastatic breast cancer make use of a compound of Formula XIII

In one embodiment, the methods of treating AR-positive metastatic breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating AR-positive metastatic breast cancer make use of 6,8-dihydroxy-2-(4-hydroxyphenyl)-4-(4-methoxyphenyl)isoquinolin-1(2H)-one (15 g), or its prodrug, isomer, tautomer, metabolite, pharmaceutically acceptable salt, polymorph, crystal, N-oxide, hydrate or any combination thereof. In yet another embodiment, the methods of to treating AR-positive metastatic breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating AR-positive metastatic breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating AR-positive metastatic breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating AR-positive metastatic breast cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating AR-positive metastatic breast cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating AR-positive metastatic breast cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating AR-positive metastatic breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating AR-positive metastatic breast cancer make use of 3-(4-(3-fluoro-4-(trifluoromethyl)phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof. In yet another embodiment, the methods of treating AR-positive metastatic breast cancer make use of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof.

In one embodiment, the methods of treating triple negative breast cancer make use of a to compound of Formula I. In another embodiment, the methods of treating triple negative breast cancer make use of a compound of Formula II. In another embodiment, the methods of treating triple negative breast cancer make use of a compound of Formula III. In another embodiment, the methods of treating triple negative breast cancer make use of a compound of Formula IV. In another embodiment, the methods of treating triple negative breast cancer make use of a compound of Formula V. In another embodiment the methods of treating triple negative breast cancer make use of a compound of Formula VI. In another embodiment, the methods of treating triple negative breast cancer make use of a compound of Formula VII. In another embodiment, the methods of treating triple negative breast cancer make use of a compound of Formula VIII. In another embodiment, the methods of treating triple negative breast cancer make use of a compound of Formula IX. In another embodiment, the methods of treating triple negative breast cancer make use of a compound of Formula X. In another embodiment, the methods of treating triple negative breast cancer make use of a compound of Formula XI. In another embodiment, the methods of treating triple negative breast cancer make use of a compound of Formula XII. In another embodiment, the methods of treating triple negative breast cancer make use of a compound of Formula XIII

In one embodiment, the methods of treating triple negative breast cancer make use of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof. In another embodiment, the methods of treating triple negative breast cancer make use of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating triple negative breast cancer make use of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating triple negative breast cancer make use of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating triple negative breast cancer make use of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating triple negative breast cancer make use of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating triple negative breast cancer make use of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In still another embodiment, the methods of treating triple negative breast cancer make use of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating triple negative breast cancer make use of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating triple negative breast cancer make use of 3-(4-(3-fluoro-4-(trifluoromethyl)phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof. In yet another embodiment, the methods of treating triple negative breast cancer make use of 4-(4-(3-fluoro-4-(trifluoromethyl)phenyl)-6-hydroxy-1-oxoisoquinolin-2(1H)-yl)-benzamide (215), or its prodrug, isomer, tautomer, metabolite, pharmaceutically acceptable salt, polymorph, crystal, N-oxide, hydrate or any combination thereof.

In one embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula I. In another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula II. In yet another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula III. In still another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of to Formula IV. In a further embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula V. In another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula VI. In yet another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula VII. In still another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula VIII. In a further embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula IX. In another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula X. In yet another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula XI. In still another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula XII. In still another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of Formula XIII

In one embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 6-hydroxy-2-(4-hydroxyphenyl)-4-phenylisoquinolin-1(2H)-one (15a), or its prodrug, isomer, tautomer, to metabolite, pharmaceutically acceptable salt, polymorph, crystal, N-oxide, hydrate or any combination thereof. In another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof. In yet another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof. In still another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof. In a further embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof. In another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof. In yet another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof. In to still another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof. In a further embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof. In another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 3-(4-(3-fluoro-4-(trifluoromethyl)phenyl)-6-hydroxy-1-oxoisoquinolin-2(1H)-yl)-benzamide (214), or its prodrug, isomer, tautomer, metabolite, pharmaceutically acceptable salt, polymorph, crystal, N-oxide, hydrate or any combination thereof. In yet another embodiment, the methods of treating breast cancer that failed to respond to SERM (tamoxifen, toremifene), aromatase inhibitor, trastuzumab (Herceptin®), exemestane (Aromasin®), bevacizumab (Avastin®), and/or fulvestrant treatment make use of a compound of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof.

In one embodiment, this invention provides a method of treating a subject suffering from uterine cancer, comprising the step of administering to said subject a compound of this invention, or its isomer, tautomer, pharmaceutically acceptable salt, pharmaceutical product, crystal, hydrate, N-oxide, prodrug, metabolite or any combination thereof, or a composition comprising the same in an amount effective to treat uterine cancer in the subject. In one embodiment, uterine cancer refers to an endometrial cancer. In another embodiment, uterine cancer refers to a cervical cancer.

In one embodiment, the compounds of this invention are useful for a) treating a subject suffering from uterine cancer; b) treating a subject suffering from metastatic uterine cancer; c) treating a subject suffering from refractory uterine cancer; d) treating a subject suffering from advanced uterine cancer; e) treating, preventing, suppressing or inhibiting metastasis in a subject suffering from uterine cancer; f) prolonging survival of a subject with uterine cancer, and/or l) to prolonging the progression-free survival of a subject with uterine cancer.

In one embodiment, a “refractory uterine cancer” is a uterine cancer that has not responded to treatment. In another embodiment, a “refractory uterine cancer” is a uterine cancer resistant to treatment. In one embodiment, refractory uterine cancer is refractory metastatic uterine cancer.

In one embodiment, this invention provides a method of delaying the progression of uterine cancer or uterine fibroids in a subject suffering from uterine cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of uterine cancer in the subject. In another embodiment, this invention provides a method of delaying the progression of advanced uterine cancer in a subject suffering from advanced uterine cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of advanced uterine cancer in the subject. In one embodiment, this invention provides a method of delaying the progression of metastatic uterine cancer in a subject suffering from metastatic uterine cancer, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of metastatic uterine cancer in the subject.

In one embodiment, this invention provides a method of treating a subject suffering from uterine fibroids, comprising the step of administering to said subject a compound of this invention, or its isomer, tautomer, pharmaceutically acceptable salt, pharmaceutical product, crystal, hydrate, N-oxide, prodrug, metabolite or any combination thereof, or a composition comprising the same in an amount effective to treat uterine fibroids in the subject. In one embodiment, uterine fibroids refer to myomas.

In one embodiment, the compounds of this invention are useful for treating a subject suffering from uterine fibroids.

In one embodiment, this invention provides a method of delaying the progression of to uterine cancer or uterine fibroids in a subject suffering from uterine cancer or uterine fibroids, comprising the step of administering to said subject a compound of this invention or its isomer, tautomer, pharmaceutically acceptable salt, pharmaceutical product, crystal, N-oxide, hydrate, prodrug or metabolite or any combination thereof, or a composition comprising the same in an amount effective to delay the progression of uterine cancer or uterine fibroids in the subject.

In one embodiment, the methods of this invention make use of compounds of Formula I or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In another embodiment, the methods of this invention make use of compounds of Formula II or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In yet another embodiment, the methods of this invention make use of compounds of Formula III or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of this invention make use of compounds of Formula IV or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In a further embodiment, the methods of this invention make use of compounds of Formula V or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In another embodiment, the methods of this invention make use of compounds of Formula VI or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In yet another embodiment, the methods of this invention make use of compounds of Formula VII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of this invention make use of compounds of Formula VIII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In a further embodiment, to the methods of this invention make use of compounds of Formula IX or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In another embodiment, the methods of this invention make use of compounds of Formula X or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In yet another embodiment, the methods of this invention make use of compounds of Formula XI or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of this invention make use of compounds of Formula XII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of this invention make use of compounds of Formula XIII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In a further embodiment, the methods of this invention make use of compounds of Formula I-XI and/or formula XII and/or formula XIII or their isomer, tautomer, pharmaceutically acceptable salt, pharmaceutical product, crystal, N-oxide, hydrate, prodrug or metabolite or any combination thereof, in combination with LH-RH agonist, in an amount effective to treat uterine cancer or uterine fibroids. In a further embodiment, the methods of this invention make use of compounds of Formula I-XI and/or formula XII and/or formula XIII or their isomer, tautomer, pharmaceutically acceptable salt, pharmaceutical product, crystal, N-oxide, hydrate, prodrug or metabolite or any combination thereof, in combination with leuprolide acetate (Lupron®) in an amount effective to treat uterine cancer or uterine fibroids.

In one embodiment, the methods of treating uterine cancer or uterine fibroids makes use of a compound of Formula I or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In another embodiment, the methods of treating uterine cancer or uterine fibroids make use of a compound of Formula II or its isomer, tautomer, pharmaceutically acceptable salt, pharmaceutical product, crystal, N-oxide, hydrate, prodrug or metabolite or any combination thereof, in an amount effective to treat uterine cancer or uterine to fibroids. In yet another embodiment, the methods of treating uterine cancer or uterine fibroids and make use of a compound of Formula III or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of treating uterine cancer or uterine fibroids and make use of a compound of Formula IV or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In a further embodiment, the methods of treating uterine cancer or uterine fibroids and make use of a compound of Formula V or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In another embodiment, the methods of treating uterine cancer or uterine fibroids and make use of a compound of Formula VI or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In yet another embodiment, the methods of treating uterine cancer or uterine fibroids and make use of a compound of Formula VII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of treating uterine cancer or uterine fibroids and make use of a compound of Formula VIII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In a further embodiment, the methods of treating uterine cancer or uterine fibroids and make use of a compound of Formula IX or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In another embodiment, the methods of treating uterine cancer or uterine fibroids and make use of a compound of Formula X or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In yet another embodiment, the methods of treating uterine cancer or uterine fibroids and make use of a compound of Formula XI or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of treating uterine cancer or uterine fibroids and make use of a compound of Formula XII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids. In still another embodiment, the methods of treating uterine cancer or uterine fibroids and make use of a compound of Formula XIII or its isomer, tautomer, pharmaceutically acceptable salt, pharmaceutical product, crystal, N-oxide, hydrate, prodrug or metabolite or any combination thereof, in an amount effective to treat uterine cancer or uterine fibroids.

In one embodiment, the methods of treating uterine cancer or uterine fibroids and make use of a compound of 6-hydroxy-2-(4-hydroxyphenyl)-4-phenylisoquinolin-1(2H)-one (15a), or its prodrug, isomer, tautomer, metabolite, pharmaceutically acceptable salt, polymorph, crystal, N-oxide, hydrate or any combination thereof, in an amount effective to treat uterine cancer or uterine fibroids.

In another embodiment, the methods of treating uterine cancer or uterine fibroids and make use of a compound of 6,8-dihydroxy-2-(4-hydroxyphenyl)-4-(4-methoxyphenyl)isoquinolin-1(2H)-one (15g), or its prodrug, isomer, tautomer, metabolite, pharmaceutically acceptable salt, polymorph, crystal, N-oxide, hydrate or any combination thereof, in an amount effective to treat uterine cancer or uterine fibroids.

In yet another embodiment, the methods of treating uterine cancer or uterine fibroids and make use of a compound of 6,8-dihydroxy-2-(4-hydroxyphenyl)-4-phenylisoquinolin-1(2H)-one (15h), or its prodrug, isomer, tautomer, metabolite, pharmaceutically acceptable salt, polymorph, crystal, N-oxide, hydrate or any combination thereof, in an amount effective to treat uterine cancer or uterine fibroids.

In still another embodiment, the methods of treating uterine cancer or uterine fibroids and make use of a compound of (E)-3-(6,8-dihydroxy-2-(4-hydroxyphenyl)-1-oxo-1,2-dihydroisoquinolin-4-yl)acrylic acid (15l), or its prodrug, isomer, tautomer, metabolite, pharmaceutically acceptable salt, polymorph, crystal, N-oxide, hydrate or any combination thereof, in an amount effective to treat uterine cancer or uterine fibroids.

In a further embodiment, the methods of treating uterine cancer or uterine fibroids and make use of a compound of 2-(4-bromomethyl)phenyl-6-hydroxy-4-(4-hydroxyphenyl)isoquinolin-1(2H)-one (11), or its prodrug, isomer, tautomer, metabolite, pharmaceutically acceptable salt, polymorph, crystal, N-oxide, hydrate or any combination thereof, in an amount effective to treat uterine cancer or uterine fibroids.

In another embodiment, the methods of treating uterine cancer or uterine fibroids and to make use of a compound of 6-hydroxy-2-(4-hydroxyphenyl)-4-(4-(trifluoromethyl)phenylisoquinolin-1(2H)-one (13), or its prodrug, isomer, tautomer, metabolite, pharmaceutically acceptable salt, polymorph, crystal, N-oxide, hydrate or any combination thereof, in an amount effective to treat uterine cancer or uterine fibroids.

In yet another embodiment, the methods of treating uterine cancer or uterine fibroids and make use of a compound of 6-hydroxy-2-(4-(hydroxymethyl)phenyl)-4-(4-hydroxyphenyl)isoquinolin-1(2H)-one (14), or its prodrug, isomer, tautomer, metabolite, pharmaceutically acceptable salt, polymorph, crystal, N-oxide, hydrate or any combination thereof, in an amount effective to treat uterine cancer or uterine fibroids.

In still another embodiment, the methods of treating uterine cancer or uterine fibroids and make use of a compound of 2-(4-(bromomethyl)-3-hydroxyphenyl)-6-hydroxy-4-(4-(trifluoromethyl)phenyl)isoquinolin-1(2H)-one (26), or its prodrug, isomer, tautomer, metabolite, pharmaceutically acceptable salt, polymorph, crystal, N-oxide, hydrate or any combination thereof, in an amount effective to treat uterine cancer or uterine fibroids.

In a further embodiment, the methods of treating uterine cancer or uterine fibroids and make use of a compound of 6-hydroxy-2-(4-hydroxyphenyl)-1-oxo-4-(3,4,5-trifluorophenyl)-1,2-dihydroisoquinoline-8-carbonitrile (85), or its prodrug, isomer, tautomer, metabolite, pharmaceutically acceptable salt, polymorph, crystal, N-oxide, hydrate or any combination thereof, in an amount effective to treat uterine cancer or uterine fibroids.

In another embodiment, the methods of treating uterine cancer or uterine fibroids and make use of a compound of 3-(4-(3-fluoro-4-(trifluoromethyl)phenyl)-6-hydroxy-1-oxoisoquinolin-2(1H)-yl)benzamide (214), or its prodrug, isomer, tautomer, metabolite, pharmaceutically acceptable salt, polymorph, crystal, N-oxide, hydrate or any combination thereof, in an amount effective to treat uterine cancer or uterine fibroids.

In yet another embodiment, the methods of treating uterine cancer or uterine fibroids and make use of a compound of 4-(4-(3-fluoro-4-(trifluoromethyl)phenyl)-6-hydroxy-1-oxoisoquinolin-2(1H)-yl)benzamide (215), or its prodrug, isomer, tautomer, metabolite, pharmaceutically acceptable salt, polymorph, crystal, N-oxide, hydrate or any combination thereof, in an amount effective to treat uterine cancer or uterine fibroids.

In one embodiment, this invention provides a method of hormone therapy comprising the step of contacting an AKR1C3 enzyme of a subject with a compound of this invention or its to isomer, tautomer, pharmaceutically acceptable salt, pharmaceutical product, crystal, hydrate, N-oxide, metabolite, prodrug or any combination thereof, or a composition comprising the same, in an amount effective to inhibit AKR1C3. In another embodiment, this invention provides a method of hormone therapy comprising the step of contacting an AKR1C3 enzyme of a subject with a compound of this invention or its isomer, tautomer, pharmaceutically acceptable salt, pharmaceutical product, crystal, hydrate, N-oxide, metabolite, prodrug or any combination thereof, or a composition comprising the same, in an amount effective to decrease androgen levels in said subject. In another embodiment, this invention provides a method of hormone therapy comprising the step of contacting an AKR1C3 enzyme of a subject with a compound of this invention or its isomer, tautomer, pharmaceutically acceptable salt, pharmaceutical product, crystal, hydrate, N-oxide, metabolite, prodrug or any combination thereof, or a composition comprising the same, in an amount effective to suppress the transcriptional activity of the androgen receptor in said subject. In a further embodiment, this invention provides a method of hormone therapy comprising the step of contacting an AKR1C3 enzyme of a subject with a compound of this invention or its isomer, tautomer, pharmaceutically acceptable salt, pharmaceutical product, crystal, hydrate, N-oxide, metabolite, prodrug or any combination thereof, or a composition comprising the same, in an amount effective to effect a change in an androgen-dependent condition

In one embodiment, this invention provides a method of hormone therapy comprising the step of contacting an AKR1C3 enzyme of a subject with a compound of this invention or its isomer, tautomer, pharmaceutically acceptable salt, pharmaceutical product, crystal, hydrate, N-oxide, metabolite, prodrug or any combination thereof, or a composition comprising the same, in an amount effective to inhibit AKR1C3. In another embodiment, this invention provides a method of hormone therapy comprising the step of contacting an AKR1C3 enzyme of a subject with a compound of this invention or its isomer, tautomer, pharmaceutically acceptable salt, pharmaceutical product, crystal, hydrate, N-oxide, metabolite, prodrug or any combination thereof, or a composition comprising the same, in an amount effective to decrease estrogen levels in said subject. In a further embodiment, this invention provides a method of hormone therapy comprising the step of contacting an AKR1C3 enzyme of a subject with a compound of this invention or its isomer, tautomer, pharmaceutically acceptable salt, pharmaceutical product, crystal, hydrate, N-oxide, metabolite, prodrug or any combination thereof, or a composition comprising the same, in an amount effective to effect a change in an estrogen-dependent condition.

In another embodiment, this invention provides for the use of a compound as herein to described, or its prodrug, analog, isomer, tautomer, metabolite, derivative, pharmaceutically acceptable salt, pharmaceutical product, polymorph, crystal, N-oxide, hydrate or any combination thereof, for treating, reducing the severity of, reducing the incidence of, or delaying the onset of lung cancer.

In another embodiment, this invention provides for the use of a compound as herein described, or its prodrug, analog, isomer, tautomer, metabolite, derivative, pharmaceutically acceptable salt, pharmaceutical product, polymorph, crystal, N-oxide, hydrate or any combination thereof, for treating, reducing the severity of, reducing the incidence of, and delaying the onset of non small cell lung cancer.

There are 3 PPAR genes described in xenopus, PPAR α, β, and γ. (Dreyer, 1992). Human PPAR-α has been recently described, although putatively described as a single gene, unlike the xenopus genes, with no closely related family members (Sher, 1993). In the Xenopus system, PPAR α, β and γ show very high amino acid identity in their DNA binding and ligand binding regions, and correspondingly have shown similar abilities to activate the same target DNA sequences, and to be activated by clofibrate and peroxisome proliferator agents (Dreyer, 1992). However, human PPAR α, β and now γ have different chromosomal locations. All species α, β, and γ show differences in A/B and D regions, and all species demonstrate that α, β, and γ have different tissue expression patterns Amino acid identity between the xenopus γ and human γ receptors, in critical regions, is high enough to expect similar function, and indeed, studies on the human PPAR α by Sher et al. (1993) show it has the same ability as xenopus and murine PPAR α to activate the same reporters. Thus, PPAR-γ, is likely to activate these reporters in a manner similar to xenopus PPAR-γ, based on the highly conserved identity in critical regions. However, differences in sequence are significant enough to expect differences in vivo.

In some embodiments, the present invention provides a method for treating, reducing the incidence, delaying the onset or progression, or reducing and/or abrogating the symptoms associated with a metabolic disease in a subject, comprising the step of administering to said subject a compound of this invention, or its isomer, tautomer, pharmaceutically acceptable salt, pharmaceutical product, crystal, hydrate, N— oxide, prodrug, metabolite or any combination thereof, or a composition comprising the same in an amount effective to treat symptoms associated with a metabolic disease in the subject.

In one embodiment, this invention provides a method of treating a subject suffering from obesity-associated metabolic disorder, comprising the step of administering to said subject a compound of this invention, or its isomer, tautomer, pharmaceutically acceptable salt, pharmaceutical product, crystal, hydrate, N-oxide, prodrug, metabolite or any combination thereof, or a composition comprising the same in an amount effective to treat obesity-associated metabolic disorder in the subject.

In one embodiment, the compounds of this invention are useful for a) treating a subject suffering from obesity; b) preventing obesity in a subject; c) treating a subject suffering from obesity-associated metabolic disorder; d) preventing obesity-associated metabolic disorder in a subject; e) inducing anti-proliferative effects in a subject; f) treating a subject suffering from a prostaglandin-associated metabolic disorders; and g) preventing prostaglandin-associated metabolic disorders.

In one embodiment, the methods of treating obesity make use of a compound of Formula I. In another embodiment, the methods of treating obesity make use of a compound of Formula II. In another embodiment, the methods of treating obesity make use of a compound of Formula III. In another embodiment, the methods of treating obesity make use of a compound of Formula IV. In another embodiment, the methods of treating obesity make use of a compound of Formula V. In another embodiment the methods of treating obesity make use of a compound of Formula VI. In another embodiment, the methods of treating obesity make use of a compound of Formula VII. In another embodiment, the methods of treating obesity make use of a compound of Formula VIII. In another embodiment, the methods of treating obesity make use of a compound of Formula IX. In another embodiment, the methods of treating obesity make use of a compound of Formula X. In another embodiment, the methods of treating obesity make use of a compound of Formula XI. In another embodiment, the methods of treating obesity make use of a compound of Formula XII. In another embodiment, the methods of treating obesity make use of a compound of Formula XIII.

In one embodiment, the methods of preventing obesity make use of a compound of Formula I. In another embodiment, the methods of preventing obesity make use of a compound of Formula II. In another embodiment, the methods of preventing obesity make use of a compound of Formula III. In another embodiment, the methods of preventing obesity make use of a compound of Formula IV. In another embodiment, the methods of preventing obesity make use of a compound of Formula V. In another embodiment the methods of preventing obesity make use of a compound of Formula VI. In another embodiment, the methods of preventing obesity make use of a compound of Formula VII. In another embodiment, the methods of preventing obesity make use of a compound of Formula VIII. In another embodiment, the methods of preventing obesity make use of a compound of Formula IX. In another embodiment, the methods of preventing obesity make use of a compound of Formula X. In another embodiment, the methods of preventing obesity make use of a compound of Formula XI. In another embodiment, the methods of preventing obesity make use of a compound of Formula XII. In another embodiment, the methods of preventing obesity make use of a compound of Formula XIII.

In one embodiment, the methods of treating obesity-associated metabolic disorder make use of a compound of Formula I. In another embodiment, the methods of treating obesity-associated metabolic disorder make use of a compound of Formula II. In another embodiment, the methods of treating obesity-associated metabolic disorder make use of a compound of Formula III. In another embodiment, the methods of treating obesity-associated metabolic disorder make use of a compound of Formula IV. In another embodiment, the methods of treating obesity-associated metabolic disorder make use of a compound of Formula V. In another embodiment the methods of treating obesity-associated metabolic disorder make use of a compound of Formula VI. In another embodiment, the methods of treating obesity-associated metabolic disorder make use of a compound of Formula VII. In another embodiment, the methods of treating obesity-associated metabolic disorder make use of a compound of Formula VIII. In another embodiment, the methods of treating obesity-associated metabolic disorder make use of a compound of Formula IX. In another embodiment, the methods of treating obesity-associated metabolic disorder make use of a compound of Formula X. In another embodiment, the methods of treating obesity-associated metabolic disorder make use of a compound of Formula XI. In another embodiment, the methods of treating obesity-associated metabolic disorder make use of a compound of Formula XII. In another embodiment, the methods of treating obesity-associated metabolic disorder make use of a compound of Formula XIII.

In one embodiment, the methods of preventing obesity-associated metabolic disorder make use of a compound of Formula I. In another embodiment, the methods of preventing obesity-associated metabolic disorder make use of a compound of Formula II. In another embodiment, the methods of preventing obesity-associated metabolic disorder make use of a compound of Formula III. In another embodiment, the methods of preventing obesity-associated metabolic disorder make use of a compound of Formula IV. In another embodiment, the methods of preventing obesity-associated metabolic disorder make use of a compound of Formula V. In another embodiment the methods of preventing obesity-associated metabolic disorder make use of a compound of Formula VI. In another embodiment, the methods of preventing obesity-associated metabolic disorder make use of a compound of Formula VII. In another embodiment, the methods of preventing obesity-associated metabolic disorder make use of a compound of Formula VIII. In another embodiment, the methods of preventing obesity-associated metabolic disorder make use of a compound of Formula IX. In another embodiment, the methods of preventing obesity-associated metabolic disorder make use of a compound of Formula X. In another embodiment, the methods of preventing obesity-associated metabolic disorder make use of a compound of Formula XI. In another embodiment, the methods of preventing obesity-associated metabolic disorder make use of a compound of Formula XII. In another embodiment, the methods of preventing obesity-associated metabolic disorder make use of a compound of Formula XIII.

In one embodiment, the methods of inducing an anti-proliferative effect make use of a compound of Formula I. In another embodiment, the methods of inducing an anti-proliferative effect make use of a compound of Formula II. In another embodiment, the methods of inducing an anti-proliferative effect make use of a compound of Formula III. In another embodiment, the methods of inducing an anti-proliferative effect make use of a compound of Formula IV. In another embodiment, the methods of inducing an anti-proliferative effect make use of a compound of Formula V. In another embodiment the methods of inducing an anti-proliferative effect make use of a compound of Formula VI. In another embodiment, the methods of inducing an anti-proliferative effect make use of a compound of Formula VII. In another embodiment, the methods of inducing an anti-proliferative effect make use of a compound of Formula VIII. In another embodiment, the methods of inducing an anti-proliferative effect make use of a compound of Formula IX. In another embodiment, the methods of inducing an anti-proliferative effect make use of a compound of Formula X. In another embodiment, the methods of inducing an anti-proliferative effect make use of a compound of Formula XI. In another embodiment, the methods of inducing an anti-proliferative effect make use of a compound of Formula XII. In another embodiment, the methods of inducing an anti-proliferative effect make use of a compound of Formula XIII.

In one embodiment, the methods of treating prostaglandin-associated metabolic disorders make use of a compound of Formula I. In another embodiment, the methods of treating prostaglandin-associated metabolic disorders make use of a compound of Formula II. In another embodiment, the methods of treating prostaglandin-associated metabolic disorders make use of a compound of Formula III. In another embodiment, the methods of treating prostaglandin-associated metabolic disorders make use of a compound of Formula IV. In another embodiment, the methods of treating prostaglandin-associated metabolic disorders make use of a compound of Formula V. In another embodiment the methods of treating prostaglandin-associated metabolic disorders make use of a compound of Formula VI. In another embodiment, the methods of treating prostaglandin-associated metabolic disorders make use of a compound of Formula VII. In another embodiment, the methods of treating prostaglandin-associated metabolic disorders make use of a compound of Formula VIII. In another embodiment, the methods of treating prostaglandin-associated metabolic disorders make use of a compound of Formula IX. In another embodiment, the methods of treating prostaglandin-associated metabolic disorders make use of a compound of Formula X. In another embodiment, the methods of treating prostaglandin-associated metabolic disorders make use of a compound of Formula XI. In another embodiment, the methods of treating prostaglandin-associated metabolic disorders make use of a compound of Formula XII. In another embodiment, the methods of treating prostaglandin-associated metabolic disorders make use of a compound of Formula XIII.

In one embodiment, the methods of preventing prostaglandin-associated metabolic disorders make use of a compound of Formula I. In another embodiment, the methods of preventing prostaglandin-associated metabolic disorders make use of a compound of Formula II. In another embodiment, the methods of preventing prostaglandin-associated metabolic disorders make use of a compound of Formula III. In another embodiment, the methods of preventing prostaglandin-associated metabolic disorders make use of a compound of Formula IV. In another embodiment, the methods of preventing prostaglandin-associated metabolic disorders make use of a compound of Formula V. In another embodiment the methods of preventing prostaglandin-associated metabolic disorders make use of a compound of Formula VI. In another embodiment, the methods of preventing prostaglandin-associated metabolic disorders make use of a compound of Formula VII. In another embodiment, the methods of preventing prostaglandin-associated metabolic disorders make use of a compound of Formula VIII. In another embodiment, the methods of preventing prostaglandin-associated metabolic disorders make use of a compound of Formula IX. In another embodiment, the methods of preventing prostaglandin-associated metabolic disorders make use of a compound of Formula X. In another embodiment, the methods of preventing prostaglandin-associated metabolic disorders make use of a compound of Formula XI. In another embodiment, the methods of preventing prostaglandin-associated metabolic disorders make use of a compound of Formula XII. In another embodiment, the methods of preventing prostaglandin-associated metabolic disorders make use of a compound of Formula XIII

As used herein, the term “obesity-associated metabolic disorder” refers, in one embodiment, to a disorder which results from, is a consequence of, is exacerbated by or is secondary to obesity. Non-limiting examples of such a disorder are osteoarthritis, Type II diabetes mellitus, increased blood to pressure, stroke, and heart disease.

As used herein, the term “prostaglandin-associated metabolic disorder” refers, in one embodiment, to a disorder which results from, is a consequence of, is exacerbated by changes in prostaglandin levels.

Cholesterol, triacylglycerol and other lipids are transported in body fluids by lipoproteins which may be classified according to their density, for example, the very low density lipoproteins (VLDL), intermediate density lipoproteins (IDL), low density lipoproteins (LDL) and high density lipoproteins (HDL).

It has been shown that high levels of LDL-Cholesterol in the blood correlate with atherosclerosis which is a progressive disease characterized in part by sedimentation of lipids in inner walls of arteries, particularly of coronary arteries. It has also been shown that a high blood level of LDL-Cholesterol correlates with coronary heart disease. Also, a negative correlation exists between blood levels of HDL cholesterol and coronary heart disease.

The level of total cholesterol in blood, which is the sum of HDL-Cholesterol, LDL-Cholesterol, VLDL-Cholesterol and chylomicron-Cholesterol, is not necessarily predictive of the risk of coronary heart disease and atherosclerosis.

The correlation between atherosclerosis and LDL cholesterol levels, however, is much higher than a similar correlation between atherosclerosis and total serum cholesterol levels.

In one embodiment, this invention provides methods of use of the compounds as herein described for improving the lipid profile and/or reducing the circulating lipid levels in a subject. In some embodiments, according to this aspect of the invention, the subject suffers from one or more conditions selected from the group consisting of: atherosclerosis and its associated diseases, premature aging, Alzheimer's disease, stroke, toxic hepatitis, viral hepatitis, peripheral vascular insufficiency, renal disease, and hyperglycemia, and the invention provides for the administration of a compound or composition comprising the same, as herein described, which in some embodiments positively affects a lipid profile in the subject, which is one means by which the method is useful in treating the indicated diseases, disorders and conditions.

In one embodiment the invention provides for the treatment of atherosclerosis and its associated diseases, such as for example, cardiovascular disorders, cerebrovascular disorders, peripheral vascular disorders, intestinal vascular disorders, or combinations thereof.

In one embodiment cardiovascular disorders comprise of hypertention (HTN), coronary artery disease (CAD) or myocardial perfusion. In another embodiment this invention provides methods to of use of the SARM compounds as herein described for promoting aortic smooth muscle cell proliferation. In another embodiment this invention provides methods of use of the compounds as herein described for treating arteriosclerosis. In another embodiment this invention provides methods of use of the compounds as herein described for lowering blood pressure. In another embodiment this invention provides methods of use of the compounds as herein described for treating cardiac diseases and disorders comprising cardiomyopathy, cardiac dysfunctions such as, myocardial infarction, cardiac hypertrophy and cognitive heart failure. In another embodiment this invention provides methods of use of the compounds as herein described for cardioprotection comprising cardioprotection in insulin resistance; treating diabetes type I ans II, metabolic syndrome, syndrome X and/or high blood pressure.

In one embodiment, the invention provides a method of treating, preventing, reducing the risk of mortality from cardiovascular and/or cerebrovascular disease in a subject, comprising administering a compound of this invention or its prodrug, analog, isomer, tautomer, metabolite, derivative, pharmaceutically acceptable salt, pharmaceutical product, polymorph, crystal, N-oxide, hydrate or any combination thereof, or a pharmaceutical composition comprising the same. In one embodiment, the method of treating cardiovascular and/or cerebrovascular disease makes use of a compound of Formula I. In another embodiment, the method of treating cardiovascular and/or cerebrovascular disease makes use of a compound of Formula II. In yet another embodiment, the method of treating cardiovascular and/or cerebrovascular disease makes use of a compound of Formula III. In still another embodiment, the method of treating cardiovascular and/or cerebrovascular disease makes use of a compound of Formula IV. In a further embodiment, the method of treating cardiovascular and/or cerebrovascular disease makes use of a compound of Formula V. In another embodiment, the method of treating cardiovascular and/or cerebrovascular disease makes use of a compound of Formula VI. In yet another embodiment, the method of treating cardiovascular and/or cerebrovascular disease makes use of a compound of Formula VII. In still another embodiment, the method of treating cardiovascular and/or cerebrovascular disease makes use of a compound of Formula VIII. In a further embodiment, the method of treating cardiovascular and/or cerebrovascular disease makes use of a compound of Formula IX. In another embodiment, the method of treating cardiovascular and/or cerebrovascular disease makes use of a compound of Formula X. In yet another embodiment, the method of treating cardiovascular and/or cerebrovascular disease makes use of a compound of Formula XI. In still another embodiment, the method of treating cardiovascular and/or cerebrovascular disease makes use of a compound of Formula XII. In still another embodiment, the method of treating cardiovascular and/or cerebrovascular disease makes use of a compound of Formula to XIII.

In another embodiment, compounds of Formulae I-XII are co-administered with HDL-elevating agents. In another embodiment, a compound of this invention is co-administered with an HDL-elevating agent. In another embodiment, HDL-elevating agents include niacin. In another embodiment the HDL-elevating agents include fibrates including gemfibrozil (Lopid), thiourea based gemfibrozil analogues, and fenofibrate (TriCor®). In another embodiment, HDL-elevating agents include statins. In another embodiment, HDL-elevating agents include 1-hydroxyalkyl-3-phenylthiourea, and analogs thereof.

In one embodiment, this invention provides a method of reducing circulating lipid levels in a subject, said method comprising administering a compound of this invention or its isomer, tautomer, prodrug, metabolite, pharmaceutically acceptable salt, hydrate, N-oxide, or any combination thereof, or a composition comprising the same. In one embodiment, the subject suffers from atherosclerosis and its associated diseases, premature aging, Alzheimer's disease, stroke, toxic hepatitis, viral hepatitis, peripheral vascular insufficiency, renal disease, hyperglycemia, or any combination thereof.

In one embodiment, this invention provides a method of treating atherosclerosis and its associated diseases, such as, for example, cardiovascular disorders, cerebrovascular disorders, peripheral vascular disorders, or intestinal vascular disorders in a subject, the method comprising the step of administering to the subject compound of this invention or its pharmaceutically acceptable salt, isomer, tautomer, hydrate, N-oxide, or any combination thereof, or a composition comprising the same.

In certain embodiments, the present invention provides methods of treating a condition. In certain embodiments, the present invention provides methods of preventing a condition. In certain embodiments, the present invention provides methods of delaying the onset of a condition. In certain embodiments, the present invention provides methods of reducing the recurrence of a condition. In certain embodiments, the present invention provides methods of reducing the severity of a condition.

In one embodiment, the methods of the present invention are useful for treating prostate cancer. In one embodiment the methods of the present invention are useful for treating PIN. In one embodiment, the methods of the present invention are useful for treating breast cancer. In one embodiment, the methods of the present invention are useful for treating acne. In one embodiment, the methods of the present invention are useful for treating alopecia.

The inhibition of cyclooxygenase (COX) enzymes (e.g., COX-1 and/or COX-2) is undesirable for compounds that act as AKR1C3 inhibitors, to act as inhibition of cyclooxygenase enzymes, especially COX-1, typically leads to unwanted side effects in a patient, such as gastric irritation, ulcers and cardiovascular problems. In one embodiment, compounds of the present invention are much more potent inhibitors of AKR1C3 than they are inhibitors of cyclooxygenase.

In one embodiment, the compounds of the present invention lack cross-reactivity with cyclooxygenase (COX) enzymes. In one embodiment, the compounds of the present invention lack cross-reactivity with COX-1 enzyme. In one embodiment, the compounds of the present invention lack cross-reactivity with COX-2 enzyme. In one embodiment, the compounds of the present invention lack cross-reactivity with COX-1 and COX-2 enzymes. The term “cross-reactivity”, as used herein, means that the ratio of inhibition of AKR1C3 to inhibition of cyclooxygenase for a compound of the present invention is greater than about 10-fold. In another embodiment, the ratio of inhibition is for example greater than about 100. In a further embodiment, the ratio of inhibition is greater than about 1000.

In certain embodiments, the methods described herein prevent or lessen typical side-effects associated with inhibition of AKR1C3 from occurring. In certain embodiments, the methods described herein prevent or lessen gastric irritation, ulcers, and/or cardiovascular problems.

In certain embodiments, a compound of this invention may be an estrogen receptor (ER) agonists. In other embodiment, a compound of this invention may be an ER antagonist. In still another embodiment, a compound of this invention may be an ER partial agonist/antagonist. In yet another embodiment, a compound of this invention is not an ER agonist. In a further embodiment, a compound of this invention is not an ER antagonist. In one embodiment, a compound of this invention is not an ER partial agonist/antagonist. In another embodiment, a compound of this invention does not bind to an ER.

In certain embodiments, an AKR1C3 inhibitor of this invention may modulate the activity of androgen receptor (AR) agonists. In other embodiments, an AKR1C3 inhibitor of this invention may modulate the activity of an AR antagonist. In still another embodiment, an AKR1C3 inhibitor of this invention may modulate the activity of a selective androgen receptor modulator (SARM). In yet another embodiment, a compound of this invention is not an AR agonist. In a further embodiment, a compound of this invention is not an AR antagonist. In one embodiment, a compound of this invention is not a SARM. In another embodiment, a compound of this invention does not bind to an AR.

In one embodiment, this invention provides a method of lowering total serum testosterone levels in a male subject comprising administering a therapeutically effective amount of an AKR1C3 inhibitor of this invention. In another embodiment, this invention provides a method of lowering serum testosterone in a male subject comprising administering a therapeutically effective amount of an AKR1C3 inhibitor of this invention. In yet another embodiment, this invention provides a method of lowering serum free testosterone in a male subject comprising administering a therapeutically effective amount of an AKR1C3 inhibitor of this invention. In still another embodiment, this invention provides a method of lowering serum levels of prostate-specific antigen (PSA) in a male subject comprising administering a therapeutically effective amount of an AKR1C3 inhibitor of this invention. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)benzamide (214), 4-(4-(3-fluoro-4-(trifluoromethyl)phenyl)-6-hydroxy-1-oxoisoquinolin-2(1H)-yl)benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of lowering total serum testosterone levels in a male subject comprising administering a therapeutically effective amount of an AKR1C3 inhibitor of this invention, wherein the lowering of total serum testosterone is independent of a reduction of serum luteinizing hormone levels. In another embodiment, this invention provides a method of lowering serum testosterone levels in a male subject comprising administering a therapeutically effective amount of an AKR1C3 inhibitor of this invention, wherein the lowering of serum free testosterone is independent of a reduction of serum luteinizing hormone levels. In yet another embodiment, this invention provides a method of lowering serum free testosterone levels in a male subject comprising administering a therapeutically effective amount of an AKR1C3 inhibitor of this invention, wherein the lowering of serum free testosterone is independent of a reduction of serum luteinizing hormone levels. In still another embodiment, this invention provides a method of lowering serum PSA levels in a male subject comprising administering a therapeutically effective amount of an AKR1C3 inhibitor of this invention, wherein the lowering of serum PSA is independent of a reduction of serum luteinizing hormone levels. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)benzamide (214), 4-(4-(3-fluoro-4-(trifluoromethyl)phenyl)-6-hydroxy-1-oxoisoquinolin-2(1H)-yl)benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of increasing survival of a subject with advanced prostate cancer comprising administering a therapeutically effective amount of an AKR1C3 inhibitor of this invention. In another embodiment, this invention provides a method of increasing survival of a subject with castration-resistant prostate cancer (CRPC) comprising administering a therapeutically effective amount of an AKR1C3 inhibitor of this invention. In one embodiment, the AKR1C3 inhibitor is a compound of formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)benzamide (214), 4-(4-(3-fluoro-4-(trifluoromethyl)phenyl)-6-hydroxy-1-oxoisoquinolin-2(1H)-yl)benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of prolonging progression-free survival of a subject with advanced prostate cancer comprising administering a therapeutically effective amount of an AKR1C3 inhibitor of this invention. In another embodiment, this invention provides a method of prolonging progression-free survival of a subject with castration-resistant prostate cancer (CRPC) comprising administering a therapeutically effective amount of an AKR1C3 inhibitor of this invention. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)-benzamide (214), 4-(4-(3-fluoro-4-(trifluoromethyl)phenyl)-6-hydroxy-1-oxoisoquinolin-2(1H)-yl)-benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of lowering total serum estradiol levels in a subject comprising administering a therapeutically effective amount of an AKR1C3 inhibitor of this invention. In another embodiment, this invention provides a method of lowering serum free estradiol in a subject comprising administering a therapeutically effective amount of an AKR1C3 inhibitor of this invention. In one embodiment, a subject is a male subject. In another embodiment, a subject is a female subject. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)benzamide (214), 4-(4-(3-fluoro-4-(trifluoromethyl)phenyl)-6-hydroxy-1-oxoisoquinolin-2(1H)-yl)benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of lowering intratumor levels of testosterone in a subject comprising administering a therapeutically effective amount of an AKR1C3 inhibitor of this invention. In one embodiment, a subject is a male subject. In another embodiment, a subject is a female subject. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)-benzamide (214), 4-(4-(3-fluoro-4-(trifluoromethyl)phenyl)-6-hydroxy-1-oxoisoquinolin-2(1H)-yl)-benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of lowering intratumor levels of DHT in a subject comprising administering a therapeutically effective amount of an AKR1C3 inhibitor of this invention. In one embodiment, a subject is a male subject. In another embodiment, a subject is a female subject. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)-benzamide (214), 4-(4-(3-fluoro-4-(trifluoromethyl)phenyl)-6-hydroxy-1-oxoisoquinolin-2(1H)-yl)-benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of lowering intratumor levels of estrogen in a subject comprising administering a therapeutically effective amount of an AKR1C3 inhibitor of this invention. In one embodiment, a subject is a male subject. In another embodiment, a subject is a female subject. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)-benzamide (214), 4-(4-(3-fluoro-4-(trifluoromethyl)phenyl)-6-hydroxy-1-oxoisoquinolin-2(1H)-yl)-benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of increasing survival of a subject with advanced breast cancer comprising administering a therapeutically effective amount of an AKR1C3 inhibitor of this invention. In another embodiment, this invention provides a method of increasing survival of a subject with refractory breast cancer comprising administering a therapeutically effective amount of an AKR1C3 inhibitor of this invention. In another embodiment, this invention provides a method of increasing survival of a subject with AR-positive or ER-positive breast cancer comprising administering a therapeutically effective amount of an AKR1C3 inhibitor of this invention. In one embodiment, the AKR1C3 inhibitor is a compound of Formula I-XI and/or to formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)-benzamide (214), 4-(4-(3-fluoro-4-(trifluoromethyl)phenyl)-6-hydroxy-1-oxoisoquinolin-2(1H)-yl)-benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of prolonging progression-free survival of a subject with advanced breast cancer comprising administering a therapeutically effective amount of an AKR1C3 inhibitor of this invention. In another embodiment, this invention provides a method of prolonging progression-free survival of a subject with refractory breast cancer comprising administering a therapeutically effective amount of an AKR1C3 inhibitor of this invention. In another embodiment, this invention provides a method of prolonging progression-free survival of a subject with AR-positive or ER-positive breast cancer comprising administering a therapeutically effective amount of an AKR1C3 inhibitor of this invention. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)benzamide (214), 4-(4-(3-fluoro-4-(trifluoromethyl)phenyl)-6-hydroxy-1-oxoisoquinolin-2(1H)-yl)benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of increasing survival of a subject with advanced uterine cancer comprising administering a therapeutically effective amount of an AKR1C3 inhibitor of this invention. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)benzamide (214), 4-(4-(3-fluoro-4-(trifluoromethyl)phenyl)-6-hydroxy-1-oxoisoquinolin-2(1H)-yl)benzamide (215), or their prodrug, isomer, tautomer metabolite, pharmaceutically acceptable salt, polymorph, crystal, N-oxide, hydrate or any combination thereof, or any embodiment thereof, as herein described.

In one embodiment, this invention provides a method of prolonging progression-free survival of a subject with advanced uterine cancer comprising administering a therapeutically effective amount of an AKR1C3 inhibitor of this invention. In one embodiment, the AKR1C3 inhibitor is a compound of Formula I-XI and/or formula XII and/or formula XIII or their prodrug, isomer, tautomer metabolite, pharmaceutically acceptable salt, polymorph, crystal, N-oxide, hydrate or any combination thereof, or any embodiment thereof, as herein described. In one embodiment, the AKR1C3 inhibitor is characterized by the structure of 6-hydroxy-2-(4-hydroxyphenyl)-4-phenylisoquinolin-1(2H)-one (15a), 6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h), (E)-3-(6,8-dihydroxy-2-(4-hydroxyphenyl)-1-oxo-1,2-dihydroisoquinolin-4-yl)acrylic acid (15l), 2-(4-bromomethyl)phenyl-6-hydroxy-4-(4-hydroxyphenyl)isoquinolin-1(2H)-one (11), 6-hydroxy-2-(4-hydroxyphenyl)-4-(4-(trifluoromethyl)phenylisoquinolin-1(2H)-one (13), 6-hydroxy-2-(4-(hydroxymethyl)phenyl)-4-(4-hydroxyphenyl)isoquinolin-1(2H)-one (14), 2-(4-(bromomethyl)-3-hydroxyphenyl)-6-hydroxy-4-(4-(trifluoromethyl)phenyl)isoquinolin-1(2H)-one (26), 6-hydroxy-2-(4-hydroxyphenyl)-1-oxo-4-(3,4,5-trifluorophenyl)-1,2-dihydroisoquinoline-8-carbonitrile (85), 3-(4-(3-fluoro-4-(trifluoromethyl)phenyl)-6-hydroxy-1-oxoisoquinolin-2(1H)-yl)-benzamide (214), 4-(4-(3-fluoro-4-(trifluoromethyl)phenyl)-6-hydroxy-1-oxoisoquinolin-2(1H)-yl)-benzamide (215), or their prodrug, isomer, tautomer metabolite, pharmaceutically acceptable salt, polymorph, crystal, N-oxide, hydrate or any combination thereof, or any embodiment thereof, as herein described.

In some embodiments, a compound of this invention is not a nuclear receptor binding agent.

In some embodiments, the compounds of the present invention are administered as a mono-therapy. In other embodiments, the compounds of the present invention are administered as part of a combination therapy. For example, compounds of this invention may be used in combination with other drugs or therapies that are used in the treatment of the diseases or conditions for which compounds of this invention are useful. In such combinations, each active ingredient can be administered either in accordance with their usual dosage range or a dose below their usual dosage range. For example, a compound of this invention may be adjunctively administered in combination with an antiandrogen, antiestrogen, anti-cancer drug, 5-alpha reductase inhibitor, aromatase inhibitor, GnRH agonist, GnRH antagonist, lyase inhibitor, progestin, prostate cancer vaccine, an agent that treats prostate cancer (docetaxel/prednisone, estramustine), an agent that treats breast cancer (Herceptin®, cyclophosphamide, methotrexate, fluorouracil, and doxorubicin (Adriamycin).

In one embodiment, the methods of the present invention comprise administering a compound of this invention as the sole active ingredient. However, also encompassed within the scope of the present invention are methods for treating a disorder that responds to an AKR1C3 inhibitor including treating and/or preventing prostate cancer, precancerous precursors of prostate adenocarcinoma, PIN, HGPIN, advanced prostate cancer, castration resistant prostate cancer, benign prostate hyperplasia (BPH), lung cancer, non-small cell lung cancer (NSCLC), acne, seborrhea, hirsuitism, baldness, alopecia, precocious puberty, adrenal hypertrophy, polycystic ovary syndrome, breast cancer, metastatic breast cancer, refractory breast cancer, AR-positive breast cancer, endometriosis, myeloma or leiomyoma which comprise administering a compound of this invention in combination with one or more therapeutic agents. These agents include, but are not limited to: selective estrogen receptor modulators (SERM), selective estrogen receptor degraders (fulvestrant), HER2 inhibitors (lapatinib, trastuzumab), bevacizumab, chemotherapeutic agents, taxanes, anthracyclines, epothilones, LHRH analogs, antiandrogens, antiestrogens, anticancer drugs, 5-alpha reductase inhibitors, aromatase inhibitors (exemestane, anastrozole, letrozole, vorozole, formestane, fadrozole), progestins, agents acting through other nuclear hormone receptors, progesterone, estrogen, PDES inhibitors, apomorphine, bisphosphonate, growth factor inhibitors (such as those that inhibit VEGF, IGF and the like VEGF-A inhibitor (bevacizumab), selective androgen receptor modulators (SARMs), or one or more additional agents that treats diseases that respond to AKR1C3 inhibitors.

Thus, in one embodiment, the methods of the present invention comprise administering the compound of this invention, in combination with a chemotherapeutic agent. In one embodiment, the chemotherapeutic agent is a taxane. In another embodiment, the chemotherapeutic agent is an anthracycline. In one embodiment, the chemotherapeutic agent is an epothilone (ixabepilone). Thus, in one embodiment, the methods of the present invention comprise administering the compound of this invention in combination with an LHRH analog, taxanes, anthracyclines, or epothilones. In another embodiment, the methods of the present invention comprise administering the compound of this invention, in combination with an antiandrogen (bicalutamide, nilutamide, flutamide, or MDV3100). In another embodiment, the methods of the present invention comprise administering a compound of this invention, in combination with an antiestrogen. In another embodiment, the methods of the present invention comprise administering a compound of this invention, in combination with an anticancer drug. In another embodiment, the methods of the present invention comprise administering a compound of this invention, in combination with a 5-alpha reductase inhibitor. In another embodiment, the methods of the present invention comprise administering a compound of this invention, in combination with an aromatase inhibitor. In another embodiment, the methods of the present invention comprise administering a compound of this invention, in combination with a progestin. In another embodiment, the methods of the present invention comprise administering a compound of this invention, in combination with an agent acting through other nuclear hormone receptors. In another embodiment, the methods of the present invention comprise administering a compound of this invention, in combination with a selective estrogen receptor modulators (SERM). In another embodiment, the methods of the present invention comprise administering a compound of this invention, in combination with an estrogen. In another embodiment, the methods of the present invention comprise administering a compound of this invention, in combination with one or more additional agents that act through another hydroxysteroid deoxygenase receptor.

In one embodiment, the methods of this invention make use of compounds of formula I-XI and/or formula XII and/or formula XIII in combination with PPARα ligands such as bezafibrate, fenofibrate, gemfibrozil. In another embodiment, the methods of this invention make use of compounds of formula I-XI and/or formula XII and/or formula XIII in combination with PPARγ ligands such as darglitazone, pioglitazone, rosiglitazone, isaglitazone, rivoglitazone, netoglitazone. In yet another embodiment, the methods of this invention make use of compounds of formula I-XI and/or formula XII and/or formula XIII in combination with Dual acting PPAR ligands, such as naveglitazar, farglitazar, tesaglitazar, ragaglitazar, oxeglitazar, PN-2034, PPAR δ. In yet another embodiment, the methods of this invention make use of compounds of formula I-XI and/or formula XII and/or formula XIII in combination with 17-ketoreductase inhibitors, 3β-DHΔ4,6-isomerase inhibitors, 313-DHA4,5-isomerase inhibitors, 17,20 desmolase inhibitors, p450c17 inhibitors, p450ssc inhibitors, 17,20-lyase inhibitors, or combinations thereof.

In one embodiment, the agent treating the endocrine system is a peroxisome proliferator-activated receptor ligand. In some embodiments, peroxisome proliferator-activated receptor ligands include but are not limited to bezafibrate, fenofibrate, gemfibrozil, darglitazone, pioglitazone, rosiglitazone, isaglitazone, rivoglitazone, netoglitazone, naveglitazar, farglitazar, tesaglitazar, ragaglitazar, oxeglitazar, or PN-2034. In one embodiment, the methods of this invention make use of compounds of formula I-XI and/or formula XII and/or formula XIII in combination with a peroxisome proliferators-activated receptor ligand. In one embodiment, the methods of this invention make use of compounds of formula I-XI and/or formula XII and/or formula XIII in combination with bezafibrate, fenofibrate, gemfibrozil, darglitazone, pioglitazone, rosiglitazone, isaglitazone, rivoglitazone, netoglitazone, naveglitazar, farglitazar, tesaglitazar, ragaglitazar, oxeglitazar, or PN-2034.

Such other drug(s) may be administered, by a route and in an amount commonly to used therefor, contemporaneously or sequentially with a compound of this invention. When a compound of this invention is used contemporaneously with one or more other drugs, a pharmaceutical unit dosage form containing such other drugs in addition to the compound of this invention may be employed. Accordingly, the pharmaceutical compositions of the present invention include those that also contain one or more other active ingredients, in addition to a compound of this invention.

By adjunctive administration is meant simultaneous administration of the compounds in the same-dosage form, simultaneous administration in separate dosage forms, and separate administration of the compounds.

In one embodiment, the methods of this invention may comprise administration of a compound of this invention at various dosages. In one embodiment, the compound of this invention is administered at a dosage of about 0.1 to about 200 mg per day. In one embodiment, the compound of this invention is administered at a dose of about 0.1 to about 10 mg, or in another embodiment, 0.1 to about 25 mg, or in another embodiment, about 0.1 to about 60 mg, or in another embodiment, about 0.5 to about 15 mg, or in another embodiment, about 0.5 to about 30 mg, or in another embodiment, about 0.5 to about 25 mg, or in another embodiment, about 0.5 to about 60 mg, or in another embodiment, about 0.75 to about 15 mg, or in another embodiment, 0.75 to about 60 mg, or in another embodiment, about 1 to about 5 mg, or in another embodiment, about 1 to about 20 mg, or in another embodiment, about 5 to about 15 mg, or in another embodiment, about 30 to about 60 mg, or in another embodiment, about 30 to about 75 mg, or in another embodiment, about 100 to about 2000 mg.

In one embodiment, the methods of this invention may comprise administration of a compound of this invention at various dosages. In one embodiment, a compound of this invention is administered at a dosage of about 0.1 mg. In another embodiment a compound of this invention is administered at a dosage of about 0.5 mg, about 1 mg, about 2.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg.

The term “treating” means to relieve, alleviate, delay, reduce, reverse, improve or prevent at least one symptom of a condition in a subject. The term “treating” may also mean to arrest, delay the onset (i.e., the period prior to clinical manifestation of a disease) and/or reduce the risk of developing or worsening a condition. The compounds of the present invention may be administered as a mono-therapy or administered as part of a combination therapy. For example, one or more of the compounds of the present invention may be co-administered or used in combination with one or more additional therapies known in the art.

An “effective amount” means the amount of a compound of this invention that, when administered to a patient (e.g., a mammal) for treating a disease, is sufficient to effect such treatment for the disease, or an amount of a compound of this invention that is sufficient for inhibiting AKR1C3 to achieve an objective of the invention. The “effective amount” will vary depending on the compound, the disease and its severity and the age, weight, etc., of the patient to be treated. In certain embodiments, the “effective amount” selectively inhibits AKR1C3.

As used herein, the term “administering” refers to bringing a subject in contact with a compound of the present invention. As used herein, administration can be accomplished in vitro, i.e. in a test tube, or in vivo, i.e. in cells or in tissues of living organisms, for example humans. In one embodiment, the present invention encompasses administering the compounds of the present invention to a subject.

A subject or patient in whom administration of the therapeutic compound is an effective therapeutic regimen for a disease or disorder is preferably a human, but can be any animal, including a laboratory animal in the context of a preclinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%.

It is to be understood that any use of any of the compounds as herein described may be used in the treatment of any disease, disorder or condition as described herein, and represents an embodiment of this invention.

In some embodiments, the term “comprise” or grammatical forms thereof, refers to the inclusion of the indicated active agent, such as the compound of this invention, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components, which exert a therapeutic effect and belong to a class of compounds distinct from that of the indicated active ingredient, by acting via a different mechanism of action, for example, and representing an embodiment of this invention, polypeptides comprising T cell epitopes present in a composition may be specifically combined with polypeptides comprising B cell epitopes. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In one embodiment, the present invention provides combined preparations. In one embodiment, the term “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with to the needs of a patient subpopulation to be treated or the needs of the single patient which different needs can be due to a particular disease, severity of a disease, age, sex, or body weight as can be readily made by a person skilled in the art.

The following examples are presented in order to more fully illustrate the preferred embodiments of the invention. They should in no way, however, be construed as limiting the broad scope of the invention. While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

All references cited herein are hereby incorporated by reference in their entirety.

### EXAMPLES

### Example 1

### Synthesis of isoquinolin-1(2H)-one AKR1C3 inhibitors

### Example 1A

### Synthesis of 6-hydroxy-2,4-bis(4-hydroxyphenyl)isoquinolin-1(2H)-one (6)

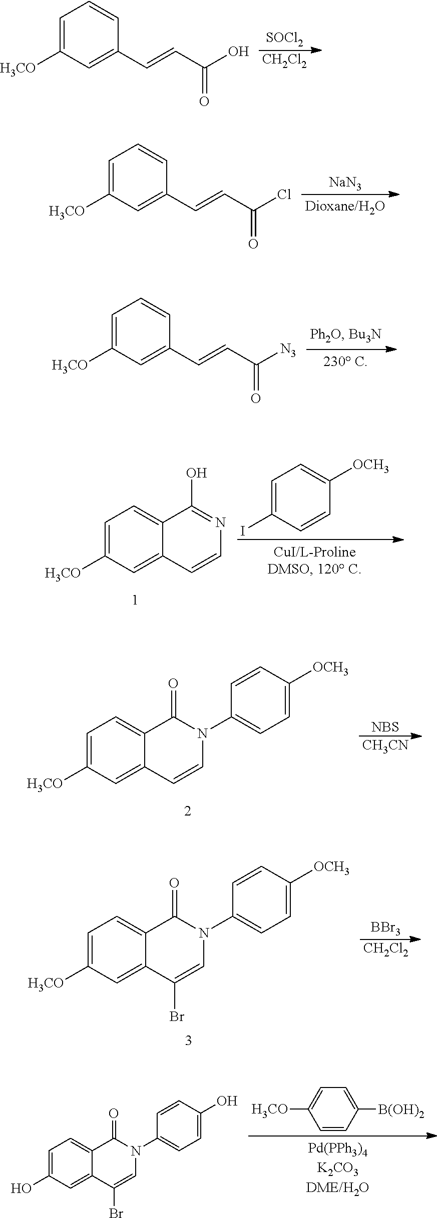

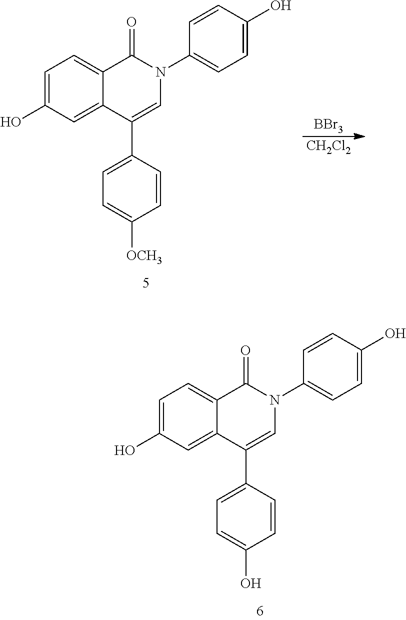 [SMILES: COc1ccc(-c2cn(-c3ccc(O)cc3)c(=O)c3ccc(O)cc23)cc1 ; "CH2Cl2" ; "BBr3" ; O=c1c2ccc(O)cc2c(-c2ccc(O)cc2)cn1-c1ccc(O)cc1]

### Synthesis of 6-methoxyisoquinoline-1-ol (1)

A mixture of 17.82 g (0.10 mol) of trans-3-methoxycinnamic acid and thionyl chloride (14.28 g, 0.12 mol) was placed in a 250 mL single-necked round-bottomed flask fitted with a stirring bar and reflux condenser. 80 mL of dry methylene chloride was added to the flask. The mixture was heated to reflux for 3 hours. Then, the solvent was removed under reduced pressure. The residue oil was dried under vacuum overnight.

The pale-yellow solid acid chloride was dissolved in 20 mL of 1,4-dioxane and added dropwise with stirring to a 0° C. suspension of 19.50 g (0.30 mol) of sodium azide in 80 mL of 1,4-dioxane/water (1:1 mixture). During the addition the temperature was maintained at 0° C. After complete addition of the acid chloride, the mixture was stirred at 0° C. for an additional hour, and then diluted with 75 mL of water. The mixture was extracted with methylene chloride (2×40 mL). The combined extracts were dried over anhydrous magnesium sulfate, filtered and concentrated to ca. 100 mL. The solution was diluted with 20 mL of phenyl ether and further concentrated to remove the remaining methylene chloride.

A 500 mL 3-necked round-bottomed flask fitted with an argon inlet, reflux condenser, additional funnel and an internal thermometer was charged with 29 mL of tributylamine and 80 mL of phenyl ether. The solution was heated to 230° C., and the acyl azide in 20 mL of phenyl ether was added dropwise with stirring over 3 hours from an addition funnel. During the addition, the reflux temperature gradually decreased to 200° C. After, completion of the addition, the distillate was collected in the addition funnel (15 mL of a 1:1 mixture of tributylamine/phenyl ether) until the temperature reached 230° C. After heating for an additional hour at 230° C., the mixture was cooled to room temperature. The mixture was then poured to 500 mL of hexanes with stirring. The solid was filtered and washed with hexanes (2×100 mL). The pale-yellow solid was recrystallized from ethyl acetate/methanol (9/1 v/v) to give a pure pale-yellow crystalline material, 15.28 g, 87.2% yield. MS: 198.1 [M+Na]⁺. ¹H NMR (DMSO-d₆, 300 MHz) δ 11.06 (s, 1H), 8.08 (d, 1H, J=8.5 Hz), 7.14-7.14 (m, 1H), 7.10 (d, 1H, J=2.5 Hz), 7.05-7.03 (m, 1H), 7.04 (dd, 1H, J₁=9.0 Hz, J₂=2.5 Hz), 6.47 (d, 1H, J=7.0 Hz), 3.86 (s, 3H).

### Synthesis of 6-methoxy-2-(4-methoxyphenyl)isoquinolin-1(2H)-one (2)

6-Methoxyisoquinoline-1-ol (1) (2.00 g, 11.42 mmol), 4-iodoanisole (4.01 g, 17.13 mmol), copper(I) iodide (0.44 g, 2.28 mmol), L-proline (0.53 g, 4.57 mmol) and anhydrous potassium carbonate (3.16 g, 22.84 mmol) were placed in a dry 250 mL three-necked round-bottomed flask fitted with a stirring bar and reflux condenser. The reaction flask was vacuumed and refilled with dry argon. 50 mL of anhydrous methyl sulfoxide was added via a syringe. The reaction mixture was stirred and heated to 130° C. for 20 hours. 50 mL of water was added to quench the reaction, and yellow solid precipitated out. The pale-yellow solid was filtered, washed with water (2×20 mL) and dried in air. This pale-yellow solid was purified by flash column chromatography (silica gel, ethyl acetate) to give a pale-yellow solid product, 2.90 g, 90.3% yield. MS: 282.2 [M+H]⁺. ¹H NMR (DMSO-d₆, 300 MHz) δ 8.14 (d, 1H, J=8.7 Hz), 7.39-7.34 (m, 3H), 7.19 (d, 1H, J=2.4 Hz), 7.13-7.03 (m, 3H), 6.62 (d, 1H, J=7.5 Hz), 3.89 (s, 3H), 3.81 (s, 3H).

### Synthesis of 4-bromo-6-methoxy-2-(4-methoxyphenyl)isoquinolin-1(2H)-one (3)

6-Methoxy-2-(4-methoxyphenyl)isoquinolin-1(2H)-one (2) (0.50 g, 1.78 mmol) was placed in a dry 250 mL single-necked round-bottomed flask fitted with a stirring bar and septa. Acetonitrile (10 mL) was added via a syringe under argon atmosphere at room temperature. N-Bromosuccinimide (0.33 g, 1.87 mmol) was added portion wise under argon atmosphere at room temperature. The reaction mixture was allowed to stir at room temperature for 2 hours. Then, 20 mL of saturated sodium bicarbonate solution was added. The mixture was extracted with ethyl acetate (3×10 mL). Organic layers were to separated, dried over anhydrous magnesium sulfate and concentrated under vacuum. The residue was purified by flash column chromatography (silica gel, hexanes/EtOAc=2/3 v/v) to give a white solid product, 0.55 g, 85.9% yield. MS: 360.4 [M+H]⁺. ¹H NMR (DMSO-d₆, 300 MHz) δ 8.14 (d, 1H, J=8.7 Hz), 7.39-7.34 (m, 3H), 7.19 (d, 1H, J=2.4 Hz), 7.13-7.03 (m, 3H), 6.62 (dd, 1H, J=7.5 Hz), 3.89 (s, 3H), 3.81 (s, 3H).

### Synthesis of 4-bromo-6-hydroxy-2-(4-hydroxyphenyl)isoquinolin-1(2H)-one (4)

4-Bromo-6-methoxy-2-(4-methoxyphenyl)isoquinolin-1(2H)-one (3) (0.22 g, 0.61 mmol) was placed in a dry 150 mL single-necked flask fitted with a stirring bar and septa. Methylene chloride (30 mL) was added via a syringe. Boron tribromide (1.83 mL of 1.0 M methylene chloride solution) was added dropwise with stirring under argon atmosphere at room temperature. The reaction mixture was allowed to stir at room temperature for 20 hours. Then, 20 mL of water was added to quench the reaction. The mixture was extracted with 50 mL of ethyl acetate. The organic layer was separated, dried over anhydrous magnesium sulfate and concentrated under vacuum. The residue was subjected to flash column chromatography (silica gel, CH₂Cl₂/MeOH=9/1 v/v) to give a white solid product, 0.10 g, 49.4% yield. MS: 334.2 [M+H]⁺. ¹H NMR (DMSO-d₆, 300 MHz) δ 10.58 (s, 1H), 9.83 (s, 1H), 8.12 (d, 1H, J=8.7 Hz), 7.71 (s, 1H), 7.22 (d, 2H, J=8.7 Hz), 7.09 (d, 1H, J=21. Hz), 7.04 (dd, 1H, J₁=8.7 Hz, J₂=2.4 Hz), 6.84 (d, 2H, J=8.7 Hz).

### Synthesis of 6-hydroxy-2-(4-hydroxyphenyl)-4-(4-methoxyphenyl)isoquinolin-1(2H)-one (5)

4-Bromo-6-hydroxy-2-(4-hydroxyphenyl)isoquinolin-1(2H)-one (4) (1.68 g, 5.06 mmol), tetrakis(triphenylphosphine)palladium (0.29 mg, 0.25 mmol), potassium carbonate (2.10 g, 15.18 mmol) and 4-methoxyphenylboronic acid (0.92 g, 6.07 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux overnight. The reaction mixture was diluted with 50 mL of water at room temperature. The mixture was extracted with ethyl acetate (5×30 mL). The extracts were combined, washed with brine (2×10 mL) and dried over anhydrous MgSO₄ followed by filtration with metal scavenger and concentration to give a yellow residue. The yellow residue was purified by flash column chromatography (silica-gel, CH₂Cl₂/MeOH=19/1 v/v) to give a white solid product, 1.54 g, 84.6% yield. MS: m/z 360.1 [M+H]⁺. ¹H NMR (DMSO-d₆, 300 MHz) δ 10.28 (s, 1H), 9.68 (s, 1H), 8.18 (d, 1H, J=8.7 Hz), 7.38 (d, 2H, J=9.0 Hz), 7.27 (d, 2H, J=8.7 Hz), 7.13 (s, 1H), 7.04 (d, 2H, J=8.7 Hz), 6.99 (dd, 1H, J₁=8.7 Hz, J₂=2.4 Hz), 6.86-6.83 (m, 3H), 3.81 (s, 3H).

### Synthesis of 6-hydroxy-2,4-bis(4-hydroxyphenyl)isoquinolin-1(2H)-one (6)

6-Hydroxy-2-(4-hydroxyphenyl)-4-(4-methoxyphenyl)isoquinolin-1(2H)-one (5) (1.54 g, 4.29 mmol) was placed in a dry and argon flushed 250 mL single-necked round-bottomed flask fitted with a stirring bar and an argon inlet. BBr₃ (21.4 mL of 1.0M CH₂Cl₂ solution, 21.4 mmol) was added via a syringe with stirring at room temperature. After stirred at room temperature overnight, the reaction was quenched by adding 50 mL of water and 5 mL of methanol. The mixture was stirred at room temperature for one hour. The white precipitate was filtered out, washed with water (2×20 mL) and dried under vacuum. The solid was recystallized from EtOAc/MeOH to give a white solid product, 1.28 g, 86.5% yield. MS: m/e 343.9 [M−H]⁻. ¹H NMR (DMSO-d₆, 300 MHz) δ 10.26 (s, 1H), 9.68 (s, 1H), 9.55 (s, 1H), 8.17 (d, 1H, J=8.7 Hz), 7.28-7.24 (m, 4H), 7.09 (s, 1H), 6.98 (dd, 1H, J₁=8.7 Hz, J₂=2.1 Hz), 6.88-6.79 (m, 5H).

### Example 1B

### Synthesis of 2-(4-bromomethyl)phenyl-6-hydroxy-4-(4-hydroxyphenyl)isoquinolin-1(2H)-one (11)

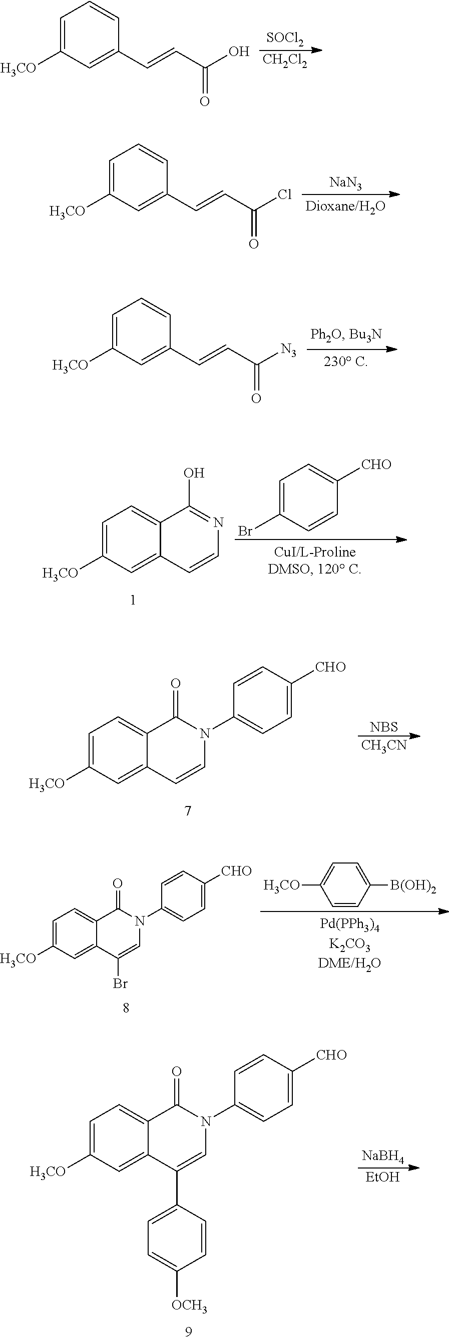

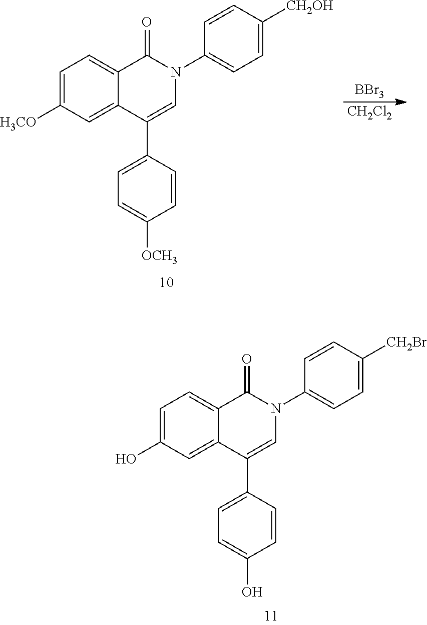

### Synthesis of 4-(6-methoxy-1-oxoisoquinolin-2(1H)-yl)benzaldehyde (7)

6-Methoxyisoquinoline-1-ol (1) (3.00 g, 17.13 mmol), 4-bromobenzaldehyde (3.80 g, 20.55 mmol), copper(I)iodide (0.65 g, 4.43 mmol), L-proline (0.79 g, 6.85 mmol) and anhydrous potassium carbonate (4.74 g, 34.26 mmol) were placed in a dry 250 mL three-necked round-bottomed flask fitted with a stirring bar and reflux condenser. The reaction flask was vacuumed and refilled with dry argon. 50 mL of anhydrous methyl sulfoxide was added via a syringe. The reaction mixture was stirred and heated to 95° C. overnight. The reaction was quenched by adding 100 mL of water at room temperature. The mixture was stirred at room temperature for 2 hours. The yellow solid was filtered out, washed with water (2×30 mL) and acetone (20 mL) and dried under vacuum. The solution was extracted with ethyl acetate (3×50 mL). The organic layers were separated, washed with brine (2×50 mL), filtered and concentrated to dryness. The solid and residue were combined and purified by flash column chromatography (silica gel, CH₂Cl₂/acetone=23/1 v/v) to give a pale-yellow solid product, 2.55 g, 53.3% yield. MS: 280.0 [M+H]⁺. ¹H NMR (DMSO-d₆, 300 MHz) δ 10.08 (s, 1H), 8.17 (d, 1H, J=8.7 Hz), 8.05 (d, 2H, J=8.4 Hz), 7.73 (d, 2H, J=8.4 Hz), 7.50 (d, 1H, J=7.5 Hz), 7.22 (d, 1H, J=2.4 Hz), 7.13 (dd, 1H, J₁=9.0 Hz, J₂=2.4 Hz), 6.70 (d, 1H, J=7.5 Hz), 3.91 (s, 3H).

### Synthesis of 4-(4-bromo-6-methoxy-1-oxoisoquinolin-2(1H)-yl)benzaldehyde (8)

4-(6-Methoxy-1-oxoisoquinolin-2(1H)-yl)benzaldehyde (7) (1.30 g, 4.65 mmol) was placed in a dry 250 mL single-necked round-bottomed flask fitted with a stirring bar. Acetonitrile (30 mL) was added via a syringe under argon atmosphere at room temperature. N-Bromosuccinimide (0.99 g, 5.59 mmol) was added in two portions under argon atmosphere at room temperature. The reaction mixture was allowed to stir at room temperature overnight. Then, the solvent was removed under reduced pressure. The residue was purified by flash column chromatography (silica gel, CH₂Cl₂/acetone=19/1 v/v) to give a pale-yellow solid product, 1.00 g, 60.2% yield. MS: 360.1 [M+H]⁺. ¹H NMR (DMSO-d₆, 300 MHz) δ 10.09 (s, 1H), 8.24 (d, 1H, J=8.7 Hz), 8.05 (d, 2H, J=8.1 Hz), 7.97 (s, 1H), 7.76 (d, 2H, J=8.1 Hz), 7.26 (dd, 1H, J₁=8.7 Hz, J₂=2.4 Hz), 7.17 (d, 1H, J=2.4 Hz), 3.96 (s, 3H).

### Synthesis of 4-(6-methoxy-4-(4-methoxyphenyl)-1-oxoisoquinolin-2(1H)-yl)benzaldehyde (9)

4-(4-bromo-6-methoxy-1-oxoisoquinolin-2(1H)-yl)benzaldehyde (8) (0.82 g, 2.29 mmol), tetrakis(triphenylphosphine)palladium (0.13 g, 0.115 mmol), potassium carbonate (0.63 g, 4.58 mmol) and 4-methoxyphenylboronic acid (0.42 g, 2.75 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux for 5 hours. The reaction mixture was diluted with 100 mL of water at room temperature. The mixture was extracted with ethyl acetate (3×50 mL). The extracts were combined, washed with brine (2×10 mL) and dried over anhydrous MgSO₄ followed by filtration with metal scavenger and concentration to give a yellow residue. The yellow residue was purified by flash column chromatography (silica-gel, CH₂Cl₂/acetone=97/3 v/v) to give a white solid product, 0.80 g, 90.9% yield. MS: m/z 408.1 [M+H]⁺. ¹H NMR (DMSO-d₆, 300 MHz) δ 10.08 (s, 1H), 8.31 (d, 1H, J=9.0 Hz), 8.04 (d, 2H, J=8.7 Hz), 7.81 (d, 2H, J=8.4 Hz), 7.46 (d, 2H, J=8.4 Hz), 7.37 (s, 1H), 7.23 (dd, 1H, J₁=8.7 Hz, J₂=2.4 Hz), 7.06 (d, 2H, J=8.7 Hz), 6.94 (d, 1H, J=2.4 Hz), 3.82 (s, 3H), 3.81 (s, 3H).

### Synthesis of 2-(4-hydroxymethyl)phenyl-6-methoxy-4-(4-methoxyphenyl)-isoquinolin-1(2H)-one (10)

4-(6-Methoxy-4-(4-methoxyphenyl)-1-oxoisoquinolin-2(1H)-yl)benzaldehyde (9) (0.74 g, 1.92 mmol) was dissolved in ethanol (30 mL) at room temperature. NaBH₄ (36 mg, 0.96 mmol) was added at room temperature. The reaction mixture was allowed to stir at room temperature for 5 hours. Then, the reaction was quenched by adding 50 mL of saturated ammonia chloride solution at room temperature. The solution was stirred at room temperature for one hour and extracted with ethyl acetate (3×50 mL). The organic layer was separated, dried over anhydrous MgSO₄, filtered and concentrated to dryness under reduced pressure. The residue was subjected to flash column chromatography (silica gel, CH₂Cl₂/MeOH=19/1 v/v) to give a white solid product, 0.68 g, 91.9% yield. MS: m/z 410.3 [M+Na]⁺. ¹H NMR (DMSO-d₆, 300 MHz) δ 8.29 (d, 1H, J=9.0 Hz), 7.50-7.39 (m, 6H), 7.25 (s, 1H), 7.20 (dd, 1H, J₁=9.0 Hz, J₂=2.4 Hz), 7.05 (d, 2H, J=9.0 Hz), 6.93 (d, 1H, J=2.4 Hz), 4.57 (s, 2H), 3.81 (s, 3H), 3.79 (s, 3H).

### Synthesis of 2-(4-bromomethyl)phenyl-6-hydroxy-4-(4-hydroxyphenyl)isoquinolin-1(2H)-one (11)

2-(4-Hydroxymethyl)phenyl-6-methoxy-4-(4-methoxyphenyl)-isoquinolin-1(2H)-one

(10) (0.45 g, 1.16 mmol) was dissolved in anhydrous methylene chloride (20 mL) in a dry 250 mL single-necked round-bottomed flask fitted with a stirring bar and sealed with a rubber stopper. BBr₃ (4.65 mL of 1M CH₂Cl₂ solution, 4.65 mmol) was added via a syringe dropwise with stirring at room temperature. The resulted solution was stirred at room temperature overnight. The reaction was quenched by adding 50 mL of water and 5 mL of methanol at 0° C. The solution was then stirred at room temperature for two hours. CH₂Cl₂ layer was separated and the aqueous layer was extracted with CH₂Cl₂/MeOH (9/1 v/v, 3×50 mL). The organic layers were combined and dried over anhydrous MgSO₄. The solvent was removed under reduced pressure. The residue was purified by column chromatography (silica-gel, CH₂Cl₂/MeOH=19/1 v/v) to give a white solid product, 0.40 g, 81.6% yield. MS: m/e 421.8 [M−H]⁻. ¹H NMR (DMSO-d₆, 300 MHz) δ 10.33 (s, 1H), 9.57 (s, 1H), 8.19 (d, 1H, J=8.7 Hz), 7.57 (d, 2H, J=8.7 Hz), 7.51 (d, 2H, J=8.7 Hz), 7.27 (d, 2H, J=8.4 Hz), 7.20 (s, 1H), 7.00 (d, 1H, J₁=8.7 Hz, J₂=2.4 Hz), 6.89-6.86 (m, 3H), 4.78 (s, 2H).

### Example 1C

### Synthesis of 6-hydroxy-2-(4-hydroxyphenyl)-4-(4-(trifluoromethyl)phenyl)isoquinolin-1(2H)-one (13)

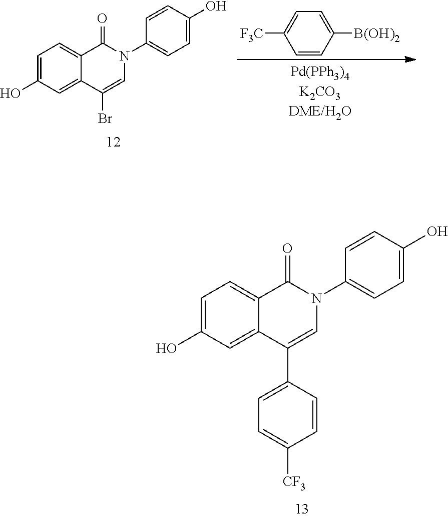 [SMILES: O=c1c2ccc(O)cc2c(Br)cn1-c1ccc(O)cc1 ; OB(O)c1ccc(C(F)(F)F)cc1 ; O=c1c2ccc(O)cc2c(-c2ccc(C(F)(F)F)cc2)cn1-c1ccc(O)cc1]

### Synthesis of 6-hydroxy-2-(4-hydroxyphenyl)-4-(4-(trifluoromethyl)phenyl)isoquinolin-1(2H)-one (13)

4-Bromo-6-hydroxy-2-(4-hydroxyphenyl)isoquinolin-1(2H)-one (12 synthesized per WO2008/091555) (1.00 g, 3.01 mmol), tetrakis(triphenylphosphine)palladium (0.17 g, 0.15 mmol), potassium carbonate (1.25 g, 9.03 mmol) and 4-trifluoromethylphenylboronic acid (0.69 g, 3.61 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux overnight. The reaction mixture was diluted with 50 mL of water at room temperature. The mixture was extracted with ethyl acetate (3×50 mL). The extracts were combined, washed with brine (2×10 mL) and dried over anhydrous MgSO₄ followed by filtration with metal scavenger and concentration to give a yellow residue. The yellow residue was purified by flash column chromatography (silica-gel, CH₂Cl₂/MeOH=19/1 v/v) to give a white solid product, 0.90 g, 75.3% yield. MS: m/e 395.9 [M−H]⁻. ¹H NMR (DMSO-d₆, 300 MHz) δ 10.35 (s, 1H), 9.71 (s, 1H), 8.20 (d, 1H, J=8.7 Hz), 7.85 (d, 2H, J=8.4 Hz), 7.72 (d, 2H, J=8.4 Hz), 7.33 (s, 1H), 7.29 (d, 2H, J=8.7 Hz), 7.02 (dd, 1H, J₁=8.7 Hz, J₂=2.4 Hz), 6.86-6.83 (m, 3H).

### Example 1D

### Synthesis of 6-hydroxy-2-(4-(hydroxymethyl)phenyl)-4-(4-hydroxyphenyl)isoquinolin-1(2H)-one (14)

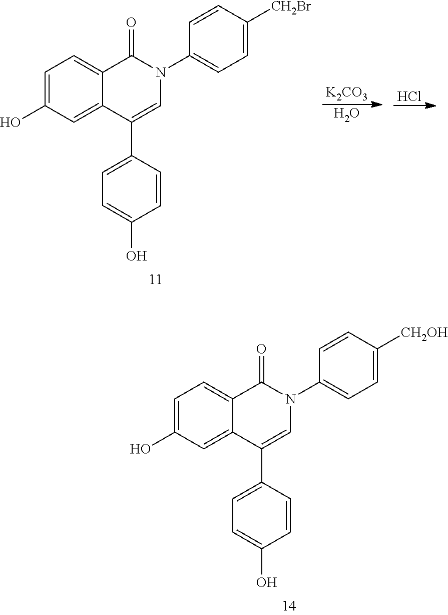

### Synthesis of 6-hydroxy-2-(4-(hydroxymethyl)phenyl)-4-(4-hydroxyphenyl)isoquinolin-1(2H)-one (14)

2-(4-Bromomethyl)phenyl-6-hydroxy-4-(4-hydroxyphenyl)isoquinolin-1(2H)-one (11) (0.45 g, 1.07 mmol) and K₂CO₃ were mixed together in 30 mL of water at room temperature. The mixture was stirred and heated to reflux for 3 hours. Then, the reaction mixture was acidified by adding 50 mL of 2N HCl solution at room temperature. The solution was extracted with ethyl acetate/MeOH (9/1 v/v). The organic layer was separated, dried over anhydrous MgSO₄, filtered and concentrated to dryness under reduced pressure. The residue was subjected to flash column chromatography (silica-gel, CH₂Cl₂/MeOH=9/1 v/v) to give a white solid product, 15 mg, 3.9% yield. MS: m/e 358.0 [M−H]⁻. ¹H NMR (DMSO-d₆, 300 MHz) δ 10.30 (s, 1H), 9.56 (s, 1H), 8.19 (d, 1H, J=8.7 Hz), 7.44-7.41 (m, 4H), 7.26 (d, 2H, J=8.4 Hz), 7.14 (s, 1H), 7.00 (dd, 1H, J₁=8.7 Hz, J₂=2.4 Hz), 6.88-6.85 (m, 3H), 5.29 (t, 1H, J=5.4 Hz), 4.56 (d, 2H, J=5.4 Hz).

### Example 1E

### Synthesis of 2-(4-(hydroxymethyl)-3-methoxyphenyl)-6-methoxy-4-(4-(trifluoromethyl)phenyl)isoquinolin-1(2H)-one (25) and 2-(4-(bromomethyl)-3-hydroxyphenyl)-6-hydroxy-4-(4-(trifluoromethyl)phenyl)isoquinolin-1(2H)-one (26)

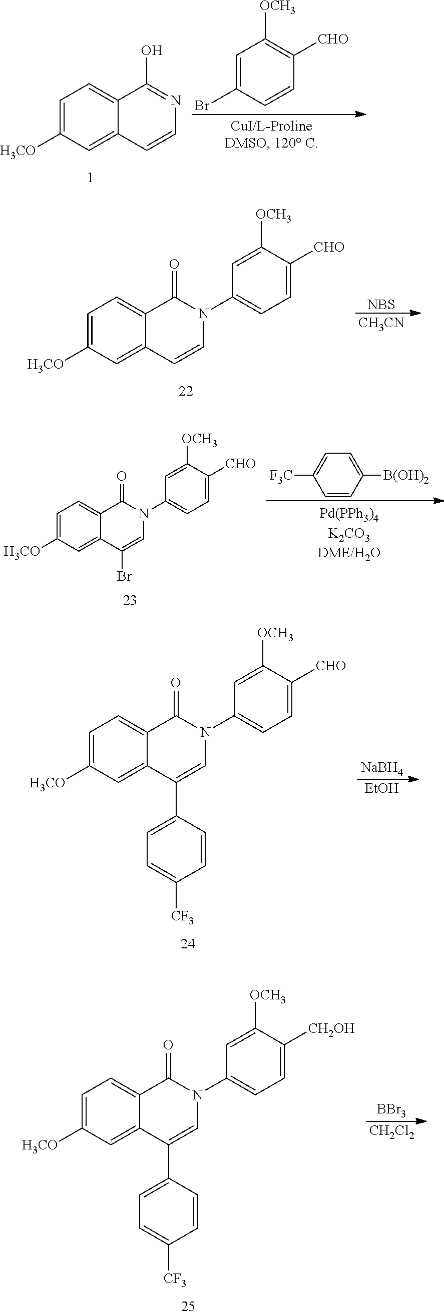

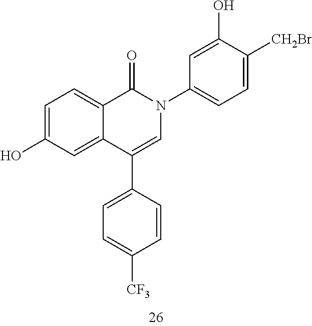

### Synthesis of 2-methoxy-4-(6-methoxy-1-oxoisoquinolin-2(1H)-yl)benzaldehyde (22)

6-Methoxyisoquinoline-1-ol (1) (1.23 g, 7.02 mmol), 4-bromo-2-methoxybenzaldehyde (1.82 g, 8.43 mmol), copper(I) iodide (0.27 g, 1.40 mmol), L-proline (0.32 g, 2.80 mmol) and anhydrous potassium carbonate (1.94 g, 14.04 mmol) were placed in a dry 250 mL three-necked round-bottomed flask fitted with a stirring bar and reflux condenser. The reaction flask was vacuumed and refilled with dry argon. 40 mL of anhydrous methyl sulfoxide was added via a syringe. The reaction mixture was stirred and heated to 100° C. overnight. The reaction was quenched by adding 100 mL of water at room temperature. The mixture was stirred at room temperature for 2 hours. The yellow solid was filtered out, washed with water (2×30 mL) and acetone (20 mL) and dried under vacuum. The solid was purified by flash column chromatography (silica gel, CH₂Cl₂/acetaone=19/1 v/v) to give a pale-yellow solid product, 1.08 g, 49.8% yield. MS: 332.2 [M+Na]⁺. ¹H NMR (DMSO-d₆, 300 MHz) δ 10.38 (s, 1H), 8.17 (d, 1H, J=8.7 Hz), 7.81 (d, 2H, J=8.1 Hz), 7.50 (d, 1H, J=7.2 Hz), 7.38 (d, 1H, J=1.8 Hz), 7.23-7.19 (m, 2H), 7.13 (dd, 1H, J₁=9.0 Hz, J₂=2.4 Hz), 6.70 (d, 1H, J=7.5 Hz), 3.96 (s, 3H), 3.91 (s, 3H).

### Synthesis of 4-(4-bromo-6-methoxy-1-oxoisoquinolin-2(1H)-yl)-2-methoxybenzaldehyde (23)

2-Methoxy-4-(6-methoxy-1-oxoisoquinolin-2(1H)-yl)benzaldehyde (22) (0.87 g, 2.81 mmol) was placed in a dry 250 mL single-necked round-bottomed flask fitted with a stirring bar. THF (30 mL) was added via a syringe under argon atmosphere at room temperature. N-Bromosuccinimide (0.60 g, 3.38 mmol) was added in two portions under argon atmosphere at room temperature. The reaction mixture was allowed to stir at room temperature for 5 hours. Then, the solvent was removed under reduced pressure. The residue was purified by flash column chromatography (silica gel, CH₂Cl₂/acetone=19/1 v/v) to give a pale-yellow solid product, 0.86 g, 78.9% yield. MS: 390.0 [M+H]⁺. ¹H NMR (DMSO-d₆, 300 MHz) δ 10.38 (s, 1H), 8.25 (d, 1H, J=9.0 Hz), 7.98 (s, 1H), 7.81 (d, 1H, J=8.0 Hz), 7.44 (s, 1H), 7.27 (d, 2H, J=9.0 Hz), 7.23 (d, 1H, J=8.0 Hz), 7.19 (s, 1H), 3.96 (s, 6H).

### Synthesis of 2-methoxy-4-(6-methoxy-1-oxo-4-(4-(trifluoromethyl)phenyl)isoquinolin-2(1H)-yl)benzaldehyde (24)

4-(4-Bromo-6-methoxy-1-oxoisoquinolin-2(1H)-yl)-2-methoxybenzaldehyde (23) (0.45 g, 1.16 mmol), tetrakis(triphenylphosphine)palladium (67 mg, 0.058 mmol), potassium carbonate (0.32 g, 2.32 mmol) and 4-trifluoromethylphenylboronic acid (0.33 g, 1.74 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux for 6 hours. The reaction mixture was diluted with 150 mL of water at room temperature. The precipitate was filtered out and dried under vacuum. The precipitate was then purified by flash column chromatography (silica-gel, CH₂Cl₂/acetone=19/1 v/v) to give a white solid product, 0.42 g, 79.8% yield. MS: m/z 476.1 [M+Na]⁺. ¹H NMR (DMSO-d₆, 300 MHz) δ 10.38 (s, 1H), 8.33 (d, 1H, J=9.0 Hz), 7.89-7.80 (m, 5H), 7.57 (s, 1H), 7.47 (d, 1H, J=1.5 Hz), 7.30-7.24 (m, 2H), 6.92 (d, 1H, J=2.4 Hz), 3.96 (s, 3H), 3.82 (s, 3H).

### Synthesis of 2-(4-(hydroxymethyl)-3-methoxyphenyl)-6-methoxy-4-(4-(trifluoromethyl)phenyl)isoquinolin-1(2H)-one (25)

2-Methoxy-4-(6-methoxy-1-oxo-4-(4-(trifluoromethyl)phenyl)isoquinolin-2(1H)-yl)benzaldehyde (24) (0.40 g, 0.88 mmol) was dissolved in ethanol (30 mL) at room temperature. NaBH₄ (33 mg, 0.88 mmol) was added at room temperature. The reaction mixture was allowed to stir at room temperature for 12 hours. Then, the reaction was quenched by adding 100 mL of saturated ammonia chloride solution at room temperature. The solution was stirred at room temperature for one hour and extracted with ethyl acetate (3×50 mL). The organic layer was separated, dried over anhydrous MgSO₄, filtered and concentrated to dryness under reduced pressure. The residue was subjected to flash column chromatography (silica gel, CH₂Cl₂/MeOH=17/3 v/v) to give a white solid product, 0.38 g, 95.0% yield. MS: m/z 478.0 [M+Na]⁺. ¹H NMR (DMSO-d₆, 300 MHz) δ 8.32 (d, 1H, J=9.0 Hz), 7.85 (d, 2H, J=8.4 Hz), 7.79 (d, 2H, J=8.4 Hz), 7.48 (d, 1H, J=7.8 Hz), 7.47 (s, 1H), 7.24 (dd, 1H, J₁=9.0 Hz, J₂=2.4 Hz), 7.13-7.07 (m, 2H), 6.93 (d, 1H, J=2.4 Hz), 5.12 (t, 1H, J=2.7 Hz), 4.54 (d, 2H, J=2.7 Hz), 3.81 (s, 3H), 3.80 (s, 3H).

### Synthesis of 2-(4-(bromomethyl)-3-hydroxyphenyl)-6-hydroxy-4-(4-(trifluoromethyl)phenyl)isoquinolin-1(2H)-one (26)

2-(4-(Hydroxymethyl)-3-methoxyphenyl)-6-methoxy-4-(4-(trifluoromethyl)phenyl)isoquinolin-1(2H)-one (25) (0.35 g, 0.0.77 mmol) was dissolved in 30 mL of anhydrous CH₂Cl₂ in a dry and argon flushed 150 mL single-necked round-bottomed flask fitted with a stirring bar and an argon inlet. BBr₃ (5.0 mL of 1.0M CH₂Cl₂ solution, 5.0 mmol) was added via a syringe with stirring at room temperature. After stirred at room temperature overnight, the reaction was quenched by adding 50 mL of water and 5 mL of methanol. The solution was stirred at room temperature for one hour. The CH₂Cl₂ layer was separated. The aqueous layer was extracted with EtOAc (3×50 mL). The organic layers were separated, combined and dried over anhydrous MgSO₄. The solvent was removed under reduced pressure. The residue was purified by column chromatography (silica-gel, CH₂Cl₂/MeOH=19/1 v/v) to give a white solid product, 0.30 g, 79.6% yield. MS: m/e 408 [M−HBr]⁻. ¹H NMR (DMSO-d₆, 300 MHz) δ 10.37 (s, 1H), 9.86 (s, 1H), 8.22 (d, 1H, J=8.7 Hz), 7.86-7.63 (m, 4H), 7.36 (s, 1H), 7.32 (d, 1H, J=8.4 Hz), 7.03 (dd, 1H, J₁=8.7 Hz, J₂=2.1 Hz), 6.94-6.82 (m, 3H), 4.43 (s, 2H).

### Example 1F

### Synthesis of 6-hydroxy-2,4-bis(4-(trifluoromethyl)phenyl)isoquinolin-1(2H)-one (30)

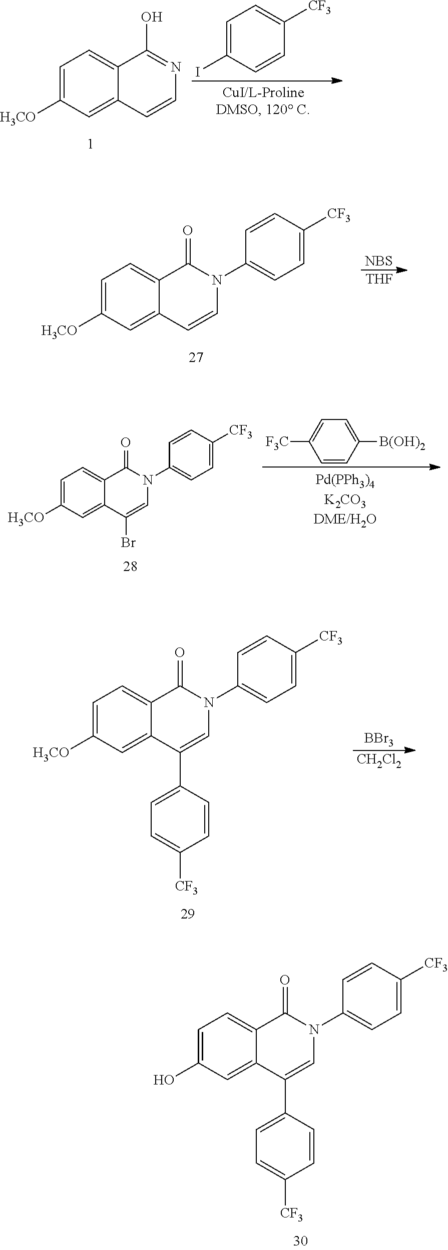

### Synthesis of 6-methoxy-2-(4-(trifluoromethyl)phenylisoquinolin-1(2H)-one (27)

6-Methoxyisoquinoline-1-ol (1) (0.86 g, 4.91 mmol), 1-iodo-4-(trifluoromethyl)benzene (1.60 g, 5.89 mmol), copper(I) iodide (0.19 g, 0.98 mmol), L-proline (0.23 g, 1.96 mmol) and anhydrous potassium carbonate (1.36 g, 9.82 mmol) were placed in a dry 250 mL three-necked round-bottomed flask fitted with a stirring bar and reflux condenser. The reaction flask was vacuumed and refilled with dry argon. 40 mL of anhydrous methyl sulfoxide was added via a syringe. The reaction mixture was stirred and heated to 100° C. overnight. The reaction was quenched by adding 150 mL of water at room temperature. The mixture was stirred at room temperature for 2 hours. The yellow solid was filtered to out, washed with water (2×30 mL) and dried under vacuum. The solid was purified by flash column chromatography (silica gel, CH₂Cl₂) to give a white solid product, 1.30 g, 82.9% yield. MS: 342.0 [M+Na]⁺. ¹H NMR (DMSO-d₆, 300 MHz) δ 8.16 (d, 1H, J=9.0 Hz), 7.90 (d, 2H, J=8.4 Hz), 7.73 (d, 2H, J=8.4 Hz), 7.50 (d, 1H, J=7.5 Hz), 7.22 (d, 1H, J=2.4 Hz), 7.13 (dd, 1H, J₁=9.0 Hz, J₂=2.4 Hz), 6.70 (d, 1H, J=7.5 Hz), 3.91 (s, 3H).

### Synthesis of 4-bromo-6-methoxy-2-(4-(trifluoromethyl)phenyl)isoquinolin-1(2H)-one (28)

6-Methoxy-2-(4-(trifluoromethyl)phenylisoquinolin-1(2H)-one (27) (1.20 g, 3.76 mmol) was placed in a dry 250 mL single-necked round-bottomed flask fitted with a stirring bar. THF (30 mL) was added via a syringe under argon atmosphere at room temperature. N-Bromosuccinimide (0.80 g, 4.51 mmol) was added in two portions under argon atmosphere at room temperature. The reaction mixture was allowed to stir at room temperature for 5 hours. Then, the solvent was removed under reduced pressure. The residue was purified by flash column chromatography (silica gel, CH₂Cl₂) to give a white solid product, 1.37 g, 91.3% yield. MS: 420.8 [M+Na]⁺. ¹H NMR (DMSO-d₆, 300 MHz) δ 8.23 (d, 1H, J=9.0 Hz), 7.97 (s, 1H), 7.90 (d, 2H, J=8.4 Hz), 7.76 (d, 2H, J=8.4 Hz), 7.25 (dd, 1H, J₁=9.0 Hz, J₂=2.4 Hz), 7.16 (d, 1H, J=2.4 Hz), 3.96 (s, 3H).

### Synthesis of 6-methoxy-2,4-bis(4-(trifluoromethyl)phenyl)isoquinolin-1(2H)-one (29)

4-Bromo-6-methoxy-2-(4-(trifluoromethyl)phenyl)isoquinolin-1(2H)-one (28) (0.53 g, 1.33 mmol), tetrakis(triphenylphosphine)palladium (77 mg, 0.067 mmol), potassium carbonate (0.37 g, 2.66 mmol) and 4-trifluoromethylphenylboronic acid (0.38 g, 2.00 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux for 6 hours. The reaction mixture was diluted with 150 mL of water at room temperature. The mixture was extracted with ethyl acetate (4×20 mL). The organic layers were combined, dried over anhydrous MgSO₄, filtered and concentrated to dryness. The residue was purified by flash column chromatography (silica-gel, CH₂Cl₂) to give a white solid product, 0.52 g, 83.9% yield. MS: m/z 464.1 [M+H]⁺. ¹H NMR (DMSO-d₆, 300 MHz) δ 8.33 (d, 1H, J=9.0 Hz), 7.92-7.79 (m, 8H), 7.57 (s, 1H), 7.26 (dd, 1H, J₁=8.7 Hz, J₂=2.4 Hz), 6.93 (d, 1H, J=2.4 Hz), 3.82 (s, 3H).

### Synthesis of 6-hydroxy-2,4-bis(4-(trifluoromethyl)phenyl)isoquinolin-1(2H)-one (30)

6-Methoxy-2,4-bis(4-(trifluoromethyl)phenyl)isoquinolin-1(2H)-one (29) (0.30 g, 0.65 mmol) was dissolved in 30 mL of anhydrous CH₂Cl₂ in a dry and argon flushed 150 mL single-necked round-bottomed flask fitted with a stirring bar and an argon inlet. BBr₃ (3.50 mL of 1.0M CH₂Cl₂ solution, 3.50 mmol) was added via a syringe with stirring at room temperature. After stirred at room temperature overnight, the reaction was quenched by adding 50 mL of water and 5 mL of methanol. The solution was stirred at room temperature for one hour. The CH₂Cl₂ layer was separated. The aqueous layer was extracted with CH₂Cl₂ (3×20 mL). The organic layers were separated, combined and dried over anhydrous MgSO₄. The solvent was removed under reduced pressure. The residue was purified by column chromatography (silica-gel, CH₂Cl₂/MeOH=19/1 v/v) to give a white solid product, 0.25 g, 86.2% yield. MS: m/e 447.9 [M−H]⁻. ¹H NMR (DMSO-d₆, 300 MHz) δ10.47 (s, 1H), 8.23 (d, 1H, J=9.0 Hz), 8.06 (d, 2H, J=8.1 Hz), 7.88 (d, 2H, J=8.7 Hz), 7.79 (d, 2H, J=8.7 Hz), 7.66 (d, 2H, J=8.1 Hz), 7.47 (s, 1H), 7.05 (dd, 1H, J₁=8.7 Hz, J₂=2.1 Hz), 6.87 (d, 1H, J=2.4 Hz)

### Example 1G

### Synthesis of 2-(4-fluorophenyl)-6-hydroxy-4-(4-(trifluoromethyl)phenyl)isoquinolin-1(2H)-one (34)

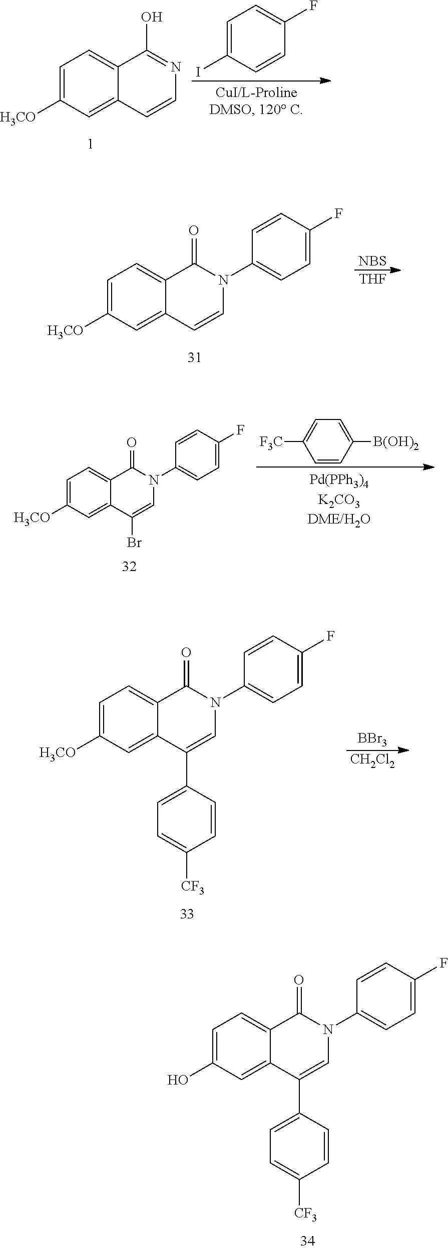

### Synthesis of 2-(4-fluorophenyl)-6-methoxyisoquinolin-1(2H)-one (31)

6-Methoxyisoquinoline-1-ol (1) (1.52 g, 8.68 mmol), 1-fluoro-4-iodobenzene (2.89 g, 13.01 mmol), copper(I) iodide (0.33 g, 1.72 mmol), L-proline (0.39 g, 3.44 mmol) and anhydrous potassium carbonate (2.40 g, 17.36 mmol) were placed in a dry 250 mL three-necked round-bottomed flask fitted with a stirring bar and reflux condenser. The reaction flask was vacuumed and refilled with dry argon. 30 mL of anhydrous methyl sulfoxide was added via a syringe. The reaction mixture was stirred and heated to 120° C. for 16 hours. The reaction was quenched by adding 30 mL of water at room temperature. The mixture was stirred at room temperature for one hour. The mixture was extracted with EtOAc (3×50 mL). The organic layers were combined, washed with brine (2×30 ml), dried over anhydrous MgSO₄, filtered and concentrated under reduced pressure. The residue was purified by flash column chromatography (silica gel, Hexanes/EtOAc=2/3 v/v) to give a white solid product, 2.0 g, 85.5% yield.

### Synthesis of 4-bromo-2-(4-fluorophenyl)-6-methoxyisoquinolin-1(2H)-one (32)

2-(4-Fluorophenyl)-6-methoxyisoquinolin-1(2H)-one (31) (1.40 g, 5.20 mmol) was placed in a dry 250 mL single-necked round-bottomed flask fitted with a stirring bar. Acetonitrile (20 mL) was added via a syringe under argon atmosphere at room temperature. N-Bromosuccinimide (0.97 g, 5.46 mmol) was added in two portions under argon atmosphere at room temperature. The reaction mixture was allowed to stir at room temperature for 4 hours. Then, the solvent was removed under reduced pressure. The residue was purified by flash column chromatography (silica gel, Hexanes/EtOAc=1/1 v/v) to give a white solid product, 1.55 g, 85.6% yield.

### Synthesis of 2-(4-fluorophenyl)-6-methoxy-4-(4-(trifluoromethyl)phenyl)isoquinolin-1(2H)-one (33)

4-Bromo-2-(4-fluorophenyl)-6-methoxyisoquinolin-1(2H)-one (32) (0.27 g, 0.78 mmol), tetrakis(triphenylphosphine)palladium (45 mg, 0.04 mmol), potassium carbonate (0.21 g, 1.55 mmol) and 4-trifluoromethylphenylboronic acid (0.22 g, 1.16 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux for 12 hours. The reaction mixture was diluted with 100 mL of water at room temperature. The mixture was extracted with ethyl acetate (3×20 mL). The organic layers were combined, dried over anhydrous MgSO₄, filtered and concentrated to dryness. The residue was purified by flash column chromatography (silica-gel, CH₂Cl₂) to give a white solid product, 0.28 g, 87.5% yield. MS: m/z 436.0 [M+Na]⁺. ¹H NMR (DMSO-d₆, 300 MHz) δ 8.31 (d, 1H, J=9.0 Hz), 7.85 (d, 2H, J=8.1 Hz), 7.78 (d, 2H, J=8.4 Hz), 7.63-7.57 (m, 2H), 7.48 (s, 1H), 7.39-7.3 (m, 2H), 7.24 (dd, 1H, J₁=9.0 Hz, J₂=2.4 Hz), 6.93 (d, 1H, J=2.4 Hz), 3.81 (s, 3H).

### Synthesis of 2-(4-fluorophenyl)-6-hydroxy-4-(4-(trifluoromethyl)phenyl)isoquinolin-1(2H)-one (34)

2-(4-Fluorophenyl)-6-methoxy-4-(4-(trifluoromethyl)phenyl)isoquinolin-1(2H)-one

(33) (0.28 g, 0.68 mmol) was dissolved in 30 mL of anhydrous CH₂Cl₂ in a dry and argon flushed 150 mL single-necked round-bottomed flask fitted with a stirring bar and an argon inlet. BBr₃ (3.50 mL of 1.0M CH₂Cl₂ solution, 3.50 mmol) was added via a syringe with stirring at room temperature. After stirred at room temperature overnight, the reaction was quenched by adding 50 mL of water and 5 mL of methanol. The solution was stirred at room temperature for one hour. The CH₂Cl₂ layer was separated. The aqueous layer was extracted with EtOAc (3×50 mL). The organic layers were separated, combined and dried over anhydrous MgSO₄. The solvent was removed under reduced pressure. The residue was purified by column chromatography (silica-gel, CH₂Cl₂/MeOH=19/1 v/v) to give a white solid product, 0.24 g, 88.9% yield. MS: m/e 397.9 [M−H]⁻. ¹H NMR (DMSO-d₆, 300 MHz) δ 10.39 (s, 1H), 8.22 (d, 1H, J=8.7 Hz), 7.86 (d, 2H, J=8.1 Hz), 7.23 (d, 2H, J=8.1 Hz), 7.61-7.55 (m, 2H), 7.41 (s, 1H), 7.39-7.32 (m, 2H), 7.04 (dd, 1H, J₁=8.7 Hz, J₂=2.1 Hz), 6.84 (d, 1H, J=2.4 Hz).

### Example 111

### Synthesis of 6-methoxy-2-(4-methoxyphenyl)-4-(4-(trifluoromethyl)phenyl)isoquinolin-1(2H)-one (35)

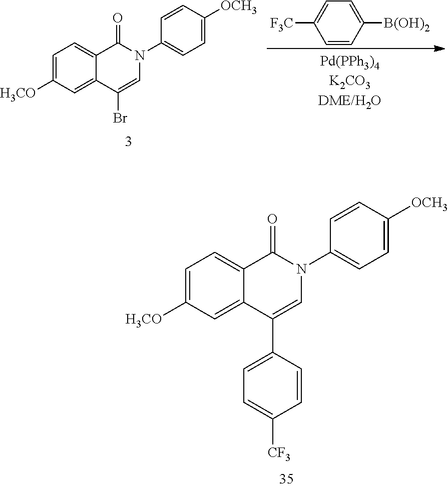 [SMILES: COc1ccc(-n2cc(Br)c3cc(OC)ccc3c2=O)cc1 ; OB(O)c1ccc(C(F)(F)F)cc1 ; COc1ccc(-n2cc(-c3ccc(C(F)(F)F)cc3)c3cc(OC)ccc3c2=O)cc1]

### Synthesis of 6-methoxy-2-(4-methoxyphenyl)-4-(4-(trifluoromethyl)phenyl)isoquinolin-1(2H)-one (35)

4-Bromo-6-methoxy-2-(4-methoxyphenyl)isoquinolin-1(2H)-one (3) (0.57 g, 1.58 mmol), tetrakis(triphenylphosphine)palladium (91 mg, 0.08 mmol), potassium carbonate (0.44 g, 3.16 mmol) and 4-trifluoromethylphenylboronic acid (0.36 g, 1.90 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux for 12 hours. The reaction mixture was diluted with 100 mL of water at room temperature. The mixture was extracted with ethyl acetate (3×50 mL). The organic layers were combined, dried over anhydrous MgSO₄, filtered and concentrated to dryness. The residue was purified by flash column chromatography (silica-gel, CH₂Cl₂/acetone=39/1 v/v) to give a white solid product, 0.54 g, 80.6% yield. MS: m/z 426.1 [M+H]⁺. ¹H NMR (DMSO-d₆, 300 MHz) δ 8.30 (d, 1H, J=9.0 Hz), 7.84 (d, 2H, J=8.7 Hz), 7.78 (d, 2H, J=8.7 Hz), 7.45 (d, 2H, J=9.0 Hz), 7.43 (s, 1H), 7.23 (dd, 1H, J₁=9.0 Hz, J₂=2.4 Hz), 7.05 (d, 2H, J=9.0 Hz), 6.92 (d, 1H, J=2.4 Hz), 3.81 (s, 3H), 3.80 (s, 3H).

### Example 11

### Synthesis of 4-(6-hydroxy-2-(4-hydroxyphenyl)-1-oxo-1,2-dihydroisoquinolin-4-yl)-N-methylbenzenesulfonamide (36)

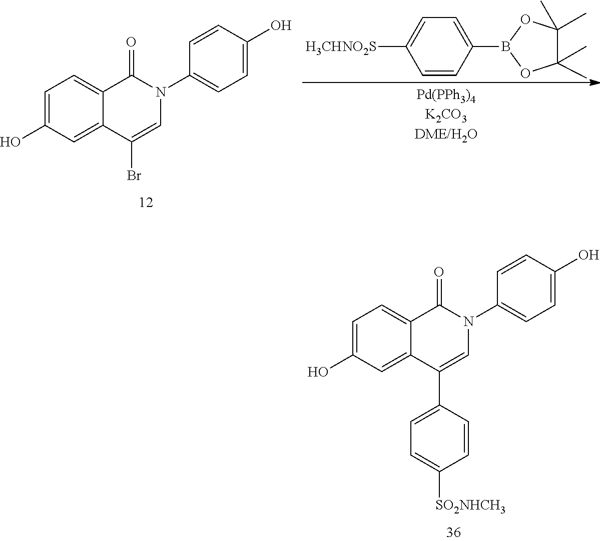

### Synthesis of 4-(6-hydroxy-2-(4-hydroxyphenyl)-1-oxo-1,2-dihydroisoquinolin-4-yl)-N-methylbenzenesulfonamide (36)

4-Bromo-6-hydroxy-2-(4-hydroxyphenyl)isoquinolin-1(2H)-one (12) (0.45 g, 1.36 mmol), tetrakis(triphenylphosphine)palladium (78 mg, 0.068 mmol), potassium carbonate (0.74 g, 5.40 mmol) and N-methyl-4-(4,4,5,5-tetramethyl-1,3,2-dioxaborolan-2-yl)benzenesulfonamide (0.48 g, 1.63 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux overnight. The reaction mixture was diluted with 100 mL of water at room temperature. The mixture was extracted with ethyl acetate (5×20 mL). The organic layers were combined, dried over anhydrous MgSO₄, filtered and concentrated to dryness. The residue was purified by flash column chromatography (silica-gel, CH₂Cl₂/acetone/MeOH=90/5/5 v/v/v) to give a white solid product, 0.35 g, 61.4% yield. MS: m/z 420.9 [M+H]⁺. ¹H NMR (DMSO-d₆, 500 MHz) δ 10.37 (s, 1H), 9.71 (s, 1H), 8.20 (d, 1H, J=9.0 Hz), 7.87 (d, 2H, J=8.5 Hz), 7.72 (d, 2H, J=8.5 Hz), 7.55 (q, 1H, J=5.0 Hz), 7.34 (s, 1H), 7.29 (d, 2H, J=8.5 Hz), 7.02 (dd, 1H, J₁=9.0 Hz, J₂=2.5 Hz), 6.86-6.85 (m, 3H), 2.47 (d, 3H, J=5.0 Hz).

### Example 1J

### Synthesis of 6-hydroxy-2-(4-hydroxyphenyl)-4-(4-(methylsulfonyl)phenyl)isoquinolin-1(2H)-one (37)

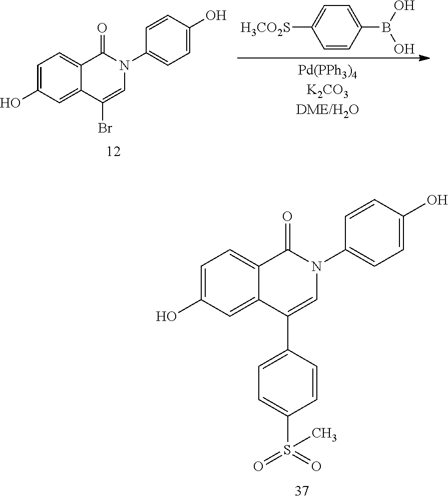

### Synthesis of 6-hydroxy-2-(4-hydroxyphenyl)-4-(4-(methylsulfonyl)phenyl)isoquinolin-1(2H)-one (37)

4-Bromo-6-hydroxy-2-(4-hydroxyphenyl)isoquinolin-1(2H)-one (12) (0.46 g, 1.39 mmol), tetrakis(triphenylphosphine)palladium (77 mg, 0.069 mmol), potassium carbonate (0.77 g, 5.54 mmol) and 4-(methylsulfonyl)phenylboronic acid (0.33 g, 1.66 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux overnight. The reaction mixture was diluted with 100 mL of water at room temperature. The mixture was extracted with ethyl acetate (5×20 mL). The organic layers were combined, dried over anhydrous MgSO₄, filtered and concentrated to dryness. The residue was purified by flash column chromatography (silica-gel, CH₂Cl₂/acetone/MeOH=90/5/5 v/v/v) to give a white solid product, 0.45 g, 80.4% yield. MS: m/z 406.0 [M−H]⁻. ¹H NMR (DMSO-d₆, 500 MHz) δ 10.38 (s, 1H), 9.71 (s, 1H), 8.21 (d, 1H, J=8.7 Hz), 8.03 (d, 2H, J=8.4 Hz), 7.77 (d, 2H, J=8.4 Hz), 7.35 (s, 1H), 7.29 (d, 2H, J=9.0 Hz), 7.03 (dd, 1H, J₁=8.7 Hz, J₂=2.1 Hz), 6.87-6.84 (m, 3H), 3.29 (s, 3H).

### Example 1K

### Synthesis of 4-(6-hydroxy-2-(4-hydroxyphenyl)-1-oxo-1,2-dihydroisoquinolin-4-yl)benzoic acid (43)

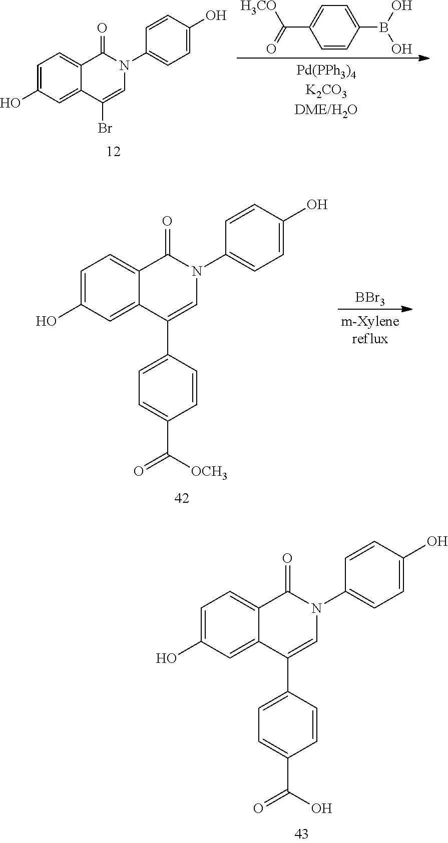 [SMILES: O=c1c2ccc(O)cc2c(Br)cn1-c1ccc(O)cc1 ; COC(=O)c1ccc(B(O)O)cc1 ; COC(=O)c1ccc(-c2cn(-c3ccc(O)cc3)c(=O)c3ccc(O)cc23)cc1 ; "BBr3" ; O=C(O)c1ccc(-c2cn(-c3ccc(O)cc3)c(=O)c3ccc(O)cc23)cc1]

### Synthesis of methyl 4-(6-hydroxy-2-(4-hydroxyphenyl)-1-oxo-1,2-dihydroisoquinolin-4-yl)benzoate (42)

4-Bromo-6-hydroxy-2-(4-hydroxyphenyl)isoquinolin-1(2H)-one (12) (0.42 g, 1.26 mmol), tetrakis(triphenylphosphine)palladium (73 mg, 0.063 mmol), potassium carbonate (0.70 g, 5.06 mmol) and 4-(methoxycarbonyl)phenylboronic acid (0.34 g, 1.90 mmol) were placed in a dry and argon flushed 250 mL three-necked round-bottomed flask fitted with a stirring bar and a reflux condenser. 1,2-Dimethoxyethane (30 mL) and water (10 mL) were added via a syringe under argon atmosphere. The reaction solution was stirred and heated to reflux overnight. The reaction mixture was diluted with 100 mL of water at room temperature. The mixture was extracted with ethyl acetate (5×20 mL). The organic layers were combined, dried over anhydrous MgSO₄, filtered and concentrated to dryness. The residue was purified by flash column chromatography (silica-gel, CH₂Cl₂/acetone/MeOH=90/5/5 v/v/v) to give a white solid product, 0.28 g, 56.0% yield. MS: m/e 385.9 [M−H]⁻. ¹H NMR (DMSO-d₆, 500 MHz) δ 10.35 (s, 1H), 9.71 (s, 1H), 8.20 (d, 1H, J=9.0 Hz), 8.06 (d, 2H, J=8.0 Hz), 7.65 (d, 2H, J=8.5 Hz), 7.31 (s, 1H), 7.28 (d, 2H, J=9.0 Hz), 7.02 (dd, 1H, J₁=8.5 Hz, J₂=2.5 Hz), 6.85 (d, 2H, J=8.5 Hz), 3.89 (s, 3H).

### Synthesis of 4-(6-hydroxy-2-(4-hydroxyphenyl)-1-oxo-1,2-dihydroisoquinolin-4-yl)benzoic acid (43)

Methyl 4-(6-hydroxy-2-(4-hydroxyphenyl)-1-oxo-1,2-dihydroisoquinolin-4-yl)benzoate (42) (0.20 g, 0.52 mmol) was suspended in 30 mL of anhydrous m-xylene in a dry and argon flushed 150 mL single-necked round-bottomed flask fitted with a stirring bar and an argon inlet. BBr₃ (0.65 g, 2.58 mmol) was added via a syringe with stirring at room temperature. After stirred and heated to 100° C. for 10 hours, the reaction was quenched by adding 50 mL of water and 5 mL of methanol. The solution was stirred at room temperature for one hour. The mixture was extracted with EtOAc (4×20 mL). The organic layers were separated, combined and dried over anhydrous MgSO₄. The solvent was removed under reduced pressure. The residue was purified by column chromatography (silica-gel, CH₂Cl₂/acetone/MeOH=8/1/1 v/v/v) to give a pale-yellow solid product, 0.12 g, 63.2% yield. MS: m/e 372.0 [M−H]⁻. ¹H NMR (DMSO-d₆, 300 MHz) δ 13.00 (s, 1H), 10.34 (s, 1H), 9.70 (s, 1H), 8.20 (d, 1H, J=9.0 Hz), 8.04 (d, 2H, J=8.4 Hz), 7.61 (d, 2H, J=8.4 Hz), 7.30-7.28 (m, 3H), 7.02 (dd, 1H, J₁=8.7 Hz, J₂=2.4 Hz), 6.87-6.84 (m, 3H).

### Example 11

### Synthesis of 2-benzyl-6-hydroxy-4-(3,4,5-trifluorophenyl)isoquinolin-1(2H)-one (75)

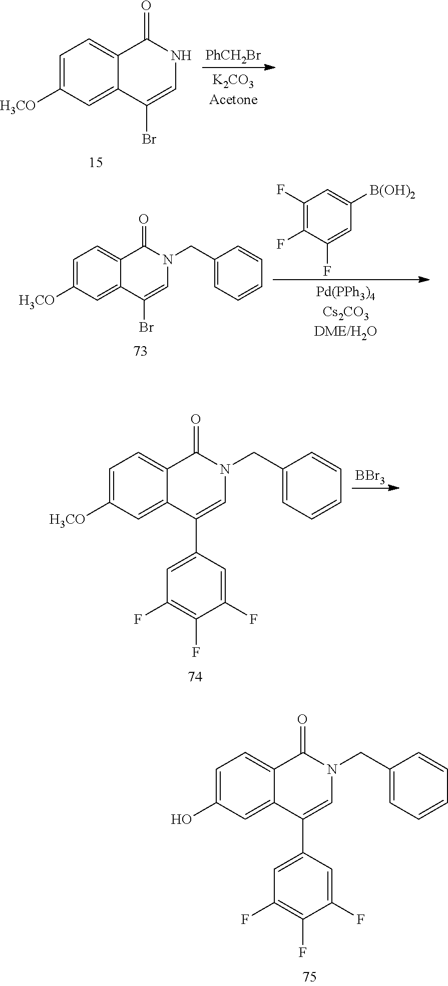

### Synthesis of 2-benzyl-4-bromo-6-methoxyisoquinolin-1(2H)-one (73)

4-Bromo-6-methoxyisoquinolin-1(2H)-one (15) (0.63 g, 2.48 mmol), benzyl bromide (1.27 g, 7.44 mmol) and anhydrous potassium carbonate (1.03 g, 7.44 mmol) were placed in a dry 250 mL three-necked round-bottomed flask fitted with a stiffing bar and reflux condenser. The reaction flask was vacuumed and refilled with dry argon. 30 mL of dry acetone was added via a syringe. The reaction mixture was stirred and heated to reflux overnight. The solvent was removed under reduced pressure. The residue was purified by flash column chromatography (silica gel, CH₂Cl₂) to give a white solid product, 0.78 g, 91.8% yield. MS: 368.1 [M+Na]⁺. ¹H NMR (DMSO-d₆, 500 MHz) δ 8.20 (d, 1H, J=9.0 Hz), 8.07 (s, 1H), 7.34-7.33 (m, 4H), 7.29-7.27 (m, 1H), 7.20 (d, 1H, J=8.5 Hz), 7.10 (s, 1H), 5.16 (s, 2H), 3.92 (s, 3H).

### Synthesis of 2-benzyl-6-methoxy-4-(3,4,5-trifluorophenyl)isoquinolin-1(2H)-one (74)

2-Benzyl-4-bromo-6-methoxyisoquinolin-1(2H)-one (73) (0.51 g, 1.48 mmol), tetrakis(triphenylphosphine)palladium (86 mg, 0.074 mmol), cesium carbonate (0.97 g, 2.96 mmol) and 3,4,5-trifluorophenylboronic acid (0.31 g, 1.78 mmol) were placed in a dry and argon flushed 150 mL three-necked round-bottomed flask fitted with a stirring bar and reflux condenser. 1,2-dimethoxyethane (30 mL) and water (10 mL) were added via a syringe under argon atmosphere. The reaction solution was stirred and heated to reflux overnight. The reaction solution was diluted by adding 100 mL of water at room temperature. The mixture was extracted with ethyl acetate (3×50 mL). The extracts were combined, washed with brine (2×10 mL) and dried over anhydrous MgSO₄ followed by filtration and concentration to give a yellow residue. The yellow residue was purified by flash column chromatography (silica-gel, CH₂Cl₂/acetone=97/3 v/v) to give a white solid product, 0.45 g, 76.8% yield. MS: m/z 396.1 [M+H]⁺. ¹H NMR (DMSO-d₆, 500 MHz) δ 8.26 (d, 1H, J=9.0 Hz), 7.70 (s, 1H), 7.49 (t, 2H, J=8.0 Hz), 7.38-7.37 (m, 2H), 7.33 (t, 2H, J=7.5 Hz), 7.28-7.26 (m, 1H), 7.18 (dd, 1H, J₁=9.0 Hz, J₂=2.0 Hz), 6.87 (d, 1H, J=2.0 Hz), 5.18 (s, 2H), 3.80 (s, 3H).

### Synthesis of 2-benzyl-6-hydroxy-4-(3,4,5-trifluorophenyl)isoquinolin-1(2H)-one (75)

2-Benzyl-6-methoxy-4-(3,4,5-trifluorophenyl)isoquinolin-1(2H)-one (74) (0.30 g, 0.76 mmol) was placed in a dry 250 mL single-necked round-bottomed flask fitted with a stirring bar and sealed with a rubber stopper. Anhydrous chlorobenzene (30 mL) was added via a syringe at room temperature. BBr₃ (0.95 g, 3.79 mmol) was added dropwise with stirring at room temperature. The resulted solution was heated to 90° C. overnight. 50 mL of water and 10 mL of methanol were added to quench the reaction at 0° C. The solution was stirred at room temperature for two hours and extracted with EtOAc (3×50 mL). The organic layers were combined and dried over anhydrous MgSO₄. The solvent was removed under reduced pressure. The residue was purified by column chromatography (silica-gel, CH₂Cl₂/MeOH=9/1 v/v) to give a white solid product, 0.25 g, 86.5% yield. MS: m/e 380.0 [M−H]⁻. ¹H NMR (DMSO-d₆, 500 MHz) δ 10.35 (s, 1H), 8.17 (d, 1H, J=8.5 Hz), 7.64 (s, 1H), 7.46 (t, 2H, J=8.0 Hz), 7.38-7.31 (m, 4H), 7.28-7.25 (m, 1H), 7.00 (d, 1H, J=8.5 Hz), 6.81 (s, 1H), 5.16 (s, 2H).

### Example 1M

### Synthesis of 6-hydroxy-2-(4-hydroxyphenyl)-4-(3,4,5-trifluorophenyl)isoquinolin-1(2H)-one (79)

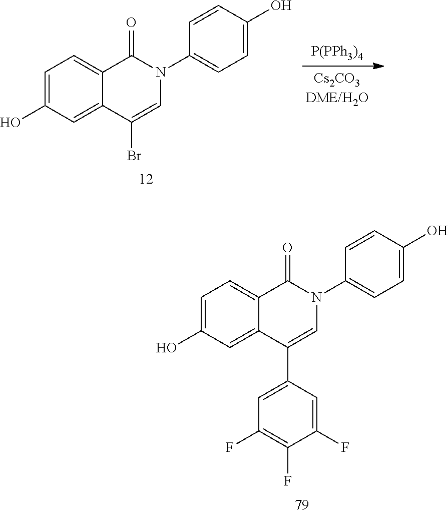 [SMILES: O=c1c2ccc(O)cc2c(Br)cn1-c1ccc(O)cc1 ; O=c1c2ccc(O)cc2c(-c2cc(F)c(F)c(F)c2)cn1-c1ccc(O)cc1]

Synthesis of 6-hydroxy-2-(4-hydroxyphenyl)-4-(3,4,5-trifluorophenyl)isoquinolin-1(2H)-one (79)

4-Bromo-6-hydroxy-2-(4-hydroxyphenyl)isoquinolin-1(2H)-one (12) (0.54 g, 1.63 mmol), tetrakis(triphenylphosphine)palladium (0.19 g, 0.16 mmol), cesium carbonate (2.12 g, 6.50 mmol) and 3,4,5-trifluorophenylboronic acid (0.43 g, 2.44 mmol) were placed in a dry and argon flushed 250 mL three-necked round-bottomed flask fitted with a stirring bar and reflux condenser. 1,2-dimethoxyethane (30 mL) and water (10 mL) were added via a syringe under argon atmosphere. The reaction solution was stirred and heated to reflux overnight. The reaction solution was diluted by adding 100 mL of water at room temperature and extracted with ethyl acetate (5×30 mL). The extracts were dried over anhydrous MgSO₄, filtered and concentrated to dryness under reduced pressure. The residue was purified by flash column chromatography (silica-gel, CH₂Cl₂/MeOH=9/1 v/v) to give a white solid product, 0.60 g, 96.3% yield. MS: m/e 381.9 [M−H]⁻. ¹H NMR (DMSO-d₆, 300 MHz) δ 10.40 (s, 1H), 9.72 (s, 1H), 8.19 (d, 1H, J=9.7 Hz), 7.56-7.46 (m, 2H), 7.34 (s, 1H), 7.30-7.26 (m, 2H), 7.02 (dd, 1H, J₁=8.7 Hz, J₂=2.4 Hz), 6.88-6.83 (m, 3H).

### Example 1N

### Synthesis of 6-hydroxy-2-(4-hydroxyphenyl)-1-oxo-4-(3,4,5-trifluorophenyl)-1,2-dihydroisoquinoline-8-carbonitrile (85)

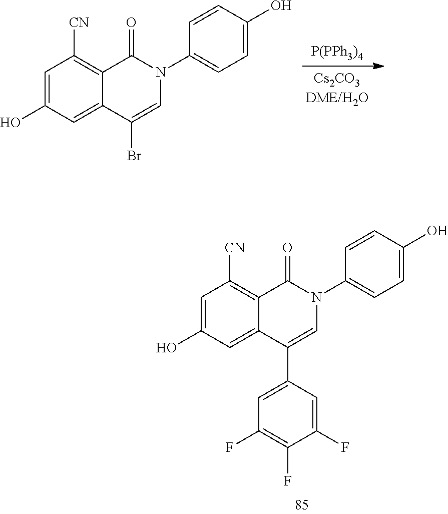 [SMILES: N#Cc1cc(O)cc2c(Br)cn(-c3ccc(O)cc3)c(=O)c12 ; N#Cc1cc(O)cc2c(-c3cc(F)c(F)c(F)c3)cn(-c3ccc(O)cc3)c(=O)c12]

### Synthesis of 6-hydroxy-2-(4-hydroxyphenyl)-1-oxo-4-(3,4,5-trifluorophenyl)-1,2-dihydroisoquinoline-8-carbonitrile (85)

4-Bromo-6-hydroxy-2-(4-hydroxyphenyl)-1-oxo-1,2-dihydroisoquinoline-8-carbonitrile (synthesized per WO2008/091555) (0.23 g, 0.64 mmol), tetrakis(triphenylphosphine)palladium (74 mg, 0.064 mmol), cesium carbonate (0.84 g, 2.58 mmol) and 3,4,5-trifluorophenylboronic acid (0.17 g, 0.97 mmol) were placed in a dry and argon flushed 250 mL three-necked round-bottomed flask fitted with a stirring bar and reflux condenser. 1,2-dimethoxyethane (30 mL) and water (10 mL) were added via a syringe under argon atmosphere. The reaction solution was stirred and heated to reflux overnight. The reaction solution was diluted by adding 100 mL of water at room temperature and extracted with ethyl acetate (5×30 mL). The extracts were dried over anhydrous MgSO₄, filtered and concentrated to dryness under reduced pressure. The residue was purified by flash column chromatography (silica-gel, CH₂Cl₂/MeOH=9/1 v/v) to give a white solid product, 80 mg, 30.8% yield. MS: m/e 407.0 [M−H]⁻. ¹H NMR (DMSO-d₆, 300 MHz) δ 11.09 (s, 1H), 9.77 (s, 1H), 7.57-7.53 (m, 2H), 7.50 (s, 1H), 7.45 (d, 1H, J=2.4 Hz), 7.30 (d, 2H, J=8.7 Hz), 7.09 (d, 1H, J=2.4 Hz), 6.87 (d, 1H, J=9.0 Hz).

### Example 1M

### Synthesis of methyl 6-hydroxy-2-(4-hydroxyphenyl)-1-oxo-4-(3,4,5-trifluorophenyl)-1,2-dihydroisoquinoline-8-carbimidate (100) and 6-hydroxy-2-(4-hydroxyphenyl)-1-oxo-4-(3,4,5-trifluorophenyl)-1,2-dihydroisoquinoline-8-carboxamide (100A)

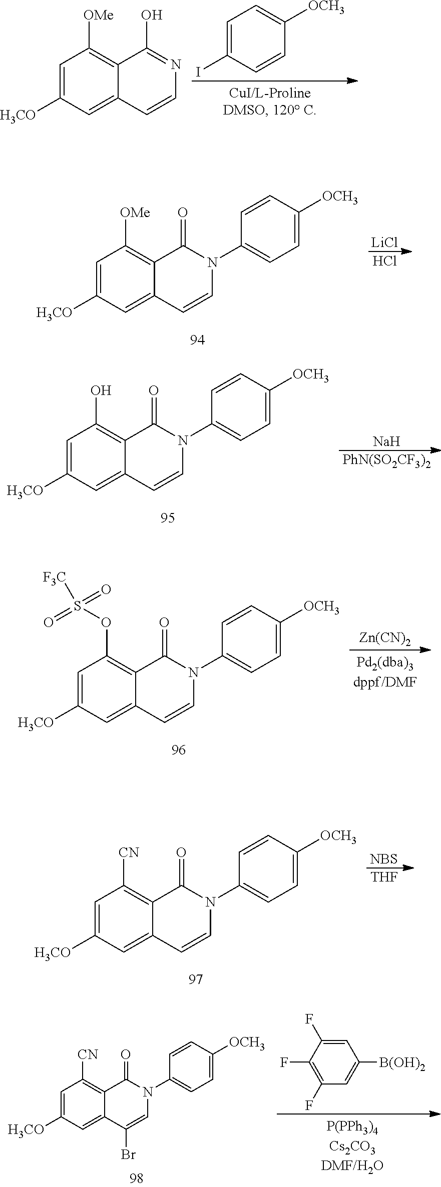

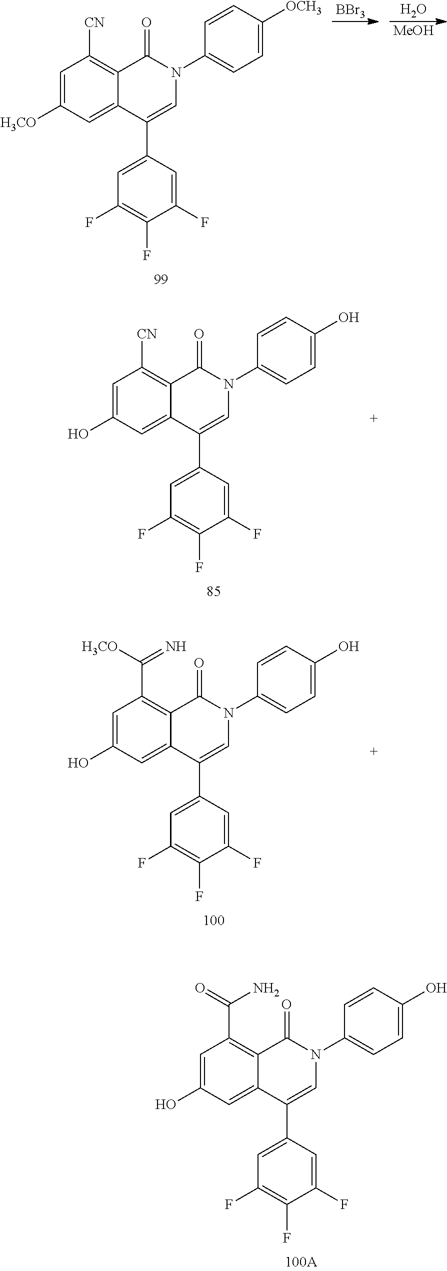

### Synthesis of 6,8-dimethoxy-2-(4-methoxyphenyl)isoquinolin-1(2H)-one (94)

6,8-Dimethoxyisoquinolin-1-ol (1.59 g, 7.75 mmol), 4-iodoanisole (2.72, 11.62 mmol), copper(I) iodide (0.30 g, 1.55 mmol), L-proline (0.36 g, 3.10 mmol) and anhydrous potassium carbonate (2.14 g, 15.50 mmol) were placed in a dry 250 mL three-necked round-bottomed flask fitted with a stirring bar and reflux condenser. The system was vacuumed and refilled with dry argon. Then, anhydrous methyl sulfoxide (50 mL) was added via a syringe under argon atmosphere. The reaction solution was stirred and heated to 120° C. for 20 hours. Water (20 mL) was added to quench the reaction. The mixture was extracted with ethyl acetate (5×20 mL). The extracts were combined, washed with brine (3×10 mL) and dried over anhydrous MgSO₄ followed by filtration and concentration to give a yellow residue. The yellow residue was purified by flash column chromatography (silica-gel, CH₂Cl₂/Acetone=19/1 v/v) to give a pale-yellow solid product, 2.12 g, 88.0% yield. MS: m/z 312.9 [M+H]⁺. ¹H NMR (DMSO-d₆, 500 MHz) δ 7.31-7.26 (m, 3H), 7.02 (d, 2H, J=8.7 Hz), 6.71 (d, 1H, J=2.4 Hz), 6.54 (d, 1H, J=2.4 Hz), 6.45 (d, 1H, J=7.8 Hz), 3.87 (s, 3H), 3.81 (s, 3H), 3.79 (s, 3H).

### Synthesis of 8-hydroxy-6-methoxy-2-(4-methoxyphenyl)isoquinolin-1(2H)-one (95)

6,8-Dimethoxy-2-(4-methoxyphenyl)isoquinolin-1(2H)-one (94) (2.25 g, 7.23 mmol) and LiCl (6.12 g, 144.54 mmol) were placed in a dry, argon flushed 150 mL three-necked flask fitted with a stirring bar and reflux condenser. Anhydrous DMF (30 mL) was added via a syringe. The reaction mixture was heated to 140° C. under vacuum for 20 hours. Then, the reaction was quenched by addition of 30 mL of 2N HCl solution. The solution was extracted with EtOAc (3×30 mL). The extracts were combined and dried over anhydrous MgSO₄. The solvent was removed under reduced pressure. The residue was purified by flash column chromatography (silica-gel, CH₂Cl₂) to give a white solid product, 1.80 g, 83.7% yield. ¹H NMR (DMSO-d₆, 500 MHz) δ 12.98 (s, 1H), 7.42-7.35 (m, 3H), 7.06 (d, 2H, J=9.0 Hz), 6.70-6.67 (m, 2H), 6.45 (d, 1H, J=2.1 Hz), 3.85 (s, 3H), 3.82 (s, 3H).

### Synthesis of 6-methoxy-2-(4-methoxyphenyl)-1-oxo-1,2-dihydroisoquinolin-8-yl trifluoromethanesulfonate (96)

8-Hydroxy-6-methoxy-2-(4-methoxyphenyl)isoquinolin-1(2H)-one (95) (2.10 g, 7.06 mmol) was dissolved in 30 mL of anhydrous dimethylformide in a 250 mL three-necked round-bottomed flask fitted with a magnetic stirring bar, an argon inlet and sealed with rubber stoppers. The solution was cooled to 0° C. in an ice-bath. Sodium hydride (0.37 g of 60% wt. in mineral oil, 9.18 mmol) was added in 4 portions under argon atmosphere. The reaction mixture was stirred at 0° C. for 30 minutes, than at room temperature for 30 minutes. After the solution was cooled to 0° C. again, N-phenyl-bis(trifluoromethanesulfonimde) (2.65 g, 7.41 mmol) was added in portions under argon protection. The reaction mixture was stirred at 0° C. for 30 minutes and at room temperature for one hour. The reaction was quenched by adding 50 mL of saturated ammonia chloride solution, and diluted with 50 mL of water. The solution was extracted with ethyl acetate (3×50 mL). The organic layers were separated, combined, washed with brine, dried over anhydrous MgSO₄, filtered and concentrated under reduced pressure. The residue was purified by flash column chromatography (silica gel, hexanes/EtOAc=1/1 v/v) to give a white solid product, 2.85 g, 94.1% yield. MS: m/z 452.1 [M+Na]⁺. ¹H NMR (DMSO-d₆, 300 MHz) δ 7.52 (d, 1H, J=7.2 Hz), 7.38 (d, 1H, J=2.4 Hz), 7.34 (d, 2H, J=9.0 Hz), 7.07 (d, 2H, J=9.0 Hz), 7.02 (d, 1H, J=1.8 Hz), 6.72 (d, 1H, J=7.5 Hz), 3.94 (s, 3H), 3.82 (s, 3H).

### Synthesis of 6-Methoxy-2-(4-methoxyphenyl)-1-oxo-1,2-dihydroisoquinoline-8-carbonitrile (97)

6-Methoxy-2-(4-methoxyphenyl)-1-oxo-1,2-dihydroisoquinolin-8-yl to trifluoromethanesulfonate (96) (0.43 g, 1.00 mmol), Zn(CN)₂ (0.14 g, 1.20 mmol), tris(dibenzylideneacetone)dipalladium (92 mg, 0.1 mmol) and 1,1′-bis(diphenylphosphino)ferrocene (0.22 g, 0.40 mmol) were placed in a dry and argon flushed 150 mL three-necked round-bottomed flask fitted with a stirring bar and reflux condenser. Then, anhydrous dimethylformide (20 mL) was added via a syringe under argon atmosphere. The reaction solution was stirred and heated to 100° C. for 4 hours. Water (20 mL) was added to quench the reaction. The mixture was extracted with ethyl acetate (4×30 mL). The extracts were combined, washed with brine (3×10 mL) and dried over anhydrous MgSO₄ followed by filtration and concentration to give a yellow residue. The yellow residue was purified by flash column chromatography (silica-gel, EtOAc/hexanes=3/2 v/v) to give a white solid product, 0.23 g, 75.2% yield. MS: m/z 307.2 [M+H]⁺. ¹H NMR (DMSO-d₆, 300 MHz) δ 7.63 (d, 1H, J=2.1 Hz), 7.54 (d, 1H, J=2.1 Hz), 7.51 (d, 1H, J=7.5 Hz), 7.38 (d, 2H, J=8.7 Hz), 7.06 (d, 2H, J=8.7 Hz), 6.71 (d, 1H, J=7.5 Hz), 3.95 (s, 3H), 3.82 (s, 3H).

### Synthesis of 4-bromo-6-methoxy-2-(4-methoxyphenyl)-1-oxo-1,2-dihydroisoquinoline-8-carbonitrile (98)

6-Methoxy-2-(4-methoxyphenyl)-1-oxo-1,2-dihydroisoquinoline-8-carbonitrile (97) (0.22 g, 0.72 mmol) and N-bromosuccinimide (0.15 g, 0.86 mmol) were placed in a dry, argon flushed 150 mL single-necked flask fitted with a stirring bar and sealed with a rubber stopper. Acetonitrile (10 mL) was added via a syringe at room temperature under argon atmosphere. After the mixture was stirred at room temperature for 4 hours, the solvent was removed under reduced pressure. The residue was purified by flash column chromatography (silica-gel, hexanes/EtOAc=2/3 v/v) to give a white solid product, 0.23 g, 83.3% yield. MS: m/z 387.1 [M+H]⁺. ¹H NMR (DMSO-d₆, 300 MHz) δ 8.01 (s, 1H), 7.81 (d, 1H, J=2.4 Hz), 7.43 (d, 1H, J=2.4 Hz), 7.42 (d, 2H, J=8.7 Hz), 7.07 (d, 2H, J=8.7 Hz), 4.02 (s, 3H), 3.82 (s, 3H).

### Synthesis of 6-methoxy-2-(4-methoxyphenyl)-1-oxo-4-(3,4,5-trifluorophenyl)-1,2-dihydroisoquinoline-8-carbonitrile (99)

4-Bromo-6-methoxy-2-(4-methoxyphenyl)-1-oxo-1,2-dihydroisoquinoline-8-carbonitrile (98) (1.00 g, 2.60 mmol), tetrakis(triphenylphosphine)palladium (0.15 g, 0.13 mmol), cesium carbonate (1.69 g, 5.19 mmol) and 3,4,5-trifluorophenylboronic acid (0.55 g, 3.12 mmol) were placed in a dry and argon flushed 250 mL three-necked round-bottomed flask fitted with a stirring bar and reflux condenser. 1,2-Dimethoxyethane (30 mL) and water (10 mL) were added via a syringe under argon atmosphere. The reaction solution was stirred and heated to reflux overnight. The reaction solution was diluted by adding 100 mL of water at room temperature and extracted with ethyl acetate (5×30 mL). The extracts were dried over anhydrous MgSO₄, filtered and concentrated to dryness under reduced pressure. The residue was purified by flash column chromatography (silica-gel, CH₂Cl₂/MeOH=9/1 v/v) to give a white solid product, 1.05 mg, 92.9% yield. MS: m/z 459.1. [M+Na]⁺. ¹H NMR (DMSO-d₆, 300 MHz) δ 7.76 (d, 1H, J=2.4 Hz), 7.58-7.52 (m, 2H), 7.47 (d, 2H, J=9.0 Hz), 7.16 (d, 1H, J=2.4 Hz), 7.07 (d, 2H, J=9.0 Hz), 3.89 (s, 3H), 3.82 (s, 3H).

### Synthesis of methyl 6-hydroxy-2-(4-hydroxyphenyl)-1-oxo-4-(3,4,5-trifluorophenyl)-1,2-dihydroisoquinoline-8-carbimidate (100) and 6-hydroxy-2-(4-hydroxyphenyl)-1-oxo-4-(3,4,5-trifluorophenyl)-1,2-dihydroisoquinoline-8-carboxamide (100A)

6-Methoxy-2-(4-methoxyphenyl)-1-oxo-4-(3,4,5-trifluorophenyl)-1,2-dihydroisoquinoline-8-carbonitrile (99) (1.00 g, 2.29 mmol) was placed in a dry and argon flushed 100 mL single-necked round-bottomed flask fitted with a stirring bar, reflux condenser and an argon inlet. Anhydrous 1,2-dichloroethane (30 mL) was added via a syringe at room temperature. BBr₃ (2.30, 9.17 mmol) was added via a syringe with stirring at room temperature. The resulted solution was heated to 80° C. for 5 hours. The reaction mixture was poured into 100 mL of ice water and 10 mL methanol. After stirred at room temperature for two hours, the solution was extracted with EtOAc (3×50 mL). The organic layers were combined and dried over anhydrous MgSO₄. The solvent was removed under reduced pressure. The residue was purified by column chromatography (silica-gel, CH₂Cl₂/MeOH=9/1 v/v) to give white solid products.

### 6-Hydroxy-2-(4-hydroxyphenyl)-1-oxo-4-(3,4,5-trifluorophenyl)-1,2-dihydroisoquinoline-8-carbonitrile (85)

0.30 g, 32.1% yield. MS: m/e 407.0 [M−H]⁻. ¹H NMR (DMSO-d₆, 500 MHz) δ 11.08 (s, 1H), 9.78 (s, 1H), 7.55-7.52 (m, 2H), 7.50 (s, 1H), 7.30 (d, 2H, J=9.0 Hz), 7.09 (d, 1H, J=2.5 Hz), 6.87 (d, 2H, J=8.5 Hz);

### Methyl 6-hydroxy-2-(4-hydroxyphenyl)-1-oxo-4-(3,4,5-trifluorophenyl)-1,2-dihydroisoquinoline-8-carbimidate (100)

0.21 g, 20.8% yield. MS: m/e 439.0 [M−H]⁻. ¹H NMR (DMSO-d₆, 500 MHz) δ 10.60 (s, 1H), 9.73 (s, 1H), 8.05 (s, 1H), 7.49 (t, 2H, J=8.0 Hz), 7.36 (s, 1H), 7.22 (d, 2H, J=9.0 Hz), 6.85 (d, 2H, J=9.0 Hz), 6.83 (d, 1H, J=2.0 Hz), 6.87 (s, 1H), 3.69 (s, 3H); and

### 6-hydroxy-2-(4-hydroxyphenyl)-1-oxo-4-(3,4,5-trifluorophenyl)-1,2-dihydroisoquinoline-8-carboxamide (100A)

0.16 g, 16.4% yield. MS: m/e 424.9 [M−H]⁻. ¹H NMR (DMSO-d₆, 500 MHz) δ 10.52 (s, 1H), 9.72 (s, 1H), 7.50 (d, 1H, J=8.5 Hz), 7.48 (d, 1H, J=8.0 Hz), 7.41 (s, 1H), 7.37 (s, 1H), 7.24 (d, 2H, J=8.5 Hz), 7.14 (s, 1H), 6.85 (d, 2H, J=8.5 Hz), 6.79 (d, 1H, J=2.0 Hz), 6.87 (d, 1H, J=2.0 Hz).

### Example 1N

### Synthesis of 4-(3-fluoro-4-(trifluoromethyl)phenyl)-6-hydroxy-2-(4-hydroxyphenyl)-1-oxo-1,2-dihydroisoquinoline-8-carbonitrile (102), methyl 4-(3-fluoro-4-(trifluoromethyl)phenyl)-6-hydroxy-2-(4-hydroxyphenyl)-1-oxo-1,2-dihydroisoquinoline-8-carbimidate (102A), and 4-(3-fluoro-4-(trifluoromethyl)phenyl)-6-hydroxy-2-(4-hydroxyphenyl)-1-oxo-1,2-dihydroisoquinoline-8-carboxamide (102B)

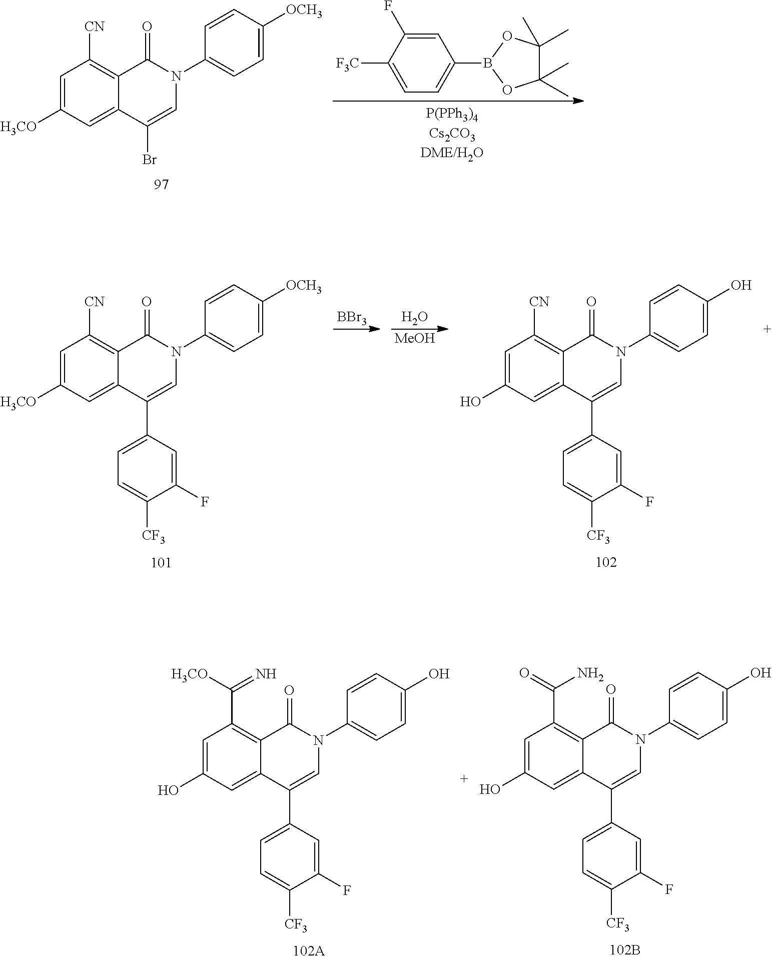

### Synthesis of 4-(3-fluoro-4-(trifluoromethyl)phenyl)-6-methoxy-2-(4-methoxyphenyl)-1-oxo-1,2-dihydroisoquinoline-8-carbonitrile (101)

4-Bromo-6-methoxy-2-(4-methoxyphenyl)-1-oxo-1,2-dihydroisoquinoline-8-carbonitrile (97) (1.00 g, 2.60 mmol), tetrakis(triphenylphosphine)palladium (0.15 g, 0.13 mmol), cesium carbonate (1.69 g, 5.19 mmol) and 2-(3-fluoro-4-(trifluoromethyl)phenyl)-4,4,5,5-tetramethyl-1,3,2-dioxaborolane (0.90 g, 3.12 mmol) were placed in a dry and argon flushed 250 mL three-necked round-bottomed flask fitted with a stiffing bar and reflux condenser. 1,2-Dimethoxyethane (30 mL) and water (10 mL) were added via a syringe under argon atmosphere. The reaction solution was stirred and heated to reflux overnight. The reaction solution was diluted by adding 100 mL of water at room temperature and extracted with ethyl acetate (5×30 mL). The extracts were dried over anhydrous MgSO₄, filtered and concentrated to dryness under reduced pressure. The residue was purified by flash column chromatography (silica-gel, CH₂Cl₂/MeOH=9/1 v/v) to give a white solid product, 1.20 mg, 98.4% yield. MS: m/z 491.1 [M+Na]⁺. ¹H NMR (DMSO-d₆, 300 MHz) δ 7.89 (t, 1H, J=8.1 Hz), 7.79-7.74 (m, 2H), 7.66 (s, 1H), 7.60 (d, 1H, J=8.4 Hz), 7.48 (d, 2H, J=9.0 Hz), 7.19 (d, 1H, J=2.7 Hz), 7.07 (d, 2H, J=9.0 Hz), 3.89 (s, 3H), 3.82 (s, 3H).

### Synthesis of 4-(3-fluoro-4-(trifluoromethyl)phenyl)-6-hydroxy-2-(4-hydroxyphenyl)-1-oxo-1,2-dihydroisoquinoline-8-carbonitrile (102), methyl 4-(3-fluoro-4-(trifluoromethyl)phenyl)-6-hydroxy-2-(4-hydroxyphenyl)-1-oxo-1,2-dihydroisoquinoline-8-carbimidate (102A), and 4-(3-fluoro-4-(trifluoromethyl)phenyl)-6-hydroxy-2-(4-hydroxyphenyl)-1-oxo-1,2-dihydroisoquinoline-8-carboxamide (102B)

6-Methoxy-2-(4-methoxyphenyl)-1-oxo-4-(3,4,5-trifluorophenyl)-1,2-dihydroisoquinoline-8-carbonitrile (101) (1.16 g, 2.48 mmol) was placed in a dry and argon flushed 100 mL single-necked round-bottomed flask fitted with a stirring bar, reflux condenser and an argon inlet. Anhydrous 1,2-dichloroethane (30 mL) was added via a syringe at room temperature. BBr₃ (2.48, 9.91 mmol) was added via a syringe with stiffing at room temperature. The resulted solution was heated to 80° C. for 5 hours. The reaction mixture was poured into 100 mL of ice water and 10 mL methanol. After stiffed at room temperature for two hours, the solution was extracted with EtOAc (3×50 mL). The organic layers were combined and dried over anhydrous MgSO₄. The solvent was removed under reduced pressure. The residue was purified by column chromatography (silica-gel, CH₂Cl₂/MeOH=9/1 v/v) to give white solid products.

### 4-(3-Fluoro-4-(trifluoromethyl)phenyl)-6-hydroxy-2-(4-hydroxyphenyl)-1-oxo-1,2-dihydroisoquinoline-8-carbonitrile (102)

0.55 g, 50.5% yield. MS: m/e 439.0 [M−H]⁻. ¹H NMR (DMSO-d₆, 500 MHz) δ 11.05 (s, 1H), 9.79 (s, 1H), 7.91 (t, 0.5H, J=8.0 Hz), 7.81 (t, 0.5H, J=8.0 Hz), 7.72 (d, 1H, J=12.0 Hz), 7.60-7.58 (m, 1H), 7.56-7.51 (m, 1H), 7.47 (d, 1H, J=2.5 Hz), 7.32 (d, 2H, J=8.5 Hz), 7.12-7.10 (m, 1H), 6.88 (d, 2H, J=9.0 Hz);

### Methyl 4-(3-fluoro-4-(trifluoromethyl)phenyl)-6-hydroxy-2-(4-hydroxyphenyl)-1-oxo-1,2-dihydroisoquinoline-8-carbimidate (102A)

0.26 g, 22.2% yield. MS: m/e 470.9 [M−H]⁻. ¹H NMR (DMSO-d₆, 500 MHz) δ 10.59 (s, 1H), 9.73 (s, 1H), 8.09 (s, 1H), 7.99 (t, 0.5H, J=8.0 Hz), 7.89 (t, 0.5H, J=8.0 Hz), 7.68 (d, 0.5H, J=12.0 Hz), 7.53-7.49 (m, 1H), 7.45-7.41 (m, 1.5H), 7.23 (d, 2H, J=8.5 Hz), 6.87-6.84 (m, 3H), 6.78 (s, 1H), 3.69 (s, 3H); and

### 6-Hydroxy-2-(4-hydroxyphenyl)-1-oxo-4-(3,4,5-trifluorophenyl)-1,2-dihydroisoquinoline-8-carboxamide (102B)

0.16 g, 14.1% yield. MS: m/e 456.9 [M−H]⁻. ¹H NMR (DMSO-d₆, 500 MHz) δ 10.51 (s, 1H), 9.72 (s, 1H), 7.99 (t, 0.5H, J=8.0 Hz), 7.90 (t, 0.5H, J=8.0 Hz), 7.68 (d, 0.5H, J=11.5 Hz), 7.54-7.49 (m, 1H), 7.45-7.42 (m, 2.5H), 7.25 (d, 2H, J=9.0 Hz), 7.15 (s, 1H), 6.86-6.81 (m, 3H), 6.78 (d, 1H, J=2.5 Hz).

### Example 1O

### Synthesis of 3-(4-(3-fluoro-4-(trifluoromethyl)phenyl)-6-hydroxy-1-oxoisoquinolin-2(1H)-yl)benzamide (214)

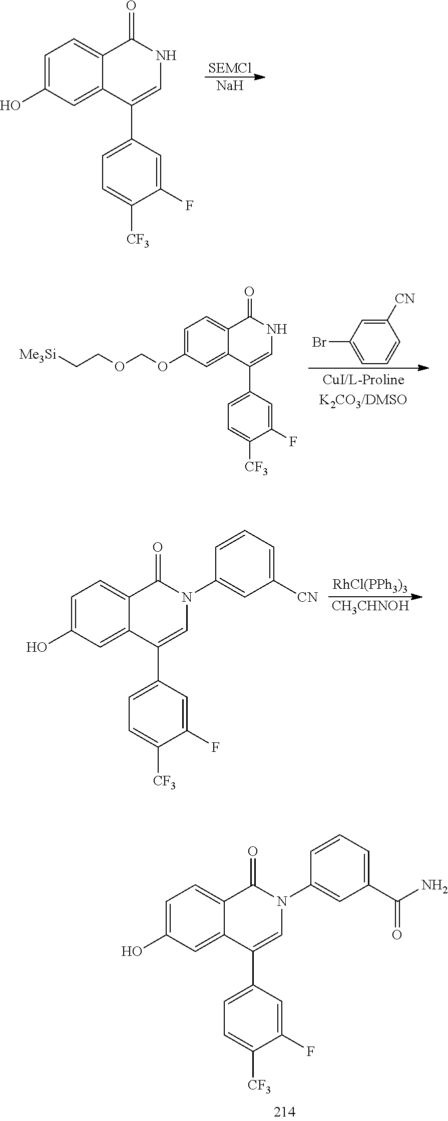

### Synthesis of 4-(3-fluoro-4-(trifluoromethyl)phenyl)-6-((2-(trimethylsilyl)ethoxy)methoxy)isoquinolin-1(2H)-one

Compound 4-(3-fluoro-4-(trifluoromethyl)phenyl)-6-hydroxyisoquinolin-1(2H)-one (0.92 g, 2.85 mmol) was dissolved in 30 mL of anhydrous DMF at room temperature under argon. NaH (0.13 g of 60% wt in mineral oil, 3.42 mmol) was added in portions at 0° C. in an ice bath. The reaction mixture was stirred at room temperature for one hour. Then, 2-(trimethylsilyl)ethoxymethyl chloride (0.81 g, 2.42 mmol) was added via a syringe at room temperature. The resulted mixture was stirred at room temperature for 5 hours. The reaction was quenched by adding 50 mL of saturated NH₄Cl solution. The mixture was extracted with ethyl acetate (3×50 mL). The extracts were dried over anhydrous MgSO₄, filtered and concentrated to dryness under reduced pressure. The residue was purified by flash column chromatography (silica gel, CH₂Cl₂/MeOH=19/1 v/v) to give a white solid product, 0.76 g, 58.9% yield. MS: 454.2 [M+H]⁺. ¹H NMR (DMSO-d₆, 400 MHz): δ 11.57 (d, 1H, J=4.0 Hz), 8.27 (d, 1H, J=8.0 Hz), 7.89 (t, 1H, J=8.0 Hz), 7.67 (d, 1H, J=12 Hz), 7.55 (d, 1H, J=8.0 Hz), 7.28 (d, 1H, J=8.0 Hz), 7.26-7.23 (m, 1H), 7.14 (d, 1H, J=4.0 Hz), 5.31 (s, 2H), 3.71 (t, 2H, J=8.0 Hz), 0.85 (t, 2H, J=8.0 Hz), −0.09 (s, 9H).

### Synthesis of 3-(4-(3-fluoro-4-(trifluoromethyl)phenyl)-6-hydroxy-1-oxoisoquinolin-2(1H)-yl)benzonitrile

4-(3-Fluoro-4-(trifluoromethyl)phenyl)-6-((2-(trimethyl silyl)ethoxy)methoxy)isoquinolin-1(2H)-one (0.59 g, 1.30 mmol), 3-bromobenzonitrile (0.28 g, 1.56 mmol), copper(I) iodide (50 mg, 0.26 mmol), L-proline (60 mg, 0.52 mmol) and anhydrous potassium carbonate (0.36 g, 2.60 mmol) were placed in a dry 250 mL three-necked round-bottomed flask fitted with a stirring bar and reflux condenser. The reaction flask was vacuumed and refilled with dry argon. 30 mL of anhydrous methyl sulfoxide was added via a syringe. The reaction mixture was stirred and heated to 100° C. for 8 hours. The reaction was quenched by adding 200 mL of water at room temperature. The mixture was stirred at room temperature for 2 hours and extracted with ethyl acetate (3×50 mL). The organic layer was separated, washed with brine (2×30 mL) and evaporated to dryness under reduced pressure. The residue was purified by flash column chromatography (silica gel, CH₂Cl₂/MeOH=9/1 v/v)) to give a yellow crude solid product, 0.30 g, 82.9% yield. This solid was used in next step reaction without further purification.

### Synthesis of 3-(4-(3-fluoro-4-(trifluoromethyl)phenyl)-6-hydroxy-1-oxoisoquinolin-2(1H)-yl)benzamide (214)

3-(4-(3-Fluoro-4-(trifluoromethyl)phenyl)-6-hydroxy-1-oxoisoquinolin-2(1H)-yl)benzonitrile (0.30 g, 0.707 mmol), acetaldehyde oxime (0.21 g, 3.51 mmol) and tris(triphenylphosphine)rhodium(I) chloride (6.5 mg, 0.0071 mmol) were mixed together in 10 mL of toluene under argon. The reaction mixture was stirred and heated to reflux for 6 hours. Then, toluene was removed. The residue was subjected to column chromatography (silica-gel, CH₂Cl₂/MeOH=9/1 v/v) to give a white solid product, 0.12 g, 38.7% yield, MS: 443.2 [M+H]⁺. ¹H NMR (DMSO-d₆, 400 MHz): δ 10.46 (s, 1H), 8.24 (d, 1H, J=8.0 Hz), 8.07 (s, 1H), 7.99 (s, 1H), 7.95-7.89 (m, 2H), 7.75-7.70 (m, 2H), 7.62-7.59 (m, 3H), 7.51 (s, 1H), 7.06 (dd, 1H, J₁=8.0 Hz, J₂=2.0 Hz), 6.89 (d, 1H, J=2.0 Hz).

### Example 1P

### Synthesis of 4-(4-(3-fluoro-4-(trifluoromethyl)phenyl)-6-hydroxy-1-oxoisoquinolin-2(1H)-yl)benzamide (215)

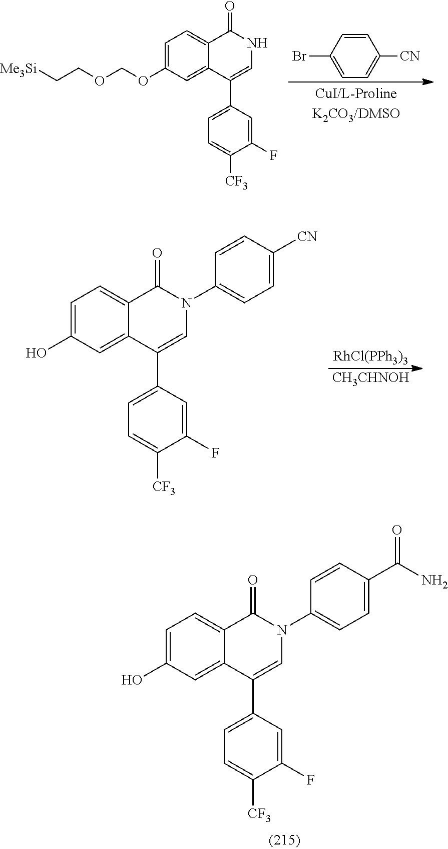 [SMILES: C[Si](C)(C)CCOCOc1ccc2c(=O)[nH]cc(-c3ccc(C(F)(F)F)c(F)c3)c2c1 ; N#Cc1ccc(Br)cc1 ; N#Cc1ccc(-n2cc(-c3ccc(C(F)(F)F)c(F)c3)c3cc(O)ccc3c2=O)cc1 ; "CH3CHNOH" ; NC(=O)c1ccc(-n2cc(-c3ccc(C(F)(F)F)c(F)c3)c3cc(O)ccc3c2=O)cc1 ; "RhCl(PPh3)3"]

### Synthesis of 4-(4-(3-fluoro-4-(trifluoromethyl)phenyl)-6-hydroxy-1-oxoisoquinolin-2(1H)-yl)benzonitrile

4-(3-Fluoro-4-(trifluoromethyl)phenyl)-6-(2-(trimethylsilyl)ethoxy)methoxy)isoquinolin-1(2H)-one (0.28 g, 0.62 mmol), 4-bromobenzonitrile (0.14 g, 0.74 mmol), copper(I) iodide (24 mg, 0.12 mmol), L-proline (28 mg, 0.25 mmol) and anhydrous potassium carbonate (0.17 g, 1.23 mmol) were placed in a dry 250 mL three-necked round-bottomed flask fitted with a stiffing bar and reflux condenser. The reaction flask was vacuumed and refilled with dry argon. 20 mL of anhydrous methyl sulfoxide was added via a syringe. The reaction mixture was stirred and heated to 100° C. for 6 hours. The reaction was quenched by adding 200 mL of water at room temperature. The mixture was stirred at room temperature for 2 hours and extracted with ethyl acetate (3×50 mL). The organic layer was separated, washed with brine (2×30 mL) and evaporated to dryness under reduced pressure. The residue was purified by flash column chromatography (silica gel, CH₂Cl₂/MeOH=9/1 v/v)) to give a yellow crude solid product, 0.20 g, 76.4% yield. This solid was used in next step reaction without further purification.

### Synthesis of 4-(4-(3-fluoro-4-(trifluoromethyl)phenyl)-6-hydroxy-1-oxoisoquinolin-2(1H)-yl)benzamide (215)

4-(4-(3-Fluoro-4-(trifluoromethyl)phenyl)-6-hydroxy-1-oxoisoquinolin-2(1H)-yl)benzonitrile (0.20 g, 0.47 mmol), acetaldehyde oxime (0.14 g, 2.35 mmol) and tris(triphenylphosphine)rhodium(I) chloride (4.35 mg, 0.0047 mmol) were mixed together in 10 mL of toluene under argon. The reaction mixture was stirred and heated to reflux for 6 hours. Then, toluene was removed. The residue was subjected to column chromatography (silica-gel, CH₂Cl₂/MeOH=9/1 v/v) to give a white solid product, 0.11 g, 52.9% yield, MS: 441.0 [M−H]⁻. ¹H NMR (DMSO-d₆, 400 MHz): δ 10.46 (s, 1H), 8.24 (d, 1H, J=8.0 Hz), 8.10 (s, 1H), 8.00 (d, 2H, J=8.0 Hz), 7.93-7.89 (m, 1H), 7.73 (d, 1H, J=12 Hz), 7.64 (d, 2H, J=8.0 Hz), 7.58-7.55 (m, 2H), 7.49 (s, 1H), 7.06 (dd, 1H, J₁=8.0 Hz, J₂=2.0 Hz), 6.88 (d, 1H, J=2.0 Hz).

### Example 1P

### Synthesis of 3-(6-hydroxy-1-oxo-4-(3,4,5-trifluorophenyl)isoquinolin-2(1H)-yl)benzonitrile (216) and 3-(6-hydroxy-1-oxo-4-(3,4,5-trifluorophenyl)isoquinolin-2(1H)-yl)benzamide) (217)

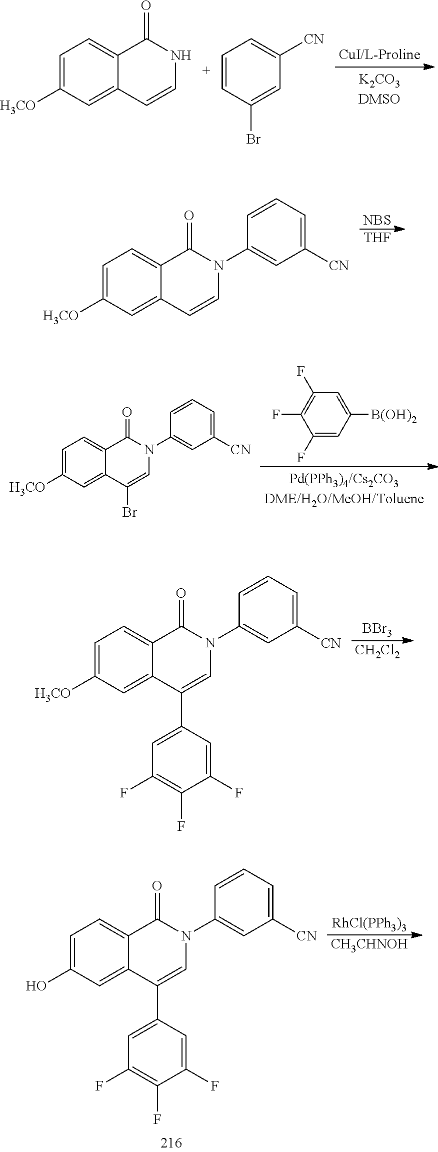

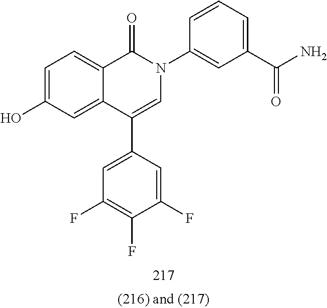 [SMILES: NC(=O)c1cccc(-n2cc(-c3cc(F)c(F)c(F)c3)c3cc(O)ccc3c2=O)c1]

### Synthesis of 3-(6-methoxy-1-oxoisoquinolin-2(1H)-yl)benzonitrile

6-Methoxyisoquinoline-1-ol (3.00 g, 17.12 mmol), 3-bromobenzonitrile (3.74 g, 20.55 mmol), copper(I) iodide (0.65 g, 3.43 mmol), L-proline (0.79 g, 6.86 mmol) and anhydrous potassium carbonate (4.73 g, 34.25 mmol) were placed in a dry 250 mL three-necked round-bottomed flask fitted with a stirring bar and reflux condenser. The reaction flask was vacuumed and refilled with dry argon. 60 mL of anhydrous methyl sulfoxide was added via a syringe. The reaction mixture was stirred and heated to 100° C. overnight. The reaction was quenched by adding 150 mL of water at room temperature. The mixture was stirred at room temperature for 2 hours. The yellow solid was filtered out, washed with water (2×30 mL) and dried under vacuum. The solid was purified by flash column chromatography (silica gel, CH₂Cl₂/acetone=19/1 v/v) to give a yellow solid product, 2.76 g, 58.4% yield.

### Synthesis of 3-(4-bromo-6-methoxy-1-oxoisoquinolin-2(1H)-yl)benzonitrile

3-(6-Methoxy-1-oxoisoquinolin-2(1H)-yl)benzonitrile (2.20 g, 7.97 mmol) was placed in a dry 250 mL single-necked round-bottomed flask fitted with a stiffing bar. THF (50 mL) was added via a syringe under argon atmosphere at room temperature. N-Bromosuccinimide (1.70 g, 9.56 mmol) was added in two portions under argon atmosphere at room temperature. The reaction mixture was allowed to stir and heated to reflux overnight. Then, the solvent was removed under reduced pressure. The residue was purified by flash column chromatography (silica gel, CH₂Cl₂/acetone=19/1 v/v) to give a white solid product, 1.78 g, 62.9% yield.

### Synthesis of 3-(6-methoxy-1-oxo-4-(3,4,5-trifluorophenyl)isoquinolin-2(1H)-yl)benzonitrile

3-(4-Bromo-6-methoxy-1-oxoisoquinolin-2(1H)-yl)benzonitrile (0.80 g, 2.25 mmol), tetrakis(triphenylphosphine)palladium (0.13 mg, 0.113 mmol), cesium carbonate (1.47 g, 4.50 mmol) and 3,4,5-trifluorophenylboronic acid (0.48 g, 2.70 mmol) were placed in a dry and argon flushed 250 mL three-necked round-bottomed flask fitted with a stirring bar and a reflux condenser. 1,2-Dimethoxyethane (30 mL), water (10 mL) and toluene (30 mL) were added via a syringe under argon atmosphere. The reaction solution was stirred and heated to reflux overnight. The reaction mixture was diluted with 150 mL of water at room temperature. The mixture was extracted with ethyl acetate (4×30 mL). The organic layers were combined, dried over anhydrous MgSO₄, filtered and concentrated to dryness. The residue was purified by flash column chromatography (silica gel, CH₂Cl₂/acetone=19/1 v/v) to give a white solid product, 0.65 g, 71.4% yield. MS: 407.2 [M+H]⁺. ¹H NMR (DMSO-d₆, 400 MHz) δ 8.30 (d, 1H, J=8.0 Hz), 8.14-8.13 (m, 1H), 7.96-7.91 (m, 2H), 7.75-7.71 (m, 1H), 7.60-7.56 (m, 3H), 7.25 (dd, 1H, J₁=8.0 Hz, J₂=4.0 Hz), 6.95 (d, 1H, J=4.0 Hz), 3.84 (s, 3H).

### Synthesis of 3-(6-hydroxy-1-oxo-4-(3,4,5-trifluorophenyl)isoquinolin-2(1H)-yl)benzonitrile (216)

3-(6-Methoxy-1-oxo-4-(3,4,5-trifluorophenyl)isoquinolin-2(1H)-yl)benzonitrile (0.40 g, 0.98 mmol) was dissolved in 30 mL of anhydrous CH₂Cl₂ in a dry and argon flushed 150 mL single-necked round-bottomed flask fitted with a stirring bar and an argon inlet. BBr₃ (2.95 mL of 1.0 M CH₂Cl₂ solution, 2.95 mmol) was added via a syringe with stirring at room temperature. After stirred at room temperature overnight, the reaction was quenched by adding 50 mL of water and 5 mL of methanol. The solution was stirred at room temperature for one hour. The CH₂Cl₂ layer was separated. The aqueous layer was extracted with CH₂Cl₂ (3×20 mL). The organic layers were separated, combined and dried over anhydrous MgSO₄. The solvent was removed under reduced pressure. The residue was purified by column chromatography (silica-gel, CH₂Cl₂/MeOH=19/1 v/v) to give a white solid product, 0.28 g, 72.5% yield. MS: 391.0 [M−H]⁻. ¹H NMR (DMSO-d₆, 400 MHz) δ 10.53 (s, 1H), 8.23 (d, 1H, J=8.0 Hz), 8.12 (s, 1H), 7.95-7.91 (m, 2H), 7.75-7.73 (m, 1H), 7.58-7.54 (m, 3H), 7.06 (d, 1H, J=8.0 Hz), 6.88 (s, 1H).

### Synthesis of 3-(6-hydroxy-1-oxo-4-(3,4,5-trifluorophenyl)isoquinolin-2(1H)-yl)benzamide (217)

3-(6-Hydroxy-1-oxo-4-(3,4,5-trifluorophenyl)isoquinolin-2(1H)-yl)benzonitrile (0.20 g, 0.51 mmol), acetaldehyde oxime (0.15 g, 2.55 mmol) and tris(triphenylphosphine)rhodium(I) chloride (4.72 mg, 0.0051 mmol) were mixed together in 20 mL of toluene under argon. The reaction mixture was stirred and heated to reflux for 8 hours. Then, toluene was removed. The residue was subjected to column chromatography (silica-gel, CH₂Cl₂/MeOH=9/1 v/v) to give a white solid product, 0.14 g, 67.0% yield, MS: 409.2 [M−H]⁻. ¹H NMR (DMSO-d₆, 400 MHz): δ 10.48 (s, 1H), 8.23 (d, 1H, J=8.0 Hz), 8.08 (s, 1H), 7.60-7.53 (m, 3H), 7.51 (s, 2H), 7.06 (dd, 1H, J₁=8.0 Hz, J₂=4.0 Hz), 6.88 (d, 1H, J=4.0 Hz).

### Example 2

### Material and Methods

The materials and methods presented here were used throughout Examples 2-7 unless stated otherwise in the Example.

Reagents:

AR antibody, PG-21, were obtained from Millipore (Billerica, Mass.), AR antibody, AR N-20, was obtained from Santa Cruz Biotechnology (Santa Cruz, Calif.), AKR1C3 mouse monoclonal antibody was obtained from Sigma (St. Louis, Mo.) and AKR1C3 rabbit polycloncal antibody was obtained from Life Technologies (Carlsbad, Calif.). Actin antibody was procured from Chemicon International (Temecula, Calif.). Accell siRNAs were purchased from Dharmacon (Lafayette, Colo.). Human PSA ELISA was procured from R&D Systems (Minneapolis, Minn.). All other reagents used were analytical grade from Fisher.

Cloning and Protein Purification:

AKR1C1-C4 cDNAs were cloned into the pCR3.1 vector backbone. AKR1C1 and AKR1C3 construct for protein purification were cloned in pGEX 4T-1. All cloned plasmids were sequenced to ensure absence of any mutations. For AKR1C3 protein production, 10 ml bacterial culture was inoculated in 1 liter of LB and protein synthesis was induced by 1 mM IPTG (Isopropyl β-D-1-Galactopyranoside) for 5 hrs at 37° C. shaking at 250 rpm. The bacterial cells were lysed by freeze thaw cycles, and the protein was purified using glutathione to sepharose beads (Amersham, Piscataway, N.J.). Point mutations were made by Quickchange site directed mutagenesis kit (Agilent Technologies, Santa Clara, Calif.). All the other plasmids were described earlier.

AKR1C3 Enzyme Activity and Thin Layer Chromatography (TLC):

Reaction mixture containing 50 μl phosphate buffer, 0.2 μl NADPH (100 mM), 0.5 μl purified AKR1C3, 0.25 μl [¹⁴C] A'dione (48 mCi/mmol; Perkin Elmer; A'dione also known as 4′dione, androstenedion, and androstene-3,17-dione) was incubated at 37° C. for 60 min. For the progesterone to 20α(OH) progesterone conversion, [¹⁴C]-A'dione was replaced with [¹⁴C]-progesterone (53 mCi/mmol; American Radiolabeled Chemicals, St. Louis, Mo.). The reaction was stopped by the addition of 400 μl ethyl acetate, vortexed, and spun for 5 mM at 2000 rpm. The upper layer was transferred to a new tube and air dried. The pellet was resuspended in 25 μl of methanol, spotted on TLC plates and fractionated in TLC chamber with 3:1 chloroform:ethyl acetate. Once the mobile phase reached the top, the plates were dried and exposed to a phosphoimager screen overnight.

Cell Culture:

All cells were obtained from ATCC (Manassas, Va.) and were grown according to the instructions provided. For the ChIP and co-immunoprecipitation assays, cells were plated in 10 cm dishes at 5 million cells per dish in medium supplemented with 1% charcoal stripped FBS (csFBS). The cells were maintained in 1% csFBS for 3 days to reduce basal occupancy of promoters with medium changed on days 1 and before treatment on day 3.

Transfection and Transactivation Assay:

For transfection, cells were plated at 100, 000 cells per well of a 24-well plate in DME+5% csFBS. The cells were transfected using Lipofectamine (Invitrogen, Carlsbad, Calif.) with indicated plasmids. The cells were treated 24 hrs after transfection as indicated in the figures and the luciferase assay (Dual luciferase assay kit, Promega) performed 48 hrs after transfection. Transfections of siRNAs (Accell siRNAs, Dharmacon Inc.) in LNCaP cells were performed in Accell siRNA delivery medium once on day 0 and then on day 3 with medium change on day 3.

Transfections of LNCaP cells with Amaxa electroporator (Amaxa Inc., Gaithersburg, Md.) were performed using solution R and program T-009 according to the manufacturer's protocol. Twenty-four hours after transfection, medium was changed and the cells were treated as indicated in the figures.

Stable LNCaP and NIH3T3 cell lines were generated by lentiviral infection of AKR1C3 cloned into pLenti U6 Pgk-puro vector as described earlier. LacZ and AR adenovirus were made at Seven Hills Bioreagents (Cincinnati, Ohio).

Co-Immunoprecipitation and Western Blotting:

Co-immunoprecipitation and western blotting were described earlier. Co-immunoprecipitation was performed with rabbit polyclonal AKR1C3 antibody in cells treated for 6 hrs.

Confocal Microscopy:

Cells were plated at 150,000 cells/well of a 24-well plate on collagen coated coverslips. Medium was replaced to RPMI+1% csFBS without phenol red and maintained in this medium for 3 days. Cells were treated for the indicated periods and were fixed and imaged using confocal microscopy (Carl Zeiss) as described earlier.

Chromatin Immunoprecipitation Assay:

ChIP assays were performed with AR N-20 and AKR1C3 rabbit polyclonal antibodies as described earlier.

Duolink Proximity Ligation Assay:

Duolink kit (O'link, Uppsala, Sweden) was purchased and was used to determine the interaction between AR and AKR1C3. Images were obtained using deconvolution fluorescent microscopy.

RNA Isolation and Gene Expression:

RNA was isolated using cell to ct kit (Applied Biosystems, Carlsbad, Calif.) and realtime PCR was performed using TaqMan primers and probes from Applied Biosystems on ABI 7900 (Applied Biosystems).

Growth Assay.

LNCaP cells were plated at 10,000 cells per well of a 96 well plate in RPMI supplemented with 1% csFBS. The cells were treated as indicated in the figures. Cell viability was measured using sulforhodamine blue (SRB) reagent.

Tumor Xenograft Experiments:

All animal protocols were approved by The University of Tennessee Animal Care and Use Research Committee. Nude mice obtained from Harlan (Indianapolis, Ind.) were housed with five animals per cage and were allowed free access to water and commercial rodent chow (Harlan Teklad 22/5 rodent diet—8640). During the course of the study, the animals were maintained on a 12 hr light:dark cycle. Xenograft experiments were performed as previously published. Briefly, a mixture of 2×106 LNCaP or VCaP cells was suspended in 0.0375 ml RPMI+10% FBS and 0.0625 ml Matrigel/animal and was injected s.c. Once the tumor size reached 100 mm³, the animals were castrated or sham operated, randomized, DHT pellets were implanted subcutaneously or not supplemented and treated as indicated in the figures. Tumor volume and body weight were measured. Tumor volume was calculated using the formula length*width*width*0.5236.

Prostate Cancer Specimen:

Section of a prostate cancer specimen with gleason score 7 (4+3) was obtained under The University of Tennessee, Institutional Review Board (IRB) approval. The specimen was subjected to Duolink's PLA with AR and AKR1C3 antibodies. For negative control, the sample was probed only with AKR1C3 antibody.

### Novel Small Molecules Inhibit AKR1C3 Activity

A novel series of AKR1C3 inhibitors was designed and synthesized.

### Example 2A

### Purified Enzyme Assays

Purified AKR1C Assays:

Purified AKR1C3 enzyme was incubated with ATP and [¹⁴C]-androstenedione (A'dione) (12 μM) in the presence of a titration of putative AKR1C3 inhibitors for 60 min At the end of incubation period, the reaction mixture was evaporated to dryness and the pellet was resuspended in methanol. Radiolabeled A'dione and testosterone were fractionated using TLC and quantified using phosphoimager.

Table 1 reports the percent of AKR1C3 inhibition at 10 μM concentration of AKR1C3 inhibitors in this purified AKR1C3 TLC based enzyme assay. Table 2 reports the same data for other compounds of the invention.

Representative results for IC₅₀ values for compounds of this invention, generated in competition with 100 nM 4′ dione in HEK-293 cells stably transfected with AKR1C3 and using Mass Spec are reported in Table 2. In addition, Table 2 presents % HSD inhibition compared with control. IC₅₀ determination method: HEK-293 cells were stably transfected with AKR1C3 (HEK-293-AKR1C3) using lentivirus and the cells were selected using puromycin. AKR1C3 expression was checked by Western blot to ensure that the cells over-express AKR1C3. HEK-293-AKR1C3 cells were plated in 24 well plates at 125,000 cells/well in DME+5% csFBS without phenol red. Medium was changed to RPMI+1% csFBS without phenol red and maintained in this medium for 3 days to reduce basal hormone levels. Medium was changed again on day 4 and were treated with 100 nM androstenedione alone or in combination with a dose response of individual inhibitor. Twenty four hours after treatment, medium was collected and injected in LC-MS/MS to detect the amount of testosterone formed by AKR1C3. The data was plotted in SigmaPlot (Systat Software Inc.) and the IC₅₀ values for inhibitors were calculated.

% AKR1C3 Inhibition Method:

Reaction mixture containing 50 μl phosphate buffer, 0.2 μl NADPH (100 mM), 0.5 μl purified AKR1C3, 0.25 [¹⁴C] A'dione (48 mCi/mmol; Perkin Elmer) was incubated at 37° C. for 60 min. The reaction was stopped by the addition of 400 μl ethyl acetate, vortexed, and spun for 5 mM at 2000 rpm. The upper layer was transferred to a new tube and air dried. The pellet was resuspended in 25 μl of methanol, spotted on TLC plates and fractionated in TLC chamber with 3:1 chloroform:ethyl acetate. Once the mobile phase reached the top, the plates were dried and exposed to a phosphoimager screen overnight.

% Liver Microsome Inhibition:

Stock solutions of prespective AKR1C3 inhibitors were prepared in DMSO at 5 mM and were incubated with liver microsomes (0.1 mg/mL). Incubations were conducted at 37° C. in a shaking water bath for 10 minutes (microsomes). Incubation durations were selected based on time course study (microsomes) or published literature (recombinant enzymes). Prepared samples were analyzed using LC-MS/MS.

TABLE 1
AKR1C3 inhibition by other compounds of this invention.

| | AKR1C3 |
|---|---|
| Cmpd # | inhibition at 10 μM |
| 6 | 0% |
| 10 | 17% |
| 11 | 55% |
| 13 | 52% |
| 25 | 0% |
| 30 | 30% |
| 34 | 10% |
| 26 | 50% |
| 35 | 0% |
| 36 | 20% |
| 37 | 45% |
| 43 | 42% |
| 79 | 44% |
| 85 | 86% |
| 100 | 20% |
| 100A | 0% |
| 102 | 10% |
| 102A | 0% |
| 102B | 0% |

TABLE 2

| | | | % inhibition | | HLM | | HLM | |
|---|---|---|---|---|---|---|---|---|
| | | | from control | | Phase I | | Phase I + II | |
| | | AKR1C3 | HSD-3 | HSD-5 | T-1/2 | CLint | T-1/2 | CLint |
| NAME | STRUCTURE | IC₅₀ (nM) | (10 uM) | (10 uM) | (min) | (ul/min/mg) | (min) | (ul/min/mg) |
| 15a | 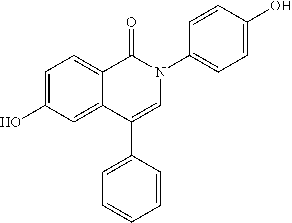 [SMILES: O=c1c2ccc(O)cc2c(-c2ccccc2)cn1-c1ccc(O)cc1] | 16084.0 | | 0.0% | 152.8 | 4.5 | 23.3 | 29.70 |

| | | | | | | |
|---|---|---|---|---|---|---|
| 15b | 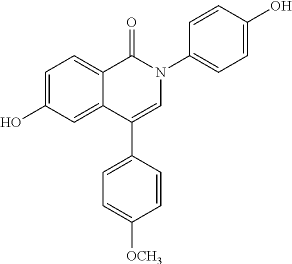 [SMILES: COc1ccc(-c2cn(-c3ccc(O)cc3)c(=O)c3ccc(O)cc23)cc1] | 47.0% | 28.3 | 24.5 | 3.7 | 188.00 |

| | | | | | | | |
|---|---|---|---|---|---|---|---|
| 15g | 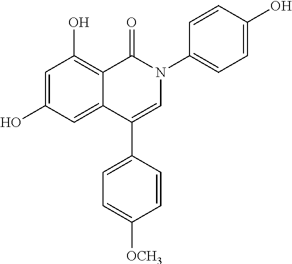 [SMILES: COc1ccc(-c2cn(-c3ccc(O)cc3)c(=O)c3c(O)cc(O)cc23)cc1] | 4523.0 | 40.0% | 16.5 19.32 | 42.1 35.8 | 1.6 | 421.90 |

| | | | | | | | |
|---|---|---|---|---|---|---|---|
| 15h | 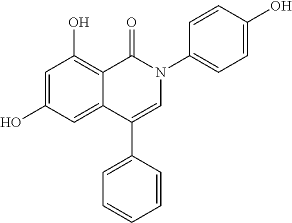 [SMILES: O=c1c2c(O)cc(O)cc2c(-c2ccccc2)cn1-c1ccc(O)cc1] | 6638.0 | 0.0% | 87.1 | 7.9 | 11.4 | 60.70 |

| | | |
|---|---|---|
| 15i | 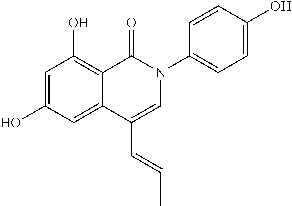 [SMILES: C/C=C/c1cn(-c2ccc(O)cc2)c(=O)c2c(O)cc(O)cc12] | 10.0% |

| | | |
|---|---|---|
| 15j | 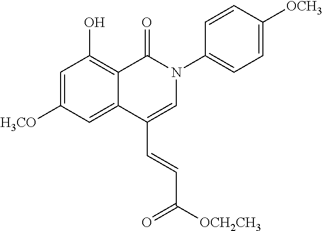 [SMILES: CCOC(=O)/C=C/c1cn(-c2ccc(OC)cc2)c(=O)c2c(O)cc(OC)cc12] | 10.0% |

| | | |
|---|---|---|
| 15k | 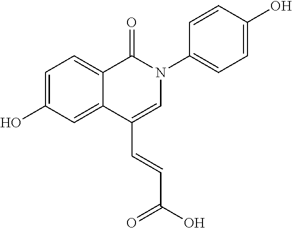 [SMILES: O=C(O)/C=C/c1cn(-c2ccc(O)cc2)c(=O)c2ccc(O)cc12] | 12.0% |

| | | |
|---|---|---|
| 15l | 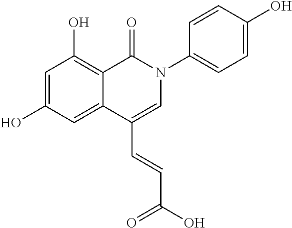 [SMILES: O=C(O)/C=C/c1cn(-c2ccc(O)cc2)c(=O)c2c(O)cc(O)cc12] | 1707.0 |

| | | | | | | |
|---|---|---|---|---|---|---|
| 6 | 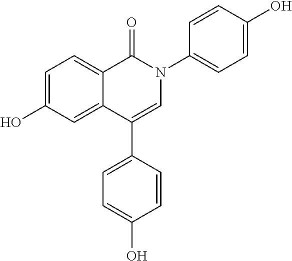 [SMILES: O=c1c2ccc(O)cc2c(-c2ccc(O)cc2)cn1-c1ccc(O)cc1] | 0.0% | | | | |
| ZW-315-047 | 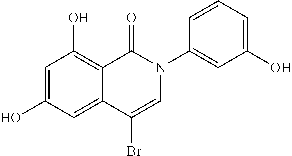 [SMILES: O=c1c2c(O)cc(O)cc2c(Br)cn1-c1cccc(O)c1] | 30.0% | 41.58, 33.55 35.5 29.3 | 16, 20.16 19.5 23.7 | 1.56, 1.5 | 442, 460 |
| 10 | 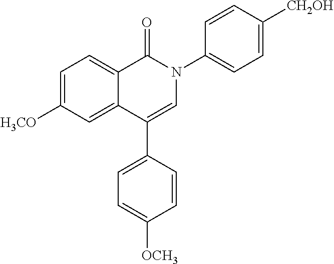 [SMILES: COc1ccc(-c2cn(-c3ccc(CO)cc3)c(=O)c3ccc(OC)cc23)cc1] | 17.0% | | | | |

| | | | | | | | |
|---|---|---|---|---|---|---|---|
| 11 | 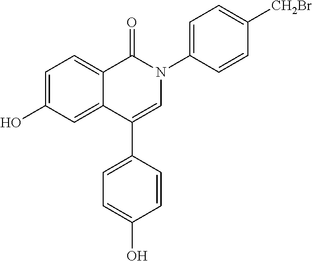 | 5180.0 | 55.0% | 8.23 5.90 | 84.1 119 | 5.0 | 137.50 |

| | | | | | | | |
|---|---|---|---|---|---|---|---|
| 13 | 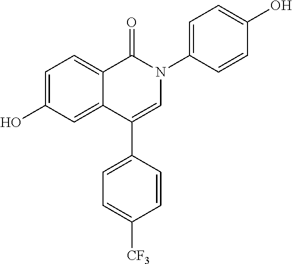 [SMILES: O=c1c2ccc(O)cc2c(-c2ccc(C(F)(F)F)cc2)cn1-c1ccc(O)cc1] | 450.8 | 52.0% | stable, 843 | <1, 0.8 | 40, 36.12 | 17, 19.1 |

| | | |
|---|---|---|
| 14 | 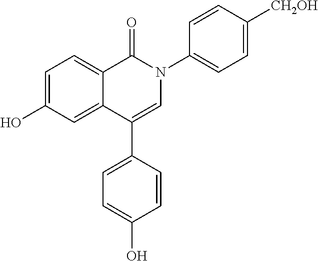 [SMILES: O=c1c2ccc(O)cc2c(-c2ccc(O)cc2)cn1-c1ccc(CO)cc1] | 1319.0 |

| | | | | | | |
|---|---|---|---|---|---|---|
| 30 | 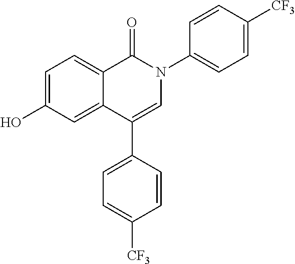 [SMILES: O=c1c2ccc(O)cc2c(-c2ccc(C(F)(F)F)cc2)cn1-c1ccc(C(F)(F)F)cc1] | 30.0% | 102.3 | 6.8 | 7.9 | 88.13 |

| | | |
|---|---|---|
| 34 | 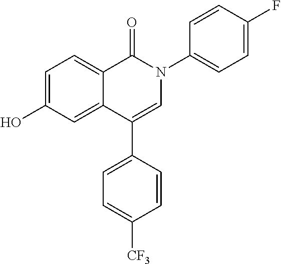 [SMILES: O=c1c2ccc(O)cc2c(-c2ccc(C(F)(F)F)cc2)cn1-c1ccc(F)cc1] | 10.0% |

| | | | |
|---|---|---|---|
| 26 | 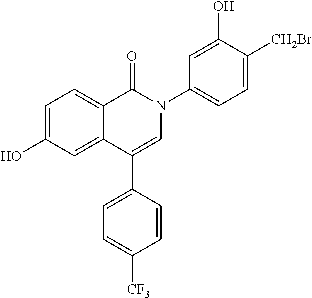 | 288.7 | 50.0% |

| | | |
|---|---|---|
| 35 | 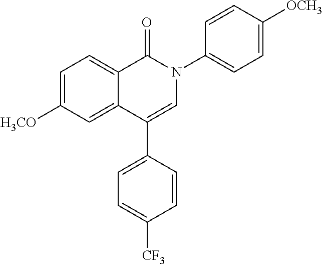 [SMILES: COc1ccc(-n2cc(-c3ccc(C(F)(F)F)cc3)c3cc(OC)ccc3c2=O)cc1] | 0.0% |

| | | |
|---|---|---|
| 36 | 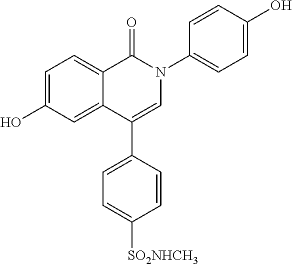 | 20.0% |

| | | | | | |
|---|---|---|---|---|---|
| 37 | 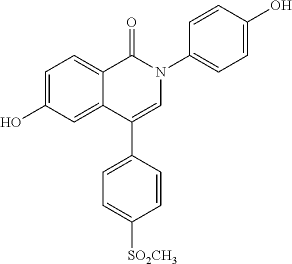 [SMILES: CS(=O)(=O)c1ccc(-c2cn(-c3ccc(O)cc3)c(=O)c3ccc(O)cc23)cc1] | 45.0% | stable | 3.8 | 183 |

| | | |
|---|---|---|
| 43 | 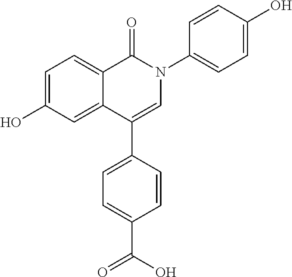 [SMILES: O=C(O)c1ccc(-c2cn(-c3ccc(O)cc3)c(=O)c3ccc(O)cc23)cc1] | 42.0% |

| | | |
|---|---|---|
| 79 | 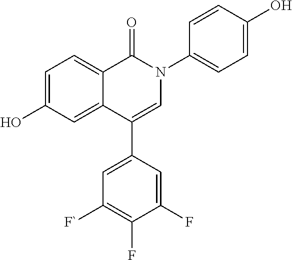 | 44.0% |

| | | | |
|---|---|---|---|
| 85 | 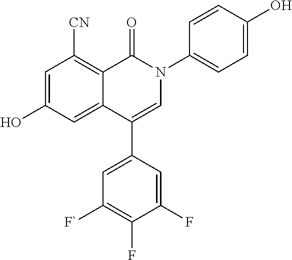 | 140.9 | 86.0% |
| 100 | | | 20.0% |
| 100A | 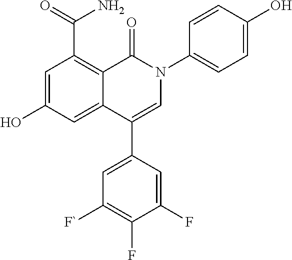 | | 0.0% |
| 102 | 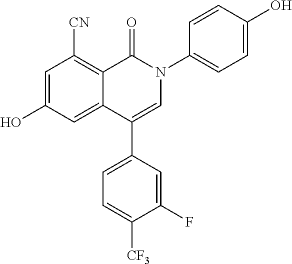 [SMILES: N#Cc1cc(O)cc2c(-c3ccc(C(F)(F)F)c(F)c3)cn(-c3ccc(O)cc3)c(=O)c12] | | 10.0% |
| 102A | 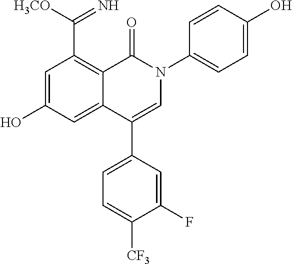 [SMILES: COC(=N)c1cc(O)cc2c(-c3ccc(C(F)(F)F)c(F)c3)cn(-c3ccc(O)cc3)c(=O)c12] | | 0.0% |
| 102B | 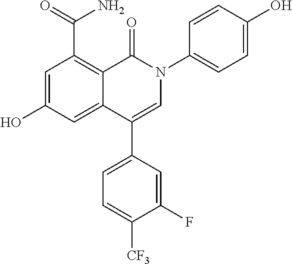 [SMILES: NC(=O)c1cc(O)cc2c(-c3ccc(C(F)(F)F)c(F)c3)cn(-c3ccc(O)cc3)c(=O)c12] | | 0.0% |
| 104A | 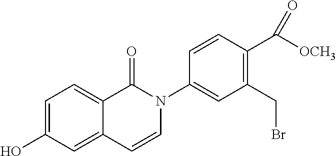 [SMILES: COC(=O)c1ccc(-n2ccc3cc(O)ccc3c2=O)cc1CBr] | | 5.0% |
| DKB-476-065 | 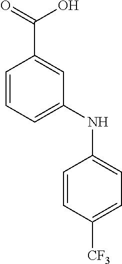 [SMILES: O=C(O)c1cccc(Nc2ccc(C(F)(F)F)cc2)c1] | 154.5 | |
| Rutin | | >10000 | |
| 3,4,5 | | >10000 | |
| trimethoxy | | | |
| cinnamic acid | | | |
| 3,4,dimethoxy | | >10000 | |
| cinnamic acid | | | |
| 214 | 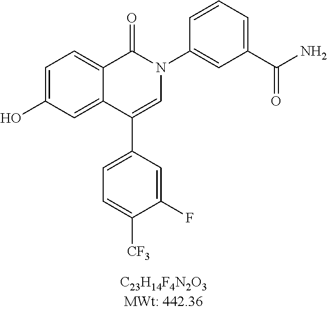 [SMILES: NC(=O)c1cccc(-n2cc(-c3ccc(C(F)(F)F)c(F)c3)c3cc(O)ccc3c2=O)c1] | 240.5 | |

### Example 3

HEK-293 cells were transfected with 0.25 ug GRE-LUC, 100 ng AR, 10 ng CMV-renilla LUC, and 50 ug pCR3.1 vector backbone or AKR1C3. Cells were treated 24 hrs after transfection and luciferase assay performed 48 hrs after transfection. Firefly luciferase values were normalized to renilla luciferase and expressed as ratio.

HEK-293 cells stably transfected with AKR1C3 were plated in 24 well plates and were maintained in RPMI+1% csFBS without phenol red for 2 days to reduce the basal steroid levels. Medium was again changed and treated with 100 nM androstenedione in the presence or absence of the indicated molecules for 24 hrs. Medium was collected at the end of incubation and the amount of steroid measured by LC-MS/MS. While, compound 215 potently inhibited AKR1C3 dependent conversion of androstenedione to testosterone, reference 1 (REF1) and reference 2 (REF2) failed to inhibit the AKR1C3 enzyme activity.

### Example 4

### AKR1C3 Androgen-Dependent AR Transactivation

### AR Transactivation as AKR1C3 Functional Assay

HEK-293 cells were tranfected with 0.25 μg GRE-LUC, 50 ng CMV hAR, 10 ng CMV-renilla luciferase and 1 μg vector or AKR1C3 using lipofectamine tranfectons reagent. Twenty-four hours after transfection the cells were treated with a titration of A'dione. Cells were harvested forty-eight hours after transfection and firefly luciferase levels were measure and normalize to renilla luciferase.

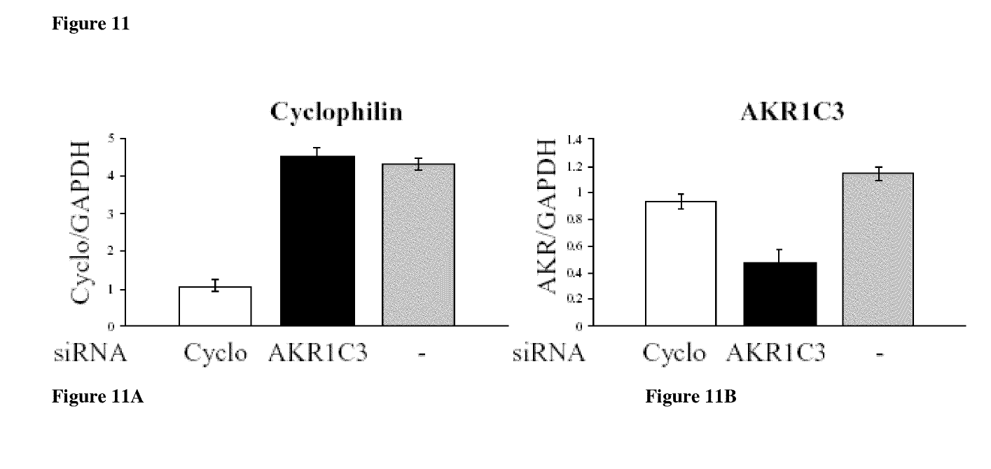

AKR1C3 converts A'dione to testosterone resulting in a ligand with stronger AR activity. FIG. 11 shows that AR activity in response to A'dione in HEK-293 cells transfected with AKR1C3 was higher than the AR activity in vector transfected cells. Over-expression of AKR1C3 significantly reduced the EC₅₀ of A'dione to transactivate AR from 415 nM to 175 nM, as shown in Table 4 below

TABLE 4

| | pCR3.1 (EC₅₀) | AKR1C3 (EC₅₀) |
|---|---|---|
| Ligand | nM | nM |
| Androstenedione | 415 | 175 |
| Testosterone | 2.3 | 0.49 |
| DHT | 0.229 | 0.115 |
| R1881 | 0.21 | 0.069 |
| SARM | 28.8 | 5.97 |

These results suggest AKR1C3 overexpression in prostate cancer may amplify or hypersensitize the AR signaling pathway.

### AKR1C3 Augments Androgen-Dependent AR Transactivation.

AR transactivation studies were carried out in AKR1C3 transfected HEK-293 cells using A'dione, three 17-hydroxy AR agonists (testosterone, DHT and R1881) and a non steroidal selective androgen receptor modulator (SARM) to confirm that the ability of AKR1C3 to amplify AR tranasctivation was selective for 17-keto steroids.

HEK-293 cells were transfected with 0.25 μg GRE-LUC, 50 ng pCR3.1 hAR, 10 ng CMV-renilla luciferase and 1 μg pCR3.1 vector backbone or pCR3.1 AKR1C3. Twenty-four hours after transfection, the cells were treated with a titration of androstenedione (A'dione), testosterone, DHT, R1881, selective androgen receptor modulator (SARM) or a SARM that is devoid of AR binding activity. Cells were harvested forty-eight hours after transfection and firefly luciferase assay was performed and normalized to renilla luciferase.

The results show that cells transfected with AKR1C3 have augmented androgen-dependent AR transactivation represent cells transfected with AKR1C3; closed circles represent cells transfected with backbone vector (pCR3.1))

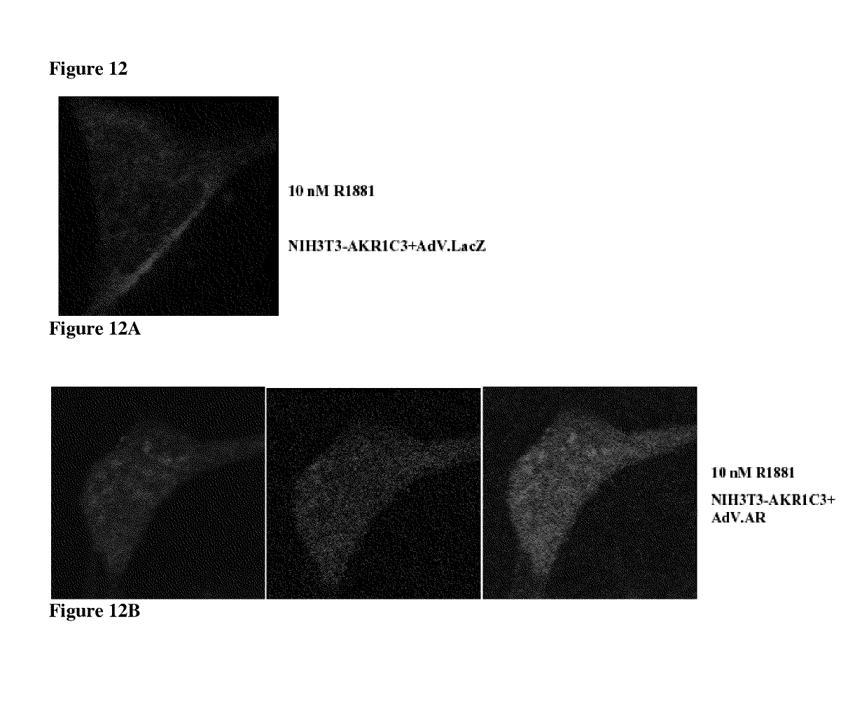

Interestingly, AKR1C3 increased AR transactivation in respone to all ligands that bind to the AR, but not the R-isomer of the SARM that is devoid of AR binding, which is not an AR ligand (FIG. 12F closed triangles). AKR1C3 not only deduced the EC₅₀ of these ligands, but also increased the maximum level of AR transactivation (Eₘₐₓ). (Table 4)

As only A'dione is a substrate for AKR1C3, the increased AR transactivation is AR ligand, i.e. androgen-dependent, not AKR1C3 ligand-dependent. The absence of the increased AR transactivation with the non-androgen (“inactive SARM”) supports this explanation. This suggests that AKR1C3 has a stimulatory effect on androgen-dependent AR transactivation which is not related to the enzyme activity of AKR1C3, which is consistent with a previously unreported co-activator to activity of AKR1C3 with respect to AR transactivation.

Increase in AR transactivation by AKR1C3 is Not Due of Increased AR Expression

HEK-293 cells were transfected with indicated concentration of AKR1C3. Total amount of transfected plasmids were normalized to 1 μg with vector pCR3.1. Twenty-four hours after transfection, the cells were treated with a titration of R1881. Cells were harvested forty-eight hours after transfection and firefly luciferase assay was performed and normalized to renilla luciferase.

Cells transfected with vector or AKR1C3 and treated with 10 nM R1881 were fractionated by SDS-PAGE and Western blots were probed for AR.

Varying the level of AKR1C3 expression showed that 0.5 μg of AKR1C3 plasmid DNA increased AR transactivation in a concentration-dependent manner without altering the expression of AR.

### Enhanced AR Transactivation of Diverse AR Ligands is not an Artifact

In order to ensure that the ability of AKR1C3 to enhance AR transactivation of diverse AR ligands was not an artifact of cell type or transfection system, control experiments using different cell types (COS-1 and NIH3T3), various transfection conditions (lipofectamine, fugene, and Amaxa electroporator), and cells stably expressing AKR1C3 using a lentivirus system were performed.

COS-1 cells were transfected with 0.25 μg GRE-LUC, 50 ng CMV hAR, 10 ng CMV-renilla luciferase and 1 μg pCR3.1, or pCR3.1-AKR1C3. Twenty-four hours after transfection the cells were treated with a titration of the indicated androgens. Cells were harvested forty-eight hours after transfection and firefly luciferase assay was performed and normalized to renilla luciferase. Results shown in the figures are representative of three experiments.

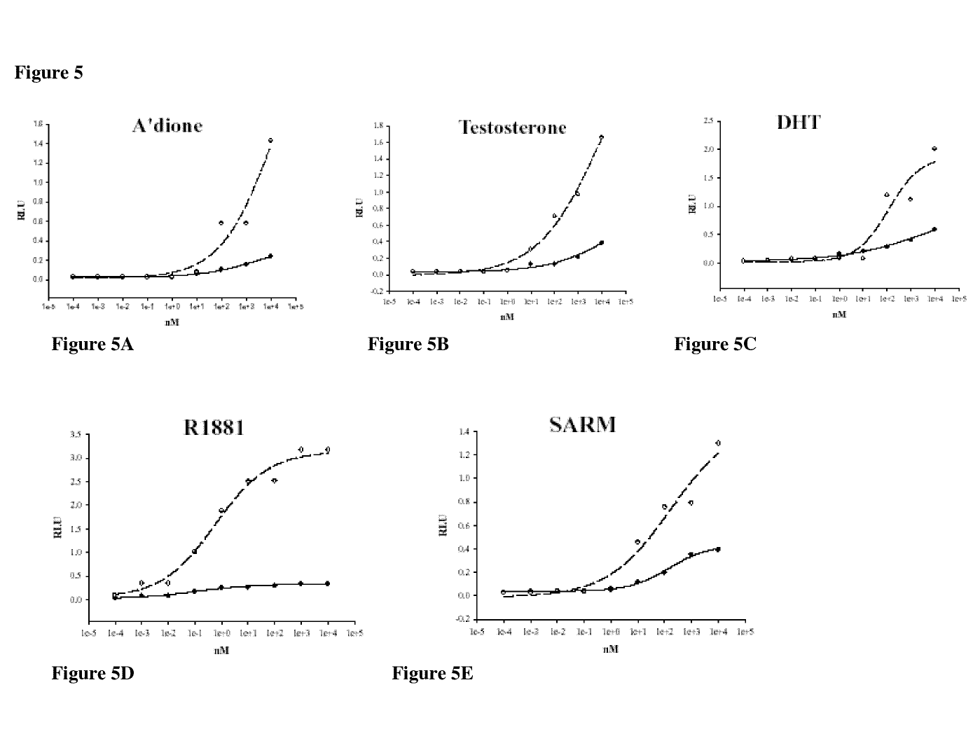

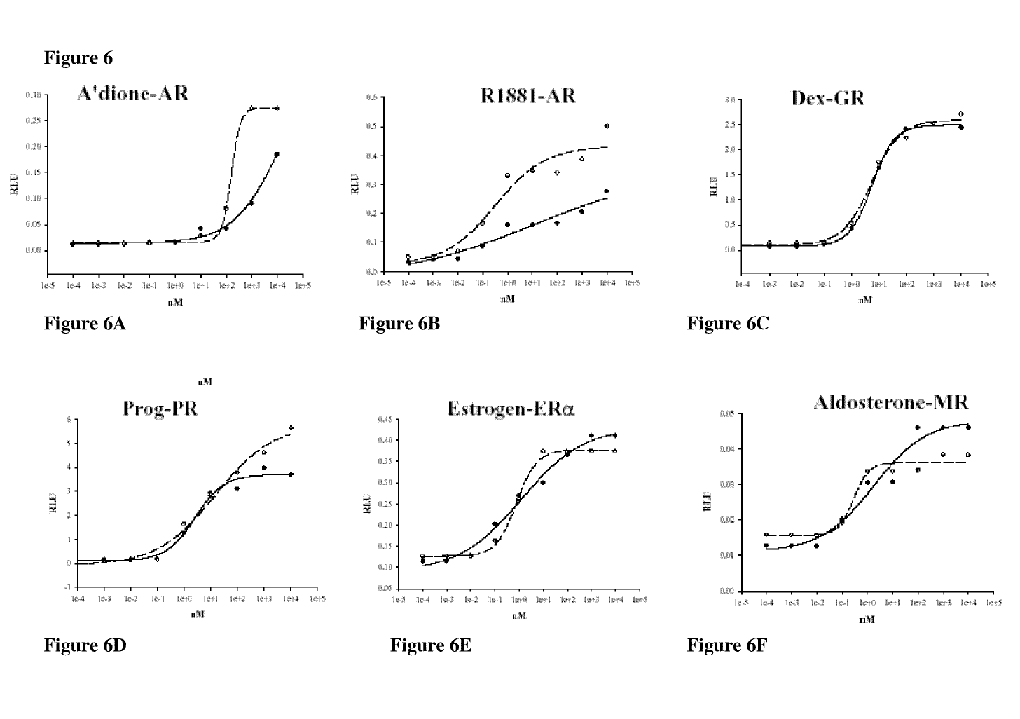

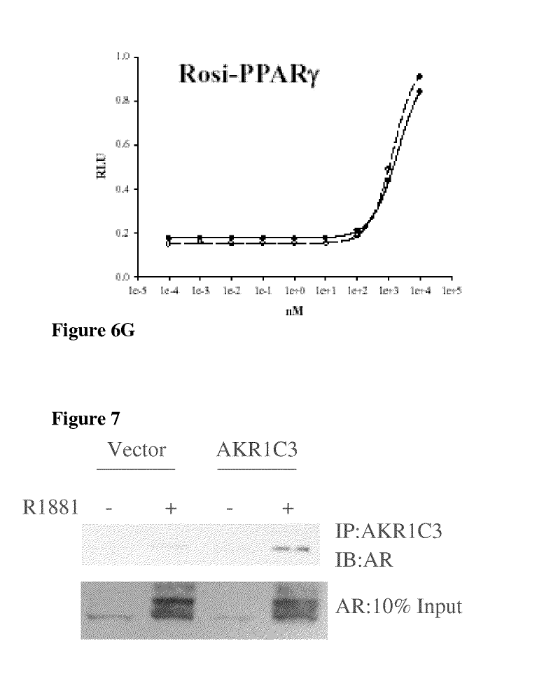

The results (FIG. 5A-E; and data not shown) demonstrated that the AR activation effect of AKR1C3 was not unique to a cell type or transfection condition.

### Specificty of Transactivation for the AR

Steroid receptors share sequence homology in many of their functional domains, facilitating their interaction with the same coactivator. Transactivation experiments were performed with glucocorticoid receptor (GR), mineralocorticoid receptor (MR), progesterone receptor (PR), estrogen receptor (ERα), and peroxisome proliferator and receptor γ (PPARγ) to determine AKR1C3's selectivity for AR.

HEK-293 cells were transfected with 0.25 μg GRE-LUC (AR, GR, PR, and MR), ERE-LUC (ERα) or PPRE-LUC (PPARγ), 50 ng respective receptors, 10 ng CMV-renilla luciferase and 1 μg pCR3.1, or pCR3.1-AKR1C3. Twenty-four hours after transfection the cells were treated with a titration of the indicated ligands. Cells were harvested forty-eight hours after transfection and firefly luciferase assay was performed and normalized to renilla luciferase.

### Example 5

### AKR1C3 Interacts with the Androgen Receptor (AR)

The ability of AKR1C3 to selectively potentiate AR function suggested that it might possibly function as an AR selective coactivator. In order to classify a protein as a coactivator, it must 1) interact with the receptor, 2) increase the activity of the receptor and 3) get recruited to the responsive element of a target gene.

To determine if AR and AKR1C3 interact, co-immunoprecipitation studies were conducted in LNCaP-AKR1C3 or LNCaP-vector cells. Cells were treated with R1881 and the protein extracts were immunoprecipitated with AKR1C3 antibody and immunoblotted for AR.

LNCaP cells stably transfected with vector or AKR1C3 were serum starved for 2 days and treated with vehicle or 10 nM R1881 for 6 hours. Cells were harvested, protein extracted, immunoprecipitated (IP) with AKR1C3 antibody and Western-blotted for AR. Western blot for AR with 10% of the total protein extract was simultaneously performed as input control.

The results shown in FIG. 7 demonstrated that AR and AKR1C3 interacted in a ligand-dependent manner in LNCaP-AKR1C3 cells, but not in LNCaP-vector cells.

### Example 6

### AKR1C3 is Recruited to PSA Enhancer

If AKR1C3 interacts with AR, it could also be recruited to the response element (ARE) of an AR target gene. LNCaP-AKR1C3 or LNCaP-vector cells were treated with R1881 or A'dione and the recruitment of AKR1C3 to the ARE on the PSA enhancer was examined using chromatin immunoprecipitation (ChIP) assay.

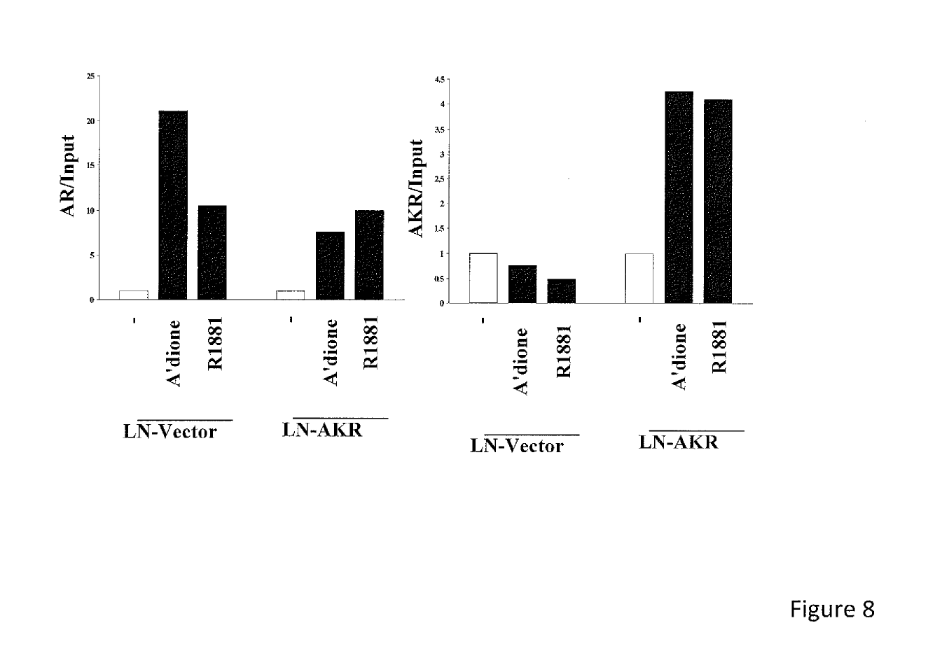

LNCaP-AKR1C3 or LNCaP-Vector cells were maintained in serum free conditions for three days. Cells were then treated with 100 nM A'dione or 10 nM R1881 for 2 hours and a chromatin immunoprecipitation (ChIP) assay was performed with AR (FIG. 8, left panel) or AKR1C3 (FIG. 8, right panel) antibodies. DNA was purified and realtime PCR was performed for PSA enhancer and normalized to input.

Results showed that AKR1C3 was recruited to the PSA enhancer both in response to its substrate, A'dione, as well as in response to R1881 (FIG. 8, right panel). Although AR was recruited to the PSA enhancer in LNCaP-vector cells (FIG. 8, left panel), the recruitment of AKR1C3 to the PSA enhancer could not be detected in LNCaP-vector cells due to its limited expression.

### Example 7

### AR and AKR1C3 Co-Localize in LNCaP Cells and in Advanced Prostate Cancer

The results presented in Examples 4 and 5 demonstrated that a steroidogenic enzyme is capable of acting as an interacting partner of a steroid receptor. Several confirmatory studies were undertaken to prove this interaction.

LNCaP cells stably transfected with lentivirus AKR1C3 were plated on coverslips and serum starved for 2 days. Cells were then treated with vehicle, 100 nM A'dione, or 10 nM R1881 for 6 hrs. Cells were fixed and immunostained with primary antibodies specific to AR and AKR1C3 and fluorescent tagged secondary antibodies. The immunofluorescent signals were captured by laser confocal microscopy.

Immunofluorescence studies in LNCaP-AKR1C3 cells performed using laser confocal microscopy (FIG. 18A) showed that both AR and AKR1C3 were cytoplasmic in the absence of AR ligands, but translocated into the nucleus upon binding of an AR ligand (R1881) or A'dione. The migratory patterns for AR and AKR1C3 overlapped substantially, supporting the idea that the two proteins interact with each other.

In order to ensure that AKR1C3's translocation is dependent on AR, and is not cell type dependent, NIH3T3 cells stably transfected with AKR1C3 were infected with adenovirus expressing Lac Z (FIG. 12A) or AR (FIG. 12B) and were treated with 10 nM R1881. Although cells were treated with R1881, AKR1C3 was cytoplasmic in the absence of AR and translocated into the nucleus only in the presence of AR, indicating the requirement for the AR presence for AKR1C3 to translocate into nucleus.

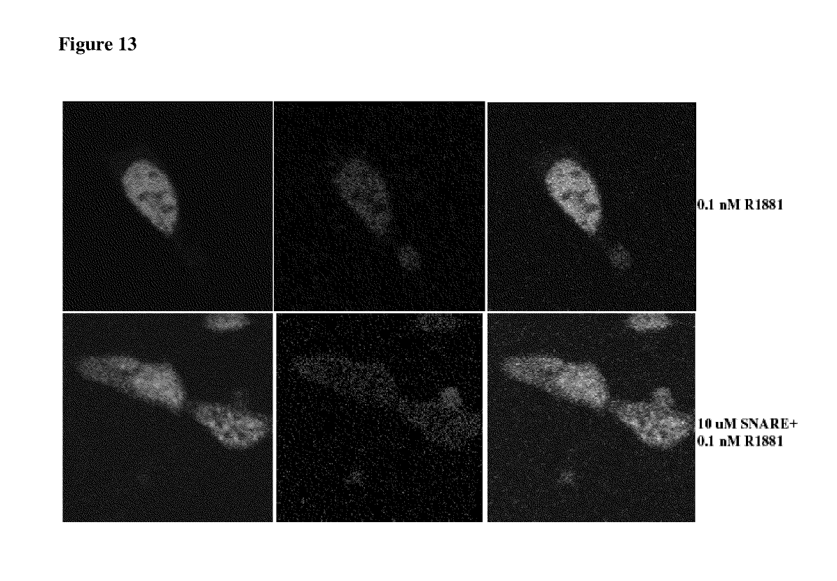

AR antagonist, SNARE-1, was known to inhibit ligand-dependent AR nuclear translocation. Therefore, the translocation of AR and AKR1C3 in response to R1881 in the presence or absence of SNARE-1 was also tested. R1881 efficiently translocated AR into the nucleus and AKR1C3 co-translocated with AR. However, when cells were treated with 10 μM SNARE-1, AR only partially translocated into the nucleus and predominantly remained in the cytoplasm. AKR1C3 followed the same pattern (FIG. 13).

The Duolink proximity ligation assay (PLA) detects protein-protein interaction by fluorescent visualization (Soderberg et al., 2006). DNA attached to the secondary antibodies is ligated and amplified and the amplified DNA fluoresces red only if the two proteins are in proximity

LNCaP-AKR1C3 or LNCaP-Vector cells were plated on coverslips, serum starved for 2 days and were treated overnight with 100 nM A'dione. Cells were fixed and incubated with AR and/or AKR1C3 antibodies. The fluorescent DNA attached to secondary antibodies was amplified and the efluorescnet DNA signal detected by deconvolution fluorescent microscopy. Nucleus was stained with DAPI.

The results of treating LNCaP-AKR1C3 cells with R1881 and subjecting them to PLA showed that while AR and AKR1C3 interaction (represented by red fluorescence) was detected in LNCaP-AKR1C3 cells (FIG. 18B), interaction was undetected in LNCaP-mock cells or in the absence of one of the antibodies (FIG. 18B lower panel).

To determine if the interaction between AR and AKR1C3 observed in prostate cancer cells is also observed in human prostate cancer, a prostate cancer specimen (Gleason sum 4+3=7) was subjected to PLA with AR and/or AKR1C3 antibody.

A tissue section from Gleason score 7 prostate cancer was subjected to Duolink proximity ligation assay with AR and AKR1C3 antibodies (FIG. 18C, positive top panels) or in the absence of AR antibody (FIG. 18C, negative control bottom panels). Nucleus was counterstained with DAPI and images captured using deconvolution microscopy. Images are representative of n=3.

Results showed that while the interaction between the two proteins (as visualized by red fluorescent spots) was clearly observed in the tissue (FIG. 18C top panel), the interaction was not detected in the absence of one of the antibodies (FIG. 18C bottom panel). These results suggest that the AR and AKR1C3 not only interact in cells, but also in advanced prostate cancer.

### Example 8

### AR Function in LNCaP Cells is Affected by AKR1C3 Expression

### Endogenous AKR1C3 Expression is Important for AR Function in LNCaP Cells.

Since the LNCaP cells express modest amounts of AKR1C3, siRNA was used to understand AKR1C3's role in AR function.

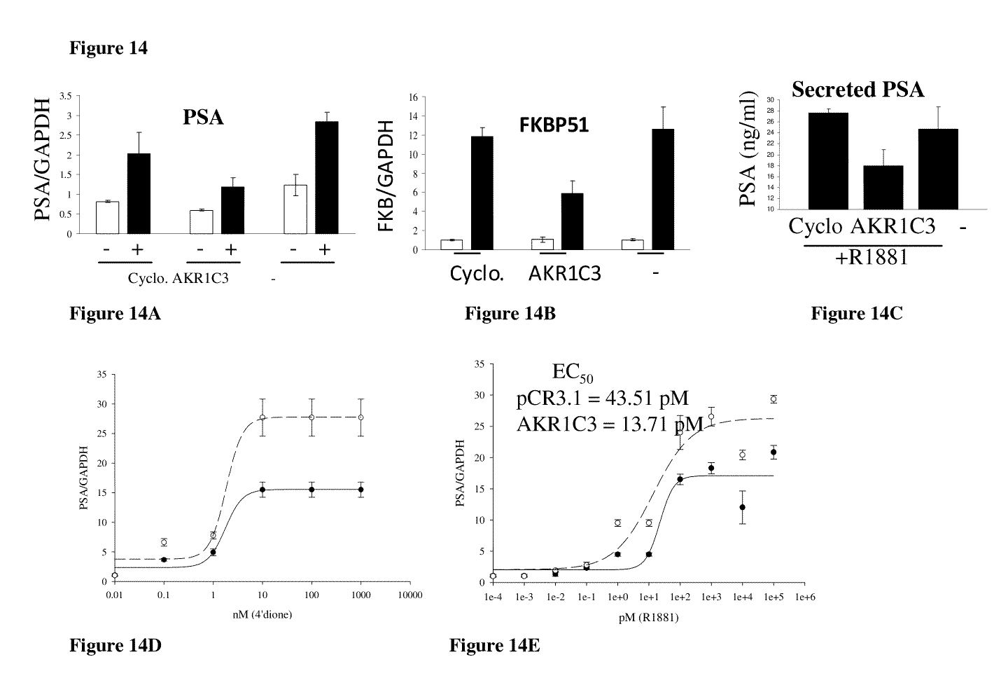

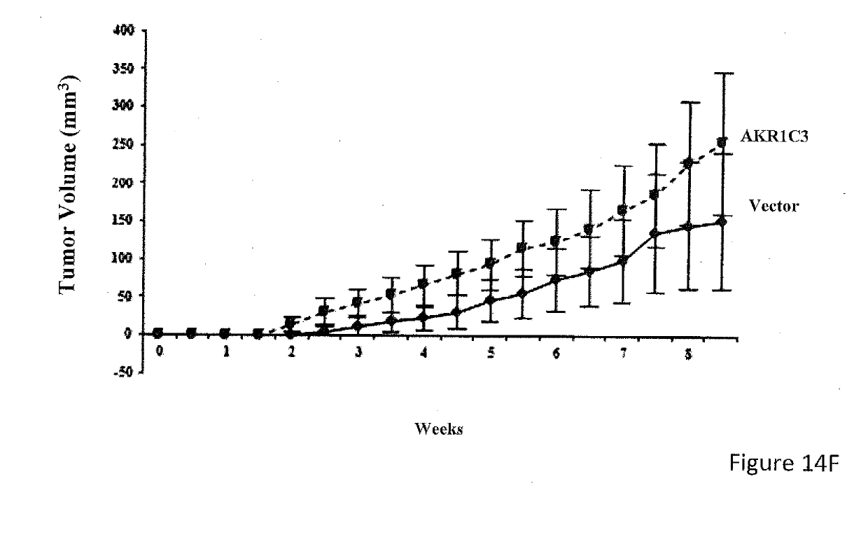

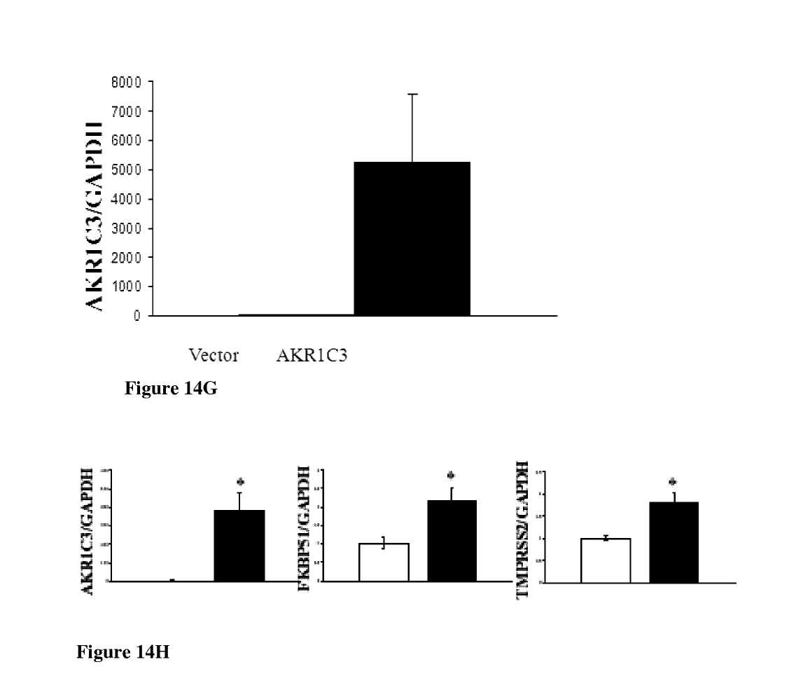

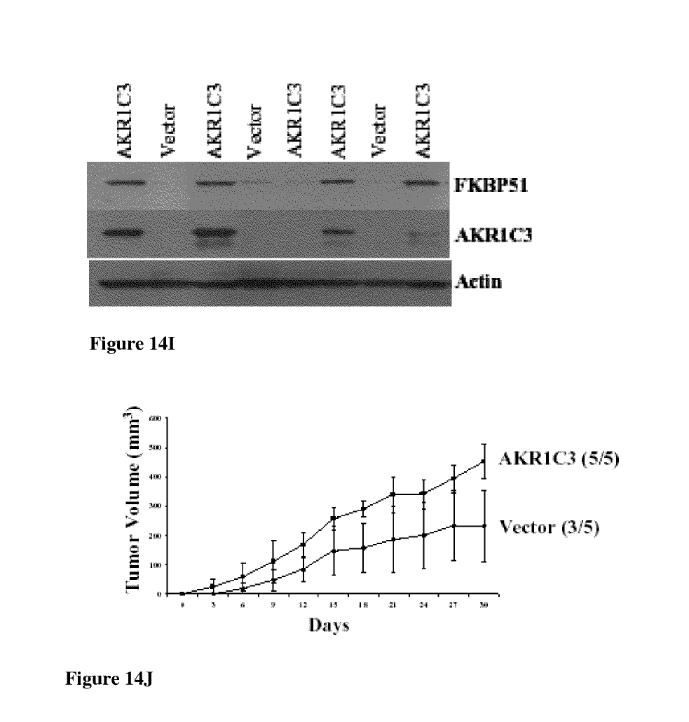

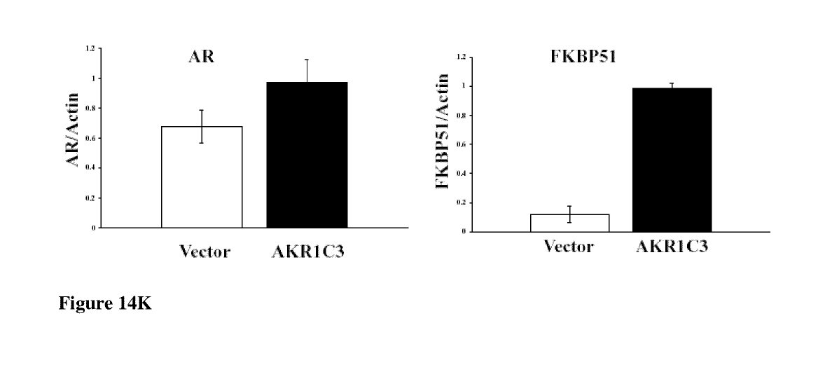

LNCaP cells were transfected with siRNA constructs for cyclophilin (Cyclo.), or AKR1C3 or with no siRNA not transfected with siRNA (−). Six days after transfection (with medium change and a second transfection after 3 days), the cells were treated with vehicle (open bars) or 0.1 nM R1881 (closed bars) for 24 12 hrs., RNA was extracted and the expression of AR targetandrogen responsive genes (PSA and FKBP51) was measured by real time PCR and normalized to GAPDH (FIGS. 14A-14B). Secreted PSA in R1881-treated cell culture medium was also measured in the medium of siRNA transfected cells treated with R1881 measured by ELISA (FIG. 14C). FIGS. 26A and 26B demonstrates the reduction in target gene expression by siRNAs.

Results shows that siRNA to AKR1C3 significantly reduced the ligand-induced expression of AR-dependent genes such as PSA and FKBP51, and PSA protein (FIGS. 14A-14C), compared to cyclophilin or no siRNA conditions.

### AKR1C3 Over-Expression Increases the Androgen-Dependent PSA Gene Expression in LNCaP Cells.

Although AKR1C3 amplified the function of AR in a transactivation system, its effects on endogenous gene expression in prostate cancer cells were not known. PSA gene expression was measured in LNCaP cells transfected with AKR1C3 using Amaxa electroporator and treated with increasing concentrations of A'dione (FIG. 14D) or R1881 (FIG. 14E).

With reference to FIGS. 14D, 14E and 14H, LNCaP cells were transfected with 10 μg vector (solid line) or AKR1C3 (broken line) using Amaxa electroporator. Cells were maintained in serum free condition for 3 days and treated for 12 hrs with the indicated concentrations of A'dione (FIG. 14D) or R1881 (FIG. 14E). EC₅₀ of R1881 induced PSA gene expression is given at the top of FIG. 14E. RNA was extracted and realtime PCR for PSA gene expression was performed and normalized to GAPDH. FIG. 14H shows AKR1C3 expression in cells transfected with AKR1C3 (closed bar) or vector (open bar; not visible in figure) transfected cells.

Results showed that AKR1C3 over-expression increased PSA gene expression in response to both A'dione and R1881 (FIGS. 14D and 14E).

### Example 9

### AKR1C3 Crystallography

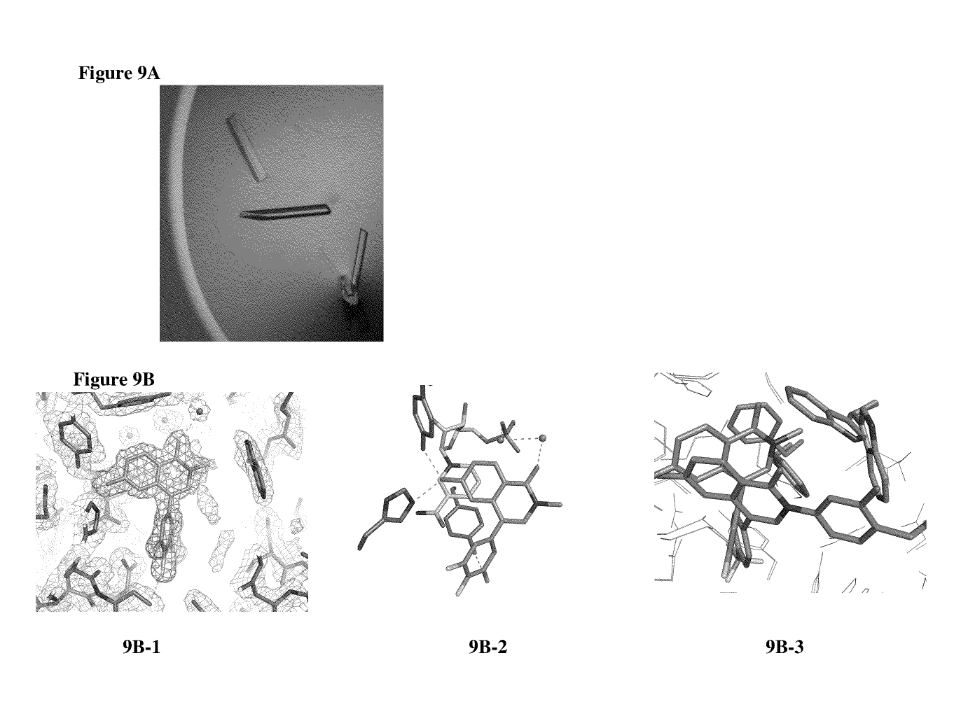

Methods. AKR1C3 was cloned into pGEX4T-1 and transformed into E. coli BL21-DE3. GST-AKR1C3 was expressed in one liter of LB media by induction with 0.1 mM IPTG at 37° C. for 4-6 hours. The cell pellet was resuspended in 10 mL lysis buffer (10% glycerol, 50 mM Tris pH 7.5, 0.5 mM EDTA, 0.8% n-octyl-β-glucoside, 0.1 mM PMSF, 1 mM NADP, 10 mM DTT, 1 mg/mL lysozyme, 10 U/μL DNase, 10 mM MgCl₂), subjected to three freeze/thaw cycles and centrifuged at 40,000×g for 20 min. The clarified lysate was incubated with 3 mL glutathione sepharose resin for 2 hrs at 4° C. and the resin was washed with PBS containing 1 mM DTT and 0.2% n-octyl-β-glucoside. The fusion protein was cleaved overnight with 500 units thrombin at 4° C. and supernatant was placed on a fast blue column in Buffer A (20 mM Tris pH 7.5, 10% glycerol, 1 mM DTT) and eluted with Buffer B (Buffer A+1M NaCl). Buffer exchange and protein concentration were performed with a Millipore 10 kD cut-off concentrator to 500 pt in a buffer containing 10 mM KH₂PO₄ pH 7.0, 1 mM EDTA, 0.5% decyl-maltoside, and 1.2 mM NADP. Drug of interest was added before crystallization trials to a final concentration of 100 μM. Best crystals were grown with reservoir solutions containing 25-33% PEG3350 and 0.1M Hepes pH 7.5 using hanging drops (FIG. 9). Crystals were cryoprotected in 35% PEG3350 and 0.1M Hepes pH 7.5. Diffraction data were collected using a Rigaku RU300 rotating anode generator and an R-axis IV++ image plate (Rigaku), and processed with Crystal Clear software (Molecular Structure Corporation). Structures were solved using molecular to replacement with the androstenedione-AKR1C3 complex (PDB code 1FX0).

Results.

Electron density maps of the AKR1C3-(cmpd 45) complex at 1.9 Å resolution (FIG. 21A, 21B) demonstrated a bifurcated hydrogen bond between the hydroxyl group of the ligand to H117 and Y55. The carbonyl group of 45 interacts with a network of water molecules in a hydrophilic region. One of the meta-fluoro groups is in hydrogen bond range with the side chain of Y216. Analogs containing an N-substituted phenyl ring such as 26 result in displacement of W277 and rearrangement of F316 to be accommodated within the binding pocket (FIG. 21C) as determined by comparing the crystal structure of the AKR1C3-(cmpd 26) complex at 1.9 Å. Such bulky N-substitutions also result in an altered binding conformation as shown.

### Example 10

### AKR1C3 and SRC-2 Synergize to Increase AR Transactivation

To determine if AKR1C3 and a bonafide coactivator, SRC-2, that is over-expressed in prostate cancer, synergistically increase AR transactivation, plasmids encoding both proteins were transfected in HEK-293 cells and AR transactivation studies were performed.

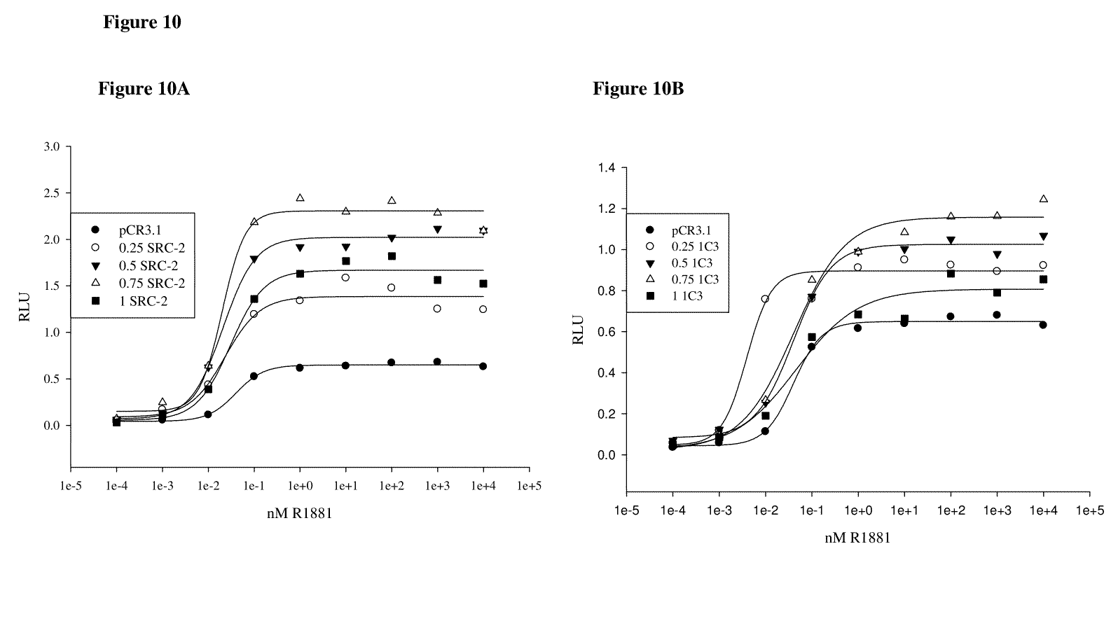

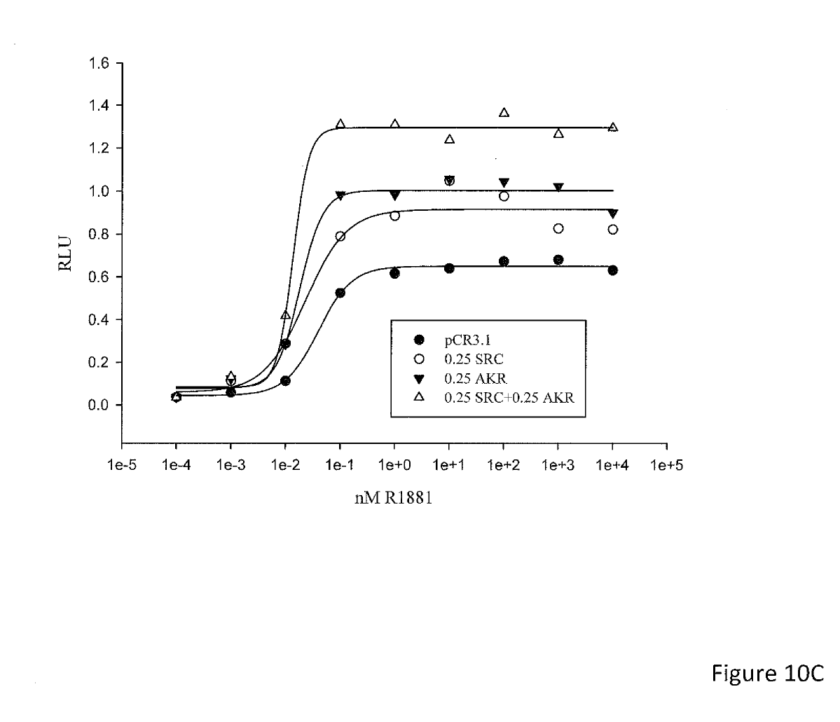

HEK-293 cells were transfected with 0.25 μg GRE-LUC, 50 ng CMV hAR, 10 ng CMV-renilla luciferase and indicated concentrations of SRC-2 (FIG. 10A), AKR1C3 (FIG. 10B) or combination of both (FIG. 25C). Twenty-four hours after transfection the cells were treated with a titration of R1881. Cells were harvested forty-eight hours after transfection and firefly luciferase was measured and normalized to renilla luciferase.

Results showed that while SRC-2 (FIG. 10A) and AKR1C3 (FIG. 25B) concentration-dependently increased AR transactivation, co-transfection of sub-optimal concentrations of the two plasmids synergistically increased AR transactivation (FIG. 25C), suggesting different interaction sites with AR. Results shown in the figures are representative of three experiments. 1C3-AKR1C3; RLU-relative light units.

### Example 11

### Weak ERα and ERβ Binding and Transactivation Activities of Representative Compounds

### Binding Method:

Recombinant ER-α or ER-β ligand binding domain (LBD) was combined with [³H]Estradiol (PerkinElmer, Waltham, Mass.) in buffer A (10 mM Tris, pH 7.4, 1.5 mM disodium to EDTA, 0.25 M sucrose, 10 mM sodium molybdate, 1 mM PMSF) to determine the equilibrium dissociation constant (K_{d}) of [³H]E₂. Protein was incubated with increasing concentrations of [³H]E₂ with and without a high concentration of unlabeled E₂ at 4° C. for 18 h in order to determine total and non-specific binding. Non-specific binding was then subtracted from total binding to determine specific binding. Ligand binding curves were analyzed by nonlinear regression with one site saturation to determine the K_{d} of E₂ (ER-α: 0.65 nM; ER-β: 1.83 nM). In addition, the concentration of [³H]E₂ required to saturate ER-α and ER-β LBD was determined to be 1-3 nM. Increasing concentrations of ER ligands (range: 10⁻¹¹ to 10⁻⁶ M) were incubated with [³H]E₂ (1-3 nM) and ER LBD (α or β) using the conditions described above.

### Transactivation Method:

HEK-293 cells were plated in 24 well plates at 125,000 cells/well in DME+5% csFBS without phenol red and transfected with 25 ng of mammalian expression plasmids for ER-alpha or ER-beta in combination with 0.25 ug ERE-LUC and 5 ng CMV-renilla luc as transfection control. Twenty four hours after transfection, the cells were treated with the indicated drugs alone or in combination with estradiol (antagonist mode) and luciferase assay performed 48 hrs after transfection. Firefly luciferase was normalized to renilla luciferase.

| | | Ki (nM) | | Agonist | | Antagonist | |
|---|---|---|---|---|---|---|---|
| NAME | STRUCTURE | ER-α | ER-β | ER-α | ER-β | ER-α | ER-β |
| 15a | 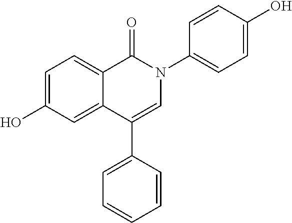 [SMILES: O=c1c2ccc(O)cc2c(-c2ccccc2)cn1-c1ccc(O)cc1] | 1779 | 129.8 | >10000 | >10000 | >10000 | 918.3 |
| 15b | 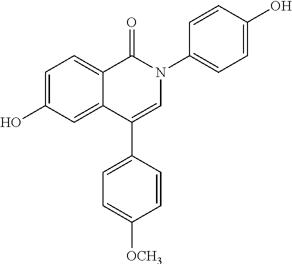 [SMILES: COc1ccc(-c2cn(-c3ccc(O)cc3)c(=O)c3ccc(O)cc23)cc1] | 2097 | 251.7 | >10000 | >10000 | >10000 | 127.6 |
| 15g | 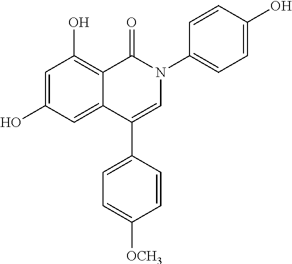 [SMILES: COc1ccc(-c2cn(-c3ccc(O)cc3)c(=O)c3c(O)cc(O)cc23)cc1] | 63.2 | 2.77 | >10000 | >10000 | 961.5 | 22.44 |
| 15h | 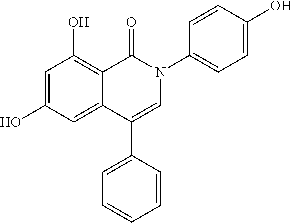 [SMILES: O=c1c2c(O)cc(O)cc2c(-c2ccccc2)cn1-c1ccc(O)cc1] | 124.4 | 3.03 | >10000 | >10000 | 2026 | 53.36 |
| 15i | 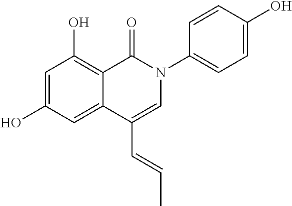 [SMILES: C/C=C/c1cn(-c2ccc(O)cc2)c(=O)c2c(O)cc(O)cc12] | 101.9 | 1.66 | >10000 >10000 | >10000 >10000 | 1968 1791.1 | 32.21 66.27 |
| 15j | 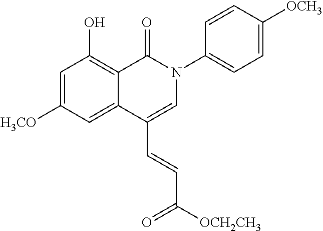 [SMILES: CCOC(=O)/C=C/c1cn(-c2ccc(OC)cc2)c(=O)c2c(O)cc(OC)cc12] | >1505.6 | >2201.4 | >10000 | >10000 | >10000 | >10000 |
| 15k | 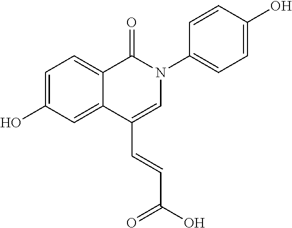 [SMILES: O=C(O)/C=C/c1cn(-c2ccc(O)cc2)c(=O)c2ccc(O)cc12] | >150.56 | >220.14 | ND | ND | ND | ND |
| 15l | 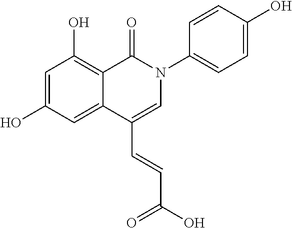 [SMILES: O=C(O)/C=C/c1cn(-c2ccc(O)cc2)c(=O)c2c(O)cc(O)cc12] | >1505.6 | 612.6 | ND | ND | ND | ND |
| 6 | 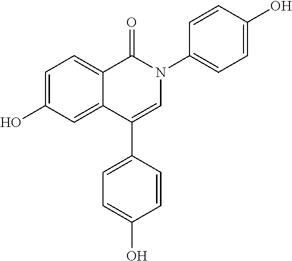 [SMILES: O=c1c2ccc(O)cc2c(-c2ccc(O)cc2)cn1-c1ccc(O)cc1] | 1419 | 56.19 | >10000 | >10000 | >10000 | 1948.4 |
| ZW-315-047 | 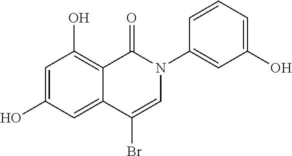 [SMILES: O=c1c2c(O)cc(O)cc2c(Br)cn1-c1cccc(O)c1] | 503.5 | 7.23 | Partial agonist | Partial agonist | 1721.2 | 2207.3 |
| 10 | 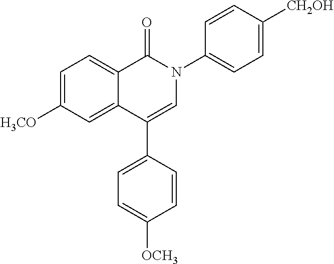 [SMILES: COc1ccc(-c2cn(-c3ccc(CO)cc3)c(=O)c3ccc(OC)cc23)cc1] | >1506 | >2201 | >10000 | >10000 | >10000 | >10000 |
| 11 | 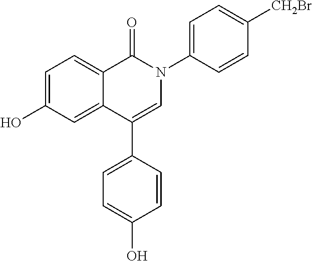 | | >2201 | >10000 | >10000 | >10000 | >10000 |
| 13 | 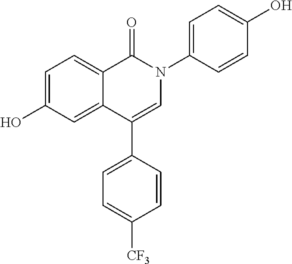 [SMILES: O=c1c2ccc(O)cc2c(-c2ccc(C(F)(F)F)cc2)cn1-c1ccc(O)cc1] | >1506 | 157.2 | >10000 | >10000 | >10000 | 676.7 |
| 14 | 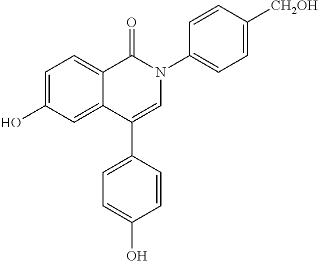 [SMILES: O=c1c2ccc(O)cc2c(-c2ccc(O)cc2)cn1-c1ccc(CO)cc1] | >1506 | >2201 | >10000 | >10000 | >10000 | >10000 |
| ZW-315-109 | | >1506 | >2201 | >10000 | >10000 | >10000 | >10000 |
| 30 | 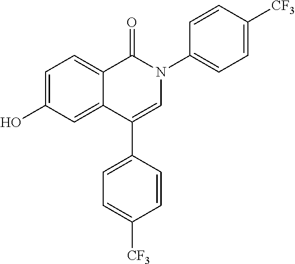 [SMILES: O=c1c2ccc(O)cc2c(-c2ccc(C(F)(F)F)cc2)cn1-c1ccc(C(F)(F)F)cc1] | >1506 | >2201 | >10000 | >10000 | >10000 | >10000 |
| 34 | 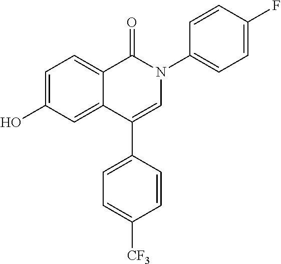 [SMILES: O=c1c2ccc(O)cc2c(-c2ccc(C(F)(F)F)cc2)cn1-c1ccc(F)cc1] | >1506 | >2201 | >10000 | >10000 | >10000 | >10000 |
| 26 | 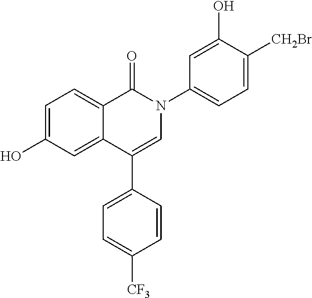 | >1506 | >2201 | >10000 | >10000 | >10000 | >10000 |
| 35 | 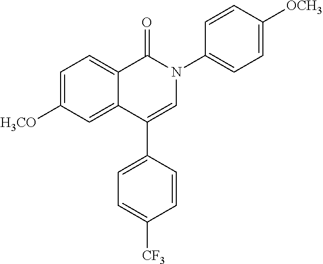 [SMILES: COc1ccc(-n2cc(-c3ccc(C(F)(F)F)cc3)c3cc(OC)ccc3c2=O)cc1] | >1506 | >2201 | >10000 | >10000 | >10000 | >10000 |
| 36 | 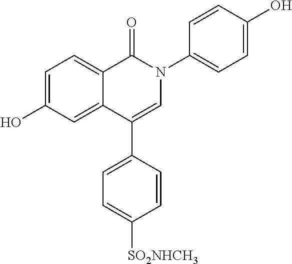 | | | >10000 | >10000 | >10000 | |
| 37 | 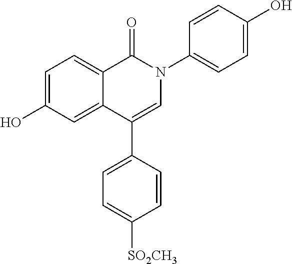 [SMILES: CS(=O)(=O)c1ccc(-c2cn(-c3ccc(O)cc3)c(=O)c3ccc(O)cc23)cc1] | | | >10000 | >10000 | >10000 | |
| 43 | 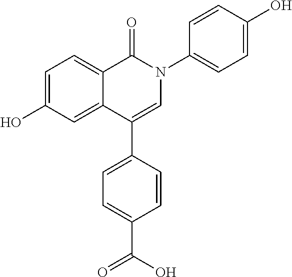 [SMILES: O=C(O)c1ccc(-c2cn(-c3ccc(O)cc3)c(=O)c3ccc(O)cc23)cc1] | | | >10000 | >10000 | >10000 | >10000 |
| 79 | 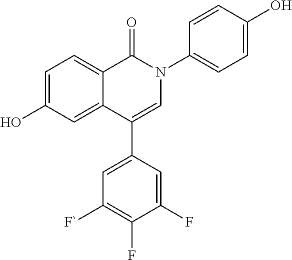 | | | 32.6% of E2 @1000 nM | 45.0% of E2 @1000 nM | 51% of E2 @10k nM | 45.3% of E2 @10k nM |
| 85 | 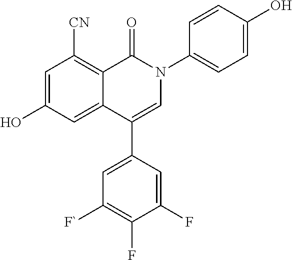 | | | 36.5% of E2 @10k nM | >10000 | >10000 | 59.7% of E2 @1000 nM |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of inhibiting a hydroxysteroid dehydrogenase in a patient in need thereof comprising administering to the patient a compound represented by the structure of formula I, or its isomer, tautomer, pharmaceutically acceptable salt, polymorph, crystal, N-oxide, hydrate or any combination thereof: 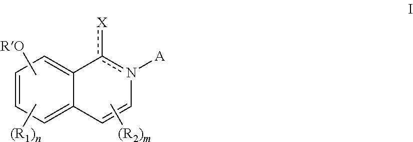
wherein
A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic rings are optionally substituted by 1 to 5 substituents independently selected from R₃ or OR″; and X is O or S; or
A is nothing, N forms a double bond with the cyclic carbon and X is OH or OCH₂CH₂-heterocycle in which the heterocycle is a 3-7 membered saturated or unsaturated substituted or unsubstituted heterocyclic ring;
R₁, R₂ and R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OHSH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring;
R is alkyl, hydrogen, haloalkyl, dihaloalkyl, trihaloalkyl, CH₂F, CHF₂, CF₃, CF₂CF₃, aryl, heteroaryl, phenyl, benzyl, -Ph-CF₃, -Ph-CH₂F, -Ph-CHF₂, -Ph-CF₂CF₃, halogen, alkenyl, CN, NO₂, or OH;
R′ is hydrogen, Alk, or COR;
R″ is hydrogen, Alk, or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 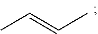
Q is SO₃H, CO₂H, CO₂R, NO₂, tetrazole, SO₂NH₂ or SO₂NHR;
n is an integer of between 1-3;
m is an integer between 1-2; and
Alk is a linear alkyl of 1-7 carbons, branched alkyl of 1-7 carbons, or cyclic alkyl of 3-8 carbons.

2. The method of claim 1, wherein said compound is represented by the structure of formula XI, or its isomer, pharmaceutically acceptable salt, pharmaceutical product, polymorph, crystal, N-oxide, ester, hydrate or any combination thereof: 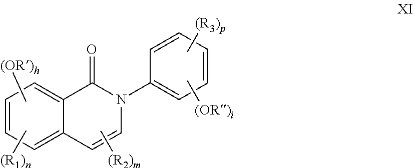
wherein:
R₁, R₂, R₃ are each, independently, hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, cycloalkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;
R is alkyl, cycloalkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
R′ is hydrogen, Alk or COR;
R″ is hydrogen, Alk or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 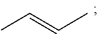
Q is SO₃H, CO₂H, CO₂R, NO₂, tetrazole, SO₂NH₂ or SO₂NHR;
h is 0, 1, 2 or 3;
i is 0, 1, 2, 3 or 4;
n is 1, 2, 3 or 4;
m is 1 or 2;
p is 0, 1, 2, 3, 4 or 5; and
Alk is a linear alkyl of 1-7 carbons, branched alkyl of 1-7 carbons, or cycloalkyl of 3-8 carbons.

3. The method of claim 2, wherein said compound is selected from:
6-hydroxy-2-(4-hydroxyphenyl)-4-phenylisoquinolin-1(2H)-one (15a),
6,8-dihydroxy-2-(4-hydroxyphenyl)-4-(4-methoxyphenyl)isoquinolin-1(2H)-one (15g),
6,8-dihydroxy-2-(4-hydroxyphenyl)-4-phenylisoquinolin-1(2H)-one (15h),
(E)-3-(6,8-dihydroxy-2-(4-hydroxyphenyl)-1-oxo-1,2-dihydroisoquinolin-4-yl)acrylic acid (15l),
2-(4-bromomethyl)phenyl-6-hydroxy-4-(4-hydroxyphenyl)isoquinolin-1(2H)-one (11),
6-hydroxy-2-(4-hydroxyphenyl)-4-(4-(trifluoromethyl)phenylisoquinolin-1(2H)-one (13),
6-hydroxy-2-(4-(hydroxymethyl)phenyl)-4-(4-hydroxyphenyl)isoquinolin-1(2H)-one (14),
2-(4-(bromomethyl)-3-hydroxyphenyl)-6-hydroxy-4-(4-(trifluoromethyl)phenyl)isoquinolin-1(2H)-one (26),
6-hydroxy-2-(4-hydroxyphenyl)-1-oxo-4-(3,4,5-trifluorophenyl)-1,2-dihydroisoquinoline-8-carbonitrile (85),
3-(4-(3-fluoro-4-(trifluoromethyl)phenyl)-6-hydroxy-1-oxoisoquinolin-2(1H)-yl)benzamide (214) and
4-(4-(3-fluoro-4-(trifluoromethyl)phenyl)-6-hydroxy-1-oxoisoquinolin-2(1H)-yl)benzamide (215).

4. The method according to claim 3, wherein said administration selectively inhibits an AKR1C3 enzyme activity.

5. A method of treating a disorder that responds to a hydroxysteroid dehydrogenase inhibitor comprising administering to a patient in need thereof, a therapeutically effective amount of compound represented by the structure of formula I, or its isomer, tautomer, pharmaceutically acceptable salt, polymorph, crystal, N-oxide, hydrate or any combination thereof: 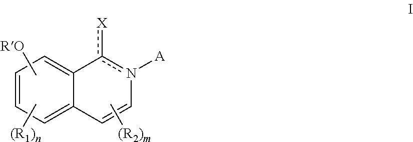
wherein
A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic rings are optionally substituted by 1 to 5 substituents independently selected from R₃ or OR″; and X is O or S; or
A is nothing, N forms a double bond with the cyclic carbon and X is OH or OCH₂CH₂-heterocycle in which the heterocycle is a 3-7 membered saturated or unsaturated substituted or unsubstituted heterocyclic ring;
R₁, R₂ and R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OHSH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring;
R is alkyl, hydrogen, haloalkyl, dihaloalkyl, trihaloalkyl, CH₂F, CHF₂, CF₃, CF₂CF₃, aryl, heteroaryl, phenyl, benzyl, -Ph-CF₃, -Ph-CH₂F, -Ph-CHF₂, -Ph-CF₂CF₃, halogen, alkenyl, CN, NO₂, or OH;
R′ is hydrogen, Alk, or COR;
R″ is hydrogen, Alk, or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 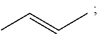
Q is SO₃H, CO₂H, CO₂R, NO₂, tetrazole, SO₂NH₂ or SO₂NHR;
n is an integer of between 1-3;
m is an integer between 1-2; and
Alk is a linear alkyl of 1-7 carbons, branched alkyl of 1-7 carbons, or cyclic alkyl of 3-8 carbons.

6. The method of claim 5, wherein said compound is represented by the structure of formula XI or its isomer, pharmaceutically acceptable salt, pharmaceutical product, polymorph, crystal, N-oxide, ester, hydrate or any combination thereof: 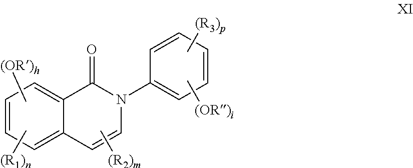
wherein:
R₁, R₂, R₃ are each, independently, hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, cycloalkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;
R is alkyl, cycloalkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
R′ is hydrogen, Alk or COR;
R″ is hydrogen, Alk or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 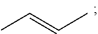
Q is SO₃H, CO₂H, CO₂R, NO₂, tetrazole, SO₂NH₂ or SO₂NHR;
h is 0, 1, 2 or 3;
i is 0, 1, 2, 3 or 4;
n is 1, 2, 3 or 4;
m is 1 or 2;
p is 0, 1, 2, 3, 4 or 5; and
Alk is a linear alkyl of 1-7 carbons, branched alkyl of 1-7 carbons, or cycloalkyl of 3-8 carbons.

7. The method of claim 6, wherein said compound is selected from:
6-hydroxy-2-(4-hydroxyphenyl)-4-phenylisoquinolin-1(2H)-one (15a),
6,8-dihydroxy-2-(4-hydroxyphenyl)-4-(4-methoxyphenyl)isoquinolin-1(2H)-one (15g),
6,8-dihydroxy-2-(4-hydroxyphenyl)-4-phenylisoquinolin-1(2H)-one (15h),
(E)-3-(6,8-dihydroxy-2-(4-hydroxyphenyl)-1-oxo-1,2-dihydroisoquinolin-4-yl)acrylic acid (15l),
2-(4-bromomethyl)phenyl-6-hydroxy-4-(4-hydroxyphenyl)isoquinolin-1(2H)-one (11),
6-hydroxy-2-(4-hydroxyphenyl)-4-(4-(trifluoromethyl)phenylisoquinolin-1(2H)-one (13),
6-hydroxy-2-(4-(hydroxymethyl)phenyl)-4-(4-hydroxyphenyl)isoquinolin-1(2H)-one (14),
2-(4-(bromomethyl)-3-hydroxyphenyl)-6-hydroxy-4-(4-(trifluoromethyl)phenyl)isoquinolin-1(2H)-one (26),
6-hydroxy-2-(4-hydroxyphenyl)-1-oxo-4-(3,4,5-trifluorophenyl)-1,2-dihydroisoquinoline-8-carbonitrile (85),
3-(4-(3-fluoro-4-(trifluoromethyl)phenyl)-6-hydroxy-1-oxoisoquinolin-2(1H)-yl)benzamide (214); and
4-(4-(3-fluoro-4-(trifluoromethyl)phenyl)-6-hydroxy-1-oxoisoquinolin-2(1H)-yl)benzamide (215).

8. The method according to claim 5, wherein said administration selectively inhibits an AKR1C3 enzyme.

9. The method according to claim 5, wherein said disorder is selected from prostate cancer, primary prostate cancer, advanced prostate cancer, metastatic prostate cancer, hormone naïve prostate cancer, refractory prostate cancer or castration resistant prostate cancer (CRPC), or any combination thereof.

10. The method according to claim 5, wherein the patient has precancerous precursors of prostate adenocarcinoma.

11. The method of claim 10, wherein the precancerous precursor of prostate adenocarcinoma is prostate intraepithelial neoplasia (PIN).

12. The method of claim 5, wherein said administration prolongs progressioin-free survival or overall survival in a man with castration-resistant prostate cancer or advanced metastatic prostate cancer.

13. The method according to claim 5, wherein said disorder is selected from breast cancer; metastatic breast cancer; advanced breast cancer; refractory breast cancer; AR-positive breast cancer; ER-positive breast cancer; AR-positive refractory breast cancer; ER-positive refractory breast cancer; AR-positive metastatic breast cancer; ER-positive metastatic breast cancer; triple negative breast cancer; and/or breast cancer that has failed SERM (tamoxifen, toremifene), aromatase inhibitor, trastuzumab, exemestane, bevacizumab, and/or fulvestrant treatment, or any combination thereof.

14. The method of claim 13, wherein said patient has breast cancer that has failed SERM (tamoxifen, toremifene), aromatase inhibitor, trastuzumab, exemestane, bevacizumab, and/or fulvestrant treatment, or any combination thereof.

15. The method of claim 13, wherein said administration prolongs progressioin-free survival or overall survival in a subject with refractory breast cancer or advanced metastatic breast cancer.

16. The method according to claim 5, wherein the disorder is selected from benign prostate hyperplasia (BPH), lung cancer, non-small cell lung cancer (NSCLC), acne, seborrhea, hirsuitism, baldness, alopecia, precocious puberty, adrenal hypertrophy, polycystic ovary syndrome, endometriosis, myeloma or leiomyoma.

17. A method of lowering serum testosterone levels in a male subject consisting essentially of administering a therapeutically effective amount of a compound represented by the structure of formula I, or its isomer, tautomer, pharmaceutically acceptable salt, polymorph, crystal, N-oxide, hydrate or any combination thereof: 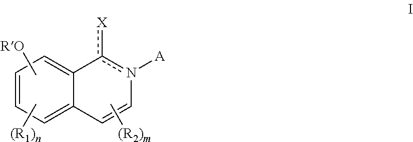
wherein
A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic rings are optionally substituted by 1 to 5 substituents independently selected from R₃ or OR″; and X is O or S; or
A is nothing, N forms a double bond with the cyclic carbon and X is OH or OCH₂CH₂-heterocycle in which the heterocycle is a 3-7 membered saturated or unsaturated substituted or unsubstituted heterocyclic ring;
R₁, R₂ and R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OHSH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring;
R is alkyl, hydrogen, haloalkyl, dihaloalkyl, trihaloalkyl, CH₂F, CHF₂, CF₃, CF₂CF₃, aryl, heteroaryl, phenyl, benzyl, -Ph-CF₃, -Ph-CH₂F, -Ph-CHF₂, -Ph-CF₂CF₃, halogen, alkenyl, CN, NO₂, or OH;
R′ is hydrogen, Alk, or COR;
R″ is hydrogen, Alk, or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 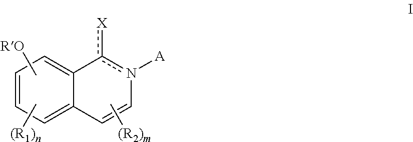
Q is SO₃H, CO₂H, CO₂R, NO₂, tetrazole, SO₂NH₂ or SO₂NHR;
n is an integer of between 1-3;
m is an integer between 1-2; and
Alk is a linear alkyl of 1-7 carbons, branched alkyl of 1-7 carbons, or cyclic alkyl of 3-8 carbons.

18. The method of claim 17, wherein said compound is represented by the structure of formula XI, or its isomer, pharmaceutically acceptable salt, pharmaceutical product, polymorph, crystal, N-oxide, ester, hydrate or any combination thereof: 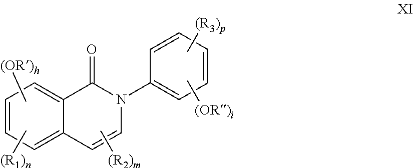
wherein:
R₁, R₂, R₃ are each, independently, hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, cycloalkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;
R is alkyl, cycloalkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
R′ is hydrogen, Alk or COR;
R″ is hydrogen, Alk or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 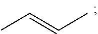
Q is SO₃H, CO₂H, CO₂R, NO₂, tetrazole, SO₂NH₂ or SO₂NHR;
h is 0, 1, 2 or 3;
i is 0, 1, 2, 3 or 4;
n is 1, 2, 3 or 4;
m is 1 or 2;
p is 0, 1, 2, 3, 4 or 5; and
Alk is a linear alkyl of 1-7 carbons, branched alkyl of 1-7 carbons, or cycloalkyl of 3-8 carbons.

19. The method of claim 2, wherein said compound is selected from:
6-hydroxy-2-(4-hydroxyphenyl)-4-phenylisoquinolin-1(2H)-one (15a),
6,8-dihydroxy-2-(4-hydroxyphenyl)-4-(4-methoxyphenyl)isoquinolin-1(2H)-one (15g), 6,8-dihydroxy-2-(4-hydroxyphenyl)-4-phenylisoquinolin-1(2H)-one (15h),
(E)-3-(6,8-dihydroxy-2-(4-hydroxyphenyl)-1-oxo-1,2-dihydroisoquinolin-4-yl)acrylic acid (15l),
2-(4-bromomethyl)phenyl-6-hydroxy-4-(4-hydroxyphenyl)isoquinolin-1(2H)-one (11),
6-hydroxy-2-(4-hydroxyphenyl)-4-(4-(trifluoromethyl)phenylisoquinolin-1(2H)-one (13),
6-hydroxy-2-(4-(hydroxymethyl)phenyl)-4-(4-hydroxyphenyl)isoquinolin-1(2H)-one (14),
2-(4-(bromomethyl)-3-hydroxyphenyl)-6-hydroxy-4-(4-(trifluoromethyl)phenyl)isoquinolin-1(2H)-one (26),
6-hydroxy-2-(4-hydroxyphenyl)-1-oxo-4-(3,4,5-trifluorophenyl)-1,2-dihydroisoquinoline-8-carbonitrile (85),
3-(4-(3-fluoro-4-(trifluoromethyl)phenyl)-6-hydroxy-1-oxoisoquinolin-2(1H)-yl)benzamide (214) and
4-(4-(3-fluoro-4-(trifluoromethyl)phenyl)-6-hydroxy-1-oxoisoquinolin-2(1H)-yl)benzamide (215).

20. The method of claim 17, wherein said serum testosterone levels are total serum testosterone levels.

21. The method of claim 17, wherein said serum testosterone levels are free serum testosterone levels

22. The method of claim 17, wherein said lowering of serum testosterone is independent of a reduction of serum luteinizing hormone levels.

23. The method of claim 17, wherein said lowering of serum testosterone lower prostate-specific antigen (PSA).

24. A method of lowering serum estradiol levels in a subject consisting essentially of administering a therapeutically effective amount of a compound represented by the structure of formula I, or its isomer, tautomer, pharmaceutically acceptable salt, polymorph, crystal, N-oxide, hydrate or any combination thereof: 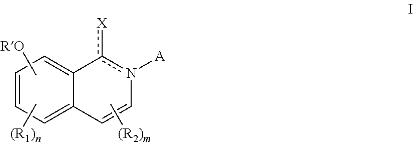
wherein
A is a 5-14 membered saturated or unsaturated, substituted or unsubstituted carbocyclic or heterocyclic ring which is optionally a fused ring system, or a combination thereof; wherein the saturated or unsaturated carbocyclic or heterocyclic rings are optionally substituted by 1 to 5 substituents independently selected from R₃ or OR″; and X is O or S; or
A is nothing, N forms a double bond with the cyclic carbon and X is OH or OCH₂CH₂-heterocycle in which the heterocycle is a 3-7 membered saturated or unsaturated substituted or unsubstituted heterocyclic ring;
R₁, R₂ and R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OHSH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle, in which the heterocycle is a 3-7 membered saturated or unsaturated, substituted or unsubstituted heterocyclic ring;
R is alkyl, hydrogen, haloalkyl, dihaloalkyl, trihaloalkyl, CH₂F, CHF₂, CF₃, CF₂CF₃, aryl, heteroaryl, phenyl, benzyl, -Ph-CF₃, -Ph-CH₂F, -Ph-CHF₂, -Ph-CF₂CF₃, halogen, alkenyl, CN, NO₂, or OH;
R′ is hydrogen, Alk, or COR;
R″ is hydrogen, Alk, or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 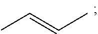
Q is SO₃H, CO₂H, CO₂R, NO₂, tetrazole, SO₂NH₂ or SO₂NHR;
n is an integer of between 1-3;
m is an integer between 1-2; and
Alk is a linear alkyl of 1-7 carbons, branched alkyl of 1-7 carbons, or cyclic alkyl of 3-8 carbons.

25. The method of claim 24, wherein said compound is represented by the structure of formula XI, or its isomer, pharmaceutically acceptable salt, pharmaceutical product, polymorph, crystal, N-oxide, ester, hydrate or any combination thereof: 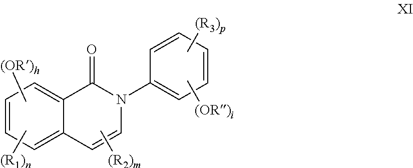
wherein:
R₁, R₂, R₃ are each, independently, hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, cycloalkyl, haloalkyl, aryl, phenyl, benzyl, protected hydroxyl, OCH₂CH₂NR₄R₅, Z-Alk-Q, Z-Alk-NR₄R₅, Z-Alk-heterocycle or OCH₂CH₂-heterocycle in which the heterocycle is a 3-7 membered saturated or unsaturated, substituted or unsubstituted heterocyclic ring;
R is alkyl, cycloalkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;
R′ is hydrogen, Alk or COR;
R″ is hydrogen, Alk or COR;
R₄ and R₅ are independently hydrogen, phenyl, benzyl, an alkyl group of 1 to 6 carbon atoms, a 3 to 7 member cycloalkyl, heterocycloalkyl, aryl or heteroaryl group;
Z is O, NH, CH₂ or 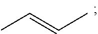
Q is SO₃H, CO₂H, CO₂R, NO₂, tetrazole, SO₂NH₂ or SO₂NHR;
h is 0, 1, 2 or 3;
i is 0, 1, 2, 3 or 4;
n is 1, 2, 3 or 4;
m is 1 or 2;
p is 0, 1, 2, 3, 4 or 5; and
Alk is a linear alkyl of 1-7 carbons, branched alkyl of 1-7 carbons, or cycloalkyl of 3-8 carbons.

26. The method of claim 24, wherein said compound is selected from:
6-hydroxy-2-(4-hydroxyphenyl)-4-phenylisoquinolin-1(2H)-one (15a),
6,8-dihydroxy-2-(4-hydroxyphenyl)-4-(4-methoxyphenyl)isoquinolin-1(2H)-one (15g),
6,8-dihydroxy-2-(4-hydroxyphenyl)-4-phenylisoquinolin-1(2H)-one (15h),
(E)-3-(6,8-dihydroxy-2-(4-hydroxyphenyl)-1-oxo-1,2-dihydroisoquinolin-4-yl)acrylic acid (15l),
2-(4-bromomethyl)phenyl-6-hydroxy-4-(4-hydroxyphenyl)isoquinolin-1(2H)-one (11),
6-hydroxy-2-(4-hydroxyphenyl)-4-(4-(trifluoromethyl)phenylisoquinolin-1(2H)-one (13),
6-hydroxy-2-(4-(hydroxymethyl)phenyl)-4-(4-hydroxyphenyl)isoquinolin-1(2H)-one (14),
2-(4-(bromomethyl)-3-hydroxyphenyl)-6-hydroxy-4-(4-(trifluoromethyl)phenyl)isoquinolin-1(2H)-one (26),
6-hydroxy-2-(4-hydroxyphenyl)-1-oxo-4-(3,4,5-trifluorophenyl)-1,2-dihydroisoquinoline-8-carbonitrile (85),
3-(4-(3-fluoro-4-(trifluoromethyl)phenyl)-6-hydroxy-1-oxoisoquinolin-2(1H)-yl)benzamide (214) and
4-(4-(3-fluoro-4-(trifluoromethyl)phenyl)-6-hydroxy-1-oxoisoquinolin-2(1H)-yl)benzamide (215).

27. The method of claim 24, wherein said serum estradiol levels are total serum estradiol levels.

28. The method of claim 24, wherein said serum estradiol levels are free serum estradiol levels.

29. The method of claim 24, wherein said lowering of serum estradiol is independent of a reduction of serum luteinizing hormone levels.

30. A compound 3-(4-(3-fluoro-4-(trifluoromethyl)phenyl)-6-hydroxy-1-oxoisoquinolin-2(1H)-yl)benzamide, or its isomer, pharmaceutically acceptable salt, pharmaceutical product, polymorph, crystal, N-oxide, ester, hydrate or any combination thereof.

31. A compound 4-(4-(3-fluoro-4-(trifluoromethyl)phenyl)-6-hydroxy-1-oxoisoquinolin-2(1H)-yl)benzamide, or its isomer, pharmaceutically acceptable salt, pharmaceutical product, polymorph, crystal, N-oxide, ester, hydrate or any combination thereof.

32. An AKR1C3 inhibitor compound of Formula XIII: 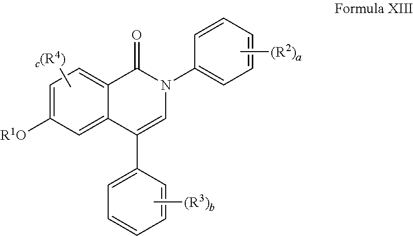
wherein
R¹ is H, alkyl or -alkylene-CO₂Rx, in which Rx is H or alkyl;
R² is H, substituted or unsubstituted alkyl, substituted or unsubstituted alkenyl, substituted or unsubstituted alkyl, substituted or unsubstituted cycloalkyl, alkoxy, haloalkyl, hydroxyl, hydroxymethyl, CONH₂, CONHR^{y}, substituted or unsubstituted alkylene-CO₂R^{y}, in which R^{y} is H or alkyl;
R³ is, in each case, independently selected from hydrogen, alkoxy, COOH, hydroxyl, halogen, haloalkyl, CF₂OMe, CONH₂, CN, carboxyl, SO₂R^{Z} or SO₂NHR^{Z} in which R^{Z} is, in each case, independently, H or alkyl;
R⁴ is, in each case, independently selected from hydrogen, alkyl, hydroxyl, halogen, haloalkyl, CN, carboxyl, CONH₂, CONHR^{Z}, SO₂R^{Z} or SO₂NHR^{Z} in which R^{Z} is, in each case, independently, H or alkyl;
a=1, 2, 3, 4 or 5;
b=1, 2, 3, 4 or 5; and
c=1, 2 or 3;
or a prodrug, isomer, tautomer, metabolite, pharmaceutically acceptable salt, polymorph, crystal, N-oxide, hydrate, or any combination thereof.

33. An AKR1C3 inhibitor compound of claim 32 wherein said inhibitor is:
6-hydroxy-2,4-bis(4-hydroxyphenyl)isoquinolin-1(2H)-one (6); 2-(4-(hydroxymethyl)phenyl)-6-methoxy-4-(4-methoxyphenyl)isoquinolin-1(2H)-one (10); I
2-(4-bromomethyl)phenyl-6-hydroxy-4-(4-hydroxyphenyl)isoquinolin-1(2H)-one (11);
6-hydroxy-2-(4-hydroxyphenyl)-4-(4-(trifluoromethyl)phenyl)isoquinolin-1(2H)-one (13);
6-hydroxy-2-(4-(hydroxymethyl)phenyl)-4-(4-hydroxyphenyl)isoquinolin-1(2H)-one (14);
2-(4-(hydroxymethyl)-3-methoxyphenyl)-6-methoxy-4-(4-(trifluoromethyl)phenyl)isoquinolin-1(2H)-one (25);
2-(4-(bromomethyl)-3-hydroxyphenyl)-6-hydroxy-4-(4-(trifluoromethyl)phenyl)isoquinolin-1(2H)-one (26);
6-hydroxy-2,4-bis(4-(trifluoromethyl)phenyl)isoquinolin-1(2H)-one (30);
2-(4-fluorophenyl)-6-hydroxy-4-(4-(trifluoromethyl)phenyl)isoquinolin-1(2H)-one (34);
6-methoxy-2-(4-methoxyphenyl)-4-(4-(trifluoromethyl)phenyl)isoquinolin-1(2H)-one (35);
4-(6-hydroxy-2-(4-hydroxyphenyl)-1-oxo-1,2-dihydroisoquinolin-4-yl)-N-methyl benzenesulfonamide (36);
6-hydroxy-2-(4-hydroxyphenyl)-4-(4-(methylsulfonyl)phenyl)isoquinolin-1(2H)-one (37);
6-hydroxy-2-(4-hydroxyphenyl)-4-(3,4,5-trifluorophenyl)isoquinolin-1(2H)-one (79);
4-(3,4,5-trifluorophenyl)isoquinolin-1(2H)-one (90);
methyl 6-hydroxy-2-(4-hydroxyphenyl)-1-oxo-4-(3,4,5-trifluorophenyl)-1,2-dihydroisoquinoline-8-carbimidate (100);
6-hydroxy-2-(4-hydroxyphenyl)-1-oxo-4-(3,4,5-trifluorophenyl)-1,2-dihydroisoquinoline-8-carboxamide (100A);
4-(3-fluoro-4-(trifluoromethyl)phenyl)-6-hydroxy-2-(4-hydroxyphenyl)-1-oxo-1,2-dihydroisoquinoline-8-carbonitrile (102);
methyl-4-(3-fluoro-4-(trifluoromethyl)phenyl)-6-hydroxy-2-(4-hydroxyphenyl)-1-oxo-1,2-dihydroisoquinoline-8-carbimidate (102A);
4-(3-fluoro-4-(trifluoromethyl)phenyl)-6-hydroxy-2-(4-hydroxyphenyl)-1-oxo-1,2-dihydroisoquinoline-8-carboxamide (102B);
6-hydroxy-2-(1-oxo-1,3-dihydroisobenzofuran-5-yl)isoquinolin-1(2H)-one (104);
methyl 2-(bromomethyl)-4-(6-hydroxy-1-oxoisoquinolin-2(1H)-yl)benzoate (104A); or
4-bromo-6,8-dihydroxy-2-(3-hydroxyphenyl)isoquinolin-1(2H)-one (213);
or their prodrug, isomer, metabolite, pharmaceutically acceptable salt, polymorph, crystal, N-oxide, hydrate or any combination thereof.
